(19) United States
(12) Patent Application Publication
(10) Pub. No.: US 2019/0110992 A1
(43) Pub. Date: Apr. 18, 2019

(54) MODIFIED RELEASE ABUSE DETERRENT DOSAGE FORMS

(71) Applicant: Grünenthal GmbH, Aachen (DE)

(72) Inventors: Carmen Stomberg, Korschenbroich (DE); Harald Paul, Würselen (DE); Klaus Wening, Köln (DE); Sebastian Schwier, Aachen (DE)

(73) Assignee: Grünenthal GmbH, Aachen (DE)

(21) Appl. No.: 16/158,745

(22) Filed: Oct. 12, 2018

(30) Foreign Application Priority Data
Oct. 13, 2017 (EP) 17196366.3

(51) Int. Cl. A61K 9/14; A61K 31/485; A61K 31/137

(52) U.S. Cl. CPC A61K 9/146; A61K 9/145; A61K 31/137; A61K 31/485

(57) ABSTRACT

The invention relates to a pharmaceutical dosage form for oral administration comprising a pharmacologically active compound; wherein a portion of said pharmacologically active compound is contained in a multitude of immediate release particles providing immediate release of the pharmacologically active compound; wherein another portion of said pharmacologically active compound is contained in at least one controlled release particle providing controlled release of the pharmacologically active compound; and wherein the breaking strength of each of the immediate release particles and/or of the at least one controlled release particle is at least 300 N.

### PRIORITY CLAIM

This application claims priority of European Patent Application No. 17196366.3 filed Oct. 17, 2017, the entire contents of which are hereby incorporated herein by reference.

### FIELD OF THE INVENTION

The invention relates to a pharmaceutical dosage form for oral administration comprising a pharmacologically active compound; wherein a portion of said pharmacologically active compound is contained in a multitude of immediate release particles providing immediate release of the pharmacologically active compound; wherein another portion of said pharmacologically active compound is contained in at least one controlled release particle providing controlled release of the pharmacologically active compound; and wherein the breaking strength of each of the immediate release particles and/or of the at least one controlled release particle is at least 300 N.

### BACKGROUND ART

Conventional drug delivery systems have focused on constant and sustained drug release with the objective of minimizing peaks and valleys of drug concentrations in the body to optimize drug efficacy and to reduce adverse effects. A reduced dosing frequency and improved patient compliance can also be expected for such drug delivery systems compared to immediate release preparations. However, for certain drugs, sustained drug delivery can be detrimental and affected by various factors.

Some drugs undergo extensive first pass metabolism and require fast drug input to saturate metabolizing enzymes in order to minimize pre-systemic metabolism. Thus, a constant and sustained oral drug delivery would result in reduced oral bioavailability. Continuous release drug plasma profiles are sometimes accompanied by a decline in the therapeutic effect of the drug such that biological tolerance can be reduced. Circadian rhythms in certain physiological functions are well established. It has been recognized that many symptoms and onset of disease occur during specific time periods of the 24 hour day, e.g., asthma and angina pectoris attacks are most frequently in the morning hours. For the treatment of local disorders, the delivery of compounds to the site of the disorder with no loss due to absorption in the small intestine is highly desirable to achieve the therapeutic effect and to minimize side effects. For compounds with gastric irritation or chemical instability in gastric fluid, the use of a sustained release preparation may exacerbate gastric irritation and chemical instability in gastric fluid. In general, drug absorption is moderately slow in the stomach, rapid in the small intestine, and sharply declining in the large intestine. Compensation for changing absorption characteristics in the gastrointestinal tract may be important for some drugs. For example, it is rational for a delivery system to pump out the drug much faster when the system reaches the distal segment of the intestine, to avoid the entombment of the drug in the feces.

Pulsed dose delivery systems, prepared as either single unit or multiple unit formulations, and which are capable of releasing the drug after a predetermined time, have been studied to address the aforementioned problematic areas for sustained release preparations. Modified-release multiparticulate oral dosage forms have transformed the active pharmaceutical ingredient (API) delivery landscape. They provide advantages such as targeted release, enteric protection, reduced dose frequency, improved efficacy and fewer side effects. However, they can also be harmful when dose dumping occurs the unintended, rapid release of the entire amount or a significant fraction of the drug. While there are other factors that can result in dose dumping, regulatory agencies have been particularly focused on the dissolution of polymers in the presence of ethanol. These guidelines necessitate new technological strategies, particularly for coated multiparticulate dosage forms. Due to the large surface area, they are more susceptible to premature drug release when taken with alcoholic beverages.

A large number of pharmacologically active substances have a potential for being abused or misused, i.e. they can be used to produce effects which are not consistent with their intended use. In particular, active substances which have a psychotropic effect are abused accordingly. To enable abuse, the corresponding dosage forms, such as tablets or capsules are crushed, for example ground by the abuser, the active substance is extracted from the thus obtained powder using a preferably aqueous liquid and after being optionally filtered through cotton wool or cellulose wadding, the resultant solution is administered parenterally, in particular intravenously. This type of dosage results in an even faster diffusion of the active substance compared to the oral abuse, with the result desired by the abuser, namely the kick. This kick or these intoxication-like, euphoric states are also reached if the powdered dosage form is administered nasally, i.e. is sniffed.

Various concepts for the avoidance of drug abuse have been developed.

It has been proposed to incorporate in dosage forms aversive agents and/or antagonists in a manner so that they only produce their aversive and/or antagonizing effects when the dosage forms are tampered with. However, the presence of such aversive agents is principally not desirable and there is a need to provide sufficient tamper-resistance without relying on aversive agents and/or antagonists.

Another concept to prevent abuse relies on the mechanical properties of the pharmaceutical dosage forms, particularly an increased breaking strength (resistance to crushing). The major advantage of such pharmaceutical dosage forms is that comminuting, particularly pulverization, by conventional means, such as grinding in a mortar or fracturing by means of a hammer, is impossible or at least substantially impeded. Thus, the pulverization, necessary for abuse, of the dosage forms by the means usually available to a potential abuser is prevented or at least complicated.

Such pharmaceutical dosage forms are useful for avoiding drug abuse of the pharmacologically active compound contained therein, as they may not be powdered by conventional means and thus, cannot be administered in powdered form, e.g. nasally. The mechanical properties, particularly the high breaking strength of these pharmaceutical dosage forms renders them tamper-resistant. In the context of such tamper-resistant pharmaceutical dosage forms it can be referred to, e.g., WO 2005/016313, WO 2005/016314, WO 2005/063214, WO 2005/102286, WO 2006/002883, WO 2006/002884, WO 2006/002886, WO 2006/082097, WO 2006/082099, and WO 2009/092601.

U.S. Pat. No. 6,322,819 B1 discloses a multiple pulsed dose drug delivery system for pharmaceutically active amphetamine salts, comprising an immediate-release component and an enteric delayed-release component wherein the enteric release coating has a defined minimum thickness and/or there is a protective layer between the pharmaceutically active amphetamine salt and the enteric release coating and/or there is a protective layer over the enteric release coating. The product can be composed of either one or a number of beads in a dosage form, including either capsule, tablet, or sachet method for administering the beads.

U.S. Pat. No. 6,344,215 relates to pharmaceutical MR (modified release) multiparticulate dosage form such as a capsule (once-a-day MR Capsule) of methylphenidate indicated for the treatment of children with attention deficit hyperactivity disorder (ADHD), which is capable of delivering a portion of the dose for rapid onset of action and the remainder of the dose in a controlled manner for about 12 hours, and which is composed of a multitude of multicoated particles made of two populations of drug layered beads, IR (immediate release) and ER (extended release) beads. The IR beads preferably are made by layering an aqueous solution comprising a drug and a binder on to non-pareil sugar spheres and then applying a seal coat to the drug coated cores. The ER beads are made by applying an extended release coating of a water insoluble dissolution rate controlling polymer such as ethylcellulose to IR beads. The MR capsules are manufactured by filling IR and ER beads in a proper ratio.

US 2006/0240105 relates to a multiparticulate modified release composition that, upon administration to a patient, delivers at least one active ingredient in a bimodal or multimodal manner. The multiparticulate modified release composition comprises a first component and at least one subsequent component; the first component comprising a first population of active ingredient containing particles and the at least one subsequent component comprising a second population of active ingredient containing particles wherein the combination of the components exhibit a bimodal or multimodal release profile.

US 2013/028972 relates to a tamper-resistant tablet comprising (i) a matrix material in an amount of more than one third of the total weight of the tablet; and (ii) a plurality of particulates in an amount of less than two thirds of the total weight of the tablet; wherein said particulates comprise a pharmacologically active compound and a polyalkylene oxide; and form a discontinuous phase within the matrix material; and method of using said tablet to treat pain and other conditions.

US 2014/356428 relates to a pharmaceutical dosage form comprising (i) at least one formed segment (S₁), which contains a first pharmacologically active compound (A₁) and provides prolonged release thereof, and (ii) at least one further segment (S₂), which contains a second pharmacologically active compound (A₂) and provides immediate release thereof, wherein the at least one formed segment (S₁) exhibits a higher breaking strength than the at least one further segment (S₂) and the at least one formed segment (S₁) exhibits a breaking strength of more than 500 N.

US 2015/190348 relates to a pharmaceutical or nutraceutical composition with a core, an inner coating layer, and an outercoating layer, wherein a pharmaceutical or a nutraceutical active ingredient is contained in the core, one or more salts of alginic acid is contained in the inner coating layer, and one or more water-insoluble polymers or copolymers is contained in the outer coating layer. In the composition, the ratio of the amount of the one or more salts of alginic acid in the inner coating layer is at least 2.5:1 by weight to the amount of the one or more water-insoluble polymers or copolymers in the outer coating layer.

WO 2005/079760 relates to a neutral poly(ethyl acrylate, methyl methacrylate) copolymer that is employed as a carrier in the manufacture of pharmaceutical formulations containing an active ingredient. The formulations are preferably made by melt extrusion, and can have rubbery characteristics and can exhibit tamper resistance.

WO 2017/178658 relates to a pharmaceutical dosage form for oral administration comprising a pharmacologically active compound; wherein a portion of said pharmacologically active compound is contained in a multitude of immediate release particles providing immediate release of the pharmacologically active compound; wherein another portion of said pharmacologically active compound is contained in at least one controlled release particle providing controlled release of the pharmacologically active compound; and wherein the breaking strength of each of the immediate release particles and/or of the at least one controlled release particle is at least 300 N.

U.S. Pat. No. 6,344,215 relates to a pharmaceutical MR (modified release) multiparticulate dosage form such as a capsule (once-a-day MR Capsule) of Methylphenidate indicated for the treatment of children with attention deficit hyperactivity disorder (ADHD), capable of delivering a portion of the dose for rapid onset of action and the remainder of the dose in a controlled manner for about 12 hours, is composed of a multitude of multicoated particles made of two populations of drug layered beads, IR (immediate release) and ER (extended release) Beads. The IR beads preferably are made by layering an aqueous solution comprising a drug and a binder on to non-pareil sugar spheres and then applying a seal coat to the drug coated cores. The ER Beads are made by applying an extended release coating of a water insoluble dissolution rate controlling polymer such as ethylcellulose to IR Beads. The MR Capsules are manufactured by filling IR and ER Beads in a proper ratio; the dose and the ratio required for an efficacious, cost effective and patient compliant treatment of children with ADHD were determined from extensive clinical investigations and in vitro-in vivo correlations performed as per FDA Guidelines, Guidance for Industry: Extended Release Oral Dosage Forms.

Schilling/McGinity (International Journal of Pharmaceutics 400 (2010) 24-31; and U.S. Pat. No. 9,192,578 B2) discloses compositions and methods for their preparation by embedding modified release multi-particulates in a matrix under preservation of the dissolution characteristics of the original modified release multi-particulates.

The properties of these tamper-resistant dosage forms, however, are not satisfactory in every respect. There is a need for tamper-resistant dosage forms that possess crush resistance and release the pharmacologically active compound according to a modified release or pulsed release. When trying to tamper the dosage form in order to prepare a formulation suitable for abuse by intravenous administration, the liquid part of the formulation that can be separated from the remainder by means of a syringe should be as less as possible, e.g. should contain not more than 10 wt.-% of the pharmacologically active compound originally contained in the dosage form.

It is an object according to the invention to provide tamper-resistant pharmaceutical dosage forms that provide rapid release of the pharmacologically active compound and that have advantages compared to the tamper-resistant pharmaceutical dosage forms of the prior art.

This object has been achieved by the subject-matter of the patent claims.

### SUMMARY OF THE INVENTION

The invention relates to a pharmaceutical dosage form for oral administration comprising a pharmacologically active compound; wherein a portion of said pharmacologically active compound is contained in a multitude of immediate release particles providing immediate release of the pharmacologically active compound; wherein another portion of said pharmacologically active compound is contained in at least one controlled release particle providing controlled release of the pharmacologically active compound; and wherein the breaking strength of each of the immediate release particles and/or of the at least one controlled release particle is at least 300 N.

It has been unexpectedly found that tamper-resistant dosage forms can be provided that release the pharmacologically active compound in a modified manner, i.e. that combine immediate release and controlled release with one another. It has been unexpectedly found that tamper-resistance of these dosage forms provides resistance against mechanical disruption, against solvent extraction as well as against dose dumping in aqueous ethanol.

Tamper-resistance with respect to dose dumping in aqueous ethanol is typically regarded as a property, wherein the in vitro release profile of the pharmacologically active compound from the pharmaceutical dosage form in ethanolic medium resembles the in vitro release profile in non-ethanolic medium, such that the in vitro release in ethanolic medium is not substantially accelerated compared to that in non-ethanolic medium. It has now been unexpectedly found that tamper-resistant dosage forms can be provided which release the pharmacologically active compound in ethanolic medium not only with an in vitro release profile that resembles the in vitro release profile in non-ethanolic medium, but which provide an in vitro release in ethanolic medium that is even substantially slower than that in non-ethanolic medium.

Furthermore, it has been unexpectedly found that two compartments (multitude of immediate release particles on the one hand and controlled release particle on the other hand) can be provided in one and the same dosage form which both provide independently of one another tamper resistant properties which in turn, however, may differ from one another.

Still further, it has been unexpectedly found that citric acid can stabilize amphetamine sulfate. In particular, it has been surprisingly found that when particles of a formulation containing citric acid are coated, the conversion of amphetamine sulfate to the free base of amphetamine is significantly reduced compared to a formulation not containing citric acid. Further, it has been surprisingly found that the formation of impurities is also reduced when citric acid is present.

Yet further, it has been unexpectedly found that citric acid can stabilize the formulations that are hot melt extruded. In particular, it has been surprisingly found that when hot melt extruding a formulation containing amphetamine sulfate and citric acid, the formation of impurities is significantly reduced compared to hot melt extruding a formulation not containing citric acid.

### BRIEF DESCRIPTION OF THE DRAWINGS

FIG. 1 illustrates the behavior of the particles contained in the pharmaceutical dosage form according to the invention when being subjected to a breaking strength test, in particular their deformability.

FIG. 2 illustrates the behavior of conventional particles when being subjected to a breaking strength test.

FIG. 3 shows the in vitro release profile of the immediate release particles of Example 1.

FIG. 4 shows the in vitro release profile of the enterically coated controlled release particles of Example 2 with a pH switch of the release medium from acidic to neutral after 2 hours.

FIG. 5 shows the in vitro release profile of the enterically coated controlled release particles of Example 3 with a pH switch of the release medium from acidic to neutral after 2 hours.

FIG. 6 shows the in vitro release profile of the controlled release particle of Example 4-1 in comparison to that of Example 4-2.

FIG. 7 shows the in vitro release profile of the dosage form of Example 5 in 40% aqueous ethanol with a pH switch of the release medium from acidic to neutral after 2 hours.

FIG. 8 shows the in vitro release profile of the dosage form of Example 6 in 40% aqueous ethanol.

FIG. 9 shows a sieve analysis of the content of the capsules according to Example 15 after milling for 2 minutes in a coffee grinder.

FIG. 10 shows the in vitro release profile of the capsules according to Example 15 in release medium without ethanol and with ethanol.

FIG. 11 shows a sieve analysis of the content of the capsules according to Example 16 after milling for 2 minutes in a coffee grinder.

FIG. 12 shows the in vitro release profile of the capsules according to Example 16 in release medium without ethanol and with ethanol.

FIG. 13 shows the mean in vitro release profile of the tablets according to Example 17.

FIG. 14 shows the mean in vitro release profile the immediate release particles of Example 18.

FIG. 15 shows the in vitro release profile of the enterically coated controlled release particles of Example 19-1 with a pH switch of the release medium from acidic to neutral after 2 hours.

FIG. 16 shows the in vitro release profile of the enterically coated controlled release particles of Example 19-2 with a pH switch of the release medium from acidic to neutral after 2 hours.

FIG. 17 shows the in vitro release profile of the enterically coated controlled release particles of Example 19-3 with a pH switch of the release medium from acidic to neutral after 2 hours.

FIG. 18 shows the in vitro release profile of the capsule 20-20 of Example 20 in different release media.

FIGS. 19 to 22 show the in vitro release profile of the capsule according to Example 26 under different conditions.

FIG. 23 shows the in vitro release profile of the capsule according to Example 27 with a pH switch of the release medium from acidic to neutral after 2 hours.

FIG. 24 shows the in vitro release profile of the capsule according to Example 28 with a pH switch of the release medium from acidic to neutral after 2 hours

FIG. 25 shows the in vitro release profile of the capsule according to Example 29 with a pH switch of the release medium from acidic to neutral after 2 hours.

FIG. 26 shows the in vitro release profile of the capsule according to Example 30 with a pH switch of the release medium from acidic to neutral after 2 hours.

FIG. 27 is an overlay plot of Figures to 20 and 23 to 26

FIG. 28 shows an overlay of the in vitro dissolution profiles in non-ethanolic medium at pH 1 of the immediate release particles according to Example 31.

FIGS. 29 and 30 show the in vitro dissolution of the particles according to Example 32 with a pH switch from pH 1 to pH 6.8 after 120 minutes in ethanolic medium and non-ethanolic medium, respectively.

FIG. 31 shows the in vitro dissolution of the particles according to Example 36 with a pH switch from pH 1 to pH 6.8 after 120 minutes in non-ethanolic medium.

### DETAILED DESCRIPTION OF THE INVENTION

The invention relates to a pharmaceutical dosage form for oral administration. As used herein, the term “pharmaceutical dosage form” refers to a pharmaceutical entity comprising a pharmacologically active compound which upon prescribed administration is to be taken orally.

Preferably, the pharmaceutical dosage from according to the invention is a capsule or a tablet. The particles that are contained in the pharmaceutical dosage form and/or the pharmaceutical dosage form as such may be film-coated.

The pharmaceutical dosage form may be compressed or molded in its manufacture, and it may be of almost any size, shape, weight, and color. Most pharmaceutical dosage forms are intended to be swallowed as a whole. However, alternatively pharmaceutical dosage forms may be dissolved in the mouth, chewed, or dissolved or dispersed in liquid or meal before swallowing. Thus, the pharmaceutical dosage form according to the invention may alternatively be adapted for buccal or lingual administration.

In a preferred embodiment, the pharmaceutical dosage form according to the invention preferably can be regarded as a MUPS formulation (multiple unit pellet system). In a preferred embodiment, the pharmaceutical dosage form according to the invention is monolithic. In another preferred embodiment, the pharmaceutical dosage form according to the invention is not monolithic. In this regard, monolithic preferably means that the pharmaceutical dosage form is formed or composed of material without joints or seams or consists of or constitutes a single unit.

In a preferred embodiment, the pharmaceutical dosage form according to the invention contains all ingredients in a dense compact unit which in comparison to capsules has a comparatively high density. In another preferred embodiment, the pharmaceutical dosage form according to the invention contains all ingredients in a capsule which in comparison to dense compact unit has a comparatively low density.

An advantage of the pharmaceutical dosage forms according to the invention is that the same particles may be mixed with excipients in different amounts to thereby produce pharmaceutical dosage forms of different strengths. Another advantage of the pharmaceutical dosage forms according to the invention is that the different particles may be mixed with one another to thereby produce pharmaceutical dosage forms of different properties, e.g. different release rates, different pharmacologically active compounds, and the like.

The pharmaceutical dosage form according to the invention comprises a pharmacologically active compound; wherein a portion of said pharmacologically active compound is contained in a multitude of immediate release particles providing immediate release of the pharmacologically active compound; and wherein another portion of said pharmacologically active compound is contained in at least one controlled release particle providing controlled release of the pharmacologically active compound.

According to a preferred embodiment of the invention, said another portion of said pharmacologically active compound is contained in a single controlled release particle or in a few controlled release particles (2, 3, 4 or more controlled release particles), wherein an individual controlled release particle is preferably substantially bigger and/or heavier than an individual immediate release particle. Preferably, said single controlled release particle or every individual controlled release particle within the group of said few controlled release particles has a total weight of at least 20 mg, more preferably of at least 50 mg, still more preferably of at least 75 mg, yet more preferably of at least 100 mg, most preferably at least 125 mg and in particular at least 150 mg. According to this embodiment, the controlled release particle(s) preferably do not comprise an enteric coating.

According to another preferred embodiment of the invention, said another portion of said pharmacologically active compound is contained in a multitude of controlled release particles, wherein an individual controlled release particle is preferably of similar size and weight compared to an individual immediate release particle.

In a preferred embodiment of the invention, the individual controlled release particles and the individual immediate release particles are not only of similar size and weight, but are not visually distinguishable from one another with the naked eye. Thus, the outer appearance (color, shape, size, surface and the like) of the controlled release particles and the immediate release particles is substantially identical such that a potential abuser would have at least substantial difficulties to manually separate the immediate release particles from the controlled release particles. This further improves tamper resistance of the pharmaceutical dosage form according to the invention.

Nonetheless, due to the different composition and morphology of the immediate release particles and the controlled release particles, a skilled person may distinguish the types of particles from one another by means of sophisticated analytical techniques which, however, are usually not available to an abuser, such as infrared spectroscopy, Raman spectroscopy, and the like. Thus, when separating the immediate release particles from the controlled release particles based upon distinction by means of such sophisticated analytical techniques, the in vitro release profile can be measured for the separated multitude of immediate release particles in the absence of the multitude of controlled release particles, and vice versa. Alternatively, even in the absence of such sophisticated analytical techniques, the in vitro release profile could even be measured for a single particle under adapted in vitro conditions (see e.g. M. Xu et al., Int. J. Pharm. 478 (2015) 318-327).

Unless expressly stated otherwise, any preferred embodiment that according to the invention is related to “particles” independently may apply to both, to the immediate release particles as well as to the controlled release particle(s).

The breaking strength of each of the immediate release particles and/or of the at least one controlled release particle is at least 300 N. For the purpose of the specification, A “and/or” B means (i) A but not B, (ii) B but not A, or (iii) A as well as B.

The pharmaceutical dosage form according to the invention contains a plurality of particles, namely a multitude of immediate release particles and at least one controlled release particle. The particles comprise a pharmacologically active compound and preferably a polyalkylene oxide. In a preferred embodiment, the immediate release particles but preferably not the at least one controlled release particle additionally comprise a disintegrant. In another preferred embodiment, the immediate release particles and preferably also the at least one controlled release particle additionally comprise a disintegrant.

Preferably, within the particles, the pharmacologically active compound is dispersed in the preferably present polyalkylene oxide and the optionally additionally present disintegrant.

For the purpose of the specification, the term “particle” refers to a discrete mass of material that is solid, e.g. at 20° C. or at room temperature or ambient temperature. Preferably a particle is solid at 20° C. Preferably, the particles are monoliths. Preferably, the pharmacologically active compound and the polyalkylene oxide are intimately homogeneously distributed in the particles so that the particles do not contain any segments where either pharmacologically active compound is present in the absence of polyalkylene oxide or where polyalkylene oxide is present in the absence of pharmacologically active compound.

When the particles are film coated, the preferably present polyalkylene oxide is preferably homogeneously distributed in the core of the pharmaceutical dosage form, i.e. the film coating preferably does not contain polyalkylene oxide, but optionally polyalkylene glycol that differs from polyalkylene oxide in its lower molecular weight. Nonetheless, the film coating as such may of course contain one or more polymers, which however, preferably differ from the polyalkylene oxide preferably contained in the core.

A portion of the pharmacologically active compound is contained in a multitude of immediate release particles (IR particles) and another portion of the pharmacologically active compound is contained in at least one controlled release particle (CR particle).

Controlled release particles (CR particles) differ from immediate release particles (IR particles) in their in vitro dissolution kinetics. While immediate release particles provide comparatively quick release of the pharmacologically active compound, controlled release particles provide comparatively slow release of the pharmacologically active compound.

For the purpose of the specification, preferred immediate release particles are referred to as “fast release particles” (FR particles), i.e. fast release particles (FR particles) can be regarded as a preferred subgroup of immediate release particles (IR particles). Fast release particles (FR particles) differ from immediate release particles (IR particles) in that they are provided with a coating slightly retarding the in vitro dissolution of the pharmacologically active compound from the fast release particles. However, the in vitro dissolution of the pharmacologically active compound from the fast release particles (FR particles) still proceeds comparatively quickly such that they can be regarded as a subgroup of immediate release particles (IR particles). The different terminology is used throughout the specification for ease of definition. Nonetheless, unless expressly stated otherwise, any definition with respect to immediate release particles (IR particles) also analogously refers to their subgroup of fast release particles (FR particles).

For the purpose of the specification, preferred controlled release particles are referred to as “prolonged release particle(s)” (PR particle(s)) or “delayed release particles” (DR particles) or “postponed release particles” (OR particles), i.e. prolonged release particle(s) (PR particle(s)), delayed release particles (DR particles), and postponed release particles (OR particles) can each be regarded as a preferred subgroup of controlled release particles (CR particles). Delayed release particles (DR particles) as well as postponed release particles (OR particles) differ from prolonged release particles (PR particles) in that they are provided with an enteric coating. The in vitro dissolution of the pharmacologically active compound from the delayed release particles (DR particles) and from the postponed release particles (OR particles) in either case commences after a certain lag time, especially after the pH value of the release medium is switched from acidic to neutral. Postponed release particles (OR particles) are preferred delayed release particles (DR particles), i.e. can be regarded as a preferred subgroup of delayed release particles (DR particles). Postponed release particles (OR particles) differ from delayed release particles (DR particles) in the composition of the enteric coating. The different terminology is used throughout the specification for ease of definition. Nonetheless, unless expressly stated otherwise, any definition with respect to controlled release particle(s) (CR particle(s)) also analogously refers to their subgroup of prolonged release particles (PR particles), their subgroup of delayed release particles (DR particles), and their subgroup of postponed release particles (OR particles). Likewise, unless expressly stated otherwise, any definition with respect to delayed release particles (DR particles) also analogously refers to their subgroup of postponed release particles (OR particles).

For the purpose of the specification, unless expressly stated otherwise, any definition with respect to particles in general also analogously refers to immediate release particles including fast release particles and controlled release particles including prolonged release particle(s), delayed release particles and postponed release particles.

The various particles according to the invention are preferably distinguished from one another by the following preferred features (particle size/weight, presence/nature/amount of coating, and in vitro dissolution profile):
the multitude of IR particles preferably have an individual particle weight of less than 20 mg, more preferably not more than 10 mg, more preferably not more than 5 mg, still more preferably not more than 2 mg; are preferably not coated with an enteric coating; and as such (i.e. in the absence of particles of other type and nature) provide immediate release of the pharmacologically active compound under in vitro conditions such that preferably in accordance with Ph. Eur. after 30 minutes in artificial gastric juice at pH 1.2 at least 70%, still more preferably at least 75 wt.-%, yet more preferably at least 80 wt.-%, even more preferably at least 85 wt.-%, most preferably at least 90 wt.-% of the pharmacologically active compound that were originally contained in said multitude of IR particles have been released;
the multitude of FR particles preferably have an individual particle weight of less than 20 mg, more preferably not more than 10 mg, more preferably not more than 5 mg, still more preferably not more than 2 mg; are preferably coated with an enteric coating, wherein the content of the dried enteric coating of the FR particles is at most 15 wt.-%, more preferably at most 12 wt.-%, based on the total weight of the FR particles; and as such (i.e. in the absence of particles of other type and nature) provide fast release of the pharmacologically active compound under in vitro conditions such that preferably in accordance with Ph. Eur. after 30 minutes in artificial gastric juice at pH 1.2 less than 70% of the pharmacologically active compound that were originally contained in said multitude of FR particles have been released; and such that after 60 minutes in artificial gastric juice at pH 1.2 at least 70%, still more preferably at least 75 wt.-%, yet more preferably at least 80 wt.-%, even more preferably at least 85 wt.-%, most preferably at least 90 wt.-% of the pharmacologically active compound that were originally contained in said multitude of IR particles have been released;
the at least one PR particle preferably has an individual particle weight of at least 20 mg, more preferably of at least 50 mg; and as such (i.e. in the absence of particles of other type and nature) provides prolonged release of the pharmacologically active compound under in vitro conditions such that preferably in accordance with Ph. Eur. after 30 minutes in artificial gastric juice at pH 1.2 less than 50%, more preferably at most 40 wt.-%, still more preferably at most 30 wt.-%, yet more preferably at most 10 wt.-% of the pharmacologically active compound that were originally contained in said at least one PR particle have been released;
the multitude of DR particles preferably have an individual particle weight of less than 20 mg, more preferably not more than 10 mg, more preferably not more than 5 mg, still more preferably not more than 2 mg; are preferably coated with an enteric coating; and as such (i.e. in the absence of particles of other type and nature) provide delayed release of the pharmacologically active compound under in vitro conditions such that preferably under in vitro conditions in accordance with Ph. Eur., when changing the release medium after 120 minutes from initially artificial gastric juice with pH 1.2 to subsequently artificial intestinal fluid with pH 6.8, after 180 minutes preferably at least 20 wt.-%, more preferably at least 22.5 wt.-%, still more preferably at least 25 wt.-%, yet more preferably at least 27.5 wt.-%, most preferably at least 30 wt.-% of the pharmacologically active compound that were originally contained in said multitude of DR particles have been released;
the multitude of OR particles preferably have an individual particle weight of less than 20 mg, more preferably not more than 10 mg, more preferably not more than 5 mg, still more preferably not more than 2 mg; are preferably coated with an enteric coating comprising a combination of first acrylate polymer and a second acrylate polymer; and as such (i.e. in the absence of particles of other type and nature) provide postponed release of the pharmacologically active compound under in vitro conditions such that preferably under in vitro conditions in accordance with Ph. Eur., when changing the release medium after 120 minutes from initially artificial gastric juice with pH 1.2 to subsequently artificial intestinal fluid with pH 6.8, after 180 minutes preferably less than 20%, more preferably at most 17.5 wt.-%, still more preferably at most 15 wt.-%, yet more preferably at most 10 wt.-% of the pharmacologically active compound that were originally contained in said multitude of OR particles have been released.

Preferably, said multitude of immediate release particles (IR particles or FR particles) and/or said at least one controlled release particle (PR particle(s) or DR particles or OR particles) independently of one another may comprise a polyalkylene oxide.

Preferably, the polyalkylene oxide is selected from polymethylene oxide, polyethylene oxide and polypropylene oxide, or copolymers thereof. Polyethylene oxide is preferred.

Preferably, the polyalkylene oxide has a weight average molecular weight of at least 200,000 g/mol, more preferably at least 500,000 g/mol. In a preferred embodiment, the polyalkylene oxide has a weight average molecular weight (M_{W}) or viscosity average molecular weight (M_{η}) of at least 750,000 g/mol, preferably at least 1,000,000 g/mol or at least 2,500,000 g/mol, more preferably in the range of 1,000,000 g/mol to 15,000,000 g/mol, and most preferably in the range of 5,000,000 g/mol to 10,000,000 g/mol. Suitable methods to determine M_{W} and M_{η□} are known to a person skilled in the art. M_{η} is preferably determined by rheological measurements, whereas M_{W} can be determined by gel permeation chromatography (GPC).

Polyalkylene oxide may comprise a single polyalkylene oxide having a particular average molecular weight, or a mixture (blend) of different polymers, such as two, three, four or five polymers, e.g., polymers of the same chemical nature but different average molecular weight, polymers of different chemical nature but same average molecular weight, or polymers of different chemical nature as well as different molecular weight.

For the purpose of the specification, a polyalkylene glycol has a molecular weight of up to 20,000 g/mol whereas a polyalkylene oxide has a molecular weight of more than 20,000 g/mol. In a preferred embodiment, the weight average over all molecular weights of all polyalkylene oxides that are contained in the pharmaceutical dosage form is at least 200,000 g/mol. Thus, polyalkylene glycols, if any, are preferably not taken into consideration when determining the weight average molecular weight of polyalkylene oxide.

The polyalkylene oxide preferably has a viscosity at 25° C. of 30 to 17,600 cP, more preferably 55 to 17,600 cP, still more preferably 600 to 17,600 cP and most preferably 4,500 to 17,600 cP, measured in a 5 wt.-% aqueous solution using a model RVF Brookfield viscosimeter (spindle no. 2/rotational speed 2 rpm); of 400 to 4,000 cP, more preferably 400 to 800 cP or 2,000 to 4,000 cP, measured on a 2 wt.-% aqueous solution using the stated viscosimeter (spindle no. 1 or 3/rotational speed 10 rpm); or of 1,650 to 10,000 cP, more preferably 1,650 to 5,500 cP, 5,500 to 7,500 cP or 7,500 to 10,000 cP, measured on a 1 wt.-% aqueous solution using the stated viscosimeter (spindle no. 2/rotational speed 2 rpm).

Polyethylene oxide that is suitable for use in the pharmaceutical dosage forms according to the invention is commercially available from Dow. For example, Polyox WSR N-12K, Polyox N-60K, Polyox WSR 301 NF or Polyox WSR 303NF may be used in the pharmaceutical dosage forms according to the invention. For details concerning the properties of these products, it can be referred to e.g. the product specification.

Preferably, the molecular weight dispersity M_{w}/Mₙ of polyalkylene oxide is within the range of 2.5±2.0, more preferably 2.5±1.5, still more preferably 2.5±1.0, yet more preferably 2.5±0.8, most preferably 2.5±0.6, and in particular 2.5±0.4.

Preferably, the content of the polyalkylene oxide is at least 25 wt.-%, more preferably at least 40 wt.-%, based on the total weight of the pharmaceutical dosage form or based on the total weight of the particles or based on the total weight of those particles that contain the polyalkylene oxide.

Preferably, the content of the polyalkylene oxide is within the range of from 25 to 80 wt.-%, more preferably 25 to 75 wt.-%, still more preferably 25 to 70 wt.-%, yet more preferably 25 to 65 wt.-%, most preferably 30 to 65 wt.-% and in particular 35 to 65 wt.-%, based on the total weight of the pharmaceutical dosage form or based on the total weight of the particles or based on the total weight of those particles that contain the polyalkylene oxide. In a preferred embodiment, the content of the polyalkylene oxide is at least 30 wt.-%, more preferably at least 35 wt.-%, still more preferably at least 40 wt.-%, yet more preferably at least 45 wt.-% and in particular at least 50 wt.-%, based on the total weight of the pharmaceutical dosage form or based on the total weight of the particles or based on the total weight of those particles that contain the polyalkylene oxide.

In a preferred embodiment, the overall content of polyalkylene oxide is within the range of 35±8 wt.-%, more preferably 35±6 wt.-%, most preferably 35±4 wt.-%, and in particular 35±2 wt.-%, based on the total weight of the pharmaceutical dosage form or based on the total weight of the particles or based on the total weight of those particles that contain the polyalkylene oxide. In another preferred embodiment, the overall content of polyalkylene oxide is within the range of 40±12 wt.-%, more preferably 40±10 wt.-%, most preferably 40±7 wt.-%, and in particular 40±3 wt.-%, based on the total weight of the pharmaceutical dosage form or based on the total weight of the particles or based on the total weight of those particles that contain the polyalkylene oxide. In still another preferred embodiment, the overall content of polyalkylene oxide is within the range of 45±16 wt.-%, more preferably 45±12 wt.-%, most preferably 45±8 wt.-%, and in particular 45±4 wt.-%, based on the total weight of the pharmaceutical dosage form or based on the total weight of the particles or based on the total weight of those particles that contain the polyalkylene oxide. In yet another preferred embodiment, the overall content of polyalkylene oxide is within the range of 50±20 wt.-%, more preferably 50±15 wt.-%, most preferably 50±10 wt.-%, and in particular 50±5 wt.-%, based on the total weight of the pharmaceutical dosage form or based on the total weight of the particles or based on the total weight of those particles that contain the polyalkylene oxide. In a further preferred embodiment, the overall content of polyalkylene oxide is within the range of 55±20 wt.-%, more preferably 55±15 wt.-%, most preferably 55±10 wt.-%, and in particular 55±5 wt.-%, based on the total weight of the pharmaceutical dosage form or based on the total weight of the particles or based on the total weight of those particles that contain the polyalkylene oxide. In still a further a preferred embodiment, the overall content of polyalkylene oxide is within the range of 60±20 wt.-%, more preferably 60±15 wt.-%, most preferably 60±10 wt.-%, and in particular 60±5 wt.-% based on the total weight of the pharmaceutical dosage form or based on the total weight of the particles or based on the total weight of those particles that contain the polyalkylene oxide. In a still further a preferred embodiment, the overall content of polyalkylene oxide is within the range of 65±20 wt.-%, more preferably 65±15 wt.-%, and most preferably 65±10 wt.-%, and in particular 65±5 wt.-%, based on the total weight of the pharmaceutical dosage form or based on the total weight of the particles or based on the total weight of those particles that contain the polyalkylene oxide.

Preferably, the relative weight ratio of the polyalkylene oxide to the pharmacologically active compound is within the range of 30:1 to 1:10, more preferably 20:1 to 1:1, still more preferably 15:1 to 5:1, yet more preferably 14:1 to 6:1, most preferably 13:1 to 7:1, and in particular 12:1 to 8:1.

Preferably, the pharmacologically active compound is dispersed in a matrix comprising the polyalkylene oxide.

In a preferred embodiment, polyalkylene oxide is homogeneously distributed in the particles. Preferably, the pharmacologically active compound and polyalkylene oxide are intimately homogeneously distributed in the particles so that the particles do not contain any segments where either pharmacologically active compound is present in the absence of polyalkylene oxide or where polyalkylene oxide is present in the absence of pharmacologically active compound.

When the particles are film coated, the polyalkylene oxide is preferably homogeneously distributed in the core of the particles, i.e. the film coating preferably does not contain polyalkylene oxide. Nonetheless, the film coating as such may of course contain one or more polymers, which however, preferably differ from the polyalkylene oxide contained in the core.

Preferably, the pharmaceutical dosage form according to the invention additionally comprises a disintegrant. The disintegrant may be contained in the multitude of immediate release particles (IR particles or FR particles) and/or in the at least one controlled release particle (PR particle(s) or DR particles or OR particles) and/or outside the particles.

Preferably, each of the immediate release particles (IR particles or FR particles) and/or each of the controlled release particle (PR particle(s) or DR particles or OR particles) comprises a disintegrant. Preferably, the content of the disintegrant is more than 5.0 wt.-%, more preferably at least 10 wt.-%, based on the total weight of the pharmaceutical dosage form or based on the total weight of the particles or based on the total weight of those particles that contain the disintegrant.

Preferably, the pharmacologically active compound is dispersed in a matrix comprising the disintegrant and optionally the polyalkylene oxide.

In a preferred embodiment, particularly when the pharmaceutical dosage form is a capsule, the pharmaceutical dosage form contains the entire amount of disintegrant within the particles, preferably within the immediate release particles (IR particles or FR particles) and/or within the at least one controlled release particle (PR particle(s) or DR particles or OR particles), i.e. outside the particles, preferably outside the immediate release particles (IR particles or FR particles) and/or outside the at least one controlled release particle (PR particle(s) or DR particles or OR particles), there is preferably no disintegrant. Furthermore, the disintegrant is preferably homogeneously distributed in the particles. Preferably, when the particles are coated, the coating does not contain disintegrant.

In another preferred embodiment, particularly when the pharmaceutical dosage form is a tablet, the pharmaceutical dosage form contains the disintegrant within the particles as well as outside the particles. In a preferred embodiment, the nature of disintegrant within the particle is identical with the nature of disintegrant outside the particles. However, different disintegrants inside the particles and outside the particles are also possible in accordance with the invention. Furthermore, the disintegrant is preferably homogeneously distributed in the particles. Preferably, when the particles are coated, the coating does not contain disintegrant.

Suitable disintegrants are known to the skilled person and are preferably selected from the group consisting of polysaccharides, starches, starch derivatives, cellulose derivatives, polyvinylpyrrolidones, acrylates, gas releasing substances, and the mixtures of any of the foregoing.

Preferred starches include but are not limited to “standard starch” (e.g. native maize starch) and pregelatinized starch (e.g. starch 1500).

Preferred starch derivatives include but are not limited to sodium starch glycolate (carboxymethyl starch sodium, e.g. Vivastar®).

Preferred cellulose derivatives include but are not limited to croscarmellose sodium (=crosslinked sodium carboxymethylcellulose; e.g. Vivasol®), carmellose calcium (calcium carboxymethylcellulose), carmellose sodium (sodium carboxymethylcellulose), low substituted carmellose sodium (low substituted sodium carboxymethylcellulose; average degree of substitution (DS) 0.20 to 0.40, Mr 80,000 to 600,000 g/mol, CAS 9004-32-4, E 466), low substituted hydroxypropylcellulose (having a content of propyl groups within the range of from 5 to 16%; CAS 9004-64-2).

Preferred acrylates include but are not limited to carbopol.

Preferred polyvinylpyrrolidones include but are not limited to crospovidone (PVP

Preferred gas releasing substances include but are not limited to sodium bicarbonate.

Preferred disintegrants include but are not limited to crosslinked sodium carboxymethylcellulose (Na-CMC) (e.g. Crosscarmellose, Vivasol®, Ac-Di-Sol®); crosslinked casein (e.g.) Esma-Spreng®; polysaccharide mixtures obtained from soybeans (e.g. Emcosoy®); maize starch or pretreated maize starch (e.g. Amijel®); alginic acid, sodium alginate, calcium alginate; polyvinylpyrrolidone (PVP) (e.g. Kollidone®, Polyplasdone®, Polydone®); crosslinked polyvinylpyrrolidone (PVP CI) (e.g. Polyplasdone® XL); starch and pretreated starch such as sodium carboxymethyl starch (=sodium starch glycolate, e.g. Explotab®, Prejel®, Primotab® ET, Starch® 1500, Ulmatryl®), and the mixtures thereof. Crosslinked polymers are particularly preferred disintegrants, especially crosslinked sodium carboxymethylcellulose(Na-CMC) or crosslinked polyvinylpyrrolidone (PVP CI).

Particularly preferred disintegrants are selected from the group consisting of
crosslinked sodium carboxymethylcellulose (Na-CMC) (e.g. Crosscarmellose, Vivasol®, Ac-Di-Sol®);
crosslinked casein (e.g. Esma-Spreng®);
alginic acid, sodium alginate, calcium alginate;
polysaccharide mixtures obtained from soybeans (e.g. Emcosoy®);
starch and pretreated starch such as sodium carboxymethyl starch (=sodium starch glycolate, e.g. Explotab®, Prejel®, Primotab® ET, Starch® 1500, Ulmatryl®);
maize starch or pretreated maize starch (e.g. Amijel®);
and mixtures of any of the foregoing.

Preferably, the content of the disintegrant is at least 6.0 wt.-%, at least 7.0 wt.-%, at least 8.0 wt.-%, at least 9.0 wt.-%, or at least 10 wt.-%, more preferably at least 12 wt.-%, still more preferably at least 14 wt.-%, yet more preferably at least 15 wt.-%, even more preferably at least 16 wt.-%, most preferably at least 18 wt.-%, and in particular at least 19 wt.-%, based on the total weight of the pharmaceutical dosage form or based on the total weight of the particles or based on the total weight of those particles that contain the disintegrant.

It has been surprisingly found that the content of disintegrant typically has an optimum at which it provides the best balance of immediate release properties on the one hand and resistance against solvent extraction on the other hand. Said optimum may vary, but preferably is within the range of from about 10 wt.-% to about 20 wt.-%, based on the total weight of the pharmaceutical dosage form or based on the total weight of the particles that contain the disintegrant.

In a preferred embodiment, the content of the disintegrant is within the range of 15±9.0 wt.-%, more preferably 15±8.5 wt.-%, still more preferably 15±8.0 wt.-%, yet more preferably 15±7.5 wt.-%, most preferably 15±7.0 wt.-%, and in particular 15±6.5 wt.-%, based on the total weight of the pharmaceutical dosage form or based on the total weight of the particles or based on the total weight of those particles that contain the disintegrant. In still another preferred embodiment, the content of the disintegrant is within the range of 15±6.0 wt.-%, more preferably 15±5.5 wt.-%, still more preferably 15±5.0 wt.-%, yet more preferably 15±4.5 wt.-%, most preferably 15±4.0 wt.-%, and in particular 15±3.5 wt.-%, based on the total weight of the pharmaceutical dosage form or based on the total weight of the particles or based on the total weight of those particles that contain the disintegrant. In another preferred embodiment, the content of the disintegrant is within the range of 15±3.0 wt.-%, more preferably 15±2.5 wt.-%, still more preferably 15±2.0 wt.-%, yet more preferably 15±1.5 wt.-%, most preferably 15±1.0 wt.-%, and in particular 15±0.5 wt.-%, based on the total weight of the pharmaceutical dosage form or based on the total weight of the particles or based on the total weight of those particles that contain the disintegrant.

In another preferred embodiment, the content of the disintegrant is within the range of 20±15 wt.-% or 20±14 wt.-%, more preferably 20±13 wt.-%, still more preferably 20±12 wt.-%, yet more preferably 20±11 wt.-%, most preferably 20±10 wt.-%, and in particular 20±9.5 wt.-%, based on the total weight of the pharmaceutical dosage form or based on the total weight of the particles or based on the total weight of those particles that contain the disintegrant. In another preferred embodiment, the content of the disintegrant is within the range of 20±9.0 wt.-%, more preferably 20±8.5 wt.-%, still more preferably 20±8.0 wt.-%, yet more preferably 20±7.5 wt.-%, most preferably 20±7.0 wt.-%, and in particular 20±6.5 wt.-%, based on the total weight of the pharmaceutical dosage form or based on the total weight of the particles or based on the total weight of those particles that contain the disintegrant. In still another preferred embodiment, the content of the disintegrant is within the range of 20±6.0 wt.-%, more preferably 20±5.5 wt.-%, still more preferably 20±5.0 wt.-%, yet more preferably 20±4.5 wt.-%, most preferably 20±4.0 wt.-%, and in particular 20±3.5 wt.-%, based on the total weight of the pharmaceutical dosage form or based on the total weight of the particles or based on the total weight of those particles that contain the disintegrant. In another preferred embodiment, the content of the disintegrant is within the range of 20±3.0 wt.-%, more preferably 20±2.5 wt.-%, still more preferably 20±2.0 wt.-%, yet more preferably 20±1.5 wt.-%, most preferably 20±1.0 wt.-%, and in particular 20±0.5 wt.-%, based on the total weight of the pharmaceutical dosage form or based on the total weight of the particles or based on the total weight of those particles that contain the disintegrant.

In still another preferred embodiment, the content of the disintegrant is within the range of 25±9.0 wt.-%, more preferably 25±8.5 wt.-%, still more preferably 25±8.0 wt.-%, yet more preferably 25±7.5 wt.-%, most preferably 25±7.0 wt.-%, and in particular 25±6.5 wt.-%, based on the total weight of the pharmaceutical dosage form or based on the total weight of the particles or based on the total weight of those particles that contain the disintegrant. In still another preferred embodiment, the content of the disintegrant is within the range of 25±6.0 wt.-%, more preferably 25±5.5 wt.-%, still more preferably 25±5.0 wt.-%, yet more preferably 25±4.5 wt.-%, most preferably 25±4.0 wt.-%, and in particular 25±3.5 wt.-%, based on the total weight of the pharmaceutical dosage form or based on the total weight of the particles or based on the total weight of those particles that contain the disintegrant. In another preferred embodiment, the content of the disintegrant is within the range of 25±3.0 wt.-%, more preferably 25±2.5 wt.-%, still more preferably 25±2.0 wt.-%, yet more preferably 25±1.5 wt.-%, most preferably 25±1.0 wt.-%, and in particular 25±0.5 wt.-%, based on the total weight of the pharmaceutical dosage form or based on the total weight of the particles or based on the total weight of those particles that contain the disintegrant.

When the pharmaceutical dosage form according to the invention contains more than a single disintegrant, e.g. a mixture of two different disintegrants, the above percentages preferably refer to the total content of disintegrants.

Preferably, the relative weight ratio of the preferably contained polyalkylene oxide to the disintegrant is within the range of 8:1 to 1:5, more preferably 7:1 to 1:4, still more preferably 6:1 to 1:3, yet more preferably 5:1 to 1:2, most preferably 4:1 to 1:1, and in particular 3:1 to 2:1.

Preferably, the relative weight ratio of the pharmacologically active compound to the disintegrant is within the range of 4:1 to 1:10, more preferably 3:1 to 1:9, still more preferably 2:1 to 1:8, yet more preferably 1:1 to 1:7, most preferably 1:2 to 1:6, and in particular 1:3 to 1:5.

The pharmaceutical dosage form may contain a single disintegrant or a mixture of different disintegrants. Preferably, the pharmaceutical dosage form contains a single disintegrant.

Preferably, the pharmaceutical dosage form according to the invention additionally comprises a gelling agent. The gelling agent may be contained in the multitude of immediate release particles (IR particles or FR particles) and/or in the at least one controlled release particle (PR particle(s) or DR particles or OR particles) and/or outside the particles.

While the gelling agent may principally contribute to the overall resistance against solvent extraction of the pharmaceutical dosage form according to the invention, it has been unexpectedly found that one or more disintegrants in comparatively high amounts in combination with one or more gelling agents are of particular advantage in this regard. It has been surprisingly found that the combination of one or more disintegrants in comparatively high amounts with one or more gelling agent is robust against variation of the pharmacologically active compound. Thus, according to the present invention exchanging a given pharmacologically active compound by another pharmacologically active compound does preferably not substantially alter the overall resistance against solvent extraction of the pharmaceutical dosage form according to the invention

As used herein the term “gelling agent” is used to refer to a compound that, upon contact with a solvent (e.g. water), absorbs the solvent and swells, thereby forming a viscous or semi-viscous substance. Preferred gelling agents are not cross-linked. This substance may moderate pharmacologically active compound release from the particles in both aqueous and aqueous alcoholic media. Upon full hydration, a thick viscous solution or dispersion is typically produced that significantly reduces and/or minimizes the amount of free solvent which can contain an amount of solubilized pharmacologically active compound, and which can be drawn into a syringe. The gel that is formed may also reduce the overall amount of pharmacologically active compound extractable with the solvent by entrapping the pharmacologically active compound within a gel structure. Thus the gelling agent may play an important role in conferring tamper-resistance to the pharmaceutical dosage forms according to the invention.

Gelling agents include pharmaceutically acceptable polymers, typically hydrophilic polymers, such as hydrogels. Representative examples of gelling agents include gums like xanthan gum, carrageenan, locust bean gum, guar, tragacanth, acaica (gum arabic), karaya, tara and gellan gum; polyethylene oxide, polyvinyl alcohol, hydroxypropylmethylcellulose, carbomers, poly(uronic) acids and mixtures thereof.

Preferably, the content of the gelling agent, preferably xanthan gum, is at least 1.0 wt.-%, more preferably at least 2.0 wt.-%, still more preferably at least 3.0 wt.-%, most preferably at least 4.0 wt.-%, based on the total weight of the pharmaceutical dosage form or based on the total weight of the particles or based on the total weight of those particles that contain the gelling agent.

Preferably, the content of the gelling agent, preferably xanthan gum, is within the range of 5.0±4.5 wt.-%, more preferably 5.0±4.0 wt.-%, still more preferably 5.0±3.5 wt.-%, yet more preferably 5.0±3.0 wt.-%, even more preferably 5.0±2.5 wt.-%, most preferably 5.0±2.0 wt.-%, and in particular 5.0±1.5 wt.-%, based on the total weight of the pharmaceutical dosage form or based on the total weight of the particles or based on the total weight of those particles that contain the gelling agent.

Preferably, the relative weight ratio of disintegrant:gelling agent is within the range of from 11:1 to 1:5, more preferably 10:1 to 1:4, still more preferably 9:1 to 1:3, yet more preferably 8:1 to 1:2, even more preferably 7:1 to 1:1, most preferably 6:1 to 2:1, and in particular 5:1 to 3:1.

The pharmaceutical dosage form and/or the particles according to the invention independently of one another may further contain additional pharmaceutical excipients conventionally contained in pharmaceutical dosage forms in conventional amounts, such as antioxidants, preservatives, lubricants, plasticizer, fillers, binders, and the like.

The skilled person will readily be able to determine appropriate further excipients as well as the quantities of each of these excipients. Specific examples of pharmaceutically acceptable carriers and excipients that may be used to formulate the pharmaceutical dosage forms according to the invention are described in the Handbook of Pharmaceutical Excipients, American Pharmaceutical Association (1986).

Preferably, the pharmaceutical dosage form and/or the particles according to the invention independently of one another may further comprise an antioxidant. Suitable antioxidants include ascorbic acid, butylated hydroxyanisole (BHA), butylated hydroxytoluene (BHT), salts of ascorbic acid, monothioglycerol, phosphorous acid, vitamin C, vitamin E and the derivatives thereof, coniferyl benzoate, nordihydroguaj aretic acid, gallus acid esters, sodium bisulfite, particularly preferably butylhydroxytoluene or butylhydroxyanisole and α-tocopherol. The antioxidant is preferably present in quantities of 0.01 wt.-% to 10 wt.-%, more preferably of 0.03 wt.-% to 5 wt.-%, most preferably of 0.05 wt.-% to 2.5 wt.-%, based on the total weight of the pharmaceutical dosage form or based on the total weight of the particles or based on the total weight of those particles that contain the antioxidant.

In a preferred embodiment, the pharmaceutical dosage form and/or the particles according to the invention independently of one another may further comprise an acid, preferably citric acid. The acid may be contained in the multitude of immediate release particles (IR particles or FR particles) and/or in the at least one controlled release particle (PR particle(s) or DR particles or OR particles) and/or outside the particles.

The amount of acid is preferably in the range of 0.01 wt.-% to 20 wt.-%, more preferably in the range of 0.02 wt.-% to 10 wt.-%, and still more preferably in the range of 0.05 wt.-% to 5 wt.-%, and most preferably in the range of 0.1 wt.-% to 1.0 wt.-%, based on the total weight of the pharmaceutical dosage form or based on the total weight of the particles or based on the total weight of those particles that contain the acid.

In a preferred embodiment, the pharmaceutical dosage form and/or the particles according to the invention independently of one another may further comprise another polymer.

Said another polymer is preferably selected from the group consisting of polyethylene, polypropylene, polyvinyl chloride, polycarbonate, polystyrene, polyvinylpyrrolidone, poly(alk)acrylate, poly(hydroxy fatty acids), such as for example poly(3-hydroxybutyrate-co-3-hydroxyvalerate) (Biopol®), poly(hydroxyvaleric acid); polycaprolactone, polyvinyl alcohol, polyesteramide, polyethylene succinate, polylactone, polyglycolide, polyurethane, polyamide, polylactide, polyacetal (for example polysaccharides optionally with modified side chains), polylactide/glycolide, polylactone, polyglycolide, polyorthoester, polyanhydride, block polymers of polyethylene glycol and polybutylene terephthalate (Polyactive®), polyanhydride (Polifeprosan), copolymers thereof, block-copolymers thereof (e.g., Poloxamer®), and mixtures of at least two of the stated polymers, or other polymers with the above characteristics. Preferably, said another polymer is selected from cellulose esters and cellulose ethers, in particular hydroxypropylmethylcellulose (HPMC).

The amount of said another polymer, preferably hydroxypropylmethylcellulose, preferably ranges from 0.1 wt.-% to 30 wt.-%, more preferably in the range of 1.0 wt.-% to 20 wt.-%, most preferably in the range of 2.0 wt.-% to 15 wt.-%, and in particular in the range of 3.5 wt.-% to 10.5 wt.-%, based on the total weight of the pharmaceutical dosage form or based on the total weight of the particles or based on the total weight of those particles that contain said another polymer.

In a preferred embodiment, the relative weight ratio of the polyalkylene oxide to said another polymer is within the range of 4.5±2:1, more preferably 4.5±1.5:1, still more preferably 4.5±1:1, yet more preferably 4.5±0.5:1, most preferably 4.5±0.2:1, and in particular 4.5±0.1:1. In another preferred embodiment, the relative weight ratio of the polyalkylene oxide to the further polymer is within the range of 8±7:1, more preferably 8±6:1, still more preferably 8±5:1, yet more preferably 8±4:1, most preferably 8±3:1, and in particular 8±2:1. In still another preferred embodiment, the relative weight ratio of the polyalkylene oxide to the further polymer is within the range of 11±8:1, more preferably 11±7:1, still more preferably 11±6:1, yet more preferably 11±5:1, most preferably 11±4:1, and in particular 11±3:1.

In another preferred embodiment, the pharmaceutical dosage form and/or the particles according to the invention do not contain any other polymer besides the polyalkylene oxide and optionally, polyethylene glycol.

In a preferred embodiment, the pharmaceutical dosage form contains at least one lubricant. Preferably, the lubricant is contained in the pharmaceutical dosage form outside the particles, i.e. the particles as such preferably do not contain lubricant. In another preferred embodiment, the pharmaceutical dosage form contains no lubricant. Especially preferred lubricants are selected from
magnesium stearate and stearic acid;
glycerides of fatty acids, including monoglycerides, diglycerides, triglycerides, and mixtures thereof; preferably of C₆ to C₂₂ fatty acids; especially preferred are partial glycerides of the C₁₆ to C₂₂ fatty acids such as glycerol behenat, glycerol palmitostearate and glycerol monostearate;
polyoxyethylene glycerol fatty acid esters, such as mixtures of mono-, di- and triesters of glycerol and di- and monoesters of macrogols having molecular weights within the range of from 200 to 4000 g/mol, e.g., macrogolglycerolcaprylocaprate, macrogolglycerollaurate, macrogolglycerolococoate, macrogolglycerollinoleate, macrogol-20-glycerolmonostearate, macrogol-6-glycerolcaprylocaprate, macrogolglycerololeate; macrogolglycerolstearate, macrogolglycerolhydroxystearate, and macrogolglycerolrizinoleate;
polyglycolyzed glycerides, such as the one known and commercially available under the trade name “Labrasol”;
fatty alcohols that may be linear or branched, such as cetylalcohol, stearylalcohol, cetylstearyl alcohol, 2-octyldodecane-1-ol and 2-hexyldecane-1-ol;
polyethylene glycols having a molecular weight between 10.000 and 60.000 g/mol; and
natural semi-synthetic or synthetic waxes, preferably waxes with a softening point of at least 50° C., more preferably 60° C., and in particular carnauba wax and bees wax.

Preferably, the amount of the lubricant ranges from 0.01 wt.-% to 10 wt.-%, more preferably in the range of 0.05 wt.-% to 7.5 wt.-%, most preferably in the range of 0.1 wt.-% to 5 wt.-%, and in particular in the range of 0.1 wt.-% to 1 wt.-%, based on the total weight of the pharmaceutical dosage form or based on the total weight of the particles or based on the total weight of those particles that contain the lubricant.

Preferably, the pharmaceutical dosage form and/or the particles according to the invention independently of one another may further comprise a plasticizer. The plasticizer improves the processability of the polyalkylene oxide. A preferred plasticizer is polyalkylene glycol, like polyethylene glycol, triacetin, fatty acids, fatty acid esters, waxes and/or microcrystalline waxes. Particularly preferred plasticizers are polyethylene glycols, such as PEG 6000 (Macrogol 6000).

Preferably, the content of the plasticizer is within the range of from 0.5 to 30 wt.-%, more preferably 1.0 to 25 wt.-%, still more preferably 2.5 wt.-% to 22.5 wt.-%, yet more preferably 5.0 wt.-% to 20 wt.-%, most preferably 6 to 20 wt.-% and in particular 7 wt.-% to 17.5 wt.-%, based on the total weight of the pharmaceutical dosage form or based on the total weight of the particles or based on the total weight of those particles that contain the plasticizer.

In a preferred embodiment, the plasticizer is a polyalkylene glycol having a content within the range of 7±6 wt.-%, more preferably 7±5 wt.-%, still more preferably 7±4 wt.-%, yet more preferably 7±3 wt.-%, most preferably 7±2 wt.-%, and in particular 7±1 wt.-%, based on the total weight of the pharmaceutical dosage form or based on the total weight of the particles or based on the total weight of those particles that contain the plasticizer. In another preferred embodiment, the plasticizer is a polyalkylene glycol having a content within the range of 10±8 wt.-%, more preferably 10±6 wt.-%, still more preferably 10±5 wt.-%, yet more preferably 10±4 wt.-%, most preferably 10±3 wt.-%, and in particular 10±2 wt.-%, based on the total weight of the pharmaceutical dosage form or based on the total weight of the particles or based on the total weight of those particles that contain the plasticizer.

In a preferred embodiment, the relative weight ratio of the polyalkylene oxide to the polyalkylene glycol is within the range of 5.4±2:1, more preferably 5.4±1.5:1, still more preferably 5.4±1:1, yet more preferably 5.4±0.5:1, most preferably 5.4±0.2:1, and in particular 5.4±0.1:1. This ratio satisfies the requirements of relative high polyalkylene oxide content and good extrudability.

Plasticizers can sometimes act as a lubricant, and lubricants can sometimes act as a plasticizer.

The shape of the particles is not particularly limited. As the IR particles and/or the FR particles and/or the CR particle(s) and/or the PR particle(s) and/or the DR particles and/or the OR particles independently of one another are preferably manufactured by hot-melt extrusion, preferred particles present in the pharmaceutical dosage forms according to the invention are generally cylindrical in shape. The diameter of such particles is therefore the diameter of their circular cross section. The cylindrical shape is caused by the extrusion process according to which the diameter of the circular cross section is a function of the extrusion die and the length of the cylinders is a function of the cutting length according to which the extruded strand of material is cut into pieces of preferably more or less predetermined length.

The suitability of cylindrical, i.e. a spherical particles for the manufacture of the pharmaceutical dosage forms according to the invention is unexpected. Typically, the aspect ratio is regarded as an important measure of the spherical shape. The aspect ratio is defined as the ratio of the maximal diameter (dₘₐₓ) and its orthogonal Feret-diameter. For aspherical particles, the aspect ratio has values above 1. The smaller the value the more spherical is the particle. Aspect ratios below 1.1 are typically considered satisfactory, aspect ratios above 1.2, however, are typically considered not suitable for the manufacture of conventional pharmaceutical dosage forms. The inventors have surprisingly found that when manufacturing the pharmaceutical dosage forms according to the invention, even particles having aspect ratios above 1.2 can be processed without difficulties and that it is not necessary to provide spherical particles. In a preferred embodiment, the aspect ratio of the particles is at most 1.40, more preferably at most 1.35, still more preferably at most 1.30, yet more preferably at most 1.25, even more preferably at most 1.20, most preferably at most 1.15 and in particular at most 1.10. In another preferred embodiment, the aspect ratio of the particles is at least 1.10, more preferably at least 1.15, still more preferably at least 1.20, yet more preferably at least 1.25, even more preferably at least 1.30, most preferably at least 1.35 and in particular at least 1.40.

### Ir Particles:

The pharmaceutical dosage form according to the invention comprises a multitude of IR particles. Preferably, each of said IR particles has an individual weight of less than 20 mg, more preferably not more than 10 mg.

The IR particles provide immediate release of the pharmacologically active compound under in vitro conditions. For the purpose of the specification, “immediate release” preferably means non-retarded release. The IR particles are designed to dissolve in the stomach within minutes.

The term “immediate release” as applied to pharmaceutical dosage forms is understood by persons skilled in the art which has structural implications for the respective pharmaceutical dosage forms. The term is defined, for example, in the current issue of the US Pharmacopoeia (USP), General Chapter 1092, “THE DISSOLUTION PROCEDURE: DEVELOPMENT AND VALIDATION”, heading “STUDY DESIGN”, “Time Points”. For immediate-release dosage forms, the duration of the procedure is typically 30 to 60 minutes; in most cases, a single time point specification is adequate for Pharmacopeia purposes. Industrial and regulatory concepts of product comparability and performance may require additional time points, which may also be required for product registration or approval. A sufficient number of time points should be selected to adequately characterize the ascending and plateau phases of the dissolution curve. According to the Biopharmaceutics Classification System referred to in several FDA Guidances, highly soluble, highly permeable drugs formulated with rapidly dissolving products need not be subjected to a profile comparison if they can be shown to release 85% or more of the active drug substance within 15 minutes. For these types of products a one-point test will suffice. However, most products do not fall into this category. Dissolution profiles of immediate-release products typically show a gradual increase reaching 85% to 100% at 30 to 45 minutes. Thus, dissolution time points in the range of 15, 20, 30, 45, and 60 minutes are usual for most immediate-release products.

Preferably, when tested alone (i.e. in the absence of particles of other type and nature), said multitude of IR particles provide immediate release of the pharmacologically active compound such that under in vitro conditions in accordance with Ph. Eur. after 30 minutes in artificial gastric juice at pH 1.2 at least 70%, still more preferably at least 75 wt.-%, yet more preferably at least 80 wt.-%, even more preferably at least 85 wt.-%, most preferably at least 90 wt.-% of the pharmacologically active compound that were originally contained in said multitude of IR particles have been released.

In a preferred embodiment, the IR particles are not coated. Preferred non-coated IR particles are hot-melt extruded. Preferred non-coated IR particles contain as the pharmacologically active compound a stimulant, preferably amphetamine or a physiologically acceptable salt thereof, more preferably amphetamine sulfate. Preferably, these non-coated IR particles comprise
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol, and
optionally an acid, preferably citric acid.

For the purpose of the specification, “optionally” in the context of the excipients means that these excipients may independently of one another be contained in the particles or not and provided that they are contained in the particles, their content in wt.-% is as specified.

Particularly preferred embodiments A¹ to A⁶ of such non-coated IR particles are summarized in the table here below (all percentages relative to the total weight of the IR particles):

| [wt.-%] | A¹ | A² | A³ | A⁴ | A⁵ | A⁶ |
|---|---|---|---|---|---|---|
| stimulant | 2.8 ± 2.6 | 2.8 ± 2.4 | 2.8 ± 2.2 | 2.8 ± 2.0 | 2.8 ± 1.8 | 2.8 ± 1.6 |
| polyethylene oxide | 56.8 ± 35.0 | 56.8 ± 30.0 | 56.8 ± 25.0 | 56.8 ± 20.0 | 56.8 ± 15.0 | 56.8 ± 10.0 |
| disintegrant | 16.1 ± 16.0 | 16.1 ± 15.0 | 16.1 ± 14.0 | 16.1 ± 13.0 | 16.1 ± 12.0 | 16.1 ± 11.0 |
| optionally, plasticizer | 23.6 ± 20.0 | 23.6 ± 18.0 | 23.6 ± 16.0 | 23.6 ± 14.0 | 23.6 ± 12.0 | 23.6 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |

Further particularly preferred embodiments B¹ to B⁶ of such non-coated IR particles are summarized in the table here below (all percentages relative to the total weight of the IR particles):

| [wt.-%] | B¹ | B² | B³ | B⁴ | B⁵ | B⁶ |
|---|---|---|---|---|---|---|
| stimulant | 8.3 ± 8.0 | 8.3 ± 7.5 | 8.3 ± 7.0 | 8.3 ± 6.5 | 8.3 ± 6.0 | 8.3 ± 5.5 |
| polyethylene oxide | 53.5 ± 35.0 | 53.5 ± 30.0 | 53.5 ± 25.0 | 53.5 ± 20.0 | 53.5 ± 15.0 | 53.5 ± 10.0 |
| disintegrant | 15.2 ± 15.0 | 15.2 ± 14.0 | 15.2 ± 13.0 | 15.2 ± 12.0 | 15.2 ± 11.0 | 15.2 ± 10.0 |
| optionally, plasticizer | 22.3 ± 20.0 | 22.3 ± 18.0 | 22.3 ± 16.0 | 22.3 ± 14.0 | 22.3 ± 12.0 | 22.3 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |

Still further particularly preferred embodiments C¹ to C⁶ of such non-coated IR particles are summarized in the table here below (all percentages relative to the total weight of the IR particles):

| [wt.-%] | C¹ | C² | C³ | C⁴ | C⁵ | C⁶ |
|---|---|---|---|---|---|---|
| stimulant | 15.0 ± 14.0 | 15.0 ± 12.0 | 15.0 ± 10.0 | 15.0 ± 8.0 | 15.0 ± 6.0 | 15.0 ± 4.0 |
| polyethylene oxide | 49.6 ± 35.0 | 49.6 ± 30.0 | 49.6 ± 25.0 | 49.6 ± 20.0 | 49.6 ± 15.0 | 49.6 ± 10.0 |
| disintegrant | 14.1 ± 12.0 | 14.1 ± 11.0 | 14.1 ± 10.0 | 14.1 ± 9.0 | 14.1 ± 8.0 | 14.1 ± 7.0 |
| optionally, plasticizer | 20.6 ± 20.0 | 20.6 ± 18.0 | 20.6 ± 16.0 | 20.6 ± 14.0 | 20.6 ± 12.0 | 20.6 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |

In another preferred embodiment, the IR particles are coated, but preferably with a non-enteric coating which does not delay in vitro dissolution.

When the IR particles are film coated with a non-enteric coating material which does not delay in vitro dissolution, the content of the dried non-enteric coating which does not delay in vitro dissolution is preferably at most 15 wt.-%, more preferably at most 14 wt.-%, still more preferably at most 13.5 wt.-%, yet more preferably at most 13 wt.-%, most preferably at most 12.5 wt.-%, and in particular at most 12 wt.-%, based on the total weight of the IR particles.

Suitable non-enteric coating materials are commercially available. Preferred non-enteric film coating materials are based on hydroxypropylmethylcellulose (e.g. Opadry®) or on polyvinylalcohol (e.g. Opadry® II). Preferred coated IR particles are hot-melt extruded. Preferred coated IR particles contain as the pharmacologically active compound a stimulant, preferably amphetamine or a physiologically acceptable salt thereof, more preferably amphetamine sulfate. Preferably, these coated IR particles comprise
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid, and
a coating, preferably a non-enteric coating which does not delay in vitro dissolution, preferably a non-enteric film coating based on hydroxypropylmethylcellulose (e.g. Opadry®) or based on polyvinylalcohol (e.g. Opadry® II).

Particularly preferred embodiments D¹ to D⁶ of such coated IR particles are summarized in the table here below (all percentages relative to the total weight of the IR particles):

| [wt.-%] | D¹ | D² | D³ | D⁴ | D⁵ | D⁶ |
|---|---|---|---|---|---|---|
| stimulant | 2.6 ± 2.5 | 2.6 ± 2.4 | 2.6 ± 2.2 | 2.6 ± 2.0 | 2.6 ± 1.8 | 2.6 ± 1.6 |
| polyethylene oxide | 52.8 ± 35.0 | 52.8 ± 30.0 | 52.8 ± 25.0 | 52.8 ± 20.0 | 52.8 ± 15.0 | 52.8 ± 10.0 |
| disintegrant | 15.0 ± 14.0 | 15.0 ± 13.0 | 15.0 ± 12.0 | 15.0 ± 11.0 | 15.0 ± 10.0 | 15.0 ± 9.0 |
| optionally, plasticizer | 20.8 ± 20.0 | 20.8 ± 18.0 | 20.8 ± 16.0 | 20.8 ± 14.0 | 20.8 ± 12.0 | 20.8 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| non-enteric coating | 7.0 ± 6.5 | 7.0 ± 6.0 | 7.0 ± 5.5 | 7.0 ± 5.0 | 7.0 ± 4.5 | 7.0 ± 4.0 |

Further particularly preferred embodiments E¹ to E⁶ of such coated IR particles are summarized in the table here below (all percentages relative to the total weight of the IR particles):

| [wt.-%] | E¹ | E² | E³ | E⁴ | E⁵ | E⁶ |
|---|---|---|---|---|---|---|
| stimulant | 7.8 ± 7.6 | 7.8 ± 7.5 | 7.8 ± 7.0 | 7.8 ± 6.5 | 7.8 ± 6.0 | 7.8 ± 5.5 |
| polyethylene oxide | 49.8 ± 35.0 | 49.8 ± 30.0 | 49.8 ± 25.0 | 49.8 ± 20.0 | 49.8 ± 15.0 | 49.8 ± 10.0 |
| disintegrant | 14.2 ± 13.0 | 14.2 ± 12.0 | 14.2 ± 11.0 | 14.2 ± 10.0 | 14.2 ± 9.0 | 14.2 ± 8.0 |
| optionally, plasticizer | 19.6 ± 19.0 | 19.6 ± 18.0 | 19.6 ± 16.0 | 19.6 ± 14.0 | 19.6 ± 12.0 | 19.6 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| non-enteric coating | 7.0 ± 6.5 | 7.0 ± 6.0 | 7.0 ± 5.5 | 7.0 ± 5.0 | 7.0 ± 4.5 | 7.0 ± 4.0 |

Still further particularly preferred embodiments F¹ to F⁶ of such coated IR particles are summarized in the table here below (all percentages relative to the total weight of the IR particles):

| [wt.-%] | F¹ | F² | F³ | F⁴ | F⁵ | F⁶ |
|---|---|---|---|---|---|---|
| stimulant | 14.0 ± 13.5 | 14.0 ± 12.0 | 14.0 ± 10.0 | 14.0 ± 8.0 | 14.0 ± 6.0 | 14.0 ± 4.0 |
| polyethylene oxide | 46.1 ± 35.0 | 46.1 ± 30.0 | 46.1 ± 25.0 | 46.1 ± 20.0 | 46.1 ± 15.0 | 46.1 ± 10.0 |
| disintegrant | 13.1 ± 12.0 | 13.1 ± 11.0 | 13.1 ± 10.0 | 13.1 ± 9.0 | 13.1 ± 8.0 | 13.1 ± 7.0 |
| optionally, plasticizer | 18.1 ± 18.0 | 18.1 ± 17.0 | 18.1 ± 16.0 | 18.1 ± 14.0 | 18.1 ± 12.0 | 18.1 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| non-enteric coating | 7.0 ± 6.5 | 7.0 ± 6.0 | 7.0 ± 5.5 | 7.0 ± 5.0 | 7.0 ± 4.5 | 7.0 ± 4.0 |

### Fr Particles:

In a preferred embodiment, the IR particles are coated with a coating which slightly retards in vitro dissolution thereby rendering the particles fast release particles (FR particles). Said coating may have a single layer or more than a single layer, e.g. two layers. Suitable coating materials for FR particles are commercially available. Preferred coating materials are enteric and/or based on an acrylate polymer.

Preferably, each of said FR particles has an individual weight of less than 20 mg, more preferably not more than 10 mg.

Preferred acrylate polymers are defined further below in connection with the enteric coating of the DR particles according to the invention. These definitions also analogously apply to the acrylate polymer that may be contained in the enteric coating of the FR particles according to the invention.

The FR particles provide fast release of the pharmacologically active compound under in vitro conditions. For the purpose of the specification, “fast release” preferably means slightly retarded release. The FR particles are designed to dissolve in the stomach within minutes but not as quick as the IR particles.

Preferably, when tested alone (i.e. in the absence of particles of other type and nature), said multitude of FR particles provide immediate release of the pharmacologically active compound such that under in vitro conditions in accordance with Ph. Eur. after 30 minutes in artificial gastric juice at pH 1.2 less than 70% of the pharmacologically active compound that were originally contained in said multitude of FR particles have been released; and such that after 60 minutes in artificial gastric juice at pH 1.2 at least 70%, still more preferably at least 75 wt.-%, yet more preferably at least 80 wt.-%, even more preferably at least 85 wt.-%, and most preferably at least 90 wt.-% of the pharmacologically active compound that were originally contained in said multitude of FR particles have been released.

Preferably, when tested alone (i.e. in the absence of particles of other type and nature), said multitude of FR particles provide immediate release of the pharmacologically active compound such that under in vitro conditions in accordance with Ph. Eur. after 30 minutes in artificial gastric juice at pH 1.2 less than 70% of the pharmacologically active compound that were originally contained in said multitude of FR particles have been released; and such that after 45 minutes in artificial gastric juice at pH 1.2 at least 70%, still more preferably at least 75 wt.-%, yet more preferably at least 80 wt.-%, even more preferably at least 85 wt.-%, and most preferably at least 90 wt.-% of the pharmacologically active compound that were originally contained in said multitude of FR particles have been released.

Preferably, the content of the dried enteric coating of the FR particles according to the invention is at most 15 wt.-%, more preferably at most 14 wt.-%, still more preferably at most 13.5 wt.-%, yet more preferably at most 13 wt.-%, most preferably at most 12.5 wt.-%, and in particular at most 12 wt.-%, based on the total weight of the FR particles. This is a significant difference of the FR particles compared to the DR particles and compared to the OR particles, respectively, which both in turn typically have a higher content of enteric coating material.

In a preferred embodiment, the acrylate polymer in the coating of the FR particles is derived from a monomer mixture comprising methacrylic acid in combination with one or two comonomers selected from methyl acrylate, methyl methacrylate and ethyl acrylate. Preferably, the methacrylic acid-ethyl acrylate copolymer has a ratio of free carboxyl groups to ester groups within the range of from 3:1 to 1:3, more preferably 2:1 to 1:2.

In another preferred embodiment, the acrylate polymer in the coating of the FR particles is derived from a monomer mixture comprising methacrylic acid in combination with methyl acrylate and methyl methacrylate. Preferably, the anionic copolymer has a ratio of free carboxyl groups to ester groups within the range of from 1:8 to 1:12, more preferably 1:9 to 1:11.

Preferably, the acrylate polymer has a weight average molecular weight of at least 50,000 g/mol, or at least 100,000 g/mol, or at least 150,000 g/mol, or at least 200,000 g/mol, or at least 250,000 g/mol. Preferably, the acrylate polymer has a weight average molecular weight of at most 500,000 g/mol, or at most 450,000 g/mol, or at most 400,000 g/mol, or at most 350,000 g/mol, or at most 300,000 g/mol. Preferably, the acrylate polymer has a weight average molecular weight within the range of from 200,000 to 400,000 g/mol, more preferably within the range of from 250,000 to 350,000 g/mol.

A particularly preferred acrylate polymer in the coating of the FR particles is a methacrylic acid-ethyl acrylate copolymer (1:1) (e.g. Eudragit® L 30 D-55).

The coating may additionally contain typical excipients such as lubricants (e.g. talcum) and/or plasticizers (e.g. triethyl citrate).

In a preferred embodiment, the FR particles contain a single coating layer. Preferred FR particles are hot-melt extruded. Preferred FR particles contain as the pharmacologically active compound a stimulant, preferably amphetamine or a physiologically acceptable salt thereof, more preferably amphetamine sulfate. Preferably, these FR particles comprise
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid, and
a coating, preferably a coating which slightly retards in vitro dissolution, preferably based on an acrylate polymer, more preferably a methacrylic acid-ethyl acrylate copolymer (e.g. Eudragit® L30-D55).

Particularly preferred embodiments G¹ to G⁶ of such FR particles are summarized in the table here below (all percentages relative to the total weight of the FR particles):

| [wt.-%] | G¹ | G² | G³ | G⁴ | G⁵ | G⁶ |
|---|---|---|---|---|---|---|
| stimulant | 2.6 ± 2.5 | 2.6 ± 2.4 | 2.6 ± 2.2 | 2.6 ± 2.0 | 2.6 ± 1.8 | 2.6 ± 1.6 |
| polyethylene oxide | 52.8 ± 35.0 | 52.8 ± 30.0 | 52.8 ± 25.0 | 52.8 ± 20.0 | 52.8 ± 15.0 | 52.8 ± 10.0 |
| disintegrant | 15.0 ± 14.0 | 15.0 ± 13.0 | 15.0 ± 12.0 | 15.0 ± 11.0 | 15.0 ± 10.0 | 15.0 ± 9.0 |
| optionally, plasticizer | 20.8 ± 20.0 | 20.8 ± 18.0 | 20.8 ± 16.0 | 20.8 ± 14.0 | 20.8 ± 12.0 | 20.8 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| acrylate polymer coating | 9.0 ± 8.5 | 9.0 ± 8.0 | 9.0 ± 7.5 | 9.0 ± 7.0 | 9.0 ± 6.5 | 9.0 ± 6.0 |

Further particularly preferred embodiments H¹ to H⁶ of such FR particles are summarized in the table here below (all percentages relative to the total weight of the FR particles):

| [wt.-%] | H¹ | H² | H³ | H⁴ | H⁵ | H⁶ |
|---|---|---|---|---|---|---|
| stimulant | 7.8 ± 7.6 | 7.8 ± 7.5 | 7.8 ± 7.0 | 7.8 ± 6.5 | 7.8 ± 6.0 | 7.8 ± 5.5 |
| polyethylene oxide | 49.8 ± 35.0 | 49.8 ± 30.0 | 49.8 ± 25.0 | 49.8 ± 20.0 | 49.8 ± 15.0 | 49.8 ± 10.0 |
| disintegrant | 14.2 ± 13.0 | 14.2 ± 12.0 | 14.2 ± 11.0 | 14.2 ± 10.0 | 14.2 ± 9.0 | 14.2 ± 8.0 |
| optionally, plasticizer | 19.6 ± 19.0 | 19.6 ± 18.0 | 19.6 ± 16.0 | 19.6 ± 14.0 | 19.6 ± 12.0 | 19.6 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| acrvlate polymer coating | 9.0 ± 8.5 | 9.0 ± 8.0 | 9.0 ± 7.5 | 9.0 ± 7.0 | 9.0 ± 6.5 | 9.0 ± 6.0 |

Still further particularly preferred embodiments I¹ to I⁶ of such FR particles are summarized in the table here below (all percentages relative to the total weight of the FR particles):

| [wt.-%] | I¹ | I² | I³ | I⁴ | I⁵ | I⁶ |
|---|---|---|---|---|---|---|
| stimulant | 14.0 ± 13.5 | 14.0 ± 12.0 | 14.0 ± 10.0 | 14.0 ± 8.0 | 14.0 ± 6.0 | 14.0 ± 4.0 |
| polyethylene oxide | 46.1 ± 35.0 | 46.1 ± 30.0 | 46.1 ± 25.0 | 46.1 ± 20.0 | 46.1 ± 15.0 | 46.1 ± 10.0 |
| disintegrant | 13.1 ± 12.0 | 13.1 ± 11.0 | 13.1 ± 10.0 | 13.1 ± 9.0 | 13.1 ± 8.0 | 13.1 ± 7.0 |
| optionally, plasticizer | 18.1 ± 18.0 | 18.1 ± 17.0 | 18.1 ± 16.0 | 18.1 ± 14.0 | 18.1 ± 12.0 | 18.1 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| acrylate polymer coating | 9.0 ± 8.5 | 9.0 ± 8.0 | 9.0 ± 7.5 | 9.0 ± 7.0 | 9.0 ± 6.5 | 9.0 ± 6.0 |

In another preferred embodiment, the FR particles contain two coating layers, i.e. an inner coating layer and an outer coating layer. Preferably, said inner coating layer and said outer coating layer are in intimate contact with one another, i.e. adjacent layers.

Preferably, the inner layer of the coating of the FR particles is a non-enteric coating which a such preferably does not delay in vitro dissolution. Preferably, the inner layer of the coating of the FR particles is a non-enteric film coating based on hydroxypropylmethylcellulose (e.g. Opadry®) or based on polyvinylalcohol (e.g. Opadry® II).

Preferably, the outer layer of the coating of the FR particles is enteric and/or based on an acrylate polymer as defined above (e.g. Eudragit® L30-D55).

Preferred FR particles are hot-melt extruded. Preferred FR particles contain as the pharmacologically active compound a stimulant, preferably amphetamine or a physiologically acceptable salt thereof, more preferably amphetamine sulfate. Preferably, these FR particles comprise
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid,
an inner coating, preferably a non-enteric film coating based on hydroxypropylmethylcellulose or based on polyvinylalcohol, and
an outer coating, preferably an outer coating which slightly retards in vitro dissolution, preferably based on an acrylate polymer, more preferably a methacrylic acid-ethyl acrylate copolymer (e.g. Eudragit® L30-D55).

Particularly preferred embodiments J¹ to J⁶ of such FR particles are summarized in the table here below (all percentages relative to the total weight of the FR particles):

| [wt.-%] | J¹ | J² | J³ | J⁴ | J⁵ | J⁶ |
|---|---|---|---|---|---|---|
| stimulant | 2.6 ± 2.5 | 2.6 ± 2.4 | 2.6 ± 2.2 | 2.6 ± 2.0 | 2.6 ± 1.8 | 2.6 ± 1.6 |
| polyethylene oxide | 52.8 ± 35.0 | 52.8 ± 30.0 | 52.8 ± 25.0 | 52.8 ± 20.0 | 52.8 ± 15.0 | 52.8 ± 10.0 |
| disintegrant | 15.0 ± 14.0 | 15.0 ± 13.0 | 15.0 ± 12.0 | 15.0 ± 11.0 | 15.0 ± 10.0 | 15.0 ± 9.0 |
| optionally, plasticizer | 20.8 ± 20.0 | 20.8 ± 18.0 | 20.8 ± 16.0 | 20.8 ± 14.0 | 20.8 ± 12.0 | 20.8 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| inner layer | 5.0 ± 4.5 | 5.0 ± 4.0 | 5.0 ± 3.5 | 5.0 ± 3.0 | 5.0 ± 2.5 | 5.0 ± 2.0 |
| non-enteric coating | | | | | | |
| outer layer acrylate | 9.0 ± 8.5 | 9.0 ± 8.0 | 9.0 ± 7.5 | 9.0 ± 7.0 | 9.0 ± 6.5 | 9.0 ± 6.0 |
| polymer coating | | | | | | |

Further particularly preferred embodiments K¹ to K⁶ of such FR particles are summarized in the table here below (all percentages relative to the total weight of the FR particles):

| [wt.-%] | K¹ | K² | K³ | K⁴ | K⁵ | K⁶ |
|---|---|---|---|---|---|---|
| stimulant | 7.8 ± 7.6 | 7.8 ± 7.5 | 7.8 ± 7.0 | 7.8 ± 6.5 | 7.8 ± 6.0 | 7.8 ± 5.5 |
| polyethylene oxide | 49.8 ± 35.0 | 49.8 ± 30.0 | 49.8 ± 25.0 | 49.8 ± 20.0 | 49.8 ± 15.0 | 49.8 ± 10.0 |
| disintegrant | 14.2 ± 13.0 | 14.2 ± 12.0 | 14.2 ± 11.0 | 14.2 ± 10.0 | 14.2 ± 9.0 | 14.2 ± 8.0 |
| optionally, plasticizer | 19.6 ± 19.0 | 19.6 ± 18.0 | 19.6 ± 16.0 | 19.6 ± 14.0 | 19.6 ± 12.0 | 19.6 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| inner layer | 5.0 ± 4.5 | 5.0 ± 4.0 | 5.0 ± 3.5 | 5.0 ± 3.0 | 5.0 ± 2.5 | 5.0 ± 2.0 |
| non-enteric coating | | | | | | |
| outer layer acrylate | 9.0 ± 8.5 | 9.0 ± 8.0 | 9.0 ± 7.5 | 9.0 ± 7.0 | 9.0 ± 6.5 | 9.0 ± 6.0 |
| polymer coating | | | | | | |

Still further particularly preferred embodiments L¹ to L⁶ of such FR particles are summarized in the table here below (all percentages relative to the total weight of the FR particles):

| [wt.-%] | L¹ | L² | L³ | L⁴ | L⁵ | L⁶ |
|---|---|---|---|---|---|---|
| stimulant | 14.0 ± 13.5 | 14.0 ± 12.0 | 14.0 ± 10.0 | 14.0 ± 8.0 | 14.0 ± 6.0 | 14.0 ± 4.0 |
| polyethylene oxide | 46.1 ± 35.0 | 46.1 ± 30.0 | 46.1 ± 25.0 | 46.1 ± 20.0 | 46.1 ± 15.0 | 46.1 ± 10.0 |
| disintegrant | 13.1 ± 12.0 | 13.1 ± 11.0 | 13.1 ± 10.0 | 13.1 ± 9.0 | 13.1 ± 8.0 | 13.1 ± 7.0 |
| optionally, plasticizer | 18.1 ± 18.0 | 18.1 ± 17.0 | 18.1 ± 16.0 | 18.1 ± 14.0 | 18.1 ± 12.0 | 18.1 ± 10.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| inner layer | 5.0 ± 4.5 | 5.0 ± 4.0 | 5.0 ± 3.5 | 5.0 ± 3.0 | 5.0 ± 2.5 | 5.0 ± 2.0 |
| non-enteric coating | | | | | | |
| outer layer acrylate | 9.0 ± 8.5 | 9.0 ± 8.0 | 9.0 ± 7.5 | 9.0 ± 7.0 | 9.0 ± 6.5 | 9.0 ± 6.0 |
| polymer coating | | | | | | |

### PR Particle(s):

The pharmaceutical dosage form according to the invention comprises at least one CR particle providing controlled release of the pharmacologically active compound.

For the purpose of the specification, “controlled release” means non-immediate release and non-fast release, respectively. Controlled release refers to time dependent release, i.e. timed release, having several distinct variants such as prolonged release (sustained release, extended release) and delayed release and postponed release. A distinction of controlled release is that it not only prolongs action but it attempts to maintain drug levels within the therapeutic window to avoid potentially hazardous peaks in drug concentration following ingestion or injection and to maximize therapeutic efficiency. Thus, controlled release can be divided in delayed release/postponed release and prolonged release (sustained release, extended). For the purpose of the specification, “prolonged release” is a mechanism to dissolve a drug over time in order to be released slower and steadier into the bloodstream.

Preferably, when tested alone (i.e. in the absence of particles of other type and nature), said at least one controlled release particle and said multitude of controlled release particles, respectively, provide controlled release of the pharmacologically active compound such that under in vitro conditions in accordance with Ph. Eur. after 30 minutes in artificial gastric juice at pH 1.2 less than 50%, more preferably at most 40 wt.-%, still more preferably at most 30 wt.-%, yet more preferably at most 10 wt.-% of the pharmacologically active compound that were originally contained in said at least one controlled release particle and said multitude of controlled release particles, respectively, have been released.

According to a preferred embodiment of the invention, the pharmaceutical dosage form comprises a single CR particle or a few CR particles (2, 3 or 4 controlled release particles), wherein an individual CR particle is preferably substantially bigger and/or heavier than an individual IR particle. Preferably, said single CR particle or every individual CR particle within the group of said few CR particles has a total weight of at least 20 mg, more preferably of at least 50 mg, still more preferably of at least 75 mg, yet more preferably of at least 100 mg, most preferably at least 125 mg and in particular at least 150 mg.

The particle(s) according to this embodiment of the invention form a subgroup of CR particle(s), namely prolonged release particle(s) (PR particle(s)). The PR particle(s) according to the invention differ from the other subgroups of CR particle(s), namely from delayed release particles (DR particles) and from postponed release particles (OR particles), in their greater size and/or weight and/or in that they are not provided with an enteric coating.

Preferably, when tested alone (i.e. in the absence of particles of other type and nature), the PR particle(s) provide(s) prolonged release of the pharmacologically active compound such that under in vitro conditions in accordance with Ph. Eur. after 30 minutes in artificial gastric juice at pH 1.2 less than 50%, more preferably at most 40 wt.-%, still more preferably at most 30 wt.-%, yet more preferably at most 10 wt.-% of the pharmacologically active compound that were originally contained in said PR particle(s) have been released.

Preferably, the PR particle or every individual PR particle within the group of the few PR particles has a total weight of at least 20 mg, more preferably of at least 50 mg, still more preferably at least 100 mg, yet more preferably at least 150 mg, most preferably at least 200 mg. In preferred embodiments, every individual PR particle within the group of the few PR particles has a total weight within the range of 150±100 mg, preferably 150±50 mg; or 200±100 mg, preferably 200±50 mg; or 250±100 mg, preferably 250±50 mg; or 300±100 mg, preferably 300±50 mg; or 350±100 mg, preferably 350±50 mg.

In a preferred embodiment, the PR particle(s) are not film coated at all.

In another preferred embodiment, the PR particle(s) are film coated, but do not comprise an enteric coating, i.e. the coating is non-enteric. Thus, the PR particle(s) according to the invention can optionally be provided, partially or completely, with a conventional coating which does not delay in vitro dissolution. The PR particle(s) according to the invention are preferably film coated with conventional film coating compositions which does not delay in vitro dissolution. These film coatings which do not delay in vitro dissolution are preferably not functional, i.e. not enteric. Suitable coating materials are commercially available and are based e.g. on polyvinyl alcohol (PVA, e.g. Opadry® pink).

When the PR particles are film coated with a non-enteric coating material which does not delay in vitro dissolution, the content of the dried non-enteric coating which does not delay in vitro dissolution is preferably at most 15 wt.-%, more preferably at most 14 wt.-%, still more preferably at most 13.5 wt.-%, yet more preferably at most 13 wt.-%, most preferably at most 12.5 wt.-%, and in particular at most 12 wt.-%, based on the total weight of the PR particles.

Drug release of the pharmacologically active compound from PR particle(s) preferably essentially relies upon erosion and diffusion from a matrix in which the pharmacologically active compound is embedded. Preferably, said matrix comprises a polyalkylene oxide. Thus, prolonged release of the pharmacologically active compound from the PR particle(s) preferably relies upon the size thereof and the corresponding extended diffusion pathways from the core into the release medium. Preferably, the prolonged release is based on matrix retardation, where the retard matrix, in which the pharmacologically active compound is embedded, preferably comprises a polyalkylene oxide, optionally in combination with additional polymers, especially cellulose ethers such as hydroxypropylmethylcellulose.

Preferred PR particles are hot-melt extruded. Preferred PR particles contain as the pharmacologically active compound a stimulant, preferably amphetamine or a physiologically acceptable salt thereof, more preferably amphetamine sulfate. Preferably, these PR particle(s) comprise(s)
a polyalkylene oxide which is a polyethylene oxide with a weight average molecular weight within the range of from 0.5 to 15 million g/mol,
optionally a plasticizer, preferably a polyethylene glycol,
optionally an antioxidant, preferably alpha-tocopherol,
optionally an acid, preferably citric acid,
optionally another polymer, preferably a cellulose ether, preferably by droxypropylmethylcellulose.

Particularly preferred embodiments M¹ to M⁶ of such PR particle(s) are summarized in the table here below (all percentages relative to the total weight of the PR particle(s)):

| [wt.-%] | M¹ | M² | M³ | M⁴ | M⁵ | M⁶ |
|---|---|---|---|---|---|---|
| stimulant | 7.5 ± 6.0 | 7.5 ± 5.0 | 7.5 ± 4.5 | 7.5 ± 4.0 | 7.5 ± 3.5 | 7.5 ± 3.0 |
| polyethylene oxide | 66 ± 40 | 66 ± 35 | 66 ± 30 | 66 ± 25 | 66 ± 20 | 66 ± 15 |
| optionally, plasticizer | 16 ± 13 | 16 ± 12 | 16 ± 11 | 16 ± 10 | 16 ± 9 | 16 ± 8 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, | 10 ± 8 | 10 ± 7 | 10 ± 6 | 10 ± 5 | 10 ± 4 | 10 ± 3 |
| cellulose ether | | | | | | |

Preferred PR particles are hot-melt extruded. Preferred PR particles contain as the pharmacologically active compound a stimulant, preferably amphetamine or a physiologically acceptable salt thereof, more preferably amphetamine sulfate. Preferably, these PR particle(s) comprise(s)
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid,
optionally another polymer, preferably a cellulose ether, preferably by droxypropylmethylcellulose.

Particularly preferred embodiments N¹ to N⁶ of such PR particle(s) are summarized in the table here below (all percentages relative to the total weight of the PR particle(s)):

| [wt.-%] | N¹ | N² | N³ | N⁴ | N⁵ | N⁶ |
|---|---|---|---|---|---|---|
| stimulant | 7.5 ± 6.0 | 7.5 ± 5.0 | 7.5 ± 4.5 | 7.5 ± 4.0 | 7.5 ± 3.5 | 7.5 ± 3.0 |
| polyethylene oxide | 66 ± 40 | 66 ± 35 | 66 ± 30 | 66 ± 25 | 66 ± 20 | 66 ± 15 |
| disintegrant | 15 ± 14 | 15 ± 13 | 15 ± 12 | 15 ± 11 | 15 ± 10 | 15 ± 9 |
| optionally, plasticizer | 16 ± 13 | 16 ± 12 | 16 ± 11 | 16 ± 10 | 16 ± 9 | 16 ± 8 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, | 10 ± 8 | 10 ± 7 | 10 ± 6 | 10 ± 5 | 10 ± 4 | 10 ± 3 |
| cellulose ether | | | | | | |

### Dr Particles:

According to another preferred embodiment of the invention, the pharmaceutical dosage form comprises a multitude of CR particle which are coated with an enteric coating thereby rendering the particles DR particles.

Preferably, each of said DR particles has an individual weight of less than 20 mg, more preferably not more than 10 mg.

The multitude of DR particles provides delayed release of the pharmacologically active compound.

For the purpose of the specification, “delayed release” refers to oral medicines that do not immediately disintegrate and release the active ingredient(s) into the body. The DR particles according to the invention are preferably enterically coated such that they dissolve in the intestine rather than the stomach.

When tested alone (i.e. in the absence of particles of other type and nature), said multitude of DR particles provide delayed release of the pharmacologically active compound such that under in vitro conditions in accordance with Ph. Eur. after 30 minutes in artificial gastric juice at pH 1.2 preferably less than 50%, more preferably at most 40 wt.-%, still more preferably at most 30 wt.-%, yet more preferably at most 10 wt.-% of the pharmacologically active compound that were originally contained in said multitude of DR particles have been released.

When tested alone (i.e. in the absence of particles of other type and nature), said multitude of DR particles provide delayed release of the pharmacologically active compound such that under in vitro conditions in accordance with Ph. Eur., when changing the release medium after 120 minutes from initially artificial gastric juice with pH 1.2 to subsequently artificial intestinal fluid with pH 6.8, after 180 minutes preferably at least 20 wt.-%, more preferably at least 22.5 wt.-%, still more preferably at least 25 wt.-%, yet more preferably at least 27.5 wt.-%, most preferably at least 30 wt.-% of the pharmacologically active compound that were originally contained in said multitude of DR particles have been released.

The DR particles according to the invention are preferably provided, partially or completely, with an enteric coating. The DR particles according to the invention are preferably film coated with conventional enteric coating compositions. Suitable enteric coating materials are commercially available, e.g. under the trademarks Eudragit®. Enteric coating compositions typically comprise polymers, plasticizers, colorants and the like. Suitable polymers include but are not limited to cellulose acetate phthalate, hydroxypropylmethylcellulose phthalate, methylacrylate methylmethacrylate copolymers, and polyvinylacetate phthalate.

Preferably, the content of the dried enteric coating of the DR particles according to the invention is at least 12 wt.-%, more preferably at least 13 wt.-%, still more preferably at least 14 wt.-%, yet more preferably at least 15 wt.-%, most preferably at least 16 wt.-%, and in particular at least 17 wt.-%, based on the total weight of the DR particles. This is a significant difference of the DR particles compared to the FR particles, which in turn typically have a lower content of enteric coating material.

Preferably, the enteric coating also provides resistance against dose dumping in aqueous ethanol. This may preferably be achieved by two layers, i.e. an inner layer and an outer layer, which are based on different coating materials. Thus, the enteric coating preferably comprises an inner layer and an outer layer. Preferably, the enteric coating consists of the inner layer and the outer layer.

In a preferred embodiment, the DR particles are first provided with a layer of a non-enteric material, e.g. based on polyvinyl alcohol or based on hydroxypropylmethylcellulose (e.g. Opadry® pink) and the enteric coating comprising inner layer and outer layer is then applied to the layer of the non-enteric material. For the purpose of the specification, such optional layer of a non-enteric material does not belong to the enteric coating (e.g. does not contribute to the total weight of the enteric coating), but is a separate coating.

Preferably, said multitude of DR particles, when being tested alone, provides an in vitro release profile measured by means of a paddle apparatus equipped without sinker at 50 rpm, 37±5° C., in 900 mL release medium, for the first 2 hours at pH 1.2 and thereafter at pH 6.8; wherein an in vitro release of 80 wt.-% of the pharmacologically active compound that was originally contained in the DR particles is achieved in ethanolic release medium at an ethanol concentration of 40 vol.-% later than in non-ethanolic release medium. Preferably, an in vitro release of 80 wt.-% of the pharmacologically active compound that was originally contained in the DR particles is achieved in ethanolic release medium at an ethanol concentration of 40 vol.-% at least 15 minutes later, more preferably at least 30 minutes later, still more preferably at least 45 minutes later, yet more preferably at least 60 minutes later, even more preferably at least 75 minutes later, most preferably at least 90 minutes later than in non-ethanolic release medium. For example, when under the given conditions an in vitro release of 80 wt.-% of the pharmacologically active compound that was originally contained in the DR particles is achieved after e.g. 157 minutes in non-ethanolic release medium, an in vitro release of 80 wt.-% of the pharmacologically active compound that was originally contained in the DR particles is achieved in ethanolic release medium at an ethanol concentration of 40 vol.-% at least 15 minutes later, i.e. is achieved not before 157+15 minutes=172 minutes.

Preferably, such coating comprises an inner layer comprising a hydrocolloid.

Hydrocolloids are a heterogeneous group of long chain polymers (polysaccharides and proteins) characterized by their property of forming viscous dispersions and/or gels when dispersed in water. For the purpose of the specification, a hydrocolloid is preferably selected from the group consisting of alginic acid, physiologically acceptable salts of alginic acid, agar, arabinoxylan, carrageenan (e.g. kappa-carrageenan), curdlan, gelatin, gellan, ß-glucan, guar, gum arabic, locust bean gum, pectin, wellan and xanthan; more preferably alginic acid, physiologically acceptable salts of alginic acid, carrageenan and xanthan; most preferably a physiologically acceptable salt of alginic acid (e.g. sodium alginate or another salt of alginic acid).

Further physiologically acceptable salts of alginic acid include the potassium salt, ammonium salt, magnesium salt and calcium salt. Preferably, the salt of alginic acid is sodium alginate. For the purpose of the specification, such inner layer belongs to the enteric coating.

Besides the alginate, preferably sodium alginate, the inner layer may comprise one or more excipients. Preferably, the inner layer comprises talcum. Preferably, the relative weight ratio of the alginate, preferably sodium alginate, to the talcum is within the range of 3:1 to 1:1, more preferably 2.5:1 to 1.5:1, still more preferably about 2:1.

Preferably, the weight content of the inner layer is at least 7.0 wt.-%, or at least 8.0 wt.-%, or at least 9.0 wt.-%, or at least 10 wt.-%, or at least 11 wt.-%, or at least 12 wt.-%, or at least 13 wt.-%, at least 14 wt.-%, or at least 15 wt.-%, or at least 16 wt.-%, or at least 17 wt.-%, or at least 18 wt.-%, or at least 19 wt.-%, based on the total weight of the DR particles. Preferably, the weight content of the inner layer is at most 27 wt.-%, or at most 26 wt.-%, or at most 25 wt.-%, or at most 24 wt.-%, or at most 23 wt.-%, or at most 22 wt.-%, at most 21 wt.-%, or at most 20 wt.-%, or at most 19 wt.-%, or at most 18 wt.-%, or at most 17 wt.-%, or at most 16 wt.-%, based on the total weight of the DR particles. Preferably, the weight content of the inner layer is within the range of from 10 to 25 wt.-%, more preferably within the range of from 15 to 20 wt.-%, based on the total weight of the DR particles.

In preferred embodiments, the weight content of the inner layer is within the range of 10±3 wt.-%, or 11±3 wt.-%, or 12±3 wt.-%, or 13±3 wt.-%, or 14±3 wt.-%, or 15±3 wt.-%, or 16±3 wt.-%, or 17±3 wt.-%, or 18±3 wt.-%, or 19±3 wt.-%, or 20±3 wt.-%, or 21±3 wt.-%, or 22±3 wt.-%, or 23±3 wt.-%, or 24±3 wt.-%, 10±2 wt.-%, or 11±2 wt.-%, or 12±2 wt.-%, or 13±2 wt.-%, or 14±2 wt.-%, or 15±2 wt.-%, or 16±2 wt.-%, or 17±2 wt.-%, or 18±2 wt.-%, or 19±2 wt.-%, or 20±2 wt.-%, or 21±2 wt.-%, or 22±2 wt.-%, or 23±2 wt.-%, or 24±2 wt.-%, 10±1 wt.-%, or 11±1 wt.-%, or 12±1 wt.-%, or 13±1 wt.-%, or 14±1 wt.-%, or 15±1 wt.-%, or 16±1 wt.-%, or 17±1 wt.-%, or 18±1 wt.-%, or 19±1 wt.-%, or 20±1 wt.-%, or 21±1 wt.-%, or 22±1 wt.-%, or 23±1 wt.-%, or 24±1 wt.-%, based on the total weight of the DR particles.

Preferably, such coating comprises an outer layer comprising an acrylate polymer. Preferably, the acrylate polymer is a random copolymer. For the purpose of the specification, such outer layer belongs to the enteric coating.

Preferably, the acrylate polymer is derived from a monomer mixture comprising methacrylic acid in combination with one or two comonomers selected from methyl acrylate, methyl methacrylate and ethyl acrylate.

In a preferred embodiment, the acrylate polymer is derived from a monomer mixture comprising methacrylic acid in combination with ethyl acrylate. Preferably, the enteric coating comprises an inner layer comprising sodium alginate or of another salt of alginic acid followed by an outer layer comprising a methacrylic acid-ethyl acrylate copolymer. Preferably, the methacrylic acid-ethyl acrylate copolymer has a ratio of free carboxyl groups to ester groups within the range of from 3:1 to 1:3, more preferably 2:1 to 1:2.

In another preferred embodiment, the acrylate polymer is derived from a monomer mixture comprising methacrylic acid in combination with methyl acrylate and methyl methacrylate. Preferably, the enteric coating comprises an inner layer comprising sodium alginate or of another salt of alginic acid followed by an outer layer comprising an anionic copolymer based on methyl acrylate, methyl methacrylate and methacrylic acid. Preferably, the anionic copolymer has a ratio of free carboxyl groups to ester groups within the range of from 1:8 to 1:12, more preferably 1:9 to 1:11.

Preferably, the acrylate polymer has a weight average molecular weight of at least 50,000 g/mol, or at least 100,000 g/mol, or at least 150,000 g/mol, or at least 200,000 g/mol, or at least 250,000 g/mol. Preferably, the acrylate polymer has a weight average molecular weight of at most 500,000 g/mol, or at most 450,000 g/mol, or at most 400,000 g/mol, or at most 350,000 g/mol, or at most 300,000 g/mol. Preferably, the acrylate polymer has a weight average molecular weight within the range of from 200,000 to 400,000 g/mol, more preferably within the range of from 250,000 to 350,000 g/mol.

Preferably, the weight content of the outer layer is at least 12 wt.-%, or at least 13 wt.-%, or at least 14 wt.-%, or at least 15 wt.-%, or at least 16 wt.-%, or at least 17 wt.-%, or at least 18 wt.-%, or at least 19 wt.-%, or at least 20 wt.-%, or at least 21 wt.-%, or at least 22 wt.-%, at least 23 wt.-%, or at least 24 wt.-%, or at least 25 wt.-%, or at least 26 wt.-%, based on the total weight of the DR particles. Preferably, the weight content of the outer layer is at most 35 wt.-%, or at most 34 wt.-%, or at most 33 wt.-%, or at most 32 wt.-%, or at most 31 wt.-%, or at most 30 wt.-%, or at most 29 wt.-%, or at most 28 wt.-%, or at most 27 wt.-%, or at most 26 wt.-%, at most 25 wt.-%, or at most 24 wt.-%, or at most 19 wt.-%, or at most 18 wt.-%, based on the total weight of the DR particles. Preferably, the weight content of the outer layer is within the range of from 15 to 35 wt.-%, more preferably within the range of from 20 to 30 wt.-%, based on the total weight of the DR particles.

In preferred embodiments, the weight content of the outer layer is within the range of 15±10 wt.-%, or 16±10 wt.-%, or 17±10 wt.-%, or 18±10 wt.-%, or 19±10 wt.-%, or 20±10 wt.-%, or 21±10 wt.-%, or 22±10 wt.-%, or 23±10 wt.-%, or 24±10 wt.-%, or 25±10 wt.-%, or 26±10 wt.-%, or 27±10 wt.-%, or 28±10 wt.-%, or 29±10 wt.-%, or 30±10 wt.-%, or 31±10 wt.-%, or 32±10 wt.-%, 15±8 wt.-%, or 16±8 wt.-%, or 17±8 wt.-%, or 18±8 wt.-%, or 19±8 wt.-%, or 20±8 wt.-%, or 21±8 wt.-%, or 22±8 wt.-%, or 23±8 wt.-%, or 24±8 wt.-%, or 25±8 wt.-%, or 26±8 wt.-%, or 27±8 wt.-%, or 28±8 wt.-%, or 29±8 wt.-%, or 30±8 wt.-%, or 31±8 wt.-%, or 32±8 wt.-%, 15±5 wt.-%, or 16±5 wt.-%, or 17±5 wt.-%, or 18±5 wt.-%, or 19±5 wt.-%, or 20±5 wt.-%, or 21±5 wt.-%, or 22±5 wt.-%, or 23±5 wt.-%, or 24±5 wt.-%, or 25±5 wt.-%, or 26±5 wt.-%, or 27±5 wt.-%, or 28±5 wt.-%, or 29±5 wt.-%, or 30±5 wt.-%, or 31±5 wt.-%, or 32±5 wt.-%, based on the total weight of the DR particles.

In preferred embodiments, the weight content of the outer layer is within the range of 15±3 wt.-%, or 16±3 wt.-%, or 17±3 wt.-%, or 18±3 wt.-%, or 19±3 wt.-%, or 20±3 wt.-%, or 21±3 wt.-%, or 22±3 wt.-%, or 23±3 wt.-%, or 24±3 wt.-%, or 25±3 wt.-%, or 26±3 wt.-%, or 27±3 wt.-%, or 28±3 wt.-%, or 29±3 wt.-%, or 30±3 wt.-%, or 31±3 wt.-%, or 32±3 wt.-%, 15±2 wt.-%, or 16±2 wt.-%, or 17±2 wt.-%, or 18±2 wt.-%, or 19±2 wt.-%, or 20±2 wt.-%, or 21±2 wt.-%, or 22±2 wt.-%, or 23±2 wt.-%, or 24±2 wt.-%, or 25±2 wt.-%, or 26±2 wt.-%, or 27±2 wt.-%, or 28±2 wt.-%, or 29±2 wt.-%, or 30±2 wt.-%, or 31±2 wt.-%, or 32±2 wt.-%, 15±1 wt.-%, or 16±1 wt.-%, or 17±1 wt.-%, or 18±1 wt.-%, or 19±1 wt.-%, or 20±1 wt.-%, or 21±1 wt.-%, or 22±1 wt.-%, or 23±1 wt.-%, or 24±1 wt.-%, or 25±1 wt.-%, or 26±1 wt.-%, or 27±1 wt.-%, or 28±1 wt.-%, or 29±1 wt.-%, or 30±1 wt.-%, or 31±1 wt.-%, or 32±1 wt.-%, based on the total weight of the DR particles.

Preferably, such enteric coating comprises an outer layer of an acrylate polymer or copolymer, which is preferably a random copolymer. Preferably, the acrylate polymer or copolymer is based on methacrylic acid in combination with one or two comonomers selected from methyl acrylate, methyl methacrylate and ethyl acrylate. Preferably, the acrylate polymer or copolymer has a weight average molecular weight within the range of from 200,000 to 400,000 g/mol, more preferably from 250,000 to 350,000 g/mol, preferably determined by size exclusion chromatography.

In a particularly preferred embodiment, such enteric coating comprises an inner layer of sodium alginate (or of another salt of alginic acid) followed by an outer layer of an acrylate polymer or copolymer, e.g. a methacrylic acid-ethyl acrylate copolymer (bipolymer), preferably random copolymer, such as a methacrylic acid-ethyl acrylate copolymer, preferably having a ratio of free carboxyl groups to ester groups within the range of from 3:1 to 1:3, more preferably from 2:1 to 1:2, in particular about 1:1; and/or preferably having a weight average molecular weight within the range of from 250,000 to 400,000 g/mol, more preferably from 300,000 to 350,000 g/mol, preferably determined by size exclusion chromatography (e.g. Eudragit® L 100-55, Acryl-EZE®, Eudragit® L 30 D-55, or PlasACRYL™HTP20).

In another particularly preferred embodiment, such enteric coating comprises an inner layer of sodium alginate (or of another salt of alginic acid) followed by an outer layer of an acrylate polymer or copolymer, e.g. an anionic copolymer based on methyl acrylate, methyl methacrylate and methacrylic acid, i.e. a methyl acrylate-methyl methacrylate-methacrylic acid copolymer (terpolymer), preferably random copolymer, preferably having a ratio of free carboxyl groups to ester groups within the range of from 1:8 to 1:12, more preferably from 1:9 to 1:11, in particular about 1:10; and/or preferably having a weight average molecular weight within the range of from 200,000 to 400,000 g/mol, more preferably from 250,000 to 300,000 g/mol, preferably determined by size exclusion chromatography (e.g. Eudragit® FS 30 D or PlasACRYL™ T20).

In still another particularly preferred embodiment, such enteric coating comprises an inner layer of sodium alginate (or of another salt of alginic acid) followed by an outer layer of an acrylate polymer or copolymer, e.g. an anionic copolymer based on methyl methacrylate and methacrylic acid, i.e. a methyl methacrylate-methacrylic acid copolymer (bipolymer), preferably random copolymer, preferably having a ratio of free carboxyl groups to ester groups within the range of from
(i) 3:1 to 1:3, more preferably from 2:1 to 1:2, in particular about 1:1 (e.g., Eudragit® L 100 or Eudragit® L 12,5); or
(ii) 2:1 to 1:4, more preferably from 1:1 to 1:3, in particular about 1:2 (e.g., Eudragit® S 100 or Eudragit® S 12,5);
   and/or in either case preferably having a weight average molecular weight within the range of from 50,000 to 200,000 g/mol, more preferably from 100,000 to 150,000 g/mol, preferably determined by size exclusion chromatography.

In preferred embodiments, such enteric coating comprises an inner layer of sodium alginate (or of another salt of alginic acid) followed by an outer layer of a mixture of two or more different acrylate polymers or copolymers, wherein said mixture preferably comprises a first acrylate copolymer and a second acrylate copolymer, which are independently selected from the group consisting of methacrylic acid-ethyl acrylate copolymers as defined above, methyl acrylate-methyl methacrylate-methacrylic acid copolymers as defined above, and methyl methacrylate-methacrylic acid copolymers as defined above; preferably wherein the relative weight ratio of the first acrylate copolymer to the second acrylate copolymer is within the range of from 10:1 to 1:10, or 10:1 to 1.1:1, or 1:10 to 1:1.1; more preferably 5:1 to 1:5, or 5:1 to 1.1:1, or 1:5 to 1:1.1; still more preferably 2:1 to 1:2, or 2:1 to 1.1:1, or 1:2 to 1:1.1. In preferred embodiments,
the first acrylate copolymer is a methacrylic acid-ethyl acrylate copolymer as defined above and the second acrylate copolymer is a methyl acrylate-methyl methacrylate-methacrylic acid copolymer as defined above; or
the first acrylate copolymer is a methacrylic acid-ethyl acrylate copolymer as defined above and the second acrylate copolymer is a methyl methacrylate-methacrylic acid copolymer as defined above; or
the first acrylate copolymer is a methyl acrylate-methyl methacrylate-methacrylic acid copolymer as defined above and the second acrylate copolymer is a methyl methacrylate-methacrylic acid copolymer as defined above.

Alternative acrylate polymers or copolymers that may be used to overcoat in inner layer of sodium alginate include but are not limited to aminoalkyl methacrylate copolymers (e.g. Eudragit® K) and ethylacrylate methylmethacrylate copolymers (e.g. Eudragit® N, such as Eudragit® NE 30 D).

Besides the acrylate polymer, the outer layer may comprise one or more excipients. Preferably, the outer layer comprises talcum. Preferably, the relative weight ratio of the acrylic polymer to the talcum is within the range of 9:1 to 4:1, more preferably 8:1 to 5:1, still more preferably about 7:1 to 6:1. Preferably, the outer layer comprises a plasticizer, preferably triethyl citrate. Preferably, the relative weight ratio of the acrylic polymer to the plasticizer is within the range of 25:1 to 15:1, more preferably 22:1 to 18:1, still more preferably about 21:1 to 19:1.

A particularly preferred enteric coating composition that provides resistance against dose dumping in aqueous ethanol is commercialized by Evonik as Eudratec® ADD. Preferably, the DR particles according to the invention are film coated with can enteric coating comprising
an inner layer of sodium alginate (or another salt of alginic acid) followed by an outer layer of an acrylate (e.g. Eudragit®) polymer, e.g. a methacrylic acid-ethyl acrylate copolymer (1:1) (e.g. Eudragit® L 30 D-55); or
an inner layer of sodium alginate (or another salt of alginic acid) followed by an outer layer of an acrylate (e.g. Eudragit®) polymer, e.g. a methacrylic acid-methyl acrylate-methyl methacrylate copolymer (1:10) (e.g. Eudragit® FS 30 D); or
an inner layer of sodium alginate (or another salt of alginic acid) followed by an outer layer of an acrylate (e.g. Eudragit®) polymer, e.g. a methyl methacrylate-methacrylic acid copolymer (1:1) (e.g., Eudragit® L 100 or Eudragit® L 12,5); or
an inner layer of sodium alginate (or another salt of alginic acid) followed by an outer layer of an acrylate (e.g. Eudragit®) polymer, e.g. a methyl methacrylate-methacrylic acid copolymer (1:2) (e.g., Eudragit® S 100 or Eudragit® S 12,5); or
an inner layer of sodium alginate (or another salt of alginic acid) followed by an outer layer of a mixture of a first acrylate (e.g. Eudragit®) polymer with a second acrylate (e.g. Eudragit®) polymer, which are independently selected from the group consisting of methacrylic acid-ethyl acrylate copolymers (1:1), methacrylic acid-methyl acrylate-methyl methacrylate copolymers (1:10), methyl methacrylate-methacrylic acid copolymers (1:1), and methyl methacrylate-methacrylic acid copolymers (1:2).

When the particles are film coated with an enteric coating material (DR particles), the content of the dried enteric coating is preferably at most 30 wt.-%, more preferably at most 29 wt.-%, still more preferably at most 28 wt.-%, yet more preferably at most 27 wt.-%, most preferably at most 26 wt.-%, and in particular at most 25 wt.-%, based on the total weight of the DR particles.

It has been surprisingly found that the in vitro release properties, especially also the in vitro release properties in ethanolic medium compared to non-ethanolic medium, can be tailored by
(i) the chemical nature of the material forming the inner layer of the enteric coating;
(ii) the absolute amount of the material forming the inner layer of the enteric coating;
(iii) the chemical nature of the material forming the outer layer of the enteric coating;
(iv) the absolute amount of the material forming the outer layer of the enteric coating; and/or
(v) the relative weight ratio of the absolute amount of the material forming the inner layer of the enteric coating to the absolute amount of the material forming the outer layer of the enteric coating.

Preferably, the weight content of the enteric coating is at least 30 wt.-%, or at least 31 wt.-%, or at least 32 wt.-%, or at least 33 wt.-%, or at least 34 wt.-%, or at least 35 wt.-%, or at least 36 wt.-%, at least 37 wt.-%, or at least 38 wt.-%, or at least 39 wt.-%, or at least 40 wt.-%, based on the total weight of the enteric coating and based on the total weight of the DR particles. Preferably, the weight content of the enteric coating is at most 50 wt.-%, or at most 49 wt.-%, or at most 48 wt.-%, or at most 47 wt.-%, or at most 46 wt.-%, or at most 45 wt.-%, at most 44 wt.-%, or at most 43 wt.-%, or at most 42 wt.-%, or at most 41 wt.-%, based on the total weight of the enteric coating and based on the total weight of the DR particles.

In preferred embodiments, the weight content of the enteric coating is within the range of 33±3 wt.-%, or 34±3 wt.-%, or 35±3 wt.-%, or 36±3 wt.-%, or 37±3 wt.-%, or 38±3 wt.-%, or 39±3 wt.-%, or 40±3 wt.-%, or 41±3 wt.-%, or 42±3 wt.-%, or 43±3 wt.-%, or 44±3 wt.-%, or 45±3 wt.-%, or 46±3 wt.-%, or 47±3 wt.-%, 33±2 wt.-%, or 34±2 wt.-%, or 35±2 wt.-%, or 36±2 wt.-%, or 37±2 wt.-%, or 38±2 wt.-%, or 39±2 wt.-%, or 40±2 wt.-%, or 41±2 wt.-%, or 42±2 wt.-%, or 43±2 wt.-%, or 44±2 wt.-%, or 45±2 wt.-%, or 46±2 wt.-%, or 47±2 wt.-%, 33±1 wt.-%, or 34±1 wt.-%, or 35±1 wt.-%, or 36±1 wt.-%, or 37±1 wt.-%, or 38±1 wt.-%, or 39±1 wt.-%, or 40±1 wt.-%, or 41±1 wt.-%, or 42±1 wt.-%, or 43±1 wt.-%, or 44±1 wt.-%, or 45±1 wt.-%, or 46±1 wt.-%, or 47±1 wt.-%, based on the total weight of the enteric coating and based on the total weight of the DR particles.

Preferably, the weight of the outer layer exceeds the weight of the inner layer.

Preferably, the relative weight ratio of the outer layer to the inner layer is within the range of from 0.8:1.0 to 1.8:1.0, more preferably 0.9:1.0 to 1.7:1.0, still more preferably 1.0:1.0 to 1.6:1.0, yet more preferably 1.1:1.0 to 1.5:1.0, even more preferably, 1.2:1.0 to 1.4:1.0, most preferably of about 1.3:1.0, based on the total weight of the outer layer and based on the total weight of the inner layer.

Preferably, the total weight of the outer layer is at least 1.5-times higher, more preferably at least 1.7-times higher, still more preferably at least 1.9-times higher than the total weight of the inner layer.

Preferred DR particles are hot-melt extruded. Preferred DR particles contain as the pharmacologically active compound a stimulant, preferably amphetamine or a physiologically acceptable salt thereof, more preferably amphetamine sulfate. Preferably, these DR particles comprises
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid,
optionally a non-enteric coating based on hydroxypropylmethylcellulose or based on polyvinylalcohol, and
an enteric coating, preferably based on an acrylate polymer or a mixture of acrylate polymers.

Particularly preferred embodiments O¹ to O⁶ of such DR particles are summarized in the table here below (all percentages relative to the total weight of the DR particles):

| [wt.-%] | O¹ | O² | O³ | O⁴ | O⁵ | O⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 45 ± 40 | 45 ± 35 | 45 ± 30 | 45 ± 25 | 45 ± 20 | 45 ± 15 |
| disintegrant | 15 ± 14 | 15 ± 13 | 15 ± 12 | 15 ± 11 | 15 ± 10 | 15 ± 9 |
| optionally, plasticizer | 15 ± 14 | 15 ± 13 | 15 ± 12 | 15 ± 11 | 15 ± 10 | 15 ± 9 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, non-enteric coating, | 5.0 ± 4.5 | 5.0 ± 4.0 | 5.0 ± 3.5 | 5.0 ± 3.0 | 5.0 ± 2.5 | 5.0 ± 2.0 |
| which does not delay | | | | | | |
| in vitro dissolution | | | | | | |
| enteric coating | 24 ± 20 | 24 ± 18 | 24 ± 16 | 24 ± 14 | 24 ± 12 | 24 ± 10 |

Preferred DR particles are hot-melt extruded. Preferred DR particles contain as the pharmacologically active compound a stimulant, preferably amphetamine or a physiologically acceptable salt thereof, more preferably amphetamine sulfate. Preferably, these DR particles comprise
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid,
optionally a non-enteric coating based on hydroxypropylmethylcellulose or based on polyvinylalcohol,
an inner enteric coating layer, preferably based on an alginate, preferably based on sodium alginate, and
an outer enteric coating layer, preferably based on an acrylate polymer or a mixture of acrylate polymers.

When the DR particles comprise a non-enteric coating as well as an enteric coating comprising inner layer and outer layer, the non-enteric coating is preferably applied first, followed by the inner layer of the enteric coating and followed by the second layer of the enteric coating, such that said second layer of the enteric coating preferably forms the outermost layer of the coated DR particles.

Particularly preferred embodiments P¹ to P⁶ of such DR particles are summarized in the table here below (all percentages relative to the total weight of the DR particles):

| [wt.-%] | P¹ | P² | P³ | P⁴ | P⁵ | P⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 45 ± 40 | 45 ± 35 | 45 ± 30 | 45 ± 25 | 45 ± 20 | 45 ± 15 |
| disintegrant | 15 ± 14 | 15 ± 13 | 15 ± 12 | 15 ± 11 | 15 ± 10 | 15 ± 9 |
| optionally, plasticizer | 15 ± 14 | 15 ± 13 | 15 ± 12 | 15 ± 11 | 15 ± 10 | 15 ± 9 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, | 5.0 ± 4.5 | 5.0 ± 4.0 | 5.0 ± 3.5 | 5.0 ± 3.0 | 5.0 ± 2.5 | 5.0 ± 2.0 |
| non-enteric coating | | | | | | |
| inner enteric coating | 16.9 ± 4.0 | 16.9 ± 3.5 | 16.9 ± 3.0 | 16.9 ± 2.5 | 16.9 ± 2.0 | 16.9 ± 1.5 |
| layer comprising alginate | | | | | | |
| outer enteric coating layer | 26.9 ± 4.0 | 26.9 ± 3.5 | 26.9 ± 3.0 | 26.9 ± 2.5 | 26.9 ± 2.0 | 26.9 ± 1.5 |
| comprising acrylic polymer | | | | | | |

Particularly preferred embodiments Q¹ to Q⁶ of such DR particles are summarized in the table here below (all percentages relative to the total weight of the DR particles):

| [wt.-%] | Q¹ | Q² | Q³ | Q⁴ | Q⁵ | Q⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 45 ± 40 | 45 ± 35 | 45 ± 30 | 45 ± 25 | 45 ± 20 | 45 ± 15 |
| disintegrant | 15 ± 14 | 15 ± 13 | 15 ± 12 | 15 ± 11 | 15 ± 10 | 15 ± 9 |
| optionally, plasticizer | 15 ± 14 | 15 ± 13 | 15 ± 12 | 15 ± 11 | 15 ± 10 | 15 ± 9 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, | 5.0 ± 4.5 | 5.0 ± 4.0 | 5.0 ± 3.5 | 5.0 ± 3.0 | 5.0 ± 2.5 | 5.0 ± 2.0 |
| non-enteric coating | | | | | | |
| inner enteric coating | 17.8 ± 4.0 | 17.8 ± 3.5 | 17.8 ± 3.0 | 17.8 ± 2.5 | 17.8 ± 2.0 | 17.8 ± 1.5 |
| layer comprising alginate | | | | | | |
| outer enteric coating layer | 23.1 ± 4.0 | 23.1 ± 3.5 | 23.1 ± 3.0 | 23.1 ± 2.5 | 23.1 ± 2.0 | 23.1 ± 1.5 |
| comprising acrylic polymer | | | | | | |

Particularly preferred embodiments R¹ to R⁶ of such DR particles are summarized in the table here below (all percentages relative to the total weight of the DR particles):

| [wt.-%] | R¹ | R² | R³ | R⁴ | R⁵ | R⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 45 ± 40 | 45 ± 35 | 45 ± 30 | 45 ± 25 | 45 ± 20 | 45 ± 15 |
| disintegrant | 15 ± 14 | 15 ± 13 | 15 ± 12 | 15 ± 11 | 15 ± 10 | 15 ± 9 |
| optionally plasticizer | 15 ± 14 | 15 ± 13 | 15 ± 12 | 15 ± 11 | 15 ± 10 | 15 ± 9 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, non-enteric coating | 4.0 ± 3.2 | 4.0 ± 2.9 | 4.0 ± 2.6 | 4.0 ± 2.3 | 4.0 ± 2.0 | 4.0 ± 1.7 |
| which does not delay | | | | | | |
| in vitro dissolution | | | | | | |
| inner enteric coating layer | 18.0 ± 13.6 | 18.0 ± 11.0 | 18.0 ± 8.4 | 18.0 ± 5.8 | 18.0 ± 3.2 | 18.0 ± 1.8 |
| comprising alginate | | | | | | |
| outer layer comprising | 34.9 ± 26.1 | 34.9 ± 22.8 | 34.9 ± 19.5 | 34.9 ± 16.2 | 34.9 ± 12.9 | 34.9 ± 9.7 |
| acrylic polymer | | | | | | |

Particularly preferred embodiments S¹ to S⁶ of such DR particles are summarized in the table here below (all percentages relative to the total weight of the DR particles):

| [wt.-%] | S¹ | S² | S³ | S⁴ | S⁵ | S⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 45 ± 40 | 45 ± 35 | 45 ± 30 | 45 ± 25 | 45 ± 20 | 45 ± 15 |
| disintegrant | 15 ± 14 | 15 ± 13 | 15 ± 12 | 15 ± 11 | 15 ± 10 | 15 ± 9 |
| optionally plasticizer | 15 ± 14 | 15 ± 13 | 15 ± 12 | 15 ± 11 | 15 ± 10 | 15 ± 9 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, non-enteric coating | 4.0 ± 3.2 | 4.0 ± 2.9 | 4.0 ± 2.6 | 4.0 ± 2.3 | 4.0 ± 2.0 | 4.0 ± 1.7 |
| which does not delay | | | | | | |
| in vitro dissolution | | | | | | |
| alginate in inner | 12.0 ± 10.0 | 12.0 ± 8.0 | 12.0 ± 6.0 | 12.0 ± 4.0 | 12.0 ± 2.0 | 12.0 ± 1.2 |
| enteric coating layer | | | | | | |
| optionally, talcum in inner | 6.0 ± 3.6 | 6.0 ± 3.0 | 6.0 ± 2.4 | 6.0 ± 1.8 | 6.0 ± 1.2 | 6.0 ± 0.6 |
| enteric coating layer | | | | | | |
| acrylic polymer in outer | 29.1 ± 23.0 | 29.1 ± 20.0 | 29.1 ± 17.0 | 29.1 ± 14.0 | 29.1 ± 11.0 | 29.1 ± 8.1 |
| enteric coating layer | | | | | | |
| optionally, plasticizer in | 1.4 ± 0.9 | 1.4 ± 0.8 | 1.4 ± 0.7 | 1.4 ± 0.6 | 1.4 ± 0.5 | 1.4 ± 0.4 |
| outer enteric coating layer | | | | | | |
| optionally, talcum in outer | 4.4 ± 2.2 | 4.4 ± 2.0 | 4.4 ± 1.8 | 4.4 ± 1.6 | 4.4 ± 1.4 | 4.4 ± 1.2 |
| enteric coating layer | | | | | | |

Preferably, each of said DR particles has an individual weight of less than 20 mg, more preferably not more than 15 mg, still more preferably not more than 10 mg, yet more preferably not more than 7.5 mg, most preferably not more than 5.0 mg and in particular not more than 2.5 mg. According to this embodiment, the pharmaceutical dosage form preferably does not comprise PR particle(s) (see above). Therefore, according to this preferred embodiment, the pharmaceutical dosage form comprises a multitude of IR particles in combination with a multitude of DR particles, but preferably neither a single nor a few PR particle(s).

### Or Particles:

In a preferred embodiment, the CR particles are coated with an enteric coating which further retards in vitro dissolution and which contains a mixture of two different acrylate polymers thereby rendering the particles postponed release particles (OR particles). Said coating may have a single layer or more than a single layer, e.g. two layers. Suitable coating materials for OR particles are commercially available.

Preferably, each of said OR particles has an individual weight of less than 20 mg, more preferably not more than 10 mg.

The multitude of OR particles provides postponed release of the pharmacologically active compound.

For the purpose of the specification, “postponed release” refers to oral medicines that do not immediately disintegrate and release the active ingredient(s) into the body. For the purpose of the specification, postponed release is preferably even more delayed than delayed release. The OR particles according to the invention are preferably enterically coated such that they dissolve in the intestine rather than the stomach.

When tested alone (i.e. in the absence of particles of other type and nature), said multitude of OR particles provide postponed release of the pharmacologically active compound such that under in vitro conditions in accordance with Ph. Eur. after 30 minutes in artificial gastric juice at pH 1.2 preferably less than 50%, more preferably at most 40 wt.-%, still more preferably at most 30 wt.-%, yet more preferably at most 10 wt.-% of the pharmacologically active compound that were originally contained in said multitude of OR particles have been released.

When tested alone (i.e. in the absence of particles of other type and nature), said multitude of OR particles provide postponed release of the pharmacologically active compound such that under in vitro conditions in accordance with Ph. Eur., when changing the release medium after 120 minutes from initially artificial gastric juice with pH 1.2 to subsequently artificial intestinal fluid with pH 6.8, after 180 minutes preferably less than 20%, more preferably at most 17.5 wt.-%, still more preferably at most 15 wt.-%, yet more preferably at most 10 wt.-% of the pharmacologically active compound that were originally contained in said multitude of OR particles have been released.

Preferably, the content of the dried enteric coating of the OR particles according to the invention is at least 12 wt.-%, more preferably at least 13 wt.-%, still more preferably at least 14 wt.-%, yet more preferably at least 15 wt.-%, most preferably at least 16 wt.-%, and in particular at least 17 wt.-%, based on the total weight of the OR particles. This is a significant difference of the OR particles compared to the FR particles, which in turn typically have a lower content of enteric coating material.

Preferably, all preferred embodiments that have been defined above for the DR particles according to the invention analogously apply to the OR particles according to the invention.

In preferred embodiments, such enteric coating of the OR particles comprises an inner layer of sodium alginate (or of another salt of alginic acid) followed by an outer layer of a mixture of two or more different acrylate polymers or copolymers, wherein said mixture preferably comprises a first acrylate polymer and a second acrylate polymer, which are independently selected from the group consisting of
methyl acrylate-methyl methacrylate-methacrylic acid copolymers as defined above,
methacrylic acid-ethyl acrylate copolymers as defined above,
methyl methacrylate-methacrylic acid copolymers as defined above.

In preferred embodiments,
the first acrylate polymer is a methyl acrylate-methyl methacrylate-methacrylic acid copolymer as defined above and the second acrylate copolymer is a methacrylic acid-ethyl acrylate copolymer as defined above; or
the first acrylate polymer is a methyl acrylate-methyl methacrylate-methacrylic acid copolymer as defined above and the second acrylate copolymer is a methyl methacrylate-methacrylic acid copolymer as defined above.

According to a particularly preferred embodiment of the OR particles according to the invention,
(i) the first acrylate polymer is an anionic copolymer based on methyl acrylate, methyl methacrylate and methacrylic acid, i.e. a methyl acrylate-methyl methacrylate-methacrylic acid copolymer (terpolymer), preferably random copolymer, preferably having a ratio of free carboxyl groups to ester groups within the range of from 1:8 to 1:12, more preferably from 1:9 to 1:11, in particular about 1:10; and/or preferably having a weight average molecular weight within the range of from 200,000 to 400,000 g/mol, more preferably from 250,000 to 300,000 g/mol, preferably determined by size exclusion chromatography (e.g. Eudragit® FS 30 D or PlasACRYL™ T20); and/or
(ii) the second acrylate polymer is a methacrylic acid-ethyl acrylate copolymer (bipolymer), preferably random copolymer, such as a methacrylic acid-ethyl acrylate copolymer, preferably having a ratio of free carboxyl groups to ester groups within the range of from 3:1 to 1:3, more preferably from 2:1 to 1:2, in particular about 1:1; and/or preferably having a weight average molecular weight within the range of from 250,000 to 400,000 g/mol, more preferably from 300,000 to 350,000 g/mol, preferably determined by size exclusion chromatography (e.g. Eudragit® L 100-55, Acryl-EZE®, Eudragit® L 30 D-55, or PlasACRYL™ HTP20).

The relative weight ratio of the first acrylate polymer to the second acrylate polymer is preferably within the range of from 81:19 to 99:1, or from 82:18 to 98:2, or from 83:17 to 97:3, or from 84:16 to 96:4, or from 85:15 to 95:5, or from 86:14 to 94:6, or from 87:13 to 93:7, or from 88:12 to 92:8, or from 89:11 to 91:9, or about 90:10.

Preferred OR particles are hot-melt extruded. Preferred OR particles contain as the pharmacologically active compound a stimulant, preferably amphetamine or a physiologically acceptable salt thereof, more preferably amphetamine sulfate. Preferably, these OR particles comprise
a polyalkylene oxide which is a polyethylene oxide with a weight average molecular weight within the range of from 0.5 to 15 million g/mol,
a disintegrant, preferably starch or pretreated starch, preferably pregelatinized starch,
optionally a plasticizer, preferably a polyethylene glycol,
optionally an antioxidant, preferably alpha-tocopherol,
optionally an acid, preferably citric acid,
optionally a non-enteric coating based on hydroxypropylmethylcellulose or based on polyvinylalcohol,
an inner enteric coating layer, preferably based on an alginate, preferably based on sodium alginate, and
an outer enteric coating layer, preferably based on a mixture of a first acrylate polymer and a second acrylate polymer, preferably wherein the first acrylate polymer is a methyl acrylate
methyl methacrylate-methacrylic acid copolymer and the second acrylate polymer is a methacrylic acid-ethyl acrylate copolymer, preferably wherein the relative weight ratio of the first acrylate polymer to the second acrylate polymer is preferably within the range of from 85:15 to 95:5, or from 87:13 to 93:7, or from 89:11 to 91:9, or about 90:10.

Particularly preferred embodiments T¹ to T⁶ of such OR particles are summarized in the table here below (all percentages relative to the total weight of the OR particles):

| [wt.-%] | T¹ | T² | T³ | T⁴ | T⁵ | T⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 35 ± 33 | 35 ± 30 | 35 ± 27 | 35 ± 24 | 35 ± 21 | 35 ± 18 |
| disintegrant | 10.0 ± 9.5 | 10.0 ± 9.0 | 10.0 ± 8.5 | 10.0 ± 8.0 | 10.0 ± 7.5 | 10.0 ± 7.0 |
| optionally, plasticizer | 10.0 ± 9.5 | 10.0 ± 9.0 | 10.0 ± 8.5 | 10.0 ± 8.0 | 10.0 ± 7.5 | 10.0 ± 7.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, | 5.0 ± 4.5 | 5.0 ± 4.0 | 5.0 ± 3.5 | 5.0 ± 3.0 | 5.0 ± 2.5 | 5.0 ± 2.0 |
| non-enteric coating | | | | | | |
| inner enteric coating | 19.0 ± 18.0 | 19.0 ± 17.0 | 19.0 ± 16.0 | 19.0 ± 15.0 | 19.0 ± 14.0 | 19.0 ± 13.0 |
| layer comprising alginate | | | | | | |
| outer enteric coating layer | 37 ± 35 | 37 ± 33 | 37 ± 30 | 37 ± 27 | 37 ± 24 | 37 ± 21 |
| comprising first acrylic polymer | | | | | | |
| and second acrylic polymer | | | | | | |

Particularly preferred embodiments U¹ to U⁶ of such OR particles are summarized in the table here below (all percentages relative to the total weight of the OR particles):

| [wt.-%] | U¹ | U² | U³ | U⁴ | U⁵ | U⁶ |
|---|---|---|---|---|---|---|
| stimulant | 9.0 ± 8.5 | 9.0 ± 8.0 | 9.0 ± 7.5 | 9.0 ± 7.0 | 9.0 ± 6.5 | 9.0 ± 6.0 |
| polyethylene oxide | 35 ± 33 | 35 ± 30 | 35 ± 27 | 35 ± 24 | 35 ± 21 | 35 ± 18 |
| disintegrant | 10.0 ± 9.5 | 10.0 ± 9.0 | 10.0 ± 8.5 | 10.0 ± 8.0 | 10.0 ± 7.5 | 10.0 ± 7.0 |
| optionally plasticizer | 10.0 ± 9.5 | 10.0 ± 9.0 | 10.0 ± 8.5 | 10.0 ± 8.0 | 10.0 ± 7.5 | 10.0 ± 7.0 |
| optionally, antioxidant | 1.00 ± 0.95 | 1.00 ± 0.95 | 1.00 ± 0.95 | 1.00 ± 0.95 | 1.00 ± 0.95 | 1.00 ± 0.95 |
| optionally, acid | 1.00 ± 0.95 | 1.00 ± 0.95 | 1.00 ± 0.95 | 1.00 ± 0.95 | 1.00 ± 0.95 | 1.00 ± 0.95 |
| optionally, non-enteric | 5.0 ± 4.5 | 5.0 ± 4.0 | 5.0 ± 3.5 | 5.0 ± 3.0 | 5.0 ± 2.5 | 5.0 ± 2.0 |
| coating which does not delay | | | | | | |
| in vitro dissolution | | | | | | |
| inner enteric coating layer | 19.0 ± 18.0 | 19.0 ± 17.0 | 19.0 ± 16.0 | 19.0 ± 15.0 | 19.0 ± 14.0 | 19.0 ± 13.0 |
| comprising alginate | | | | | | |
| first acrylic polymer in | 19.8 ± 19.0 | 19.8 ± 17.0 | 19.8 ± 15.0 | 19.8 ± 13.0 | 19.8 ± 11.0 | 19.8 ± 9.0 |
| outer enteric coating layer | | | | | | |
| second acrylic polymer in | 2.2 ± 2.1 | 2.2 ± 2.0 | 2.2 ± 1.9 | 2.2 ± 1.8 | 2.2 ± 1.7 | 2.2 ± 1.6 |
| outer enteric coating layer | | | | | | |

Preferably, each of said OR particles has an individual weight of less than 20 mg, more preferably not more than 15 mg, still more preferably not more than 10 mg, yet more preferably not more than 7.5 mg, most preferably not more than 5.0 mg and in particular not more than 2.5 mg. According to this embodiment, the pharmaceutical dosage form preferably does not comprise OR particle(s) (see above). Therefore, according to this preferred embodiment, the pharmaceutical dosage form comprises a multitude of IR particles in combination with a multitude of OR particles, but preferably neither a single nor a few PR particle(s).

Combinations of Different Particles with One Another

As will be explained in more detail hereinafter, in preferred embodiments the present invention relates to combinations of
(i) a multitude of immediate release particles with at least one prolonged release particle (IR particles+PR particle);
(ii) a multitude of immediate release particles with a multitude of delayed release particles (IR particles+DR particles);
(iii) a multitude of immediate release particles with a multitude of postponed release particles (IR particles+OR particles);
(iv) a multitude of fast release particles with at least one prolonged release particle (FR particles+PR particle);
(v) a multitude of fast release particles with a multitude of delayed release particles (FR particles+DR particles); and
(vi) a multitude of fast release particles with a multitude of postponed release particles (FR particles+OR particles).

According to a preferred embodiment, the pharmaceutical dosage form according to the invention comprises a multitude of IR particles in combination with a single or a few PR particle(s), but preferably neither FR particles nor DR particles nor OR particles.

Preferably, the IR particles are in accordance with any of above embodiments A¹ to A⁶, or B¹ to B⁶, or C¹ to C⁶, or D¹ to D⁶, or E¹ to E⁶, or F¹ to F⁶, whereas the PR particle(s) are in accordance with any of above embodiments M¹ to M⁶, or N¹ to N⁶. Preferred individualized combinations of embodiments are: A¹+M¹, A¹+M², A¹+M³, A¹+M⁴, A¹+M⁵, A¹+M⁶; A²+M², A²+M³, A²+M⁴, A²+M⁵, A²+M⁶; A³+M¹, A³+M², A³+M³, A³+M⁴, A³+M⁵, A³+M⁶; A⁴+M¹, A⁴+M², A⁴+M³, A⁴+M⁴, A⁴+M⁵, A⁴+M⁶; A⁵+M¹, A⁵+M², A⁵+M³, A⁵+M⁴, A⁵+M⁵, A⁵+M⁶; A⁶+M¹, A⁶+M², A⁶+M³, A⁶+M⁴, A⁶+M⁵, A⁶+M⁶; A¹+N¹, A¹+N², A¹+N³, A¹+N⁴, A¹+N⁵, A¹+N⁶; A²+N¹, A²+N², A²+N³, A²+N⁴, A²+N⁵, A²+N⁶; A³+N¹, A³+N², A³+N³, A³+N⁴, A³+N⁵, A³+N⁶; A⁴+N¹, A⁴+N², A⁴+N³, A⁴+N⁴, A⁴+N⁵, A⁴+N⁶; A⁵+N¹, A⁵+N², A⁵+N³, A⁵+N⁴, A⁵+N⁵, A⁵+N⁶; A⁶+N¹, A⁶+N², A⁶+N³, A⁶+N⁴, A⁶+N⁵, A⁶+N⁶; B¹+M², B¹+M³, B¹+M⁴, B¹+M⁵, B¹+M⁶; B²+M¹, B²+M², B²+M³, B²+M⁴, B²+M⁵, B²+M⁶; B³+M¹, B³+M², B³+M³, B³+M⁴, B³+M⁵, B³+M⁶; B⁴+M¹, B⁴+M², B⁴+M³, B⁴+M⁴, B⁴+M⁵, B⁴+M⁶; B⁵+M¹, B⁵+M², B⁵+M³, B⁵+M⁴, B⁵+M⁵, B⁵+M⁶; B⁶+M¹, B⁶+M², B⁶+M³, B⁶+M⁴, B⁶+M⁵, B⁶+M⁶; B¹+N¹, B¹+N², B¹+N³, B¹+N⁴, B¹+N⁵, B¹+N⁶; B²+N¹, B²+N², B²+N³, B²+N⁴, B²+N⁵, B²+N⁶; B³+N¹, B³+N², B³+N³, B³+N⁴, B³+N⁵, B³+N⁶; B⁴+N¹, B⁴+N², B⁴+N³, B⁴+N⁴, B⁴+N⁵, B⁴+N⁶; B⁵+N¹, B⁵+N², B⁵+N³, B⁵+N⁴, B⁵+N⁵, B⁵+N⁶; B⁶+N¹, B⁶+N², B⁶+N³, B⁶+N⁴, B⁶+N⁵, B⁶+N⁶; C¹+M¹, C¹+M², C¹+M³, C¹+M⁴, C¹+M⁵, C¹+M⁶; C²+M¹, C²+M², C²+M³, C²+M⁴, C²+M⁵, C²+M⁶; C³+M¹, C³+M², C³+M³, C³+M⁴, C³+M⁵, C³+M⁶; C⁴+M¹, C⁴+M², C⁴+M³, C⁴+M⁴, C⁴+M⁵, C⁴+M⁶; C⁵+M¹, C⁵+M², C⁵+M³, C⁵+M⁴, C⁵+M⁵, C⁵+M⁶; C⁶+M¹, C⁶+M², C⁶+M³, C⁶+M⁴, C⁶+M⁵, C⁶+M⁶; C¹+N¹, C¹+N², C¹+N³, C¹+N⁴, C¹+N⁵, C¹+N⁶; C²+N¹, C²+N², C²+N³, C²+N⁴, C²+N⁵, C²+N⁶; C³+N¹, C³+N², C³+N³, C³+N⁴, C³+N⁵, C³+N⁶; C⁴+N¹, C⁴+N², C⁴+N³, C⁴+N⁴, C⁴+N⁵, C⁴+N⁶; C⁵+N¹, C⁵+N², C⁵+N³, C⁵+N⁴, C⁵+N⁵, C⁵+N⁶; C⁶+N¹, C⁶+N², C⁶+N³, C⁶+N⁴, C⁶+M⁵, C⁶+M⁶; D¹+M¹, D¹+M², D¹+M³, D¹+M⁴, D¹+M⁵, D¹+M⁶; D²+M¹, D²+M², D²+M³, D²+M⁴, D²+M⁵, D²+M⁶; D³+M¹, D³+M², D³+M³, D³+M⁴, D³+M⁵, D³+M⁶; D⁴+M¹, D⁴+M², D⁴+M³, D⁴+M⁴, D⁴+M⁵, D⁴+M⁶; D⁵+M¹, D⁵+M², D⁵+M³, D⁵+M⁴, D⁵+M⁵, D⁵+M⁶; D⁶+M¹, D⁶+M², D⁶+M³, D⁶+M⁴, D⁶+M⁵, D⁶+M⁶; D¹+N¹, D¹+N², D¹+N³, D¹+N⁴, D¹+N⁵, D¹+N⁶; D²+N¹, D²+N², D²+N³, D²+N⁴, D²+N⁵, D²+N⁶; D³+N¹, D³+N², D³+N³, D³+N⁴, D³+N⁵, D³+N⁶; D⁴+N¹, D⁴+N², D⁴+N³, D⁴+N⁴, D⁴+N⁵, D⁴+N⁶; D⁵+N¹, D⁵+N², D⁵+N³, D⁵+N⁴, D⁵+N⁵, D⁵+N⁶; D⁶+N¹, D⁶+N², D⁶+N³, D⁶+N⁴, D⁶+N⁵, D⁶+N⁶; E¹+M¹, E¹+M², E¹+M³, E¹+M⁴, E¹+M⁵, E¹+M⁶; E²+M¹, E²+M², E²+M³, E²+M⁴, E²+M⁵, E²+M⁶; E³+M¹, E³+M², E³+M³, E³+M⁴, E³+M⁵, E³+M⁶; E⁴+M¹, E⁴+M², E⁴+M³, E⁴+M⁴, E⁴+M⁵, E⁴+M⁶; E⁵+M¹, E⁵+M², E⁵+M³, E⁵+M⁴, E⁵+M⁵, E⁵+M⁶; E⁶+M¹, E⁶+M², E⁶+M³, E⁶+M⁴, E⁶+M⁵, E⁶+M⁶; E¹+N¹, E¹+N², E¹+N³, E¹+N⁴, E¹+N⁵, E¹+N⁶; E²+N¹, E²+N², E²+N³, E²+N⁴, E²+N⁵, E²+N⁶; E³+N¹, E³+N², E³+N³, E³+N⁴, E³+N⁵, E³+N⁶; E⁴+N¹, E⁴+N², E⁴+N³, E⁴+N⁴, E⁴+N⁵, E⁴+N⁶; E⁵+N¹, E⁵+N², E⁵+N³, E⁵+N⁴, E⁵+N⁵, E⁵+N⁶; E⁶+N¹, E⁶+N², E⁶+N³, E⁶+N⁴, E⁶+N⁵, E⁶+N⁶; F¹+M¹, F¹+M², F¹+M³, F¹+M⁴, F¹+M⁵, F¹+M⁶; F²+M¹, F²+M², F²+M³, F²+M⁴, F²+M⁵, F²+M⁶; F³+M¹, F³+M², F³+M³, F³+M⁴, F³+M⁵, F³+M⁶; F⁴+M¹, F⁴+M², F⁴+M³, F⁴+M⁴, F⁴+M⁵, F⁴+M⁶; F⁵+M¹, F⁵+M², F⁵+M³, F⁵+M⁴, F⁵+M⁵, F⁵+M⁶; F⁶+M¹, F⁶+M², F⁶+M³, F⁶+M⁴, F⁶+M⁵, F⁶+M⁶; F¹+N¹, F¹+N², F¹+N³, F¹+N⁴, F¹+N⁵, F¹+N⁶; F²+N¹, F²+N², F²+N³, F²+N⁴, F²+N⁵, F²+N⁶; F³+N¹, F³+N², F³+N³, F³+N⁴, F³+N⁵, F³+N⁶; F⁴+N¹, F⁴+N², F⁴+N³, F⁴+N⁴, F⁴+N⁵, F⁴+N⁶; F⁵+N¹, F⁵+N², F⁵+N³, F⁵+N⁴, F⁵+N⁵, F⁵+N⁶; F⁶+N¹, F⁶+N², F⁶+N³, F⁶+N⁴, F⁶+N⁵, and F⁶+N⁶.

Preferably, in any of the above combinations, the relative weight ratio of said multitude of IR particles to said at least one PR particle is within the range of from 5:95 to 95:5, more preferably of from 10:90 to 90:10, still more preferably of from 15:85 to 85:15, yet more preferably of from 20:80 to 80:20, most preferably of from 25:75 to 75:25.

According to a preferred embodiment, the pharmaceutical dosage form according to the invention comprises a multitude of IR particles in combination with a multitude of DR particles, but preferably neither FR particles nor PR particles nor OR particles.

Preferably, the PR particles are in accordance with any of above embodiments A¹ to A⁶, or B¹ to B⁶, or C¹ to C⁶, or D¹ to D⁶, or E¹ to E⁶, or F¹ to F⁶, whereas the DR particle(s) are in accordance with any of above embodiments O¹ to O⁶, or P¹ to P⁶, or Q¹ to Q⁶, or R¹ to R⁶, or S¹ to S⁶. Preferred individualized combinations of embodiments are: A¹+O¹, A¹+O², A¹+O³, A¹+O⁴, A¹+O⁵, A¹+O⁶; A²+O¹, A²+O², A²+O³, A²+O⁴, A²+O⁵, A²+O⁶; A³+O¹, A³+O², A³+O³, A³+O⁴, A³+O⁵, A³+O⁶; A⁴+O¹, A⁴+O², A⁴+O³, A⁴+O⁴, A⁴+O⁵, A⁴+O⁶; A⁵+O¹, A⁵+O², A⁵+O³, A⁵+O⁴, A⁵+O⁵, A⁵+O⁶; A⁶+O¹, A⁶+O², A⁶+O³, A⁶+O⁴, A⁶+O⁵, A⁶+O⁶; A¹+P¹, A¹+P², A¹+P³, A¹+P⁴, A¹+P⁵, A¹+P⁶; A²+P¹, A²+P², A²+P³, A²+P⁴, A²+P⁵, A²+P⁶; A³+P¹, A³+P², A³+P³, A³+P⁴, A³+P⁵, A³+P⁶; A⁴+P¹, A⁴+P², A⁴+P³, A⁴+P⁴, A⁴+P⁵, A⁴+P⁶; A⁵+P¹, A⁵+P², A⁵+P³, A⁵+P⁴, A⁵+P⁵, A⁵+P⁶; A⁶+P¹, A⁶+P², A⁶+P³, A⁶+P⁴, A⁶+P⁵, A⁶+P⁶; A¹+Q¹, A¹+Q², A¹+Q³, A¹+Q⁴, A¹+Q⁵, A¹+Q⁶; A²+Q¹, A²+Q², A²+Q³, A²+Q⁴, A²+Q⁵, A²+Q⁶; A³+Q¹, A³+Q², A³+Q³, A³+Q⁴, A³+Q⁵, A³+Q⁶; A⁴+Q¹, A⁴+Q², A⁴+Q³, A⁴+Q⁴, A⁴+Q⁵, A⁴+Q⁶; A⁵+Q¹, A⁵+Q², A⁵+Q³, A⁵+Q⁴, A⁵+Q⁵, A⁵+Q⁶; A⁶+Q¹, A⁶+Q², A⁶+Q³, A⁶+Q⁴, A⁶+Q⁵, A⁶+Q⁶; A¹+R¹, A¹+R², A¹+R³, A¹+R⁴, A¹+R⁵, A¹+R⁶; A²+R¹, A²+R², A²+R³, A²+R⁴, A²+R⁵, A²+R⁶; A³+R¹, A³+R², A³+R³, A³+R⁴, A³+R⁵, A³+R⁶; A⁴+R¹, A⁴+R², A⁴+R³, A⁴+R⁴, A⁴+R⁵, A⁴+R⁶; A⁵+R¹, A⁵+R², A⁵+R³, A⁵+R⁴, A⁵+R⁵, A⁵+R⁶; A⁶+R¹, A⁶+R², A⁶+R³, A⁶+R⁴, A⁶+R⁵, A⁶+R⁶; A¹+S¹, A¹+S², A¹+S³, A¹+S⁴, A¹+S⁵, A¹+S⁶; A²+S¹, A²+S², A²+S³, A²+S⁴, A²+S⁵, A²+S⁶; A³+S¹, A³+S², A³+S³, A³+S⁴, A³+S⁵, A³+S⁶; A⁴+S¹, A⁴+S², A⁴+S³, A⁴+S⁴, A⁴+S⁵, A⁴+S⁶; A⁵+S¹, A⁵+S², A⁵+S³, A⁵+S⁴, A⁵+S⁵, A⁵+S⁶; A⁶+S¹, A⁶+S², A⁶+S³, A⁶+S⁴, A⁶+S⁵, A⁶+S⁶; B¹+O¹, B¹+O², B¹+O³, B¹+O⁴, B¹+O⁵, B¹+O⁶; B²+O¹, B²+O², B²+O³, B²+O⁴, B²+O⁵, B²+O⁶; B³+O¹, B³+O², B³+O³, B³+O⁴, B³+O⁵, B³+O⁶; B⁴+O¹, B⁴+O², B⁴+O³, B⁴+O⁴, B⁴+O⁵, B⁴+O⁶; B⁵+O¹, B⁵+O², B⁵+O³, B⁵+O⁴, B⁵+O⁵, B⁵+O⁶; B⁶+O¹, B⁶+O², B⁶+O³, B⁶+O⁴; B+O⁵, B+O⁶; B¹+P¹, B¹+P², B¹+P³, B¹+P⁴, B¹+P⁵, B¹+P⁶; B²+P¹, B²+P², B²+P³, B²+P⁴, B²+P⁵, B²+P⁶; B³+P¹, B³+P², B³+P³, B³+P⁴, B³+P⁵, B³+P⁶; B⁴+P¹, B⁴+P², B⁴+P³, B⁴+P⁴, B⁴+B⁵, B⁴⁺⁶; B⁵+P¹, B⁵+P², B⁵+P³, B⁵+P⁴, B⁵+P⁵, B⁵+P⁶; B⁶+P¹, B⁶+P², B⁶+P³, B⁶+P⁴, B⁶+P⁵, B⁶+P⁶; B¹+Q¹, B¹+Q², B¹+Q³, B¹+Q⁴, B¹+Q⁵, B¹+Q⁶; B²+Q¹, B²+Q², B²+Q³, B²+Q⁴, B²+Q⁵, B²+Q⁶; B³+Q¹, B³+Q², B³+Q³, B³+Q⁴, B³+Q⁵, B³+Q⁶; B⁴+Q¹, B⁴+Q², B⁴+Q³, B⁴+Q⁴, B⁴+Q⁵, B⁴+Q⁶; B⁵+Q¹, B⁵+Q², B⁵+Q³, B⁵+Q⁴, B⁵+Q⁵, B⁵+Q⁶; B⁶+Q¹, B⁶+Q², B⁶+Q³, B⁶+Q⁴; B⁶+Q⁵, B⁶+Q⁶; B¹+R¹, B¹+R², B¹+R³, B¹+R⁴, B+R⁵, B+R⁶; B²+R¹, B²+R², B²+R³, B²+R⁴, B²+R⁵, B²+R⁶; B³+R¹, B³+R², B³+R³, B³+R⁴, B³+R⁵, B³+R⁶; B⁴+R¹, B⁴+R², B⁴+R³, B⁴+R⁴, B⁴++R⁵, B⁴+R⁶; B⁵+R¹, B⁵+R², B⁵+R³, B⁵+R⁴, B⁵+R⁵, B⁵+R⁶; B⁶+R¹, B⁶+R², B⁶+R³, B⁶+R⁴, B⁶+R⁵, B⁶+R⁶; B¹+S¹, B¹+S², B¹+S³, B¹+S⁴, B¹+S⁵, B¹+S⁶; B²+S¹, B²+S², B²+S³, B²+S⁴, B²+S⁵, B²+S⁶; B³+S¹, B³+S², B³+S³, B³+S⁴, B³+S⁵, B³+S⁶; B⁴+S¹, B⁴+S², B⁴+S³, B⁴+S⁴, B⁴+S⁵, B⁴+S⁶; B⁵+S¹, B⁵+S², B⁵+S³, B⁵+S⁴, B⁵+S⁵, B⁵+S⁶; B⁶+S¹, B⁶+S², B⁶+S³, B⁶+S⁴, B⁶+S⁵, B⁶+S⁶; C¹+O¹, C¹+O², C¹+O³, C¹+O⁴; C¹+O⁵, C¹+O⁶; C²+O¹, C²+O², C²+O³, C²+O⁴; C²+O⁵, C²+O⁶; C³+O¹, C³+O², C³+O³, C³+O⁴; C³+O⁵, C³+O⁶; C⁴⁺⁰¹, C⁴+O², C⁴+O³, C⁴+O⁴; C⁴+O⁵, C⁴+O⁶; C⁵+O¹, C⁵+O², C⁵+O³, C⁵+O⁴; C⁵+O⁵, C⁵+O⁶; C⁶+O¹, C⁶+O², C⁶+O³, C⁶+O⁴, C⁶+O⁵, C⁶+O⁶; C¹+P¹, C¹+P², C¹+P³, C¹+P⁴, C¹+P⁵, C¹+P⁶; C²+P¹, C²+P², C²+P³, C²+P⁴, C²+P⁵, C²+P⁶; C³+P¹, C³+P², C²+P³; C³+P⁴, C³+P⁵, C³+P³; C³++P⁴, C³+P⁵, C³+S⁶; C⁴+P¹, C⁴+P², C⁴+P³, C⁴+P⁴, C⁴+P⁵, C⁴+P⁶; C⁵+P¹, C⁵+P², C⁵+P³, C⁵+P⁴, C⁵+P⁵, C⁵+P⁶; C⁶+P¹, C⁶+P², C⁶+P³, C⁶+P⁴, C⁶+P⁵, C⁶+P⁶; C¹+Q¹, C¹+Q², C¹+Q³, C¹+Q⁴, C¹+Q⁵, C¹+Q⁶; C²+Q¹, C²+Q², C²+Q³, C²+Q⁴, C²+Q⁵, C²+Q⁶; C³+Q¹, C³+Q², C³+Q³, C³+Q⁴, C³+Q⁵, C³+Q⁶; C⁴+Q¹, C⁴+Q², C⁴+Q³, C⁴+Q⁴, C⁴+Q⁵, C⁴+Q⁶; C⁵+Q¹, C⁵+Q², C⁵+Q³, C⁵+Q⁴, C⁵+Q⁵, C⁵+Q⁶; C⁶+Q¹, C⁶+Q², C⁶+Q³, C⁶+Q⁴, C⁶+Q⁵, C⁶+Q⁶; C¹+R¹, C¹+R², C¹+R³, C¹+R⁴, C¹+R⁵, C¹+R⁶; C²+R¹, C²+R², C²+R³, C²+R⁴, C⁴+R⁵, C⁴+R⁶; C⁵+R¹, C⁵+R², C⁵+R³, C⁵+R⁴, C⁵+R⁵, C⁵+R⁶; C⁶+R¹, C⁶+R², C⁶+R³, C⁴+R⁴, C⁴+R⁵, C⁴+R⁶; C⁵+R¹, C⁵+R², C⁵+R³, C⁵+R⁴, C⁵+R⁵, C⁵+R⁶; C⁶+R¹, C⁶+R², C⁶+R³, C⁶+R⁴, C⁶+R⁵, C⁶+R⁶; C¹+S¹, C¹+S², C¹+S³, C¹+S⁴, C¹+S⁵, C¹+S⁶; C²+S¹, C²+S², C²+S³, C²+S⁴, C²+S⁵, C²+S⁶; C³+S¹, C³+S², C³+S³, C³+S⁴, C³+S⁵, C³+S⁶; C⁴+S¹, C⁴+S², C⁴+S³, C⁴+S⁴, C⁴+S⁵, C⁴+S⁶; C⁵+S¹, C⁵+S², C⁵+S³, C⁵+S⁴, C⁵+S⁵, C⁵+S⁶; C⁶+S¹, C⁶+S², C⁶+S³, C⁶+S⁴, C⁶+S⁵, C⁶+S⁶; D¹+O¹, D¹+O², D¹+O³, D¹+O⁴, D¹+O⁵, D¹+O⁶; D⁶+O¹, D⁶+O², D⁶+O³, D²+O⁴, D²+O⁵, D²+O⁶; D³+O¹, D³+O², D³+O³, D³+O⁴, D³+O⁵, D³+O⁶; D⁴+O¹, D⁴+O², D⁴+O³, D⁴+O⁴, D⁴+O⁵, D⁴+O⁶; D⁵+O¹, D⁵+O², D⁵+O³, D⁵+O⁴, D⁵+O⁵, D⁵+O⁶; D⁶+O¹, D⁶+O², D⁶+O³, D⁶+O⁴, D⁶+O⁵, D⁶+O⁶; D¹+P¹, D¹+P², D¹+P³, D¹+P⁴, D¹+P⁵, D¹+P⁶; D²+P¹, D²+P², D²+P³, D²+P⁴, D²+P⁵, D²+P⁶; D³+P¹, D³+P², D³+P³, D³+P⁴, D³+P⁵, D³+P⁶; D⁴+P¹, D⁴+P², D⁴+P³, D⁴+P⁴, D⁴+P⁵, D⁴+P⁶; D⁵+P¹, D⁵+P², D⁵+P³, D⁵+P⁴, D⁵+P⁵, D⁵+P⁶; D⁶+P¹, D⁶+P², D⁶+P³, D⁶+P⁴, D⁶+P⁵, D⁶+P⁶; D¹+Q¹, D¹+Q², D¹+Q³, D¹+Q⁴, D¹+Q⁵, D¹+Q⁶; D²+Q¹, D²+Q², D²+Q³, D²+Q⁴, D²+Q⁵, D²+Q⁶; D³+Q¹, D³+Q², D³+Q³, D³+Q⁴, D³+Q⁵, D³+Q⁶; D⁴+Q¹, D⁴+Q², D⁴+Q³, D⁴+Q⁴, D⁴+Q⁵, D⁴+Q⁶; D⁵+Q¹, D⁵+Q², D⁵+Q³, D⁵+Q⁴, D⁵+Q⁵, D⁵+Q⁶; D⁶+Q¹, D⁶+Q², D⁶+Q³, D⁶+Q⁴, D⁶+Q⁵, D⁶+Q⁶; D¹+R¹, D¹+R², D¹+R³, D¹+R⁴, D¹+R⁵, D¹+R⁶; D²+R¹, D²+R², D²+R³, D²+R⁴, D²+R⁵, D²+R⁶; D³+R¹, D³+R², D³+R³, D³+R⁴, D³+R⁵, D³+R⁶; D⁴+R¹, D⁴+R², D⁴+R³, D⁴+R⁴, D⁴+R⁵, D⁴+R⁶; D⁵+R¹, D⁵+R², D⁵+R³, D⁵+R⁴, D⁵+R⁵, D⁵+R⁶; D⁶+R¹, D⁶+R², D⁶+R³, D⁶+R⁴, D⁶+R⁵, D⁶+R⁶; D¹+S¹, D¹+S², D¹+S³, D¹+S⁴, D¹+S⁵, D¹+S⁶; D²+S¹, D²+S², D²+S³, D²+S⁴, D²+S⁵, D²+S⁶; D³+S¹, D³+S², D³+S³, D³+S⁴, D³+S⁵, D³+S⁶; D⁴+S¹, D⁴+S², D⁴+S³, D⁴+S⁴, D⁴+S⁵, D⁴+S⁶; D⁵+S¹, D⁵+S², D⁵+S³, D⁵+S⁴, D⁵+S⁵, D⁵+S⁶; D⁶+S¹, D⁶+S², D⁶+S³, D⁶+S⁴, D⁶+S⁵, D⁶+S⁶; E¹+O¹, E¹+O², E¹+O³, E¹+O⁴, E¹+O⁵, E¹+O⁶; E²+O¹, E²+O², E²+O³, E²+O⁴, E²+O⁵, E²+O⁶; E³+O¹, E³+O², E³+O³, E³+O⁴, E³+O⁵, E³+O⁶; E⁴+O¹, E⁴+O², E⁴+O³, E⁴+O⁴, E⁴+O⁵, E⁴+O⁶; E⁵+O¹, E⁵+O², E⁵+O³, E⁵+O⁴, E⁵+O⁵, E⁵+O⁶; E⁶+O¹, E⁶+O², E⁶+O³, E⁶+O⁴, E⁶+O⁵, E⁶+O⁶; E¹+P¹, E¹+P², E¹+P³, E¹+P⁴, E¹+P⁵, E¹+P⁶; E²+P¹, E²+P², E²+P³, E²+P⁴, E²+P⁵, E²+P⁶; E³+P¹, E³+P², E³+P³, E³+P⁴, E³+P⁵, E³+P⁶; E⁴+P¹, E⁴+P², E⁴+P³, E⁴+P⁴, E⁴+P⁵, E⁴+P⁶; E⁵+P¹, E⁵+P², E⁵+P³, E⁵+P⁴, E⁵+P⁵, E⁵+P⁶; E⁶+P¹, E⁶+P², E⁶+P³, E⁶+P⁴, E⁶+P⁵, E⁶+P⁶; E¹+Q¹, E¹+Q², E¹+Q³, E¹+Q⁴, E¹+Q⁵, E¹+Q⁶; E²+Q¹, E²+Q², E²+Q³, E²+Q⁴, E²+Q⁵, E²+Q⁶; E³+Q¹, E³+Q², E³+Q³, E³+Q⁴, E³+Q⁵, E³+Q⁶; E⁴+Q¹, E⁴+Q², E⁴+Q³, E⁴+Q⁴, E⁴+Q⁵, E⁴+Q⁶; E⁵+Q¹, E⁵+Q², E⁵+Q³, E⁵+Q⁴, E⁵+Q⁵, E⁵+Q⁶; E⁶+Q¹, E⁶+Q², E⁶+Q³, E⁶+Q⁴, E⁶+Q⁵, E⁶+Q⁶; E¹+R¹, E¹+R², E¹+R³, E¹+R⁴, E¹+R⁵, E¹+R⁶; E²+R¹, E²+R², E²+R³, E²+R⁴, E²+R⁵, E²+R⁶; E³+R¹, E³+R², E³+R³, E³+R⁴, E³+R⁵, E³+R⁶; E⁴+R¹, E⁴+R², E⁴+R³, E⁴+R⁴, E⁴+R⁵, E⁴+R⁶; E⁵+R¹, E⁵+R², E⁵+R³, E⁵+R⁴, E⁵+R⁵, E⁵+R⁶; E⁶+R¹, E⁶+R², E⁶+R³, E⁶+R⁴, E⁶+R⁵, E⁶+R⁶; E¹+S¹, E¹+S², E¹+S³, E¹+S⁴, E¹+S⁵, E¹+S⁶; E²+S¹, E²+S², E²+S³, E²+S⁴, E²+S⁵, E²+S⁶; E³+S¹, E³+S², E³+S³, E³+S⁴, E³+S⁵, E³+S⁶; E⁴+S¹, E⁴+S², E⁴+S³, E⁴+S⁴, E⁴+S⁵, E⁴+S⁶; E⁵+S¹, E⁵+S², E⁵+S³, E⁵+S⁴, E⁵+S⁵, E⁵+S⁶; E⁶+S¹, E⁶+S², E⁶+S³, E⁶+S⁴, E⁶+S⁵, E⁶+S⁶; F¹+O¹, F¹+O², F¹+O³, F¹+O⁴, F¹+O⁵, F¹+O⁶; F²+O¹, F²+O², F²+O³, F²+O⁴, F²+O⁵, F²+O⁶; F³+O¹, F³+O², F³+O³, F³+O⁴, F³+O⁵, F³+O⁶; F⁴+O¹, F⁴+O², F⁴+O³, F⁴+O⁴, F⁴+O⁵, F⁴+O⁶; F⁵+O¹, F⁵+O², F⁵+O³, F⁵+O⁴, F⁵+O⁵, F⁵+O⁶; F⁶+O¹, F⁶+O², F⁶+O³, F⁶+O⁴, F⁶+O⁵, F⁶+O⁶; F¹+P¹, F¹+P², F¹+P³, F¹+P⁴, F¹+P⁵, F¹+P⁶; F²+P¹, F²+P², F²+P³, F²+P⁴, F²+P⁵, F²+P⁶; F³+P¹, F³+P², F³+P³, F³+P⁴, F³+P⁵, F³+P⁶; F⁴+P¹, F⁴+P², F⁴+P³, F⁴+P⁴, F⁴+P⁵, F⁴+P⁶; F⁵+P¹, F⁵+P², F⁵+P³, F⁵+P⁴, F⁵+P⁵, F⁵+P⁶; F⁶+P¹, F⁶+P², F⁶+P³, F⁶+P⁴, F⁶+P⁵, F⁶+P⁶; F¹+Q¹, F¹+Q², F¹+Q³, F¹+Q⁴, F¹+Q⁵, F¹+Q⁶; F²+Q¹, F²+Q², F²+Q³, F²+Q⁴, F²+Q⁵, F²+Q⁶; F³+Q¹, F³+Q², F³+Q³, F³+Q⁴, F³+Q⁵, F³+Q⁶; F⁴+Q¹, F⁴+Q², F⁴+Q³, F⁴+Q⁴, F⁴+Q⁵, F⁴+Q⁶; F⁵+Q¹, F⁵+Q², F⁵+Q³, F⁵+Q⁴, F⁵+Q⁵, F⁵+Q⁶; F⁶+Q¹, F⁶+Q², F⁶+Q³, F⁶+Q⁴, F⁶+Q⁵, F⁶+Q⁶; F¹+R¹, F¹+R², F¹+R³, F¹+R⁴, F¹+R⁵, F¹+R⁶; F²+R¹, F²+R², F²+R³, F²+R⁴, F²+R⁵, F²+R⁶; F³+R¹, F³+R², F³+R³, F³+R⁴, F³+R⁵, F³+R⁶; F⁴+R¹, F⁴+R², F⁴+R³, F⁴+R⁴, F⁴+R⁵, F⁴+R⁶; F⁵+R¹, F⁵+R², F⁵+R³, F⁵+R⁴, F⁵+R⁵, F⁵+R⁶; F⁶+R¹, F⁶+R², F⁶+R³, F⁶+R⁴, F⁶+R⁵, F⁶+R⁶; F¹+S¹, F¹+S², F¹+S³, F¹+S⁴, F¹+S⁵, F¹+S⁶; F²+S¹, F²+S², F²+S³, F²+S⁴, F²+S⁵, F²+S⁶; F³+S¹, F³+S², F³+S³, F³+S⁴, F³+S⁵, F³+S⁶; F⁴+S¹, F⁴+S², F⁴+S³, F⁴+S⁴, F⁴+S⁵, F⁴+S⁶; F⁵+S¹, F⁵+S², F⁵+S³, F⁵+S⁴, F⁵+S⁵, F⁵+S⁶; F⁶+S¹, F⁶+S², F⁶+S³, F⁶+S⁴, F⁶+S⁵, and F⁶+S⁶.

Preferably, in any of the above combinations, the relative weight ratio of said multitude of IR particles to said multitude of DR particles is within the range of from 5:95 to 95:5, more preferably of from 10:90 to 90:10, still more preferably of from 15:85 to 85:15, yet more preferably of from 20:80 to 80:20, most preferably of from 25:75 to 75:25.

According to a preferred embodiment, the pharmaceutical dosage form according to the invention comprises a multitude of IR particles in combination with a multitude of OR particles, but preferably neither FR particles nor PR particles nor DR particles.

Preferably, the IR particles are in accordance with any of above embodiments A¹ to A⁶, or B¹ to B⁶, or C¹ to C⁶, or D¹ to D⁶, or E¹ to E⁶, or F¹ to F⁶, whereas the OR particle(s) are in accordance with any of above embodiments T¹ to T⁶, or U¹ to U⁶. Preferred individualized combinations of embodiments are: A¹+T¹, A¹+T², A¹+T³, A¹+T⁴, A¹+T⁵, A¹+T⁶; A²+T¹, A²+T², A²+T³, A²+T⁴, A²+T⁵, A²+T⁶; A³+T¹, A³+T², A³+T³, A³+T⁴, A³+T⁵, A³+T⁶; A⁴+T¹, A⁴+T², A⁴+T³, A⁴+T⁴, A⁴+T⁵, A⁴+T⁶; A⁵+T¹, A⁵+T², A⁵+T³, A⁵+T⁴, A⁵+T⁵, A⁵+T⁶; A⁶+T¹, A⁶+T², A⁶+T³, A⁶+T⁴, A⁶+T⁵, A⁶+T⁶; A¹+U¹, A¹+U², A¹+U³, A¹+U⁴, A¹+U⁵, A¹+U⁶; A²+U¹, A²+U², A²+U³, A²+U⁴, A²+U⁵, A²+U⁶; A³+U¹, A³+U², A³+U³, A³+U⁴, A³+U⁵, A³+U⁶; A⁴+U¹, A⁴+U², A⁴+U³, A⁴+U⁴, A⁴+U⁵, A⁴+U⁶; A⁵+U¹, A⁵+U², A⁵+U³, A⁵+U⁴, A⁵+U⁵, A⁵+U⁶; A⁶+U¹, A⁶+U², A⁶+U³, A⁶+U⁴, A⁶+U⁵, A⁶+U⁶; B¹+T¹, B¹+T², B¹+T³, B¹+T⁴, B¹+T⁵, B¹+T⁶; B²+T¹, B²+T², B²+T³, B²+T⁴, B²+T⁵, B²+T⁶; B³+T¹, B³+T², B³+T³, B³+T⁴, B³+T⁵, B³+T⁶; B⁴+T¹, B⁴+T², B⁴+T³, B⁴+T⁴, B⁴+T⁵, B⁴+T⁶; B⁵+T¹, B⁵+T², B⁵+T³, B⁵+T⁴, B⁵+T⁵, B⁵+T⁶; B⁶+T¹, B⁶+T², B⁶+T³, B⁶+T⁴, B⁶+T⁵, B⁶+T⁶; B¹+U¹, B¹+U², B¹+U³, B¹+U⁴, B¹+U⁵, B¹+U⁶; B²+U¹, B²+U², B²+U³, B²+U⁴, B²+U⁵, B²+U⁶; B³+U¹, B³+U², B³+U³, B³+U⁴, B³+U⁵, B³+U⁶; B⁴+U¹, B⁴+U², B⁴+U³, B⁴+U⁴, B⁴+U⁵, B⁴+U⁶; B⁵+U¹, B⁵+U², B⁵+U³, B⁵+U⁴, B⁵+U⁵, B⁵+U⁶; B⁶+U¹, B⁶+U², B⁶+U³, B⁶+U⁴, B⁶+U⁵, B⁶+U⁶; C¹+T¹, C¹+T², C¹+T³, C¹+T⁴, C¹+T⁵, C¹+T⁶; C²+T¹, C²+T², C²+T³, C²+T⁴, C²+T⁵, C²+T⁶; C³+T¹, C³+T², C³+T³, C³+T⁴, C³+T⁵, C³+T⁶; C⁴+T¹, C⁴+T², C⁴+T³, C⁴+T⁴, C⁴+T⁵, C⁴+T⁶; C⁵+T¹, C⁵+T², C⁵+T³, C⁵+T⁴, C⁵+T⁵, C⁵+T⁶; C⁶+T¹, C⁶+T², C⁶+T³, C⁶+T⁴, C⁶+T⁵, C⁶+T⁶; C¹+U¹, C¹+U², C¹+U³, C¹+U⁴, C¹+U⁵, C¹+U⁶; C²+U¹, C²+U², C²+U³, C²+U⁴, C²+U⁵, C²+U⁶; C³+U¹, C³+U², C³+U³, C³+U⁴, C³+U⁵, C³+U⁶; C⁴+U¹, C⁴+U², C⁴+U³, C⁴+U⁴, C⁴+U⁵, C⁴+U⁶; C⁵+U¹, C⁵+U², C⁵+U³, C⁵+U⁴, C⁵+U⁵, C⁵+U⁶; C⁶+U¹, C⁶+U², C⁶+U³, C⁶+U⁴, C⁶+U⁵, C⁶+U⁶; D¹+T¹, D¹+T², D¹+T³, D¹+T⁴, D¹+T⁵, D¹+T⁶; D²+T¹, D²+T², D²+T³, D²+T⁴, D²+T⁵, D²+T⁶; D³+T¹, D³+T², D³+T³, D³+T⁴, D³+T⁵, D³+T⁶; D⁴+T¹, D⁴+T², D⁴+T³, D⁴+T⁴, D⁴+T⁵, D⁴+T⁶; D⁵+T¹, D⁵+T², D⁵+T³, D⁵+T⁴, D⁵+T⁵, D⁵+T⁶; D⁶+T¹, D⁶+T², D⁶+T³, D⁶+T⁴, D⁶+T⁵, D⁶+T⁶; D¹+U¹, D¹+U², D¹+U³, D¹+U⁴, D¹+U⁵, D¹+U⁶; D²+U¹, D²+U², D²+U³, D²+U⁴, D²+U⁵, D²+U⁶; D³+U¹, D³+U², D³+U³, D³+U⁴, D³+U⁵, D³+U⁶; D⁴+U¹, D⁴+U², D⁴+U³, D⁴+U⁴, D⁴+U⁵, D⁴+U⁶; D⁵+U¹, D⁵+U², D⁵+U³, D⁵+U⁴, D⁵+U⁵, D⁵+U⁶; D⁶+U¹, D⁶+U², D⁶+U³, D⁶+U⁴, D⁶+U⁵, D⁶+U⁶; E¹+T¹, E¹+T², E¹+T³, E¹+T⁴, E¹+T⁵, E¹+T⁶; E²+T¹, E²+T², E²+T³, E²+T⁴, E²+T⁵, E²+T⁶; E³+T¹, E³+T², E³+T³, E³+T⁴, E³+T⁵, E³+T⁶; E⁴+T¹, E⁴+T², E⁴+T³, E⁴+T⁴, E⁴+T⁵, E⁴+T⁶; E⁵+T¹, E⁵+T², E⁵+T³, E⁵+T⁴, E⁵+T⁵, E⁵+T⁶; E⁶+T¹, E⁶+T², E⁶+T³, E⁶+T⁴, E⁶+T⁵, E⁶+T⁶; E¹+U¹, E¹+U², E¹+U³, E¹+U⁴, E¹+U⁵, E¹+U⁶; E²+U¹, E²+U², E²+U³, E²+U⁴, E²+U⁵, E²+U⁶; E³+U¹, E³+U², E³+U³, E³+U⁴, E³+U⁵, E³+U⁶; E⁴+U¹, E⁴+U², E⁴+U³, E⁴+U⁴, E⁴+U⁵, E⁴+U⁶; E⁵+U¹, E⁵+U², E⁵+U³, E⁵+U⁴, E⁵+U⁵, E⁵+U⁶; E⁶+U¹, E⁶+U², E⁶+U³, E⁶+U⁴, E⁶+U⁵, E⁶+U⁶; F¹+T¹, F¹+T², F¹+T³, F¹+T⁴, F¹+T⁵, F¹+T⁶; F²+T¹, F²+T², F²+T³, F²+T⁴, F²+T⁵, F²+T⁶; F³+T¹, F³+T², F³+T³, F³+T⁴, F³+T⁵, F³+T⁶; F⁴+T¹, F⁴+T², F⁴+T³, F⁴+T⁴, F⁴+T⁵, F⁴+T⁶; F⁵+T¹, F⁵+T², F⁵+T³, F⁵+T⁴, F⁵+T⁵, F⁵+T⁶; F⁶+T¹, F⁶+T², F⁶+T³, F⁶+T⁴, F⁶+T⁵, F⁶+T⁶; F¹+U¹, F¹+U², F¹+U³, F¹+U⁴, F¹+U⁵, F¹+U⁶; F²+U¹, F²+U², F²+U³, F²+U⁴, F²+U⁵, F²+U⁶; F³+U¹, F³+U², F³+U³, F³+U⁴, F³+U⁵, F³+U⁶; F⁴+U¹, F⁴+U², F⁴+U³, F⁴+U⁴, F⁴+U⁵, F⁴+U⁶; F⁵+U¹, F⁵+U², F⁵+U³, F⁵+U⁴, F⁵+U⁵, F⁵+U⁶; F⁶+U¹, F⁶+U², F⁶+U³, F⁶+U⁴, F⁶+U⁵, and F⁶+U⁶.

Preferably, in any of the above combinations, the relative weight ratio of said multitude of IR particles to said at least one OR particle is within the range of from 5:95 to 95:5, more preferably of from 10:90 to 90:10, still more preferably of from 15:85 to 85:15, yet more preferably of from 20:80 to 80:20, most preferably of from 25:75 to 75:25.

According to a preferred embodiment, the pharmaceutical dosage form according to the invention comprises a multitude of FR particles in combination with a single or a few PR particle(s), but preferably neither IR particles nor DR particles nor OR particles.

Preferably, the FR particles are in accordance with any of above embodiments G¹ to G⁶, or H¹ to H⁶, or I¹ to I⁶, or J¹ to J⁶, or K¹ to K⁶, or L¹ to L⁶, whereas the PR particle(s) are in accordance with any of above embodiments M¹ to M⁶, or N¹ to N⁶. Preferred individualized combinations of embodiments are: G¹+M¹, G¹+M², G¹+M³, G¹+M⁴, G¹+M⁵, G¹+M⁶; G²+M¹, G²+M², G²+M³, G²+M⁴, G²+M⁵, G²+M⁶; G³+M¹, G³+M², G³+M³, G³+M⁴, G³+M⁵, G³+M⁶; G⁴+M¹, G⁴+M², G⁴+M³, G⁴+M⁴, G⁴+M⁵, G⁴+M⁶; G⁵+M¹, G⁵+M², G⁵+M³, G⁵+M⁴, G⁵+M⁵, G⁵+M⁶; G⁶+M¹, G⁶+M², G⁶+M³, G⁶+M⁴, G⁶+M⁵, G⁶+M⁶; G¹+N¹, G¹+N², G¹+N³, G¹+N⁴, G¹+N⁵, G¹+N⁶; G²+N¹, G²+N², G²+N³, G²+N⁴, G²+N⁵, G²+N⁶; G³+N¹, G³+N², G³+N³, G³+N⁴, G³+N⁵, G³+N⁶; G⁴+N¹, G⁴+N², G⁴+N³, G⁴+N⁴, G⁴+N⁵, G⁴+N⁶; G⁵+N¹, G⁵+N², G⁵+N³, G⁵+N⁴, G⁵+N⁵, G⁵+N⁶; G⁶+N¹, G⁶+N², G⁶+N³, G⁶+N⁴, G⁶+N⁵, G⁶+N⁶; H¹+M¹, H¹+M², H¹+M³, H¹+M⁴, H¹+M⁵, H¹+M⁶; H²+M¹, H²+M², H²+M³, H²+M⁴, H²+M⁵, H²+M⁶; H³+M¹, H³+M², H³+M³, H³+M⁴, H³+M⁵, H³+M⁶; H⁴+M¹, H⁴+M², H⁴+M³, H⁴+M⁴, H⁴+M⁵, H⁴+M⁶; H⁵+M¹, H⁵+M², H⁵+M³, H⁵+M⁴, H⁵+M⁵, H⁵+M⁶; H⁶+M¹, H⁶+M², H⁶+M³, H⁶+M⁴, H⁶+M⁵, H⁶+M⁶; H¹+N¹, H¹+N², H¹+N³, H¹+N⁴, H¹+N⁵, H¹+N⁶; H²+N¹, H²+N², H²+N³, H²+N⁴, H²+N⁵, H²+N⁶; H³+N¹, H³+N², H³+N³, H³+N⁴, H³+N⁵, H³+N⁶; H⁴+N¹, H⁴+N², H⁴+N³, H⁴+N⁴, H⁴+N⁵, H⁴+N⁶; H⁵+N¹, H⁵+N², H⁵+N³, H⁵+N⁴, H⁵+N⁵, H⁵+N⁶; H⁶+N¹, H⁶+N², H⁶+N³, H⁶+N⁴, H⁶+N⁵, H⁶+N⁶; I¹+M¹, I¹+M², I¹+M³, I¹+M⁴, I¹+M⁵, I¹+M⁶; I²+M¹, I²+M², I²+M³, I²+M⁴, I²+M⁵, I²+M⁶; I³+M¹, I³+M², I³+M³, I³+M⁴, I³+M⁵, I³+M⁶; I⁴+M¹, I⁴+M², I⁴+M³, I⁴+M⁴, I⁴+M⁵, I⁴+M⁶; I⁵+M¹, I⁵+M², I⁵+M³, I⁵+M⁴, I⁵+M⁵, I⁵+M⁶; I⁶+M¹, I⁶+M², I⁶+M³, I⁶+M⁴, I⁶+M⁵, I⁶+M⁶; I¹+N¹, I¹+N², I¹+N³, I¹+N⁴, I¹+N⁵, I¹+N⁶; I²+N¹, I²+N², I²+N³, I²+N⁴, I²+N⁵, I²+N⁶; I³+N¹, I³+N², I³+N³, I³+N⁴, I³+N⁵, I³+N⁶; I⁴+N¹, I⁴+N², I⁴+N³, I⁴+N⁴, I⁴+N⁵, I⁴+N⁶; I⁵+N¹, I⁵+N², I⁵+N³, I⁵+N⁴, I⁵+N⁵, I⁵+N⁶; I⁶+N¹, I⁶+N², I⁶+N³, I⁶+N⁴, I⁶+N⁵, I⁶+N⁶; J¹+M¹, J¹+M², J¹+M³, J¹+M⁴, J¹+M⁵, J¹+M⁶; J²+M¹, J²+M², J²+M³, J²+M⁴, J²+M⁵, J²+M⁶; J³+M¹, J³+M², J³+M³, J³+M⁴, J³+M⁵, J³+M⁶; J⁴+M¹, J⁴+M², J⁴+M³, J⁴+M⁴, J⁴+M⁵, J⁴+M⁶; J⁵+M¹, J⁵+M², J⁵+M³, J⁵+M⁴, J⁵+M⁵, J⁵+M⁶; J⁶+M¹, J⁶+M², J⁶+M³, J⁶+M⁴, J⁶+M⁵, J⁶+M⁶; J¹+N¹, J¹+N², J¹+N³, J¹+N⁴, J¹+N⁵, J¹+N⁶; J²+N¹, J²+N², J²+N³, J²+N⁴, J²+N⁵, J²+N⁶; J³+N¹, J³+N², J³+N³, J³+N⁴, J³+N⁵, J³+N⁶; J⁴+N¹, J⁴+N², J⁴+N³, J⁴+N⁴, J⁴+N⁵, J⁴+N⁶; J⁵+N¹, J⁵+N², J⁵+N³, J⁵+N⁴, J⁵+N⁵, J⁵+N⁶; J⁶+N¹, J⁶+N², J⁶+N³, J⁶+N⁴, J⁶+N⁵, J⁶+N⁶; K¹+M¹, K¹+M², K¹+M³, K¹+M⁴, K¹+M⁵, K¹+M⁶; K²+M¹, K²+M², K²+M³, K²+M⁴, K²+M⁵, K²+M⁶; K³+M¹, K³+M², K³+M³, K³+M⁴, K³+M⁵, K³+M⁶; K⁴+M¹, K⁴+M², K⁴+M³, K⁴+M⁴, K⁴+M⁵, K⁴+M⁶; K⁵+M¹, K⁵+M², K⁵+M³, K⁵+M⁴, K⁵+M⁵, K⁵+M⁶; K⁶+M¹, K⁶+M², K⁶+M³, K⁶+M⁴, K⁶+M⁵, K⁶+M⁶; K¹+N¹, K¹+N², K¹+N³, K¹+N⁴, K¹+N⁵, K¹+N⁶; K²+N¹, K²+N², K²+N³, K²+N⁴, K²+N⁵, K²+N⁶; K³+N¹, K³+N², K³+N³, K³+N⁴, K³+N⁵, K³+N⁶; K⁴+N¹, K⁴+N², K⁴+N³, K⁴+N⁴, K⁴+N⁵, K⁴+N⁶; K⁵+N¹, K⁵+N², K⁵+N³, K⁵+N⁴, K⁵+N⁵, K⁵+N⁶; K⁶+N¹, K⁶+N², K⁶+N³, K⁶+N⁴, K⁶+N⁵, K⁶+N⁶; L¹+M¹, L¹+M², L¹+M³, L¹+M⁴, L¹+M⁵, L¹+M⁶; L²+M¹, L²+M², L²+M³, L²+M⁴, L²+M⁵, L²+M⁶; L³+M¹, L³+M², L³+M³, L³+M⁴, L³+M⁵, L³+M⁶; L⁴+M¹, L⁴+M², L⁴+M³, L⁴+M⁴, L⁴+M⁵, L⁴+M⁶; L⁵+M¹, L⁵+M², L⁵+M³, L⁵+M⁴, L⁵+M⁵, L⁵+M⁶; L⁶+M¹, L⁶+M², L⁶+M³, L⁶+M⁴, L⁶+M⁵, L⁶+M⁶; L¹+N¹, L¹+N², L¹+N³, L¹+N⁴, L¹+N⁵, L¹+N⁶; L²+N¹, L²+N², L²+N³, L²+N⁴, L²+N⁵, L²+N⁶; L³+N¹, L³+N², L³+N³, L³+N⁴, L³+N⁵, L³+N⁶; L⁴+N¹, L⁴+N², L⁴+N³, L⁴+N⁴, L⁴+N⁵, L⁴+N⁶; L⁵+N¹, L⁵+N², L⁵+N³, L⁵+N⁴, L⁵+N⁵, L⁵+N⁶; L⁶+N¹, L⁶+N², L⁶+N³, L⁶+N⁴, L⁶+N⁵, and L⁶+N⁶.

Preferably, in any of the above combinations, the relative weight ratio of said multitude of FR particles to said at least one PR particle is within the range of from 5:95 to 95:5, more preferably of from 10:90 to 90:10, still more preferably of from 15:85 to 85:15, yet more preferably of from 20:80 to 80:20, most preferably of from 25:75 to 75:25.

According to a preferred embodiment, the pharmaceutical dosage form according to the invention comprises a multitude of FR particles in combination with a multitude of DR particles, but preferably neither IR particles nor PR particles nor OR particles.

Preferably, the FR particles are in accordance with any of above embodiments G¹ to G⁶, or H¹ to H⁶, or I¹ to I⁶, or J¹ to J⁶, or K¹ to K⁶, or L¹ to L⁶, whereas the DR particle(s) are in accordance with any of above embodiments O¹ to O⁶, or P¹ to P⁶, or Q¹ to Q⁶, or R¹ to R⁶, or S¹ to S⁶. Preferred individualized combinations of embodiments are: G¹+O¹, G¹+O², G¹+O³, G¹+O⁴, G¹+O⁵, G¹+O⁶; G²+O¹, G²+O², G²+O³, G²+O⁴, G²+O⁵, G²+O⁶; G³+O¹, G³+O², G³+O³, G³+O⁴, G³+O⁵, G³+O⁶; G⁴+O¹, G⁴+O², G⁴+O³, G⁴+O⁴, G⁴+O⁵, G⁴+O⁶; G⁵+O¹, G⁵+O², G⁵+O³, G⁵+O⁴, G⁵+O⁵, G⁵+O⁶; G⁶+O¹, G⁶+O², G⁶+O³, G⁶+O⁴, G⁶+O⁵, G⁶+O⁶; G¹+P¹, G¹+P², G¹+P³, G¹+P⁴, G¹+P⁵, G¹+P⁶; G²+P¹, G²+P², G²+P³, G²+P⁴, G²+P⁵, G²+P⁶; G³+P¹, G³+P², G³+P³, G³+P⁴, G³+P⁵, G³+P⁶; G⁴+P¹, G⁴+P², G⁴+P³, G⁴+P⁴, G⁴+P⁵, G⁴+P⁶; G⁵+P¹, G⁵+P², G⁵+P³, G⁵+P⁴, G⁵+P⁵, G⁵+P⁶; G⁶+P¹, G⁶+P², G⁶+P³, G⁶+P⁴, G⁶+P⁵, G⁶+P⁶; G¹+Q¹, G¹+Q², G¹+Q³, G¹+Q⁴, G¹+Q⁵, G¹+Q⁶; G²+Q¹, G²+Q², G²+Q³, G²+Q⁴, G²+Q⁵, G²+Q⁶; G³+Q¹, G³+Q², G³+Q³, G³+Q⁴, G³+Q⁵, G³+Q⁶; G⁴+Q¹, G⁴+Q², G⁴+Q³, G⁴+Q⁴, G⁴+Q⁵, G⁴+Q⁶; G⁵+Q¹, G⁵+Q², G⁵+Q³, G⁵+Q⁴, G⁵+Q⁵, G⁵+Q⁶; G⁶+Q¹, G⁶+Q², G⁶+Q³, G⁶+Q⁴, G⁶+Q⁵, G⁶+Q⁶; G¹+R¹, G¹+R², G¹+R³, G¹+R⁴, G¹+R⁵, G¹+R⁶; G²+R¹, G²+R², G²+R³, G²+R⁴, G²+R⁵, G²+R⁶; G³+R¹, G³+R², G³+R³, G³+R⁴, G³+R⁵, G³+R⁶; G⁴+R¹, G⁴+R², G⁴+R³, G⁴+R⁴, G⁴+R⁵, G⁴+R⁶; G⁵+R¹, G⁵+R², G⁵+R³, G⁵+R⁴, G⁵+R⁵, G⁵+R⁶; G⁶+R¹, G⁶+R², G⁶+R³, G⁶+R⁴, G⁶+R⁵, G⁶+R⁶; G¹+S¹, G¹+S², G¹+S³, G¹+S⁴, G¹+S⁵, G¹+S⁶; G²+S¹, G²+S², G²+S³, G²+S⁴, G²+S⁵, G²+S⁶; G³+S¹, G³+S², G³+S³, G³+S⁴, G³+S⁵, G³+S⁶; G⁴+S¹, G⁴+S², G⁴+S³, G⁴+S⁴, G⁴+S⁵, G⁴+S⁶; G⁵+S¹, G⁵+S², G⁵+S³, G⁵+S⁴, G⁵+S⁵, G⁵+S⁶; G⁶+S¹, G⁶+S², G⁶+S³, G⁶+S⁴, G⁶+S⁵, G⁶+S⁶; H¹+O¹, H¹+O², H¹+O³, H¹+O⁴, H¹+O⁵, H¹+O⁶; H²+O¹, H²+O², H²+O³, H²+O⁴, H²+O⁵, H²+O⁶; H³+O¹, H³+O², H³+O³, H³+O⁴, H³+O⁵, H³+O⁶; H⁴+O¹, H⁴+O², H⁴+O³, H⁴+O⁴, H⁴+O⁵, H⁴+O⁶; H⁵+O¹, H⁵+O², H⁵+O³, H⁵+O⁴, H⁵+O⁵, H⁵+O⁶; H⁶+O¹, H⁶+O², H⁶+O³, H⁶+O⁴, H⁶+O⁵, H⁶+O⁶; H¹+P¹, H¹+P², H¹+P³, H¹+P⁴, H¹+P⁵, H¹+P⁶; H²+P¹, H²+P², H²+P³, H²+P⁴, H²+P⁵, H²+P⁶; H³+P¹, H³+P², H³+P³, H³+P⁴, H³+P⁵, H³+P⁶; H⁴+P¹, H⁴+P², H⁴+P³, H⁴+P⁴, H⁴+P⁵, H⁴+P⁶; H⁵+P¹, H⁵+P², H⁵+P³, H⁵+P⁴, H⁵+P⁵, H⁵+P⁶; H⁶+P¹, H⁶+P², H⁶+P³, H⁶+P⁴, H⁶+P⁵, H⁶+P⁶; H¹+Q¹, H¹+Q², H¹+Q³, H¹+Q⁴, H¹+Q⁵, H¹+Q⁶; H²+Q¹, H²+Q², H²+Q³, H²+Q⁴, H²+Q⁵, H²+Q⁶; H³+Q¹, H³+Q², H³+Q³, H³+Q⁴, H³+Q⁵, H³+Q⁶; H⁴+Q¹, H⁴+Q², H⁴+Q³, H⁴+Q⁴, H⁴+Q⁵, H⁴+Q⁶; H⁵+Q¹, H⁵+Q², H⁵+Q³, H⁵+Q⁴, H⁵+Q⁵, H⁵+Q⁶; H⁶+Q¹, H⁶+Q², H⁶+Q³, H⁶+Q⁴, H⁶+Q⁵, H⁶+Q⁶; H¹+R¹, H¹+R², H¹+R³, H¹+R⁴, H¹+R⁵, H¹+R⁶; H²+R¹, H²+R², H²+R³, H²+R⁴, H²+R⁵, H²+R⁶; H³+R¹, H³+R², H³+R³, H³+R⁴, H³+R⁵, H³+R⁶; H⁴+R¹, H⁴+R², H⁴+R³, H⁴+R⁴, H⁴+R⁵, H⁴+R⁶; H⁵+R¹, H⁵+R², H⁵+R³, H⁵+R⁴, H⁵+R⁵, H⁵+R⁶; H⁶+R¹, H⁶+R², H⁶+R³, H⁶+R⁴, H⁶+R⁵, H⁶+R⁶; H¹+S¹, H¹+S², H¹+S³, H¹+S⁴, H¹+S⁵, H¹+S⁶; H²+S¹, H²+S², H²+S³, H²+S⁴, H²+S⁵, H²+S⁶; H³+S¹, H³+S², H³+S³, H³+S⁴, H³+S⁵, H³+S⁶; H⁴+S¹, H⁴+S², H⁴+S³, H⁴+S⁴, H⁴+S⁵, H⁴+S⁶; H⁵+S¹, H⁵+S², H⁵+S³, H⁵+S⁴, H⁵+S⁵, H⁵+S⁶; H⁶+S¹, H⁶+S², H⁶+S³, H⁶+S⁴, H⁶+S⁵, H⁶+S⁶; I¹+O¹, I¹+O², I¹+O³, I¹+O⁴, I¹+O⁵, I¹+O⁶; I²+O¹, I²+O², I²+O³, I²+O⁴, I²+O⁵, I²+O⁶; I³+O¹, I³+O², I³+O³, I³+O⁴, I³+O⁵, I³+O⁶; I⁴+O¹, I⁴+O², I⁴+O³, I⁴+O⁴, I⁴+O⁵, I⁴+O⁶; I⁵+O¹, I⁵+O², I⁵+O³, I⁵+O⁴, I⁵+O⁵, I⁵+O⁶; I⁶+O¹, I⁶+O², I⁶+O³, I⁶+O⁴, I⁶+O⁵, I⁶+O⁶; I¹+P¹, I¹+P², I¹+P³, I¹+P⁴, I¹+P⁵, I¹+P⁶; I²+P¹, I²+P², I²+P³, I²+P⁴, I²+P⁵, I²+P⁶; I³+P¹, I³+P², I³+P³, I³+P⁴, I³+P⁵, I³+P⁶; I⁴+P¹, I⁴+P², I⁴+P³, I⁴+P⁴, I⁴+P⁵, I⁴+P⁶; I⁵+P¹, I⁵+P², I⁵+P³, I⁵+P⁴, I⁵+P⁵, I⁵+P⁶; I⁶+P¹, I⁶+P², I⁶+P³, I⁶+P⁴, I⁶+P⁵, I⁶+P⁶; I¹+Q¹, I¹+Q², I¹+Q³, I¹+Q⁴, I¹+Q⁵, I¹+Q⁶; I²+Q¹, I²+Q², I²+Q³, I²+Q⁴, I²+Q⁵, I²+Q⁶; I³+Q¹, I³+Q², I³+Q³, I³+Q⁴, I³+Q⁵, I³+Q⁶; I⁴+Q¹, I⁴+Q², I⁴+Q³, I⁴+Q⁴, I⁴+Q⁵, I⁴+Q⁶; I⁵+Q¹, I⁵+Q², I⁵+Q³, I⁵+Q⁴, I⁵+Q⁵, I⁵+Q⁶; I⁶+Q¹, I⁶+Q², I⁶+Q³, I⁶+Q⁴, I⁶+Q⁵, I⁶+Q⁶; I¹+R¹, I¹+R², I¹+R³, I¹+R⁴, I¹+R⁵, I¹+R⁶; I²+R¹, I²+R², I²+R³, I²+R⁴, I²+R⁵, I²+R⁶; I³+R¹, I³+R², I³+R³, I³+R⁴, I³+R⁵, I³+R⁶; I⁴+R¹, I⁴+R², I⁴+R³, I⁴+R⁴, I⁴+R⁵, I⁴+R⁶; I⁵+R¹, I⁵+R², I⁵+R³, I⁵+R⁴, I⁵+R⁵, I⁵+R⁶; I⁶+R¹, I⁶+R², I⁶+R³, I⁶+R⁴, I⁶+R⁵, I⁶+R⁶; I¹+S¹, I¹+S², I¹+S³, I¹+S⁴, I¹+S⁵, I¹+S⁶; I²+S¹, I²+S², I²+S³, I²+S⁴, I²+S⁵, I²+S⁶; I³+S¹, I³+S², I³+S³, I³+S⁴, I³+S⁵, I³+S⁶; I⁴+S¹, I⁴+S², I⁴+S³, I⁴+S⁴, I⁴+S⁵, I⁴+S⁶; I⁵+S¹, I⁵+S², I⁵+S³, I⁵+S⁴, I⁵+S⁵, I⁵+S⁶; I⁶+S¹, I⁶+S², I⁶+S³, I⁶+S⁴, I⁶+S⁵, I⁶+S⁶; J¹+O¹, J¹+O², J¹+O³, J¹+O⁴, J¹+O⁵, J¹+O⁶; J²+O¹, J²+O², J²+O³, J²+O⁴, J²+O⁵, J²+O⁶; J³+O¹, J³+O², J³+O³, J³+O⁴, J³+O⁵, J³+O⁶; J⁴+O¹, J⁴+O², J⁴+O³, J⁴+O⁴, J⁴+O⁵, J⁴+O⁶; J⁵+O¹, J⁵+O², J⁵+O³, J⁵+O⁴, J⁵+O⁵, J⁵+O⁶; J⁶+O¹, J⁶+O², J⁶+O³, J⁶+O⁴, J⁶+O⁵, J⁶+O⁶; J¹+P¹, J¹+P², J¹+P³, J¹+P⁴, J¹+P⁵, J¹+P⁶; J²+P¹, J²+P², J²+P³, J²+P⁴, J²+P⁵S J²+P⁶; J³+P¹, J³+P², J³+P³, J³+P⁴, J³+P⁵, J³+P⁶; J⁴+P¹, J⁴+P², J⁴+P³, J⁴+P⁴, J⁴+P⁵, J⁴+P⁶; J⁵+P¹, J⁵+P², J⁵+P³, J⁵+P⁴, J⁵+P⁵, J⁵+P⁶; J⁶+P¹, J⁶+P², J⁶+P³, J⁶+P⁴, J⁶+P⁵, J⁶+P⁶; J¹+Q¹, J¹+Q², J¹+Q³, J¹+Q⁴, J¹+Q⁵, J¹+Q⁶; J²+Q¹, J²+Q², J²+Q³, J²+Q⁴, J²+Q⁵, J²+Q⁶; J³+Q¹, J³+Q², J³+Q³, J³+Q⁴, J³+Q⁵, J³+Q⁶; J⁴+Q¹, J⁴+Q², J⁴+Q³, J⁴+Q⁴, J⁴+Q⁵, J⁴+Q⁶; J⁵+Q¹, J⁵+Q², J⁵+Q³, J⁵+Q⁴, J⁵+Q⁵, J⁵+Q⁶; J⁶+Q¹, J⁶+Q², J⁶+Q³, J⁶+Q⁴, J⁶+Q⁵, J⁶+Q⁶; J¹+R¹, J¹+R², J¹+R³, J¹+R⁴, J¹+R⁵, J¹+R⁶; J²+R¹, J²+R², J²+R³, J²+R⁴, J²+R⁵, J²+R⁶; J³+R¹, J³+R², J³+R³, J³+R⁴, J³+R⁵, J³+R⁶; J⁴+R¹, J⁴+R², J⁴+R³, J⁴+R⁴, J⁴+R⁵, J⁴+R⁶; J⁵+R¹, J⁵+R², J⁵+R³, J⁵+R⁴, J⁵+R⁵, J⁵+R⁶; J⁶+R¹, J⁶+R², J⁶+R³, J⁶+R⁴, J⁶+R⁵, J⁶+R⁶; J¹+S¹, J¹+S², J¹+S³, J¹+S⁴, J¹+S⁵, J¹+S⁶; J²+S¹, J²+S², J²+S³, J²+S⁴, J²+S⁵, J²+S⁶; J³+S¹, J³+S², J³+S³, J³+S⁴, J³+S⁵, J³+S⁶; J⁴+S¹, J⁴+S², J⁴+S³, J⁴+S⁴, J⁴+S⁵, J⁴+S⁶; J⁵+S¹, J⁵+S², J⁵+S³, J⁵+S⁴, J⁵+S⁵, J⁵+S⁶; J⁶+S¹, J⁶+S², J⁶+S³, J⁶+S⁴, J⁶+S⁵, J⁶+S⁶; K¹+O¹, K¹+O², K¹+O³, K¹+O⁴, K¹+O⁵, K¹+O⁶; K²+O¹, K²+O², K²+O³, K²+O⁴, K²+O⁵, K²+O⁶; K³+O¹, K³+O², K³+O³, K³+O⁴, K³+O⁵, K³+O⁶; K⁴+O¹, K⁴+O², K⁴+O³, K⁴+O⁴, K⁴+O⁵, K⁴+O⁶; K⁵+O¹, K⁵+O², K⁵+O³, K⁵+O⁴, K⁵+O⁵, K⁵+O⁶; K⁶+O¹, K⁶+O², K⁶+O³, K⁶+O⁴, K⁶+O⁵, K⁶+O⁶; K¹+P¹, K¹+P², K¹+P³, K¹+P⁴, K¹+P⁵, K¹+P⁶; K²+P¹, K²+P², K²+P³, K²+P⁴, K²+P⁵, K²+P⁶; K³+P¹, K³+P², K³+P³, K³+P⁴, K³+P⁵, K³+P⁶; K⁴+P¹, K⁴+P², K⁴+P³, K⁴+P⁴, K⁴+P⁵, K⁴+P⁶; K⁵+P¹, K⁵+P², K⁵+P³, K⁵+P⁴, K⁵+P⁵, K⁵+P⁶; K⁶+P¹, K⁶+P², K⁶+P³, K⁶+P⁴, K⁶+P⁵, K⁶+P⁶; K¹+Q¹, K¹+Q², K¹+Q³, K¹+Q⁴, K¹+Q⁵, K¹+Q⁶; K²+Q¹, K²+Q², K²+Q³, K²+Q⁴, K²+Q⁵, K²+Q⁶; K³+Q¹, K³+Q², K³+Q³, K³+Q⁴, K³+Q⁵, K³+Q⁶; K⁴+Q¹, K⁴+Q², K⁴+Q³, K⁴+Q⁴, K⁴+Q⁵, K⁴+Q⁶; K⁵+Q¹, K⁵+Q², K⁵+Q³, K⁵+Q⁴, K⁵+Q⁵, K⁵+Q⁶; K⁶+Q¹, K⁶+Q², K⁶+Q³, K⁶+Q⁴, K⁶+Q⁵, K⁶+Q⁶; K¹+R¹, K¹+R², K¹+R³, K¹+R⁴, K¹+R⁵, K¹+R⁶; K²+R¹, K²+R², K²+R³, K²+R⁴, K²+R⁵, K²+R⁶; K³+R¹, K³+R², K³+R³, K³+R⁴, K³+R⁵, K³+R⁶; K⁴+R¹, K⁴+R², K⁴+R³, K⁴+R⁴, K⁴+R⁵, K⁴+R⁶; K⁵+R¹, K⁵+R², K⁵+R³, K⁵+R⁴, K⁵+R⁵, K⁵+R⁶; K⁶+R¹, K⁶+R², K⁶+R³, K⁶+R⁴, K⁶+R⁵, K⁶+R⁶; K¹+S¹, K¹+S², K¹+S³, K¹+S⁴, K¹+S⁵, K¹+S⁶; K²+S¹, K²+S², K²+S³, K²+S⁴, K²+S⁵, K²+S⁶; K³+S¹, K³+S², K³+S³, K³+S⁴, K³+S⁵, K³+S⁶; K⁴+S¹, K⁴+S², K⁴+S³, K⁴+S⁴, K⁴+S⁵, K⁴+S⁶; K⁵+S¹, K⁵+S², K⁵+S³, K⁵+S⁴, K⁵+S⁵, K⁵+S⁶; K⁶+S¹, K⁶+S², K⁶+S³, K⁶+S⁴, K⁶+S⁵, K⁶+S⁶; L¹+O¹, L¹+O², L¹+O³, L¹+O⁴, L¹+O⁵, L¹+O⁶; L²+O¹, L²+O², L²+O³, L²+O⁴, L²+O⁵, L²+O⁶; L³+O¹, L³+O², L³+O³, L³+O⁴, L³+O⁵, L³+O⁶; L⁴+O¹, L⁴+O², L⁴+O³, L⁴+O⁴, L⁴+O⁵, L⁴+O⁶; L⁵+O¹, L⁵+O², L⁵+O³, L⁵+O⁴, L⁵+O⁵, L⁵+O⁶; L⁶+O¹, L⁶+O², L⁶+O³, L⁶+O⁴, L⁶+O⁵, L⁶+O⁶; L¹+P¹, L¹+P², L¹+P³, L¹+P⁴, L¹+P⁵, L¹+P⁶; L²+P¹, L²+P², L²+P³, L²+P⁴, L²+P⁵, L²+P⁶; L³+P¹, L³+P², L³+P³, L³+P⁴, L³+P⁵, L³+P⁶; L⁴+P¹, L⁴+P², L⁴+P³, L⁴+P⁴, L⁴+P⁵, L⁴+P⁶; L⁵+P¹, L⁵+P², L⁵+P³, L⁵+P⁴, L⁵+P⁵, L⁵+P⁶; L⁶+P¹, L⁶+P², L⁶+P³, L⁶+P⁴, L⁶+P⁵, L⁶+P⁶; L¹+Q¹, L¹+Q², L¹+Q³, L¹+Q⁴, L¹+Q⁵, L¹+Q⁶; L²+Q¹, L²+Q², L²+Q³, L²+Q⁴, L²+Q⁵, L²+Q⁶; L³+Q¹, L³+Q², L³+Q³, L³+Q⁴, L³+Q⁵, L³+Q⁶; L⁴+Q¹, L⁴+Q², L⁴+Q³, L⁴+Q⁴, L⁴+Q⁵, L⁴+Q⁶; L⁵+Q¹, L⁵+Q², L⁵+Q³, L⁵+Q⁴, L⁵+Q⁵, L⁵+Q⁶; L⁶+Q¹, L⁶+Q², L⁶+Q³, L⁶+Q⁴, L⁶+Q⁵, L⁶+Q⁶; L¹+R¹, L¹+R², L¹+R³, L¹+R⁴, L¹+R⁵, L¹+R⁶; L²+R¹, L²+R², L²+R³, L²+R⁴, L²+R⁵, L²+R⁶; L³+R¹, L³+R², L³+R³, L³+R⁴, L³+R⁵, L³+R⁶; L⁴+R¹, L⁴+R², L⁴+R³, L⁴+R⁴, L⁴+R⁵, L⁴+R⁶; L⁵+R¹, L⁵+R², L⁵+R³, L⁵+R⁴, L⁵+R⁵, L⁵+R⁶; L⁶+R¹, L⁶+R², L⁶+R³, L⁶+R⁴, L⁶+R⁵, L⁶+R⁶; L¹+S¹, L¹+S², L¹+S³, L¹+S⁴, L¹+S⁵, L¹+S⁶; L²+S¹, L²+S², L²+S³, L²+S⁴, L²+S⁵, L²+S⁶; L³+S¹, L³+S², L³+S³, L³+S⁴, L³+S⁵, L³+S⁶; L⁴+S¹, L⁴+S², L⁴+S³, L⁴+S⁴, L⁴+S⁵, L⁴+S⁶; L⁵+S¹, L⁵+S², L⁵+S³, L⁵+S⁴, L⁵+S⁵, L⁵+S⁶; L⁶+S¹, L⁶+S², L⁶+S³, L⁶+S⁴, L⁶+S⁵, and L⁶+S⁶.

Preferably, in any of the above combinations, the relative weight ratio of said multitude of FR particles to said multitude of DR particles is within the range of from 5:95 to 95:5, more preferably of from 10:90 to 90:10, still more preferably of from 15:85 to 85:15, yet more preferably of from 20:80 to 80:20, most preferably of from 25:75 to 75:25.

According to a preferred embodiment, the pharmaceutical dosage form according to the invention comprises a multitude of FR particles in combination with a multitude of OR particles, but preferably neither IR particles nor PR particles nor DR particles.

Preferably, the FR particles are in accordance with any of above embodiments G¹ to G⁶, or H¹ to H⁶, or I¹ to I⁶, or J¹ to J⁶, or K¹ to K⁶, or L¹ to L⁶, whereas the OR particle(s) are in accordance with any of above embodiments T¹ to T⁶, or U¹ to U⁶. Preferred individualized combinations of embodiments are: G¹+T¹, G¹+T², G¹+T³, G¹+T⁴, G¹+T⁵, G¹+T⁶; G²+T¹, G²+T², G²+T³, G²+T⁴, G²+T⁵, G²+T⁶; G³+T¹, G³+T², G³+T³, G³+T⁴, G³+T⁵, G³+T⁶; G⁴+T¹, G⁴+T², G⁴+T³, G⁴+T⁴, G⁴+T⁵, G⁴+T⁶; G⁵+T¹, G⁵+T², G⁵+T³, G⁵+T⁴, G⁵+T⁵, G⁵+T⁶; G⁶+T¹, G⁶+T², G⁶+T³, G⁶+T⁴, G⁶+T⁵, G⁶+T⁶; G¹+U¹, G¹+U², G¹+U³, G¹+U⁴, G¹+U⁵, G¹+U⁶; G²+U¹, G²+U², G²+U³, G²+U⁴, G²+U⁵, G²+U⁶; G³+U¹, G³+U², G³+U³, G³+U⁴, G³+U⁵, G³+U⁶; G⁴+U¹, G⁴+U², G⁴+U³, G⁴+U⁴, G⁴+U⁵, G⁴+U⁶; G⁵+U¹, G⁵+U², G⁵+U³, G⁵+U⁴, G⁵+U⁵, G⁵+U⁶; G⁶+U¹, G⁶+U², G⁶+U³, G⁶+U⁴, G⁶+U⁵, G⁶+U⁶; H¹+T¹, H¹+T², H¹+T³, H¹+T⁴, H¹+T⁵, H¹+T⁶; H²+T¹, H²+T², H²+T³, H²+T⁴, H²+T⁵, H²+T⁶; H³+T¹, H³+T², H³+T³, H³+T⁴, H³+T⁵, H³+T⁶; H⁴+T¹, H⁴+T², H⁴+T³, H⁴+T⁴, H⁴+T⁵, H⁴+T⁶; H⁵+T¹, H⁵+T², H⁵+T³, H⁵+T⁴, H⁵+T⁵, H⁵+T⁶; H⁶+T¹, H⁶+T², H⁶+T³, H⁶+T⁴, H⁶+T⁵, H⁶+T⁶; H¹+U¹, H¹+U², H¹+U³, H¹+U⁴, H¹+U⁵, H¹+U⁶; H²+U¹, H²+U², H²+U³, H²+U⁴, H²+U⁵, H²+U⁶; H³+U¹, H³+U², H³+U³, H³+U⁴, H³+U⁵, H³+U⁶; H⁴+U¹, H⁴+U², H⁴+U³, H⁴+U⁴, H⁴+U⁵, H⁴+U⁶; H⁵+U¹, H⁵+U², H⁵+U³, H⁵+U⁴, H⁵+U⁵, H⁵+U⁶; H⁶+U¹, H⁶+U², H⁶+U³, H⁶+U⁴, H⁶+U⁵, H⁶+U⁶; I¹+T¹, I¹+T², I¹+T³, I¹+T⁴, I¹+T⁵, I¹+T⁶; I²+T¹, I²+T², I²+T³, I²+T⁴, I²+T⁵, I²+T⁶; I³+T¹, I³+T², I³+T³, I³+T⁴, I³+T⁵, I³+T⁶; I⁴+T¹, I⁴+T², I⁴+T³, I⁴+T⁴, I⁴+T⁵, I⁴+T⁶; I⁵+T¹, I⁵+T², I⁵+T³, I⁵+T⁴, I⁵+T⁵, I⁵+T⁶; I⁶+T¹, I⁶+T², I⁶+T³, I⁶+T⁴, I⁶+T⁵, I⁶+T⁶; I¹+U¹, I¹+U², I¹+U³, I¹+U⁴, I¹+U⁵, I¹+U⁶; I²+U¹, I²+U², I²+U³, I²+U⁴, I²+U⁵, I²+U⁶; I³+U¹, I³+U², I³+U³, I³+U⁴, I³+U⁵, I³+U⁶; I⁴+U¹, I⁴+U², I⁴+U³, I⁴+U⁴, I⁴+U⁵, I⁴+U⁶; I⁵+U¹, I⁵+U², I⁵+U³, I⁵+U⁴, I⁵+U⁵, I⁵+U⁶; I⁶+U¹, I⁶+U², I⁶+U³, I⁶+U⁴, I⁶+U⁵, I⁶+U⁶; J¹+T¹, J¹+T², J¹+T³, J¹+T⁴, J¹+T⁵, J¹+T⁶; J²+T¹, J²+T², J²+T³, J²+T⁴, J²+T⁵, J²+T⁶; J³+T¹, J³+T², J³+T³, J³+T⁴, J³+T⁵, J³+T⁶; J⁴+T¹, J⁴+T², J⁴+T³, J⁴+T⁴, J⁴+T⁵, J⁴+T⁶; J⁵+T¹, J⁵+T², J⁵+T³, J⁵+T⁴, J⁵+T⁵, J⁵+T⁶; J⁶+T¹, J⁶+T², J⁶+T³, J⁶+T⁴, J⁶+T⁵, J⁶+T⁶; J¹+U¹, J¹+U², J¹+U³, J¹+U⁴, J¹+U⁵, J¹+U⁶; J²+U¹, J²+U², J²+U³, J²+U⁴, J²+U⁵, J²+U⁶; J³+U¹, J³+U², J³+U³, J³+U⁴, J³+U⁵, J³+U⁶; J⁴+U¹, J⁴+U², J⁴+U³, J⁴+U⁴, J⁴+U⁵, J⁴+U⁶; J⁵+U¹, J⁵+U², J⁵+U³, J⁵+U⁴, J⁵+U⁵, J⁵+U⁶; J⁶+U¹, J⁶+U², J⁶+U³, J⁶+U⁴, J⁶+U⁵, J⁶+U⁶; K¹+T¹, K¹+T², K¹+T³, K¹+T⁴, K¹+T⁵, K¹+T⁶; K²+T¹, K²+T², K²+T³, K²+T⁴, K²+T⁵, K²+T⁶; K³+T¹, K³+T², K³+T³, K³+T⁴, K³+T⁵, K³+T⁶; K⁴+T¹, K⁴+T², K⁴+T³, K⁴+T⁴, K⁴+T⁵, K⁴+T⁶; K⁵+T¹, K⁵+T², K⁵+T³, K⁵+T⁴, K⁵+T⁵, K⁵+T⁶; K⁶+T¹, K⁶+T², K⁶+T³, K⁶+T⁴, K⁶+T⁵, K⁶+T⁶; K¹+U¹, K¹+U², K¹+U³, K¹+U⁴, K¹+U⁵, K¹+U⁶; K²+U¹, K²+U², K²+U³, K²+U⁴, K²+U⁵, K²+U⁶; K³+U¹, K³+U², K³+U³, K³+U⁴, K³+U⁵, K³+U⁶; K⁴+U¹, K⁴+U², K⁴+U³, K⁴+U⁴, K⁴+U⁵, K⁴+U⁶; K⁵+U¹, K⁵+U², K⁵+U³, K⁵+U⁴, K⁵+U⁵, K⁵+U⁶; K⁶+U¹, K⁶+U², K⁶+U³, K⁶+U⁴, K⁶+U⁵, K⁶+U⁶; L¹+T¹, L¹+T², L¹+T³, L¹+T⁴, L¹+T⁵, L¹+T⁶; L²+T¹, L²+T², L²+T³, L²+T⁴, L²+T⁵, L²+T⁶; L³+T¹, L³+T², L³+T³, L³+T⁴, L³+T⁵, L³+T⁶; L⁴+T¹, L⁴+T², L⁴+T³, L⁴+T⁴, L⁴+T⁵, L⁴+T⁶; L⁵+T¹, L⁵+T², L⁵+T³, L⁵+T⁴, L⁵+T⁵, L⁵+T⁶; L⁶+T¹, L⁶+T², L⁶+T³, L⁶+T⁴, L⁶+T⁵, L⁶+T⁶; L¹+U¹, L¹+U², L¹+U³, L¹+U⁴, L¹+U⁵, L¹+U⁶; L²+U¹, L²+U², L²+U³, L²+U⁴, L²+U⁵, L²+U⁶; L³+U¹, L³+U², L³+U³, L³+U⁴, L³+U⁵, L³+U⁶; L⁴+U¹, L⁴+U², L⁴+U³, L⁴+U⁴, L⁴+U⁵, L⁴+U⁶; L⁵+U¹, L⁵+U², L⁵+U³, L⁵+U⁴, L⁵+U⁵, L⁵+U⁶; L⁶+U¹, L⁶+U², L⁶+U³, L⁶+U⁴, L⁶+U⁵, and L⁶+U⁶.

Preferably, in any of the above combinations, the relative weight ratio of said multitude of FR particles to said at least one OR particle is within the range of from 5:95 to 95:5, more preferably of from 10:90 to 90:10, still more preferably of from 15:85 to 85:15, yet more preferably of from 20:80 to 80:20, most preferably of from 25:75 to 75:25.

The IR particles and/or FR particles and/or DR particles and/or OR particles independently of one another are of macroscopic size, i.e. typically have an average particle size of at least 50 μm, more preferably at least 100 μm, still more preferably at least 150 μm or at least 200 μm, yet more preferably at least 250 μm or at least 300 μm, most preferably at least 400 μm or at least 500 μm, and in particular at least 550 μm or at least 600 μm.

The IR particles and/or FR particles and/or DR particles and/or OR particles independently of one another have an average diameter is within the range of from 100 μm to 1500 μm, preferably 200 μm to 1500 μm, more preferably 300 μm to 1500 μm, still more preferably 400 μm to 1500 μm, most preferably 500 μm to 1500 μm, and in particular 600 μm to 1500 μm.

Preferred IR particles and/or FR particles and/or DR particles and/or OR particles independently of one another have an average length and average diameter of 1000 μm or less. When the particles are manufactured by extrusion technology, the “length” of particles is the dimension of the particles that is parallel to the direction of extrusion. The “diameter” of particles is the largest dimension that is perpendicular to the direction of extrusion.

Particularly preferred IR particles and/or FR particles and/or DR particles and/or OR particles independently of one another have an average diameter of less than 1000 μm, more preferably less than 800 μm, still more preferably of less than 650 μm. Especially preferred IR particles and/or FR particles and/or DR particles and/or OR particles independently of one another have an average diameter of less than 700 μm, particularly less than 600 μm, still more particularly less than 500 μm, e.g. less than 400 μm. Particularly preferred IR particles and/or FR particles and/or DR particles and/or OR particles independently of one another have an average diameter in the range 200 to 1000 μm, more preferably 400 to 800 μm, still more preferably 450 to 700 μm, yet more preferably 500 to 650 μm, e.g. 500 to 600 μm. Further preferred IR particles and/or FR particles and/or DR particles and/or OR particles independently of one another have an average diameter of between 300 μm and 400 μm, of between 400 μm and 500 μm, or of between 500 μm and 600 μm, or of between 600 μm and 700 μm or of between 700 μm and 800 μm.

Preferred IR particles and/or FR particles and/or DR particles and/or OR particles independently of one another have an average length of less than 1000 μm, preferably an average length of less than 800 μm, still more preferably an average length of less than 650 μm, e.g. a length of 800 μm, 700 μm 600 μm, 500 μm, 400 μm or 300 μm. Especially preferred IR particles and/or FR particles and/or DR particles and/or OR particles independently of one another have an average length of less than 700 μm, particularly less than 650 μm, still more particularly less than 550 μm, e.g. less than 450 μm. Particularly preferred IR particles and/or FR particles and/or DR particles and/or OR particles independently of one another therefore have an average length in the range 200-1000 μm, more preferably 400-800 μm, still more preferably 450-700 μm, yet more preferably 500-650 μm, e.g. 500-600 μm. The minimum average length of the IR particles and/or FR particles and/or DR particles and/or OR particles independently of one another is determined by the cutting step and may be, e.g. 500 μm, 400 μm, 300 μm or 200 μm.

In a preferred embodiment, the IR particles and/or FR particles and/or DR particles and/or OR particles independently of one another have (i) an average diameter of 1000±300 μm, more preferably 1000±250 μm, still more preferably 1000±200 μm, yet more preferably 1000±150 μm, most preferably 1000±100 μm, and in particular 1000±50 μm; and/or (ii) an average length of 1000±300 μm, more preferably 1000±250 μm, still more preferably 1000±200 μm, yet more preferably 1000±150 μm, most preferably 1000±100 μm, and in particular 1000±50 μm.

The size of IR particles and/or FR particles and/or DR particles and/or OR particles independently of one another may be determined by any conventional procedure known in the art, e.g. laser light scattering, sieve analysis, light microscopy or image analysis.

Preferably, the multitude of IR particles and/or the multitude of FR particles and/or the multitude of DR particles and/or the multitude of OR particles independently of one another has an arithmetic average weight, in the following referred to as “aaw”, wherein at least 70%, more preferably at least 75%, still more preferably at least 80%, yet more preferably at least 85%, most preferably at least 90% and in particular at least 95% of the individual particles contained in said plurality of particles has an individual weight within the range of aaw±30%, more preferably aaw±25%, still more preferably aaw±20%, yet more preferably aaw±15%, most preferably aaw±10%, and in particular aaw±5%. For example, if the pharmaceutical dosage form according to the invention contains a plurality of 100 IR particles and aaw of said plurality of IR particles is 1.00 mg, at least 75 individual IR particles (i.e. 75%) have an individual weight within the range of from 0.70 to 1.30 mg (1.00 mg±30%).

Preferably, the content of the particles independently of one another is at most 95 wt.-% or at most 90 wt.-%, more preferably at most 85 wt.-% or at most 80 wt.-%, still more preferably at most 75 wt.-% or at most 70 wt.-%, yet more preferably at most 65 wt.-% or at most 60 wt.-%, most preferably at most 55 wt.-% or at most 50 wt.-%, and in particular at most 45 wt.-% or at most 40 wt.-%, based on the total weight of the pharmaceutical dosage form.

Preferably, the content of the particles independently of one another is at least 2.5 wt.-%, at least 3.0 wt.-%, at least 3.5 wt.-% or at least 4.0 wt.-%; more preferably at least 4.5 wt.-%, at least 5.0 wt.-%, at least 5.5 wt.-% or at least 6.0 wt.-%; most preferably at least 6.5 wt.-%, at least 7.0 wt.-%, at least 7.5 wt.-% or at least 8.0 wt.-%; and in particular at least 8.5 wt.-%, at least 9.0 wt.-%, at least 9.5 wt.-% or at least 10 wt.-%; based on the total weight of the pharmaceutical dosage form.

In a preferred embodiment, the content of the particles independently of one another is within the range of 10±7.5 wt.-%, more preferably 10±5.0 wt.-%, still more preferably 10±4.0 wt.-%, yet more preferably 10±3.0 wt.-%, most preferably 10±2.0 wt.-%, and in particular 10±1.0 wt.-%, based on the total weight of the pharmaceutical dosage form. In another preferred embodiment, the content of the particles independently of one another is within the range of 15±12.5 wt.-%, more preferably 15±10 wt.-%, still more preferably 15±8.0 wt.-%, yet more preferably 15±6.0 wt.-%, most preferably 15±4.0 wt.-%, and in particular 15±2.0 wt.-%, based on the total weight of the pharmaceutical dosage form. In still another preferred embodiment, the content of the particles independently of one another is within the range of 20±17.5 wt.-%, more preferably 20±15 wt.-%, still more preferably 20±12.5 wt.-%, yet more preferably 20±10 wt.-%, most preferably 20±7.5 wt.-%, and in particular 20±5 wt.-%, based on the total weight of the pharmaceutical dosage form. In yet another preferred embodiment, the content of the particles independently of one another is within the range of 25±17.5 wt.-%, more preferably 25±15 wt.-%, still more preferably 25±12.5 wt.-%, yet more preferably 25±10 wt.-%, most preferably 25±7.5 wt.-%, and in particular 25±5 wt.-%, based on the total weight of the pharmaceutical dosage form. In another preferred embodiment, the content of the particles independently of one another is within the range of 30±17.5 wt.-%, more preferably 30±15 wt.-%, still more preferably 30±12.5 wt.-%, yet more preferably 30±10 wt.-%, most preferably 30±7.5 wt.-%, and in particular 30±5 wt.-%, based on the total weight of the pharmaceutical dosage form. In still another preferred embodiment, the particles independently of one another is within the range of 35±17.5 wt.-%, more preferably 35±15 wt.-%, still more preferably 35±12.5 wt.-%, yet more preferably 35±10 wt.-%, most preferably 35±7.5 wt.-%, and in particular 35±5 wt.-%, based on the total weight of the pharmaceutical dosage form. In another preferred embodiment, the particles independently of one another is within the range of 40±17.5 wt.-%, more preferably 40±15 wt.-%, still more preferably 40±12.5 wt.-%, yet more preferably 40±10 wt.-%, most preferably 40±7.5 wt.-%, and in particular 40±5 wt.-%, based on the total weight of the pharmaceutical dosage form.

The pharmacologically active compound is not particularly limited. In a preferred embodiment, the particles and the pharmaceutical dosage form contain only a single pharmacologically active compound. In another preferred embodiment, the particles and the pharmaceutical dosage form contain a combination of two or more pharmacologically active compounds.

Preferably, pharmacologically active compound is an active ingredient with potential for being abused. Active ingredients with potential for being abused are known to the person skilled in the art and comprise e.g. tranquilizers, stimulants, barbiturates, narcotics, opioids or opioid derivatives.

Preferably, the pharmacologically active compound exhibits psychotropic action.

In a preferred embodiment, the pharmacologically active compound is an opioid. According to the ATC index, opioids are divided into natural opium alkaloids, phenylpiperidine derivatives, diphenylpropylamine derivatives, benzomorphan derivatives, oripavine derivatives, morphinan derivatives and others. Preferred opioids include but are not limited to oxycodone, oxymorphone, hydrocodone, hydromorphone, morphine, tapentadol, tramadol and the physiologically acceptable salts thereof.

In another preferred embodiment, the pharmacologically active compound is a stimulant. Stimulants are psychoactive drugs that induce temporary improvements in either mental or physical functions or both. Examples of these kinds of effects may include enhanced wakefulness, locomotion, and alertness. Preferred stimulants are phenylethylamine derivatives. According to the ATC index, stimulants are contained in different classes and groups, e.g. psychoanaleptics, especially psychostimulants, agents used for ADHD and nootropics, particularly centrally acting sympathomimetics; and e.g. nasal preparations, especially nasal decongestants for systemic use, particularly sympathomimetics.

Preferably, the pharmacologically active compound belongs to the group of psychoanaleptics [ATC N06]. Preferably, the pharmacologically active compound belongs to the group of psychostimulants, agents used for ADHD, and nootropics [ATC N06B].

Preferably, the pharmacologically active compound belongs to the group of centrally acting sympathomimetics [ATC N06BA]. Preferably the pharmacologically active compound is selected from the group consisting of amphetamine, dexamphetamine, metamphetamine, methylphenidate, pemoline, fencamfamin, modafinil, fenozolone, atomoxetine, fenetylline, dexmethylphenidate, lisdexamphetamine, armodafinil, and the physiologically acceptable salts of any of the foregoing.

In a preferred embodiment, the pharmacologically active compound is a stimulant selected from the group consisting of amphetamine, dex-amphetamine (dextroamphetamine), dex-methylphenidate, atomoxetine, caffeine, ephedrine, phenylpropanolamine, phenylephrine, fencamphamin, fenozolone, fenetylline, methylenedioxymethamphetamine (MDMA), methylenedioxypyrovalerone (MDPV), prolintane, lisdexamfetamine, mephedrone, meth-amphetamine, methylphenidate, modafinil, nicotine, pemoline, phenylpropanolamine, propylhexedrine, dimethylamylamine, and pseudoephedrine.

In a particularly preferred embodiment, the pharmacologically active compound is amphetamine or a physiologically acceptable salt thereof, preferably amphetamine sulfate and/or amphetamine aspartate, such as amphetamine aspartate monohydrate.

In another particularly preferred embodiment, the pharmacologically active compound is dextroamphetamine or a physiologically acceptable salt thereof, preferably dextroamphetamine saccharate or dextroamphetamine sulfate.

In still another particularly preferred embodiment, the pharmacologically active compound is lisdexamfetamin or a physiologically acceptable salt thereof.

In another preferred embodiment, the pharmacologically active compound is amphetamine sulfate and the pharmaceutical dosage form does not contain any other salt of amphetamine.

In yet another particularly preferred embodiment, the pharmacologically active compound is methylphenidate or a physiologically acceptable salt thereof.

In even another particularly preferred embodiment, the pharmacologically active compound is dexmethylphenidate or a physiologically acceptable salt thereof.

Preferably, said pharmacologically active compound is the only pharmacologically active compound contained in the pharmaceutical dosage form.

However, it is also possible that the pharmaceutical dosage form comprises a combination of more than a single pharmacologically active compound.

A preferred combination comprises
amphetamine or a physiologically acceptable salt of amphetamine or a combination of more than one physiologically acceptable salt of amphetamine with
dextroamphetamine or a physiologically acceptable salt of dextroamphetamine or a combination of more than one physiologically acceptable salt of dextroamphetamine.

Another preferred combination comprises
methylphenidate or a physiologically acceptable salt of methylphenidate or a combination of more than one physiologically acceptable salt of methylphenidate with
dexmethylphenidate or a physiologically acceptable salt of dexmethylphenidate or a combination of more than one physiologically acceptable salt of dexmethylphenidate.

The pharmaceutical dosage form according to the invention preferably contains no antagonists for the pharmacologically active compound, preferably no antagonists against psychotropic substances.

Further, the pharmaceutical dosage form according to the invention preferably also contains no bitter substance. Bitter substances and the quantities effective for use may be found in US-2003/0064099 A1, the corresponding disclosure of which should be deemed to be the disclosure of the present application and is hereby introduced as a reference. Examples of bitter substances are aromatic oils, such as peppermint oil, eucalyptus oil, bitter almond oil, menthol, fruit aroma substances, aroma substances from lemons, oranges, limes, grapefruit or mixtures thereof, and/or denatonium benzoate.

The pharmaceutical dosage form according to the invention accordingly preferably contains neither antagonists for the pharmacologically active compound nor bitter substances.

Preferably, the total amount of the pharmacologically active compound contained in the pharmaceutical dosage form is contained in the multitude of immediate release particles (i.e. IR particles or FR particles) and the at least one controlled release particle (i.e. PR particle(s), DR particles or OR particles).

Preferably, 15 wt.-% to 85 wt.-%, more preferably 20 wt.-% to 80 wt.-%, still more preferably 25 wt.-% to 75 wt.-%, yet more preferably 30 wt.-% to 70 wt.-%, even more preferably 35 wt.-% to 65 wt.-%, most preferably 40 wt.-% to 60 wt.-%, and in particular 45 wt.-% to 55 wt.-% of the total amount of the pharmacologically active compound, which is contained in the pharmaceutical dosage form, is contained in said multitude of immediate release particles (i.e. IR particles or FR particles).

Preferably, 15 wt.-% to 85 wt.-%, more preferably 20 wt.-% to 80 wt.-%, still more preferably 25 wt.-% to 75 wt.-%, yet more preferably 30 wt.-% to 70 wt.-%, even more preferably 35 wt.-% to 65 wt.-%, most preferably 40 wt.-% to 60 wt.-%, and in particular 45 wt.-% to 55 wt.-% of the total amount of the pharmacologically active compound, which is contained in the pharmaceutical dosage form, is contained in said at least one controlled release particle (i.e. PR particle(s), DR particles or OR particles).

The content of the pharmacologically active compound in the particles and in the pharmaceutical dosage form, respectively, preferably amounts to 3 to 75 wt.-%, more preferably 5 to 70 wt.-%, still more preferably 7.5 to 65 wt.-%, based on the total weight of the pharmaceutical dosage form or based on the total weight of the particles.

Preferably, the content of the pharmacologically active compound is at least 25 wt.-%, more preferably at least 30 wt.-%, still more preferably at least 35 wt.-%, yet more preferably at least 40 wt.-%, most preferably at least 45 wt.-%, based on the total weight of the pharmaceutical dosage form or based on the total weight of the particles.

Preferably, the content of the pharmacologically active compound is at most 70 wt.-%, more preferably at most 65 wt.-%, still more preferably at most 60 wt.-%, yet more preferably at most 55 wt.-%, most preferably at most 50 wt.-%, based on the total weight of the pharmaceutical dosage form or based on the total weight of the particles.

In a preferred embodiment, the content of the pharmacologically active compound is within the range of 35±30 wt.-%, more preferably 35±25 wt.-%, still more preferably 35±20 wt.-%, yet more preferably 35±15 wt.-%, most preferably 35±10 wt.-%, and in particular 35±5 wt.-%, based on the total weight of the pharmaceutical dosage form or based on the total weight of the particles. In another preferred embodiment, the content of the pharmacologically active compound is within the range of 45±30 wt.-%, more preferably 45±25 wt.-%, still more preferably 45±20 wt.-%, yet more preferably 45±15 wt.-%, most preferably 45±10 wt.-%, and in particular 45±5 wt.-%, based on the total weight of the pharmaceutical dosage form or based on the total weight of the particles. In still another preferred embodiment, the content of the pharmacologically active compound is within the range of 55±30 wt.-%, more preferably 55±25 wt.-%, still more preferably 55±20 wt.-%, yet more preferably 55±15 wt.-%, most preferably 55±10 wt.-%, and in particular 55±5 wt.-%, based on the total weight of the pharmaceutical dosage form or based on the total weight of the particles.

The content of the pharmacologically active compound in the pharmaceutical dosage form is not particularly limited. The pharmacologically active compound is present in the pharmaceutical dosage form in a therapeutically effective amount. The amount that constitutes a therapeutically effective amount varies according to the active ingredients being used, the condition being treated, the severity of said condition, the patient being treated, and the frequency of administration. The skilled person may readily determine an appropriate amount of pharmacologically active compound to include in a pharmaceutical dosage form.

The dose of the pharmacologically active compound which is adapted for administration preferably is in the range of 0.1 mg to 500 mg, more preferably in the range of 1.0 mg to 400 mg, even more preferably in the range of 5.0 mg to 300 mg, and most preferably in the range of 10 mg to 250 mg. In a preferred embodiment, the total amount of the pharmacologically active compound that is contained in the pharmaceutical dosage form is within the range of from 0.01 to 200 mg, more preferably 0.1 to 190 mg, still more preferably 1.0 to 180 mg, yet more preferably 1.5 to 160 mg, most preferably 2.0 to 100 mg and in particular 2.5 to 80 mg.

Preferably, the content of the pharmacologically active compound is at least 0.5 wt.-%, based on the total weight of the pharmaceutical dosage form or based on the total weight of the particles.

Preferably, the content of the pharmacologically active compound is within the range of from 0.01 to 80 wt.-%, more preferably 0.1 to 50 wt.-%, still more preferably 1 to 25 wt.-%, based on the total weight of the pharmaceutical dosage form or based on the total weight of the particles.

In a preferred embodiment, the content of pharmacologically active compound is within the range of from 0.50±0.45 wt.-%, or 0.75±0.70 wt.-%, or 1.00±0.90 wt.-%, or 1.25±1.20 wt.-%, or 1.50±1.40 wt.-%, or 1.75±1.70 wt.-%, or 2.00±1.90 wt.-%, or 2.25±2.20 wt.-%, or 2.50±2.40 wt.-%; more preferably 0.50±0.40 wt.-%, or 0.75±0.60 wt.-%, or 1.00±0.80 wt.-%, or 1.25±1.10 wt.-%, or 1.50±1.25 wt.-%, or 1.75±1.50 wt.-%, or 2.00±1.75 wt.-%, or 2.25±2.00 wt.-%, or 2.50±2.25 wt.-%; still more preferably 0.50±0.35 wt.-%, or 0.75±0.50 wt.-%, or 1.00±0.70 wt.-%, or 1.25±1.00 wt.-%, or 1.50±1.15 wt.-%, or 1.75±1.30 wt.-%, or 2.00±1.50 wt.-%, or 2.25±1.90 wt.-%, or 2.50±2.10 wt.-%; yet more preferably 0.50±0.30 wt.-%, or 0.75±0.40 wt.-%, or 1.00±0.60 wt.-%, or 1.25±0.80 wt.-%, or 1.50±1.00 wt.-%, or 1.75±1.10 wt.-%, or 2.00±1.40 wt.-%, or 2.25±1.60 wt.-%, or 2.50±1.80 wt.-%; even more preferably 0.50±0.25 wt.-%, or 0.75±0.30 wt.-%, or 1.00±0.50 wt.-%, or 1.25±0.60 wt.-%, or 1.50±0.80 wt.-%, or 1.75±0.90 wt.-%, or 2.00±1.30 wt.-%, or 2.25±1.40 wt.-%, or 2.50±1.50 wt.-%; most preferably 0.50±0.20 wt.-%, or 0.75±0.25 wt.-%, or 1.00±0.40 wt.-%, or 1.25±0.50 wt.-%, or 1.50±0.60 wt.-%, or 1.75±0.70 wt.-%, or 2.00±1.10 wt.-%, or 2.25±1.20 wt.-%, or 2.50±1.30 wt.-%; and in particular 0.50±0.15 wt.-%, or 0.75±0.20 wt.-%, or 1.00±0.30 wt.-%, or 1.25±0.40 wt.-%, or 1.50±0.50 wt.-%, or 1.75±0.60 wt.-%, or 2.00±0.70 wt.-%, or 2.25±0.80 wt.-%, or 2.50±0.90 wt.-%; in each case based on the total weight of the pharmaceutical dosage form.

In a preferred embodiment, the content of pharmacologically active compound is within the range of from 2.0±1.9 wt.-%, or 2.5±2.4 wt.-%, or 3.0±2.9 wt.-%, or 3.5±3.4 wt.-%, or 4.0±3.9 wt.-%, or 4.5±4.4 wt.-%, or 5.0±4.9 wt.-%, or 5.5±5.4 wt.-%, or 6.0±5.9 wt.-%; more preferably 2.0±1.7 wt.-%, or 2.5±2.2 wt.-%, or 3.0±2.6 wt.-%, or 3.5±3.1 wt.-%, or 4.0±3.5 wt.-%, or 4.5±4.0 wt.-%, or 5.0±4.4 wt.-%, or 5.5±4.9 wt.-%, or 6.0±5.3 wt.-%; still more preferably 2.0±1.5 wt.-%, or 2.5±2.0 wt.-%, or 3.0±2.3 wt.-%, or 3.5±2.8 wt.-%, or 4.0±3.1 wt.-%, or 4.5±3.6 wt.-%, or 5.0±3.9 wt.-%, or 5.5±4.4 wt.-%, or 6.0±4.7 wt.-%; yet more preferably 2.0±1.3 wt.-%, or 2.5±1.8 wt.-%, or 3.0±2.0 wt.-%, or 3.5±2.5 wt.-%, or 4.0±2.7 wt.-%, or 4.5±3.2 wt.-%, or 5.0±3.4 wt.-%, or 5.5±3.9 wt.-%, or 6.0±4.1 wt.-%; even more preferably 2.0±1.1 wt.-%, or 2.5±1.6 wt.-%, or 3.0±1.7 wt.-%, or 3.5±2.2 wt.-%, or 4.0±2.4 wt.-%, or 4.5±2.8 wt.-%, or 5.0±2.9 wt.-%, or 5.5±3.4 wt.-%, or 6.0±3.5 wt.-%; most preferably 2.0±0.9 wt.-%, or 2.5±1.4 wt.-%, or 3.0±1.4 wt.-%, or 3.5±1.9 wt.-%, or 4.0±2.1 wt.-%, or 4.5±2.4 wt.-%, or 5.0±2.4 wt.-%, or 5.5±2.9 wt.-%, or 6.0±2.9 wt.-%; and in particular 2.0±0.7 wt.-%, or 2.5±1.2 wt.-%, or 3.0±1.1 wt.-%, or 3.5±1.6 wt.-%, or 4.0±1.8 wt.-%, or 4.5±2.0 wt.-%, or 5.0±1.9 wt.-%, or 5.5±2.4 wt.-%, or 6.0±2.3 wt.-%; in each case based on the total weight of the particles.

In a preferred embodiment, the content of pharmacologically active compound is within the range of from 10±6 wt.-%, more preferably 10±5 wt.-%, still more preferably 10±4 wt.-%, most preferably 10±3 wt.-%, and in particular 10±2 wt.-%, based on the total weight of the pharmaceutical dosage form or based on the total weight of the particles. In another preferred embodiment, the content of pharmacologically active compound is within the range of from 15±6 wt.-%, more preferably 15±5 wt.-%, still more preferably 15±4 wt.-%, most preferably 15±3 wt.-%, and in particular 15±2 wt.-%, based on the total weight of the pharmaceutical dosage form or based on the total weight of the particles. In a further preferred embodiment, the content of pharmacologically active compound is within the range of from 20±6 wt.-%, more preferably 20±5 wt.-%, still more preferably 20±4 wt.-%, most preferably 20±3 wt.-%, and in particular 20±2 wt.-%, based on the total weight of the pharmaceutical dosage form or based on the total weight of the particles. In another preferred embodiment, the content of pharmacologically active compound is within the range of from 25±6 wt.-%, more preferably 25±5 wt.-%, still more preferably 25±4 wt.-%, most preferably 25±3 wt.-%, and in particular 25±2 wt.-%, based on the total weight of the pharmaceutical dosage form or based on the total weight of the particles.

In a preferred embodiment, the pharmacologically active compound is contained in the pharmaceutical dosage form in an amount of 2.5±1 mg, 5.0±2.5 mg, 7.5±5 mg, 10±5 mg, 20±5 mg, 30±5 mg, 40±5 mg, 50±5 mg, 60±5 mg, 70±5 mg, 80±5 mg, 90±5 mg, 100±5 mg, 110±5 mg, 120±5 mg, 130±5, 140±5 mg, 150±5 mg, 160±5 mg, 170±5 mg, 180±5 mg, 190±5 mg, 200±5 mg, 210±5 mg, 220±5 mg, 230±5 mg, 240±5 mg, 250±5 mg, 260±5 mg, 270±5 mg, 280±5 mg, 290±5 mg, or 300±5 mg. In another preferred embodiment, the pharmacologically active compound is contained in the pharmaceutical dosage form in an amount of 2.5±1 mg, 5.0±2.5 mg, 7.5±2.5 mg, 10±2.5 mg, 15±2.5 mg, 20±2.5 mg, 25±2.5 mg, 30±2.5 mg, 35±2.5 mg, 40±2.5 mg, 45±2.5 mg, 50±2.5 mg, 55±2.5 mg, 60±2.5 mg, 65±2.5 mg, 70±2.5 mg, 75±2.5 mg, 80±2.5 mg, 85±2.5 mg, 90±2.5 mg, 95±2.5 mg, 100±2.5 mg, 105±2.5 mg, 110±2.5 mg, 115±2.5 mg, 120±2.5 mg, 125±2.5 mg, 130±2.5 mg, 135±2.5 mg, 140±2.5 mg, 145±2.5 mg, 150±2.5 mg, 155±2.5 mg, 160±2.5 mg, 165±2.5 mg, 170±2.5 mg, 175±2.5 mg, 180±2.5 mg, 185±2.5 mg, 190±2.5 mg, 195±2.5 mg, 200±2.5 mg, 205±2.5 mg, 210±2.5 mg, 215±2.5 mg, 220±2.5 mg, 225±2.5 mg, 230±2.5 mg, 235±2.5 mg, 240±2.5 mg, 245±2.5 mg, 250±2.5 mg, 255±2.5 mg, 260±2.5 mg, or 265±2.5 mg.

The pharmaceutical dosage form according to the invention has preferably a total weight in the range of 0.01 to 1.5 g, more preferably in the range of 0.05 to 1.2 g, still more preferably in the range of 0.1 g to 1.0 g, yet more preferably in the range of 0.2 g to 0.9 g, and most preferably in the range of 0.3 g to 0.8 g. In a preferred embodiment, the total weight of the pharmaceutical dosage form is within the range of 500±450 mg, more preferably 500±300 mg, still more preferably 500±200 mg, yet more preferably 500±150 mg, most preferably 500±100 mg, and in particular 500±50 mg. In another preferred embodiment, the total weight of the pharmaceutical dosage form is within the range of 600±450 mg, more preferably 600±300 mg, still more preferably 600±200 mg, yet more preferably 600±150 mg, most preferably 600±100 mg, and in particular 600±50 mg. In still another preferred embodiment, the total weight of the pharmaceutical dosage form is within the range of 700±450 mg, more preferably 700±300 mg, still more preferably 700±200 mg, yet more preferably 700±150 mg, most preferably 700±100 mg, and in particular 700±50 mg. In yet another preferred embodiment, the total weight of the pharmaceutical dosage form is within the range of 800±450 mg, more preferably 800±300 mg, still more preferably 800±200 mg, yet more preferably 800±150 mg, most preferably 800±100 mg, and in particular 800±50 mg.

In a preferred embodiment, the pharmaceutical dosage form according to the invention is a round pharmaceutical dosage form, preferably having a diameter of e.g. 11 mm or 13 mm. Pharmaceutical dosage forms of this embodiment preferably have a diameter in the range of 1 mm to 30 mm, in particular in the range of 2 mm to 25 mm, more in particular 5 mm to 23 mm, even more in particular 7 mm to 13 mm; and a thickness in the range of 1.0 mm to 12 mm, in particular in the range of 2.0 mm to 10 mm, even more in particular from 3.0 mm to 9.0 mm, even further in particular from 4.0 mm to 8.0 mm.

In another preferred embodiment, the pharmaceutical dosage form according to the invention is an oblong pharmaceutical dosage form, preferably having a length of e.g. 17 mm and a width of e.g. 7 mm. In preferred embodiments, the pharmaceutical dosage form according to the invention has a length of e.g. 22 mm and a width of e.g. 7 mm; or a length of 23 mm and a width of 7 mm; whereas these embodiments are particularly preferred for capsules.

Pharmaceutical dosage forms of this embodiment preferably have a lengthwise extension (longitudinal extension) of 1 mm to 30 mm, in particular in the range of 2 mm to 25 mm, more in particular 5 mm to 23 mm, even more in particular 7 mm to 20 mm; a width in the range of 1 mm to 30 mm, in particular in the range of 2 mm to 25 mm, more in particular 5 mm to 23 mm, even more in particular 7 mm to 13 mm; and a thickness in the range of 1.0 mm to 12 mm, in particular in the range of 2.0 mm to 10 mm, even more in particular from 3.0 mm to 9.0 mm, even further in particular from 4.0 mm to 8.0 mm.

The pharmaceutical dosage forms according to the invention can optionally be provided, partially or completely, with a conventional coating. The pharmaceutical dosage forms according to the invention are preferably film coated with conventional film coating compositions. Suitable coating materials are commercially available, e.g. under the trademarks Opadry® and Eudragit®.

Examples of suitable materials include cellulose esters and cellulose ethers, such as methylcellulose (MC), hydroxypropylmethylcellulose (HPMC), hydroxypropylcellulose (HPC), hydroxyethylcellulose (HEC), sodium carboxymethylcellulose (Na-CMC), poly(meth)-acrylates, such as aminoalkylmethacrylate copolymers, methacrylic acid methylmethacrylate copolymers, methacrylic acid methylmethacrylate copolymers; vinyl polymers, such as polyvinylpyrrolidone, polyvinyl alcohol, polyvinylacetate; and natural film formers.

In a particularly preferred embodiment, the coating is water-soluble. In a preferred embodiment, the coating is based on polyvinyl alcohol, such as polyvinyl alcohol-partially hydrolyzed, and may additionally contain polyethylene glycol, such as macrogol 3350, and/or pigments. In another preferred embodiment, the coating is based on hydroxypropylmethylcellulose, preferably hypromellose type 2910 having a viscosity of 3 to 15 mPas.

The coating can be resistant to gastric juices and dissolve as a function of the pH value of the release environment. By means of this coating, it is possible to ensure that the pharmaceutical dosage form according to the invention passes through the stomach undissolved and the active compound is only released in the intestines. The coating which is resistant to gastric juices preferably dissolves at a pH value of between 5 and 7.5.

The coating can also be applied e.g. to improve the aesthetic impression and/or the taste of the pharmaceutical dosage forms and the ease with which they can be swallowed. Coating the pharmaceutical dosage forms according to the invention can also serve other purposes, e.g. improving stability and shelf-life. Suitable coating formulations comprise a film forming polymer such as, for example, polyvinyl alcohol or hydroxypropylmethylcellulose, e.g. hypromellose, a plasticizer such as, for example, a glycol, e.g. propylene glycol or polyethylene glycol, an opacifier, such as, for example, titanium dioxide, and a film smoothener, such as, for example, talc. Suitable coating solvents are water as well as organic solvents. Examples of organic solvents are alcohols, e.g. ethanol or isopropanol, ketones, e.g. acetone, or halogenated hydrocarbons, e.g. methylene chloride. Coated pharmaceutical dosage forms according to the invention are preferably prepared by first making the cores and subsequently coating said cores using conventional techniques, such as coating in a coating pan.

In a preferred embodiment, the pharmaceutical dosage form according to the invention is a tablet, wherein the particles are contained in a matrix of a matrix material. In the following, this preferred embodiment is referred to as the “preferred tablet according to the invention”.

The preferred tablet according to the invention comprises subunits having different morphology and properties, namely drug-containing particles and matrix material, wherein the particles form a discontinuous phase within the matrix material. The particles typically have mechanical properties that differ from the mechanical properties of the matrix material. Preferably, the particles have a higher mechanical strength than the matrix material. The particles within the preferred tablet according to the invention can be visualized by conventional means such as solid state nuclear magnetic resonance spectroscopy, raster electron microscopy, terahertz spectroscopy, infrared spectroscopy, Raman spectroscopy and the like.

In the preferred tablet according to the invention, the particles are incorporated in a matrix material. From a macroscopic perspective, the matrix material preferably forms a continuous phase in which the particles are embedded as discontinuous phase.

Preferably, the matrix material is a homogenous coherent mass, preferably a homogeneous mixture of solid constituents, in which the particles are embedded thereby spatially separating the particles from one another. While it is possible that the surfaces of particles are in contact or at least in very close proximity with one another, the plurality of particles preferably cannot be regarded as a single continuous coherent mass within the preferred tablet according to the invention.

In other words, the preferred tablet according to the invention comprises
the immediate release particles (i.e. IR particles or FR particles) as volume element(s) of a first type in which the pharmacologically active compound, the optionally present polyalkylene oxide and the optionally present disintegrant are contained, preferably homogeneously,
the at least one controlled release particle (i.e. PR particle(s), DR particles or OR particles) as volume element(s) of a second type in which the pharmacologically active compound and the optionally present polyalkylene oxide are contained, preferably homogeneously, and
the matrix material as volume element of a third type differing from the material that forms the particles, preferably containing neither pharmacologically active compound nor polyalkylene oxide, but optionally polyethylene glycol which differs from polyethylene oxide in its molecular weight.

A purpose of the matrix material in the preferred tablet according to the invention is to ensure rapid disintegration and subsequent release of the pharmacologically active compound from the disintegrated preferred tablet according to the invention, i.e. from the particles. Thus, the matrix material preferably does not contain any excipient that might have a retardant effect on disintegration and drug release, respectively. Thus, the matrix material preferably does not contain any polymer that is typically employed as matrix material in prolonged release formulations.

The preferred tablet according to the invention preferably comprises the matrix material in an amount of more than one third of the total weight of the preferred tablet according to the invention. Thus, the polyalkylene oxide that is contained in the particles of the preferred tablet according to the invention is preferably not also contained in the matrix material.

Preferably, the pharmacologically active compound which is contained in the particles of the preferred tablet according to the invention is preferably not also contained in the matrix material. Thus, in a preferred embodiment, the total amount of pharmacologically active compound contained in the preferred tablet according to the invention is present in the particles which form a discontinuous phase within the matrix material; and the matrix material forming a continuous phase does not contain any pharmacologically active compound.

Preferably, the content of the matrix material is at least 35 wt.-%, at least 37.5 wt.-% or at least 40 wt.-%; more preferably at least 42.5 wt.-%, at least 45 wt.-%, at least 47.5 wt.-% or at least 50 wt.-%; still more preferably at least 52.5 wt.-%, at least 55 wt.-%, at least 57.5 wt.-% or at least 60 wt.-%; yet more preferably at least 62.5 wt.-%, at least 65 wt.-%, at least 67.5 wt.-% or at least 60 wt.-%; most preferably at least 72.5 wt.-%, at least 75 wt.-%, at least 77.5 wt.-% or at least 70 wt.-%; and in particular at least 82.5 wt.-%, at least 85 wt.-%, at least 87.5 wt.-% or at least 90 wt.-%; based on the total weight of the preferred tablet according to the invention.

Preferably, the content of the matrix material is at most 90 wt.-%, at most 87.5 wt.-%, at most 85 wt.-%, or at most 82.5 wt.-%; more preferably at most 80 wt.-%, at most 77.5 wt.-%, at most 75 wt.-% or at most 72.5 wt.-%; still more preferably at most 70 wt.-%, at most 67.5 wt.-%, at most 65 wt.-% or at most 62.5 wt.-%; yet more preferably at most 60 wt.-%, at most 57.5 wt.-%, at most 55 wt.-% or at most 52.5 wt.-%; most preferably at most 50 wt.-%, at most 47.5 wt.-%, at most 45 wt.-% or at most 42.5 wt.-%; and in particular at most 40 wt.-%, at most 37.5 wt.-%, or at most 35 wt.-%; based on the total weight of the preferred tablet according to the invention.

In a preferred embodiment, the content of the matrix material is within the range of 40±5 wt.-%, more preferably 40±2.5 wt.-%, based on the total weight of the preferred tablet according to the invention. In another preferred embodiment, the content of the matrix material is within the range of 45±10 wt.-%, more preferably 45±7.5 wt.-%, still more preferably 45±5 wt.-%, and most preferably 45±2.5 wt.-%, based on the total weight of the preferred tablet according to the invention. In still another preferred embodiment, the content of the matrix material is within the range of 50±10 wt.-%, more preferably 50±7.5 wt.-%, still more preferably 50±5 wt.-%, and most preferably 50±2.5 wt.-%, based on the total weight of the preferred tablet according to the invention. In yet another preferred embodiment, the content of the matrix material is within the range of 55±10 wt.-%, more preferably 55±7.5 wt.-%, still more preferably 55±5 wt.-%, and most preferably 55±2.5 wt.-%, based on the total weight of the preferred tablet according to the invention.

Preferably, the matrix material is a mixture, preferably a homogeneous mixture of at least two different constituents, more preferably of at least three different constituents. In a preferred embodiment, all constituents of the matrix material are homogeneously distributed in the continuous phase that is formed by the matrix material.

In a preferred embodiment, the pharmaceutical dosage form according to the invention is adapted for oral administration once daily. In another preferred embodiment, the pharmaceutical dosage form according to the invention is adapted for oral administration twice daily. In still another preferred embodiment, the pharmaceutical dosage form according to the invention is adapted for administration thrice daily. In yet another preferred embodiment, the pharmaceutical dosage form according to the invention is adapted for oral administration more frequently than thrice daily, for example 4 times daily, 5 times daily, 6 times daily, 7 times daily or 8 times daily.

For the purpose of the specification, “twice daily” means equal or nearly equal time intervals, i.e., every 12 hours, or different time intervals, e.g., 8 and 16 hours or 10 and 14 hours, between the individual administrations.

For the purpose of the specification, “thrice daily” means equal or nearly equal time intervals, i.e., every 8 hours, or different time intervals, e.g., 6, 6 and 12 hours; or 7, 7 and 10 hours, between the individual administrations.

Preferably, the pharmaceutical dosage form according to the invention has under in vitro conditions a disintegration time measured in accordance with Ph. Eur. of at most 5 minutes, more preferably at most 4 minutes, still more preferably at most 3 minutes, yet more preferably at most 2.5 minutes, most preferably at most 2 minutes and in particular at most 1.5 minutes.

It has been surprisingly found that oral dosage forms can be designed that provide the best compromise between tamper-resistance, disintegration time and drug release, drug load, processability (especially tablettability) and patient compliance.

Tamper-resistance and drug release antagonize each other. While smaller particles should typically show a faster release of the pharmacologically active compound, tamper-resistance requires some minimal size of the particles in order to effectively prevent abuse, e.g. i.v. administration. The larger the particles are the less they are suitable for being abused nasally. The smaller the particles are the faster gel formation occurs. Thus, drug release on the one hand and tamper-resistance on the other hand can be optimized by finding the best compromise.

The pharmaceutical dosage form according to the invention is preferably tamper-resistant.

As used herein, the term “tamper-resistant” refers to pharmaceutical dosage forms that are resistant to conversion into a form suitable for misuse or abuse, particular for nasal and/or intravenous administration, by conventional means such as grinding in a mortar or crushing by means of a hammer. In this regard, the pharmaceutical dosage forms as such may be crushable by conventional means. However, the particles contained in the pharmaceutical dosage forms according to the invention preferably exhibit mechanical properties such that they cannot be pulverized by conventional means any further. As the particles are of macroscopic size and contain the pharmacologically active compound, they cannot be administered nasally thereby rendering the pharmaceutical dosage forms tamper-resistant. Preferably, when trying to tamper the dosage form in order to prepare a formulation suitable for abuse by intravenous administration, the liquid part of the formulation that can be separated from the remainder by means of a syringe is as less as possible, preferably it contains not more than 20 wt.-%, more preferably not more than 15 wt.-%, still more preferably not more than 10 wt.-%, and most preferably not more than 5 wt.-% of the originally contained pharmacologically active compound. Preferably, this property is tested by (i) dispensing a pharmaceutical dosage form that is either intact or has been manually comminuted by means of two spoons in 5 ml of purified water, (ii) heating the liquid up to its boiling point, (iii) boiling the liquid in a covered vessel for 5 min without the addition of further purified water, (iv) drawing up the hot liquid into a syringe (needle 21 G equipped with a cigarette filter), (v) determining the amount of the pharmacologically active compound contained in the liquid within the syringe.

Further, when trying to disrupt the pharmaceutical dosage forms by means of a hammer or mortar, the particles tend to adhere to one another thereby forming aggregates and agglomerates, respectively, which are larger in size than the untreated particles.

Preferably, tamper-resistance is achieved based on the mechanical properties of the particles so that comminution is avoided or at least substantially impeded. According to the invention, the term comminution means the pulverization of the particles using conventional means usually available to an abuser, for example a pestle and mortar, a hammer, a mallet or other conventional means for pulverizing under the action of force. Thus, tamper-resistance preferably means that pulverization of the particles using conventional means is avoided or at least substantially impeded.

Preferably, the mechanical properties of the particles according to the invention, particularly their breaking strength and deformability, substantially rely on the presence and spatial distribution of polyalkylene oxide, although their mere presence does typically not suffice in order to achieve said properties. The advantageous mechanical properties of the particles according to the invention may not automatically be achieved by simply processing pharmacologically active compound, polyalkylene oxide, and optionally further excipients by means of conventional methods for the preparation of pharmaceutical dosage forms. In fact, usually suitable apparatuses must be selected for the preparation and critical processing parameters must be adjusted, particularly pressure/force, temperature and time. Thus, even if conventional apparatuses are used, the process protocols usually must be adapted in order to meet the required criteria.

In general, the particles exhibiting the desired properties may be obtained only if, during preparation of the particles,
suitable components
in suitable amounts
   are exposed to
a sufficient pressure
at a sufficient temperature
for a sufficient period of time.

Thus, regardless of the apparatus used, the process protocols must be adapted in order to meet the required criteria. Therefore, the breaking strength and deformability of the particles is separable from the composition.

The particles contained in the pharmaceutical dosage form according to the invention have a breaking strength of at least 300 N, preferably of at least 400 N, or at least 500 N, preferably at least 600 N, more preferably at least 700 N, still more preferably at least 800 N, yet more preferably at least 1000 N, most preferably at least 1250 N and in particular at least 1500 N.

In order to verify whether a particle exhibits a particular breaking strength of e.g. 300 N or 500 N it is typically not necessary to subject said particle to forces much higher than 300 N and 500 N, respectively. Thus, the breaking strength test can usually be terminated once the force corresponding to the desired breaking strength has been slightly exceeded, e.g. at forces of e.g. 330 N and 550 N, respectively.

The “breaking strength” (resistance to crushing) of a pharmaceutical dosage form and of a particle is known to the skilled person. In this regard it can be referred to, e.g., W. A. Ritschel, Die Tablette, 2. Auflage, Editio Cantor Verlag Aulendorf, 2002; H Liebermann et al., Pharmaceutical dosage forms: Pharmaceutical dosage forms, Vol. 2, Informa Healthcare; 2 edition, 1990; and Encyclopedia of Pharmaceutical Technology, Informa Healthcare; 1 edition.

For the purpose of the specification, the breaking strength is preferably defined as the amount of force that is necessary in order to fracture the particle (=breaking force). Therefore, for the purpose of the specification a particle does preferably not exhibit the desired breaking strength when it breaks, i.e., is fractured into at least two independent parts that are separated from one another. In another preferred embodiment, however, the particle is regarded as being broken if the force decreases by 50% (threshold value) of the highest force measured during the measurement (see below).

The particles according to the invention are distinguished from conventional particles that can be contained in pharmaceutical dosage forms in that, due to their breaking strength, they cannot be pulverized by the application of force with conventional means, such as for example a pestle and mortar, a hammer, a mallet or other usual means for pulverization, in particular devices developed for this purpose (tablet crushers). In this regard “pulverization” means crumbling into small particles. Avoidance of pulverization virtually rules out oral or parenteral, in particular intravenous or nasal abuse.

Conventional particles typically have a breaking strength well below 200 N.

The breaking strength of conventional round pharmaceutical dosage forms/particles may be estimated according to the following empirical formula: Breaking Strength [in N]=10×Diameter Of The Pharmaceutical dosage form/Particle [in mm]. Thus, according to said empirical formula, a round pharmaceutical dosage form/particle having a breaking strength of at least 300 N would require a diameter of at least 30 mm). Such a particle, however, could not be swallowed, let alone a pharmaceutical dosage form containing a plurality of such particles. The above empirical formula preferably does not apply to the particles according to the invention, which are not conventional but rather special.

Further, the actual mean chewing force is 220 N (cf., e.g., P. A. Proeschel et al., J Dent Res, 2002, 81(7), 464-468). This means that conventional particles having a breaking strength well below 200 N may be crushed upon spontaneous chewing, whereas the particles according to the invention may preferably not.

Still further, when applying a gravitational acceleration of 9.81 m/s², 300 N correspond to a gravitational force of more than 30 kg, i.e. the particles according to the invention can preferably withstand a weight of more than 30 kg without being pulverized.

Methods for measuring the breaking strength of a pharmaceutical dosage form are known to the skilled artisan. Suitable devices are commercially available.

For example, the breaking strength (resistance to crushing) can be measured in accordance with the Eur. Ph. 5.0, 2.9.8 or 6.0, 2.09.08 “Resistance to Crushing of Pharmaceutical dosage forms”. The test is intended to determine, under defined conditions, the resistance to crushing of pharmaceutical dosage forms and particles, respectively, measured by the force needed to disrupt them by crushing. The apparatus consists of 2 jaws facing each other, one of which moves towards the other. The flat surfaces of the jaws are perpendicular to the direction of movement. The crushing surfaces of the jaws are flat and larger than the zone of contact with the pharmaceutical dosage form and particle, respectively. The apparatus is calibrated using a system with a precision of 1 Newton. The pharmaceutical dosage form and particle, respectively, is placed between the jaws, taking into account, where applicable, the shape, the break-mark and the inscription; for each measurement the pharmaceutical dosage form and particle, respectively, is oriented in the same way with respect to the direction of application of the force (and the direction of extension in which the breaking strength is to be measured). The measurement is carried out on 10 pharmaceutical dosage forms and particles, respectively, taking care that all fragments have been removed before each determination. The result is expressed as the mean, minimum and maximum values of the forces measured, all expressed in Newton.

A similar description of the breaking strength (breaking force) can be found in the USP. The breaking strength can alternatively be measured in accordance with the method described therein where it is stated that the breaking strength is the force required to cause a pharmaceutical dosage form and particle, respectively, to fail (i.e., break) in a specific plane. The pharmaceutical dosage forms and particles, respectively, are generally placed between two platens, one of which moves to apply sufficient force to the pharmaceutical dosage form and particle, respectively, to cause fracture. For conventional, round (circular cross-section) pharmaceutical dosage forms and particles, respectively, loading occurs across their diameter (sometimes referred to as diametral loading), and fracture occurs in the plane. The breaking force of pharmaceutical dosage forms and particles, respectively, is commonly called hardness in the pharmaceutical literature; however, the use of this term is misleading. In material science, the term hardness refers to the resistance of a surface to penetration or indentation by a small probe. The term crushing strength is also frequently used to describe the resistance of pharmaceutical dosage forms and particle, respectively, to the application of a compressive load. Although this term describes the true nature of the test more accurately than does hardness, it implies that pharmaceutical dosage forms and particles, respectively, are actually crushed during the test, which is often not the case.

Alternatively, the breaking strength (resistance to crushing) can be measured in accordance with WO 2008/107149, which can be regarded as a modification of the method described in the Eur. Ph. The apparatus used for the measurement is preferably a “Zwick Z 2.5” materials tester, Fₘₐₓ=2.5 kN with a maximum draw of 1150 mm, which should be set up with one column and one spindle, a clearance behind of 100 mm and a test speed adjustable between 0.1 and 800 mm/min together with testControl software. A skilled person knows how to properly adjust the test speed, e.g. to 10 mm/min, 20 mm/min, or 40 mm/min, for example. Measurement is performed using a pressure piston with screw-in inserts and a cylinder (diameter 10 mm), a force transducer, Fₘₐₓ. 1 kN, diameter=8 mm, class 0.5 from 10 N, class 1 from 2 N to ISO 7500-1, with manufacturer's test certificate M according to DIN 55350-18 (Zwick gross force Fₘₐₓ=1.45 kN) (all apparatus from Zwick GmbH & Co. KG, Ulm, Germany) with Order No BTC-FR 2.5 TH. D09 for the tester, Order No BTC-LC 0050N. P01 for the force transducer, Order No BO 70000 S₀₆ for the centering device.

When using the testControl software (testXpert V10.11), the following exemplified settings and parameters have revealed to be useful: LE-position: clamping length 150 mm. LE-speed: 500 mm/min, clamping length after pre-travel: 195 mm, pre-travel speed: 500 mm/min, no pre-force control pre-force: pre-force 1N, pre-force speed 10 mm/min sample data: no sample form, measuring length traverse distance 10 mm, no input required prior to testing testing/end of test; test speed: position-controlled 10 mm/min, delay speed shift: 1, force shut down threshold 50% Fₘₐₓ, no force threshold for break-tests, no max length variation, upper force limit: 600N expansion compensation: no correction of measuring length actions after testing: LE to be set after test, no unload of sample TRS: data memory: TRS distance interval until break 1 TRS time interval 0.1 s, TRS force interval 1N machine; traverse distance controller: upper soft end 358 mm, lower soft end 192 mm lower test space. Parallel arrangement of the upper plate and the ambos should be ensured—these parts must not touch during or after testing. After testing, a small gap (e.g. 0.1 or 0.2 mm) should still be present between the two brackets in intimated contact with the tested particle, representing the remaining thickness of the deformed particle.

In a preferred embodiment, the particle is regarded as being broken if it is fractured into at least two separate pieces of comparable morphology. Separated matter having a morphology different from that of the deformed particle, e.g. dust, is not considered as pieces qualifying for the definition of breaking.

The particles according to the invention preferably exhibit mechanical strength over a wide temperature range, in addition to the breaking strength (resistance to crushing) optionally also sufficient hardness, yield strength, fatigue strength, impact resistance, impact elasticity, tensile strength, compressive strength and/or modulus of elasticity, optionally also at low temperatures (e.g. below −24° C., below −40° C. or possibly even in liquid nitrogen), for it to be virtually impossible to pulverize by spontaneous chewing, grinding in a mortar, pounding, etc. Thus, preferably, the comparatively high breaking strength of the particle according to the invention is maintained even at low or very low temperatures, e.g., when the pharmaceutical dosage form is initially chilled to increase its brittleness, for example to temperatures below −25° C., below −40° C. or even in liquid nitrogen.

The particle according to the invention is characterized by a certain degree of breaking strength. This does not mean that the particle must also exhibit a certain degree of hardness. Hardness and breaking strength are different physical properties. Therefore, the tamper-resistance of the pharmaceutical dosage form does not necessarily depend on the hardness of the particles. For instance, due to its breaking strength, impact strength, elasticity modulus and tensile strength, respectively, the particles can preferably be deformed, e.g. plastically, when exerting an external force, for example using a hammer, but cannot be pulverized, i.e., crumbled into a high number of fragments. In other words, the particles according to the invention are characterized by a certain degree of breaking strength, but not necessarily also by a certain degree of form stability.

Therefore, in the meaning of the specification, a particle that is deformed when being exposed to a force in a particular direction of extension but that does not break (plastic deformation or plastic flow) is preferably to be regarded as having the desired breaking strength in said direction of extension.

Defining the mechanical properties of particles in terms of their breaking strength (breaking force, force upon break, crushing strength) has advantages compared to other parameters such as tensile strength, because said other parameters depend upon the outer shape of the particles, whereas the breaking strength can be determined independently. In the case of ideal break curve when the ultimate tensile strength and the tensile strength of the particle are equal, the tensile strength can be calculated based upon the breaking strength. The equation for tensile strength that takes into consideration diameter and the width of the root face as the contact surface of the force reads:

$$σ = \frac{2 \cdot P}{π \cdot D \cdot t}$$

wherein σ=tensile strength (N/mm²); P=force upon break (N); t=width of root face (mm); D=diameter (mm).

However, prerequisites for the strict physical validity of this equation are as follows: homogeneity of the particles, deformation according to Hooke's law in the same manner for tension and pressure, only elastic or brittle behavior, only point-type supporting surfaces. A different empirically determined equation is necessary for cambered particles:

$$σ = \frac{10 P}{π {D}^{2}} {( 2.84 \frac{t}{D} - 0.126 \frac{t}{W} + 3.15 \frac{W}{D} + 0.01 )}^{- 1}$$

wherein D=diameter; P=force upon break; t=thickness overall; W=thickness of the central cylinder.

Preferred particles present in the pharmaceutical dosage forms according to the invention are those having a suitable tensile strength as determined by a test method currently accepted in the art. Further preferred particles are those having a Youngs Modulus as determined by a test method of the art. Still further preferred particles are those having an acceptable elongation at break.

Irrespective of whether the particles according to the invention have an increased breaking strength or nor, the particles according to the invention preferably exhibit a certain degree of deformability. The particles contained in the pharmaceutical dosage form according to the invention preferably have a deformability such that they show an increase, preferably a substantially steady increase of the force at a corresponding decrease of the displacement in the force-displacement-diagram when being subjected to a breaking strength test as described above.

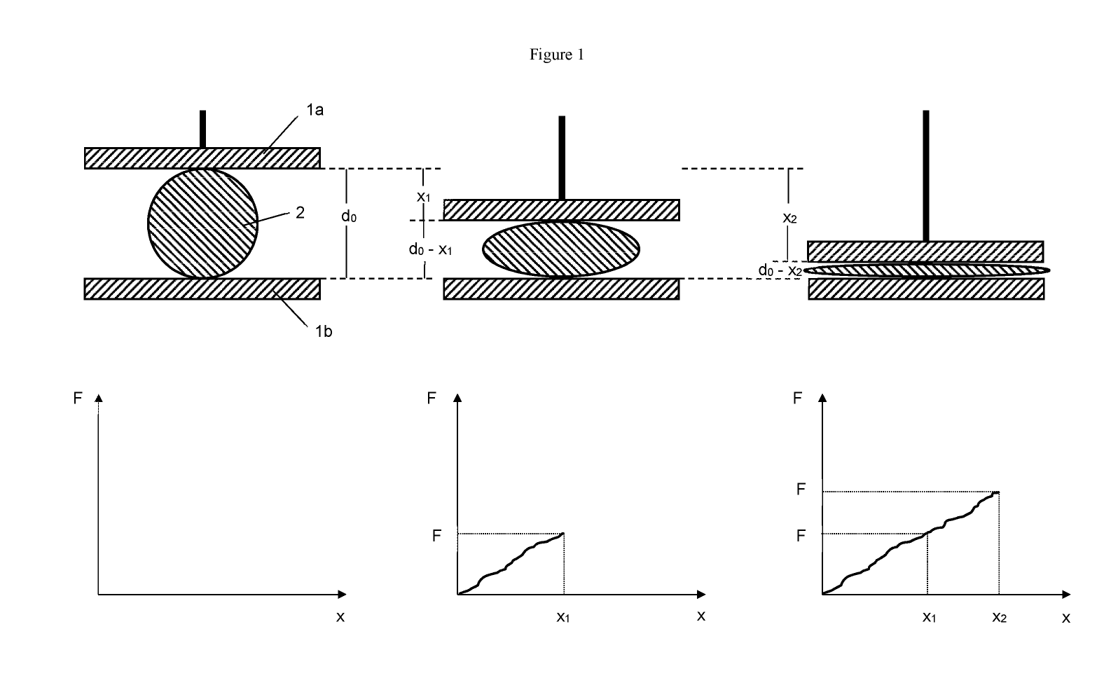

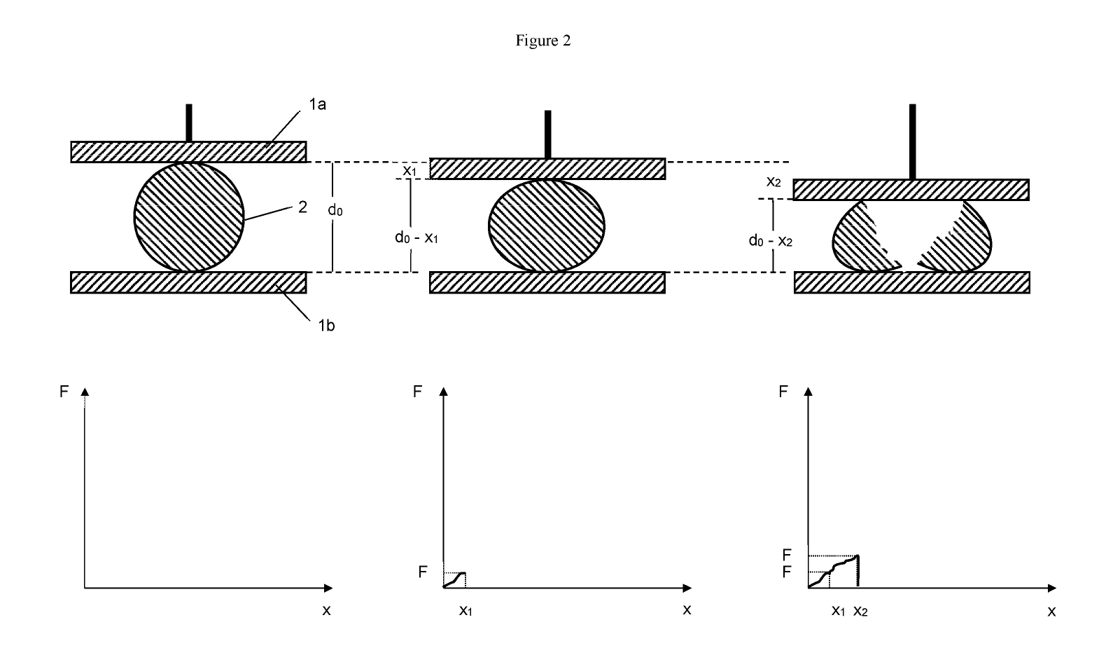

This mechanical property, i.e. the deformability of the individual particles, is illustrated in FIGS. 1 and 2.

FIG. 1 schematically illustrates the measurement and the corresponding force-displacement-diagram. In particular, FIG. 1A shows the initial situation at the beginning of the measurement. The sample particle (2) is placed between upper jaw (1a) and lower jaw (1b) which each are in intimate contact with the surface of the particle (2). The initial displacement do between upper jaw (1a) and lower jaw (1b) corresponds to the extension of the particle orthogonal to the surfaces of upper jaw (1a) and lower jaw (1b). At this time, no force is exerted at all and thus, no graph is displayed in the force-displacement-diagram below. When the measurement is commenced, the upper jaw is moved in direction of lower jaw (1b), preferably at a constant speed. FIG. 1B shows a situation where due to the movement of upper jaw (1a) towards lower jaw (1b) a force is exerted on particle (2). Because of its deformability, the particle (2) is flattened without being fractured. The force-displacement-diagram indicates that after a reduction of the displacement do of upper jaw (1a) and lower jaw (1b) by distance x₁, i.e. at a displacement of d₁=d₀−x₁, a force F₁ is measured. FIG. 1C shows a situation where due to the continuous movement of upper jaw (1a) towards lower jaw (1b), the force that is exerted on particle (2) causes further deformation, although the particle (2) does not fracture. The force-displacement-diagram indicates that after a reduction of the displacement do of upper jaw (1a) and lower jaw (1b) by distance x₂, i.e. at a displacement of d₂=d₀−x₂, a force F₂ is measured. Under these circumstances, the particle (2) has not been broken (fractured) and a substantially steady increase of the force in the force-displacement-diagram is measured.

In contrast, FIG. 2 schematically illustrates the measurement and the corresponding force-displacement-diagram of a conventional comparative particle not having the degree of deformability as the particles according to the invention. FIG. 2A shows the initial situation at the beginning of the measurement. The comparative sample particle (2) is placed between upper jaw (1a) and lower jaw (1b) which each are in intimate contact with the surface of the comparative particle (2). The initial displacement do between upper jaw (1a) and lower jaw (1b) corresponds to the extension of the comparative particle orthogonal to the surfaces of upper jaw (1a) and lower jaw (1b). At this time, no force is exerted at all and thus, no graph is displayed in the force-displacement-diagram below. When the measurement is commenced, the upper jaw is moved in direction of lower jaw (1b), preferably at a constant speed. FIG. 2B shows a situation where due to the movement of upper jaw (1a) towards lower jaw (1b) a force is exerted on comparative particle (2). Because of some deformability, the comparative particle (2) is slightly flattened without being fractured. The force-displacement-diagram indicates that after a reduction of the displacement do of upper jaw (1a) and lower jaw (1b) by distance x₁, i.e. at a displacement of d₁=d₀−x₁, a force F₁ is measured. FIG. 2C shows a situation where due to the continuous movement of upper jaw (1a) towards lower jaw (1b), the force that is exerted on particle (2) causes sudden fracture of the comparative particle (2). The force-displacement-diagram indicates that after a reduction of the displacement do of upper jaw (1a) and lower jaw (1b) by distance x₂, i.e. at a displacement of d₂=d₀−x₂, a force F₂ is measured that suddenly drops when the particle fractures. Under these circumstances, the particle (2) has been broken (fractured) and no steady increase of the force in the force-displacement-diagram is measured. The sudden drop (decrease) of the force can easily be recognized and does not need to be quantified for the measurement. The steady increase in the force-displacement-diagram ends at displacement d₂=d₀−x₂ when the particle breaks.

In a preferred embodiment, the particles contained in the pharmaceutical dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), preferably at least until the displacement d of upper jaw (1a) and lower jaw (1b) has been reduced to a value of 90% of the original displacement do (i.e. d=0.9·d₀), preferably to a displacement d of 80% of the original displacement do, more preferably to a displacement d of 70% of the original displacement do, still more preferably to a displacement d of 60% of the original displacement do, yet more preferably to a displacement d of 50% of the original displacement do, even more preferably to a displacement d of 40% of the original displacement do, most preferably to a displacement d of 30% of the original displacement do, and in particular to a displacement d of 20% of the original displacement do, or to a displacement d of 15% of the original displacement do, to a displacement d of 10% of the original displacement do, or to a displacement d of 5% of the original displacement do.

In another preferred embodiment, the particles contained in the pharmaceutical dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), preferably at least until the displacement d of upper jaw (1a) and lower jaw (1b) has been reduced to 0.80 mm or 0.75 mm, preferably 0.70 mm or 0.65 mm, more preferably 0.60 mm or 0.55 mm, still more preferably 0.50 mm or 0.45 mm, yet more preferably 0.40 mm or 0.35 mm, even more preferably 0.30 mm or 0.25 mm, most preferably 0.20 mm or 0.15 mm and in particular 0.10 or 0.05 mm.

In still another preferred embodiment, the particles contained in the pharmaceutical dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), at least until the displacement d of upper jaw (1a) and lower jaw (1b) has been reduced to 50% of the original displacement do (i.e. d=d₀/2), whereas the force measured at said displacement (d=d₀/2) is at least 25 N or at least 50 N, preferably at least 75 N or at least 100 N, still more preferably at least 150 N or at least 200 N, yet more preferably at least 250 N or at least 300 N, even more preferably at least 350 N or at least 400 N, most preferably at least 450 N or at least 500 N, and in particular at least 625 N, or at least 750 N, or at least 875 N, or at least 1000 N, or at least 1250 N, or at least 1500 N.

In another preferred embodiment, the particles contained in the pharmaceutical dosage form according to the invention have a deformability such that they show an increase, preferably a substantially steady increase of the force at a corresponding decrease of the displacement in the force-displacement-diagram when being subjected to a breaking strength test as described above (“Zwick Z 2.5” materials tester, constant speed), at least until the displacement d of upper jaw (1a) and lower jaw (1b) has been reduced by at least 0.1 mm, more preferably at least 0.2 mm, still more preferably at least 0.3 mm, yet more preferably at least 0.4 mm, even more preferably at least 0.5 mm, most preferably at least 0.6 mm, and in particular at least 0.7 mm, whereas the force measured at said displacement is within the range of from 5.0 N to 250 N, more preferably from 7.5 N to 225 N, still more preferably from 10 N to 200 N, yet more preferably from 15 N to 175 N, even more preferably from 20 N to 150 N, most preferably from 25 N to 125 N, and in particular from 30 N to 100 N.

In yet another embodiment, the particles contained in the pharmaceutical dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at most 90% of the original displacement do (i.e. d≤0.9·d₀), preferably at most 80% of the original displacement do (i.e. d≤0.8·d₀), more preferably at most 70% of the original displacement do (i.e. d≤0.7·d₀), still more preferably at most 60% of the original displacement do (i.e. d≤0.6·d₀), yet more preferably at most 50% of the original displacement do (i.e. d≤0.5·d₀), even more preferably at most 40% of the original displacement do (i.e. d≤0.4·d₀), most preferably at most 30% of the original displacement do (i.e. d≤0.3·d₀), and in particular at most 20% of the original displacement do (i.e. d≤0.2·d₀), or at most 15% of the original displacement do (i.e. d≤0.15·d₀), at most 10% of the original displacement do (i.e. d≤0.1·d₀), or at most 5% of the original displacement do (i.e. d≤0.05·d₀).

Preferably, the particles contained in the pharmaceutical dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at most 0.80 mm or at most 0.75 mm, preferably at most 0.70 mm or at most 0.65 mm, more preferably at most 0.60 mm or at most 0.55 mm, still more preferably at most 0.50 mm or at most 0.45 mm, yet more preferably at most 0.40 mm or at most 0.35 mm, even more preferably at most 0.30 mm or at most 0.25 mm, most preferably at most 0.20 mm or at most 0.15 mm and in particular at most 0.10 or at most 0.05 mm.

In another embodiment, the particles contained in the pharmaceutical dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at least 5% of the original displacement d₀ (i.e. d≥0.05·d₀), preferably at least 10% of the original displacement do (i.e. d≥0.1·d₀), more preferably at least 15% of the original displacement do (i.e. d≥0.15·d₀), still more preferably at least 20% of the original displacement do (i.e. d≥0.2·d₀), yet more preferably at least 30% of the original displacement do (i.e. d≥0.3·d₀), even more preferably at least 40% of the original displacement do (i.e. d≥0.4·d₀), most preferably at least 50% of the original displacement do (i.e. d≥0.5·d₀), and in particular at least 60% of the original displacement do (i.e. d≥0.6·d₀), or at least 70% of the original displacement do (i.e. d≥0.7·d₀), at least 80% of the original displacement do (i.e. d≥0.8·d₀), or at least 90% of the original displacement do (i.e. d≥0.9·d₀).

Preferably, the particles contained in the pharmaceutical dosage form according to the invention have a deformability such that they are deformed without being fractured when subjected to a constant force of e.g. 50 N, 100 N, 200 N, 300 N, 400 N, 500 N or 600 N in a breaking strength test as described above (“Zwick Z 2.5” materials tester, constant force), until the displacement d of upper jaw (1a) and lower jaw (1b) is reduced so that no further deformation takes place at said constant force, whereas at this equilibrated state the displacement d of upper jaw (1a) and lower jaw (1b) is at least 0.05 mm or at least 0.10 mm, preferably at least 0.15 mm or at least 0.20 mm, more preferably at least 0.25 mm or at least 0.30 mm, still more preferably at least 0.35 mm or at least 0.40 mm, yet more preferably at least 0.45 mm or at least 0.50 mm, even more preferably at least 0.55 mm or at least 0.60 mm, most preferably at least 0.65 mm or at least 0.70 mm and in particular at least 0.75 or at least 0.80 mm.

Preferably, the release profile, the drug and the pharmaceutical excipients of the pharmaceutical dosage form according to the invention are stable upon storage, preferably upon storage at elevated temperature, e.g. 40° C., for 3 months in sealed containers.

In connection with the release profile “stable” means that when comparing the initial release profile with the release profile after storage, at any given time point the release profiles deviate from one another by not more than 20%, more preferably not more than 15%, still more preferably not more than 10%, yet more preferably not more than 7.5%, most preferably not more than 5.0% and in particular not more than 2.5%.

In connection with the drug and the pharmaceutical excipients “stable” means that the pharmaceutical dosage forms satisfy the requirements of EMEA concerning shelf-life of pharmaceutical products.

Suitable in vitro conditions are known to the skilled artisan. In this regard it can be referred to, e.g., the Eur. Ph. Preferably, the release profile is measured under the following conditions: Paddle apparatus equipped without sinker, 50 rpm, 37±5° C., 900 mL simulated gastric fluid pH 1.2 (or pH 1) which after 2 hours is replaced by intestinal fluid pH 6.8 (phosphate buffer) (or pH 7). In a preferred embodiment, the rotational speed of the paddle is increased to 75 rpm.

Preferably, the pharmaceutical dosage form as such provides an in vitro release profile measured by means of a paddle apparatus equipped without sinker at 50 rpm, 37±5° C., in 900 mL release medium, for the first 2 hours at pH 1.2 and thereafter at pH 6.8; wherein an in vitro release of 80 wt.-% of the pharmacologically active compound that was originally contained in the pharmaceutical dosage form is achieved in ethanolic release medium at an ethanol concentration of 40 vol.-% later than in non-ethanolic release medium. Preferably, an in vitro release of 80 wt.-% of the pharmacologically active compound that was originally contained in the pharmaceutical dosage form is achieved in ethanolic release medium at an ethanol concentration of 40 vol.-% at least 15 minutes later, more preferably at least 30 minutes later, still more preferably at least 45 minutes later, yet more preferably at least 60 minutes later, even more preferably at least 75 minutes later, most preferably at least 90 minutes later than in non-ethanolic release medium.

Preferably, the pharmaceutical dosage form according to the invention provides an in vitro release profile measured by means of a paddle apparatus equipped without sinker at 50 rpm, 37±5° C., in 900 mL release medium, for the first 2 hours at pH 1.2 and thereafter at pH 6.8; such that after 3 hours
in non-ethanolic release medium at least X wt.-% of the pharmacologically active compound that was originally contained in the pharmaceutical dosage form have been released and
in ethanolic release medium at an ethanol concentration of 40 vol.-% less than X wt.-% of the pharmacologically active compound that was originally contained in the pharmaceutical dosage form have been released;
   wherein in either case X means 60, or 62, or 64, or 66, or 68, or 70, or 72, or 74, or 76, or 78, or 80, or 82, or 84, or 86, or 88, or 90, or 92, or 94, or 96.

In a preferred embodiment of the pharmaceutical dosage form according to the invention, the immediate release particles (i.e. IR particles or FR particles) and/or the at least one controlled release particle (i.e. PR particle(s), DR particles or OR particles) are hot melt-extruded.

Thus, the particles according to the invention are preferably prepared by melt-extrusion, although also other methods of thermoforming may be used in order to manufacture the particles according to the invention such as press-molding at elevated temperature or heating of particles that were manufactured by conventional compression in a first step and then heated above the softening temperature of the polyalkylene oxide in the particles in a second step to form hard pharmaceutical dosage forms. In this regards, thermoforming means the forming, or molding of a mass after the application of heat. In a preferred embodiment, the particles are thermoformed by hot-melt extrusion.

In a preferred embodiment, the particles are prepared by hot melt-extrusion, preferably by means of a twin-screw-extruder. Melt extrusion preferably provides a melt-extruded strand that is preferably cut into monoliths, which are then optionally compressed and formed into particles. Preferably, compression is achieved by means of a die and a punch, preferably from a monolithic mass obtained by melt extrusion. If obtained via melt extrusion, the compressing step is preferably carried out with a monolithic mass exhibiting ambient temperature, that is, a temperature in the range from 20 to 25° C. The strands obtained by way of extrusion can either be subjected to the compression step as such or can be cut prior to the compression step. This cutting can be performed by usual techniques, for example using rotating knives or compressed air, at elevated temperature, e.g. when the extruded stand is still warm due to hot-melt extrusion, or at ambient temperature, i.e. after the extruded strand has been allowed to cool down. When the extruded strand is still warm, singulation of the extruded strand into extruded particles is preferably performed by cutting the extruded strand immediately after it has exited the extrusion die. It is possible to subject the extruded strands to the compression step or to the cutting step when still warm, that is more or less immediately after the extrusion step. The extrusion is preferably carried out by means of a twin-screw extruder.

The particles of the pharmaceutical dosage form according to the invention may be produced by different processes, the particularly preferred of which are explained in greater detail below. Several suitable processes have already been described in the prior art. In this regard it can be referred to, e.g., WO 2005/016313, WO 2005/016314, WO 2005/063214, WO 2005/102286, WO 2006/002883, WO 2006/002884, WO 2006/002886, WO 2006/082097, and WO 2006/082099.

In general, the process for the production of the particles according to the invention preferably comprises the following steps:
(a) mixing all ingredients;
(b) optionally pre-forming the mixture obtained from step (a), preferably by applying heat and/or force to the mixture obtained from step (a), the quantity of heat supplied preferably not being sufficient to heat the polyalkylene oxide up to its softening point;
(c) hardening the mixture by applying heat and force, it being possible to supply the heat during and/or before the application of force and the quantity of heat supplied being sufficient to heat the polyalkylene oxide at least up to its softening point; and thereafter allowing the material to cool and removing the force
(d) optionally singulating the hardened mixture; and
(e) optionally providing a film coating.

Heat may be supplied directly, e.g. by contact or by means of hot gas such as hot air, or with the assistance of ultrasound; or is indirectly supplied by friction and/or shear. Force may be applied and/or the particles may be shaped for example by direct tableting or with the assistance of a suitable extruder, particularly by means of a screw extruder equipped with one or two screws (single-screw-extruder and twin-screw-extruder, respectively) or by means of a planetary gear extruder.

The final shape of the particles may either be provided during the hardening of the mixture by applying heat and force (step (c)) or in a subsequent step (step (e)). In both cases, the mixture of all components is preferably in the plastified state, i.e. preferably, shaping is performed at a temperature at least above the softening point of the polyalkylene oxide. However, extrusion at lower temperatures, e.g. ambient temperature, is also possible and may be preferred.

In a preferred embodiment, the mixture of ingredients is heated and subsequently compressed under conditions (time, temperature and pressure) sufficient in order to achieve the desired mechanical properties, e.g. in terms of breaking strength and the like. This technique may be achieved e.g. by means of a tableting tool which is either heated and/or which is filled with the heated mixture that is subsequently compressed without further supply of heat or with simultaneous additional supply of heat.

In another preferred embodiment, the mixture of ingredients is heated and simultaneously compressed under conditions (time, temperature and pressure) sufficient in order to achieve the desired mechanical properties, e.g. in terms of breaking strength and the like. This technique may be achieved e.g. by means of an extruder with one or more heating zones, wherein the mixture is heated and simultaneously subjected to extrusion forces finally resulting in a compression of the heated mixture.

In still another embodiment, the mixture of ingredients is compressed under ambient conditions at sufficient pressure and subsequently heated (cured) under conditions (time, temperature) sufficient in order to achieve the desired mechanical properties, e.g. in terms of breaking strength and the like. This technique may be achieved e.g. by means of a curing oven in which the compressed articles are cured for a sufficient time at a sufficient temperature, preferably without exerting any further pressure. Such process is further described e.g. in US 2009/0081290.

A particularly preferred process for the manufacture of the particles according to the invention involves hot-melt extrusion. In this process, the particles according to the invention are produced by thermoforming with the assistance of an extruder, preferably without there being any observable consequent discoloration of the extrudate.

This process is characterized in that
a) all components are mixed,
b) the resultant mixture is heated in the extruder at least up to the softening point of the polyalkylene oxide and extruded through the outlet orifice of the extruder by application of force,
c) the still plastic extrudate is singulated and formed into the particles or
d) the cooled and optionally reheated singulated extrudate is formed into the particles.

Mixing of the components according to process step a) may also proceed in the extruder.

The components may also be mixed in a mixer known to the person skilled in the art. The mixer may, for example, be a roll mixer, shaking mixer, shear mixer or compulsory mixer.

The, preferably molten, mixture which has been heated in the extruder at least up to the softening point of polyalkylene oxide is extruded from the extruder through a die with at least one bore, preferably a multitude of bores.

The process according to the invention requires the use of suitable extruders, preferably screw extruders. Screw extruders which are equipped with two screws (twin-screw-extruders) are particularly preferred.

Preferably, extrusion is performed in the absence of water, i.e., no water is added. However, traces of water (e.g., caused by atmospheric humidity) may be present.

The extruder preferably comprises at least two temperature zones, with heating of the mixture at least up to the softening point of the polyalkylene oxide proceeding in the first zone, which is downstream from a feed zone and optionally mixing zone. The throughput of the mixture is preferably from 1.0 kg to 15 kg/hour. In a preferred embodiment, the throughput is from 0.5 kg/hour to 3.5 kg/hour. In another preferred embodiment, the throughput is from 4 to 15 kg/hour.

In a preferred embodiment, the die head pressure is within the range of from 25 to 200 bar. The die head pressure can be adjusted inter alia by die geometry, temperature profile, extrusion speed, number of bores in the dies, screw configuration, first feeding steps in the extruder, and the like.

The die geometry or the geometry of the bores is freely selectable. The die or the bores may accordingly exhibit a round, oblong or oval cross-section, wherein the round cross-section preferably has a diameter of 0.1 mm to 2 mm, preferably of 0.5 mm to 0.9 mm. Preferably, the die or the bores have a round cross-section. The casing of the extruder used according to the invention may be heated or cooled. The corresponding temperature control, i.e. heating or cooling, is so arranged that the mixture to be extruded exhibits at least an average temperature (product temperature) corresponding to the softening temperature of the polyalkylene oxide and does not rise above a temperature at which the pharmacologically active compound to be processed may be damaged. Preferably, the temperature of the mixture to be extruded is adjusted to below 180° C., preferably below 150° C., but at least to the softening temperature of polyalkylene oxide. Typical extrusion temperatures are 120° C. and 150° C.

In a preferred embodiment, the extruder torque is within the range of from 30 to 95%. Extruder torque can be adjusted inter alia by die geometry, temperature profile, extrusion speed, number of bores in the dies, screw configuration, first feeding steps in the extruder, and the like.

After extrusion of the molten mixture and optional cooling of the extruded strand or extruded strands, the extrudates are preferably singulated. This singulation may preferably be performed by cutting up the extrudates by means of revolving or rotating knives, wires, blades or with the assistance of laser cutters.

Preferably, intermediate or final storage of the optionally singulated extrudate or the final shape of the particles according to the invention is performed under oxygen-free atmosphere which may be achieved, e.g., by means of oxygen-scavengers.

The singulated extrudate may be press-formed into particles in order to impart the final shape to the particles.

The application of force in the extruder onto the at least plasticized mixture is adjusted by controlling the rotational speed of the conveying device in the extruder and the geometry thereof and by dimensioning the outlet orifice in such a manner that the pressure necessary for extruding the plasticized mixture is built up in the extruder, preferably immediately prior to extrusion. The extrusion parameters which, for each particular composition, are necessary to give rise to a pharmaceutical dosage form with desired mechanical properties, may be established by simple preliminary testing.

For example but not limiting, extrusion may be performed by means of a twin-screw-extruder type ZSE 18 or ZSE27 (Leistritz, Nurnberg, Germany), screw diameters of 18 or 27 mm. Screws having eccentric or blunt ends may be used. A heatable die with a round bore or with a multitude of bores each having a diameter of 0.2, 0.3, 0.4, 0.5, 0.6, 0.7, 0.8, 0.9 or 1.0 mm may be used. For a twin-screw-extruder type ZSE 18, the extrusion parameters may be adjusted e.g. to the following values: rotational speed of the screws: 120 rpm; delivery rate 2 kg/h for a ZSE 18 or 5 kg/h, 10 kg/h, or even 20 kg/h and more for a ZSE27; product temperature: in front of die 125° C. and behind die 135° C.; and jacket temperature: 110° C. The throughput can generally be increased by increasing the number of dies at the extruder outlet.

Preferably, extrusion is performed by means of twin-screw-extruders or planetary-gear-extruders, twin-screw extruders (co-rotating or contra-rotating) being particularly preferred.

The particles according to the invention are preferably produced by thermoforming with the assistance of an extruder without any observable consequent discoloration of the extrudates. The particles may be produced e.g. by means of a Micro Pelletizer (Leistritz, Nurnberg, Germany).

The process for the preparation of the particles according to the invention is preferably performed continuously. Preferably, the process involves the extrusion of a homogeneous mixture of all components. It is particularly advantageous if the thus obtained intermediate, e.g. the strand obtained by extrusion, exhibits uniform properties. Particularly desirable are uniform density, uniform distribution of the active compound, uniform mechanical properties, uniform porosity, uniform appearance of the surface, etc. Only under these circumstances the uniformity of the pharmacological properties, such as the stability of the release profile, may be ensured and the amount of rejects can be kept low.

Preferably, the particles according to the invention can be regarded as “extruded pellets”. The term “extruded pellets” has structural implications which are understood by persons skilled in the art. A person skilled in the art knows that pelletized dosage forms can be prepared by a number of techniques, including:
drug layering on nonpareil sugar or microcrystalline cellulose beads,
spray drying,
spray congealing,
rotogranulation,
hot-melt extrusion,
   spheronization of low melting materials, or
   extrusion-spheronization of a wet mass.

Accordingly, “extruded pellets” can be obtained either by hot-melt extrusion or by extrusion-spheronization.

“Extruded pellets” can be distinguished from other types of pellets, as extruded pellets typically have a different shape. The shape of the extruded pellets is typically more cut-rod-like than perfectly globated round.

“Extruded pellets” can be distinguished from other types of pellets because they are structurally different. For example, drug layering on nonpareils yields multilayered pellets having a core, whereas extrusion typically yields a monolithic mass comprising a homogeneous mixture of all ingredients. Similarly, spray drying and spray congealing typically yield spheres, whereas extrusion typically yields cylindrical extrudates which can be subsequently spheronized.

The structural differences between “extruded pellets” and “agglomerated pellets” are significant because they may affect the release of active substances from the pellets and consequently result in different pharmacological profiles. Therefore, a person skilled in the pharmaceutical formulation art would not consider “extruded pellets” to be equivalent to “agglomerated pellets”.

The pharmaceutical dosage forms according to the invention may be prepared by any conventional method. Preferably, however, the pharmaceutical dosage forms are prepared by compression. Thus, particles as hereinbefore defined are preferably mixed, e.g. blended and/or granulated (e.g. wet granulated), with matrix material and the resulting mix (e.g. blend or granulate) is then compressed, preferably in moulds, to form pharmaceutical dosage forms. It is also envisaged that the particles herein described may be incorporated into a matrix using other processes, such as by melt granulation (e.g. using fatty alcohols and/or water-soluble waxes and/or water-insoluble waxes) or high shear granulation, followed by compression.

When the pharmaceutical dosage forms according to the invention are manufactured by means of an eccentric press, the compression force is preferably within the range of from 5 to 15 kN. When the pharmaceutical dosage forms according to the invention are manufactured by means of a rotating press, the compression force is preferably within the range of from 5 to 40 kN, in certain embodiments >25 kN, in other embodiments 13 kN.

The pharmaceutical dosage forms according to the invention may optionally comprise a coating, e.g. a cosmetic coating. The coating is preferably applied after formation of the pharmaceutical dosage form. The coating may be applied prior to or after the curing process. Preferred coatings are Opadry® coatings available from Colorcon. Other preferred coating are Opaglos® coatings, also commercially available from Colorcon.

The pharmaceutical dosage form according to the invention is characterized by excellent storage stability. Preferably, after storage for 6 months, 3 months, 2 months, or 4 weeks at 40° C. and 75% rel. humidity, the content of pharmacologically active compound amounts to at least 98.0%, more preferably at least 98.5%, still more preferably at least 99.0%, yet more preferably at least 99.2%, most preferably at least 99.4% and in particular at least 99.6%, of its original content before storage. Suitable methods for measuring the content of the pharmacologically active compound in the pharmaceutical dosage form are known to the skilled artisan. In this regard it is referred to the Eur. Ph. or the USP, especially to reversed phase HPLC analysis. Preferably, the pharmaceutical dosage form is stored in closed, preferably sealed containers.

The particles and pharmaceutical dosage forms according to the invention may be used in medicine, e.g. as an analgesic. The particles and pharmaceutical dosage forms are therefore particularly suitable for the treatment or management of attention deficit hyperactivity disorder (ADHD) or narcolepsy (sudden and uncontrollable attacks of drowsiness and sleepiness). In such pharmaceutical dosage forms, the pharmacologically active compound is preferably an analgesic.

A further aspect according to the invention relates to the pharmaceutical dosage form as described above for use in the treatment of attention deficit hyperactivity disorder (ADHD) or narcolepsy (sudden and uncontrollable attacks of drowsiness and sleepiness). A further aspect of the invention relates to the use of a pharmacologically active compound for the manufacture of a pharmaceutical dosage form according to the invention for use in the treatment of attention deficit hyperactivity disorder (ADHD) or narcolepsy (sudden and uncontrollable attacks of drowsiness and sleepiness). Another aspect of the invention relates to a method for treating attention deficit hyperactivity disorder (ADHD) or narcolepsy (sudden and uncontrollable attacks of drowsiness and sleepiness) in a subject in need of such treatment, comprising orally administering a pharmaceutical dosage form according to the invention.

The subjects to which the pharmaceutical dosage forms according to the invention can be administered are not particularly limited. Preferably, the subjects are animals, more preferably human beings.

A further aspect according to the invention relates to the use of a pharmaceutical dosage form as described above for avoiding or hindering the abuse of the pharmacologically active compound contained therein.

A further aspect according to the invention relates to the use of a pharmaceutical dosage form as described above for avoiding or hindering the unintentional overdose of the pharmacologically active compound contained therein.

In this regard, the invention also relates to the use of a pharmacologically active compound as described above and/or a polyalkylene oxide as described above for the manufacture of the pharmaceutical dosage form according to the invention for the prophylaxis and/or the treatment of a disorder, thereby preventing an overdose of the pharmacologically active compound, particularly due to comminution of the pharmaceutical dosage form by mechanical action.

### EXAMPLES

The following examples further illustrate the invention but are not to be construed as limiting its scope:

### General Operation Procedures

As a general operation procedure 1, powder mixtures of various ingredients were manufactured by weighing (10 kg balance), sieving (1.0 mm hand sieve) and blending. The thus obtained powder mixtures were then hot-melt extruded (twin-screw extruder, Leistritz ZSE 18, blunt ends of kneading elements, and extrusion diameter of 8×0.8 mm). The extrudates were pelletized (LMP) and then analyzed. The particulates according to Examples 1-16 were prepared according to general operation procedure 1.

As a general operation procedure 2, tablets were prepared by weighing, sieving (1.0 mm hand sieve), blending (LM40 mixer) and pressing (Korsch EK0 press) powder mixtures of various ingredients. The thus obtained tablets were sintered in a drying cabinet at 90° C. for 2 hours and then analyzed. The tablets according to Example 17 were prepared according to general operation procedure 2.

In vitro dissolution was tested in accordance with USP (apparatus II), in 600 ml 0.1 M HCl (pH 1) at 75 rpm (n=3). In some examples, the pH of the release medium was switched from acidic to neutral after 60 minutes or 120 minutes. In some examples, the medium was ethanolic (40 vol.-%).

Resistance against solvent extraction was tested by dispensing particles in 5 ml of boiling water. After boiling for 5 minutes the liquid was drawn up into a syringe (needle 21 G equipped with a cigarette filter), and the amount of the pharmacologically active compound contained in the liquid within the syringe was determined via HPLC.

The test was performed on the extrudates as such but not on capsules or tablets containing such extrudates, as this test more relevant with respect to drug abuse. The other constituents of dosage forms (e.g. capsules or tablets) typically make it even more difficult for the abuser to tamper with the dosage form, e.g. by blocking the filters of syringes and the like. Thus, in the course of tampering, abusers frequently initially separate the drug containing subunits of dosage forms (here extrudates) from the remainder of the dosage forms in order to facilitate subsequent abuse, e.g. by extraction. Accordingly, it is more significant to evaluate tamper resistance of the extrudates instead of the overall dosage forms.

Capsules providing modified release (MR) or amphetamine sulfate as pharmacologically active compound were manufactured by combining immediate release particles and controlled release particle(s) with one another.

### Example 1—Immediate Release Particles Coated with Non-Enteric Coating which does not Delay In Vitro Dissolution

Pellets providing immediate release of amphetamine sulfate were manufactured by hot-melt extrusion. The thus obtained extruded pellets were coated with a non-functional (non-enteric) protection coating which does not delay in vitro dissolution to avoid sticking of pellets.

The pellets (multitude of immediate release particles) contained 20 mg amphetamine sulfate. The IR particles had the following composition:

| per pellets [mg] | substance | amount [wt.-%] |
|---|---|---|
| 20.00 | amphetamine sulfate | 14.89 |
| 61.19 | polyethylene oxide 7 mio. | 45.56 |
| 14.57 | polyethylene glycol 6000 | 10.85 |
| 0.24 | alpha tocopherol | 0.18 |
| 24.00 | starch 1500 | 17.87 |
| 14.30 | Opadry ® II clear | 10.65 |
| 134.30 | | 100.00 |

Opadry ® II clear: a non-enteric coating which does not delay in vitro dissolution.

Powder mixtures of the ingredients were manufactured and subsequently hot-melt extruded under the following extrusion conditions:

| | |
|---|---|
| Speed screw [rpm] | 100 |
| Feed rate [g/min] | 16.66 |
| Melt pressure [bar] | 90-185 |
| melt temperature discharge | 140-145 |
| [° C.] | |

The average individual total weight of a single particle was below 2.0 mg.

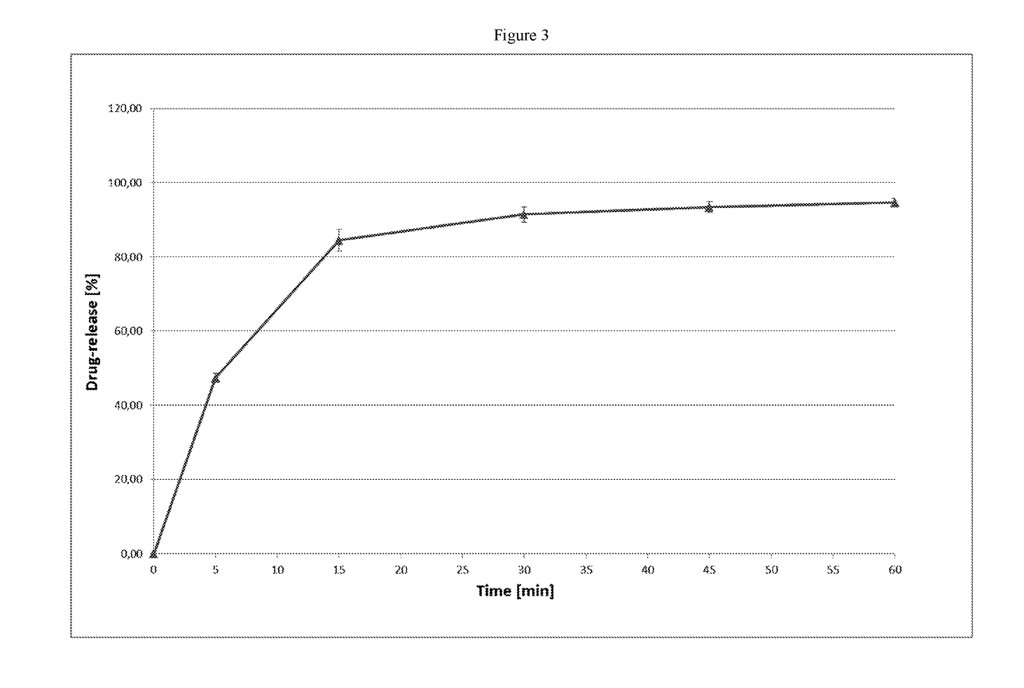

The in vitro release profile of the 20 mg IR particles with non-functional coat is shown in FIG. 3.

### Example 2—Controlled Release Particles Comprising Enteric Coating Providing Delayed Release

In accordance with Example 1, 20 mg DR particles were manufactured comprising a functional, i.e. enteric coating. The DR particles had the following composition:

| per pellets [mg] | substance | amount [wt.-%] |
|---|---|---|
| 20.00 | amphetamine sulfate | 12.71 |
| 61.19 | polyethylene oxide 7 mio. | 38.88 |
| 14.57 | polyethylene glycol 6000 | 9.26 |
| 0.24 | alpha tocopherol | 0.15 |
| 24.00 | starch 1500 | 15.25 |
| 37.40 | DR Coating | 23.76 |
| | (Eudragit ® L30-D55 + 3.3% TEC) | |
| 157.40 | | 100.00 |

Eudragit® L30-D55 is a commercially available enteric coating material. Triethylcitrate (TEC) is conventionally used as plasticizer.

The average individual total weight of a single particle was below 2.0 mg.

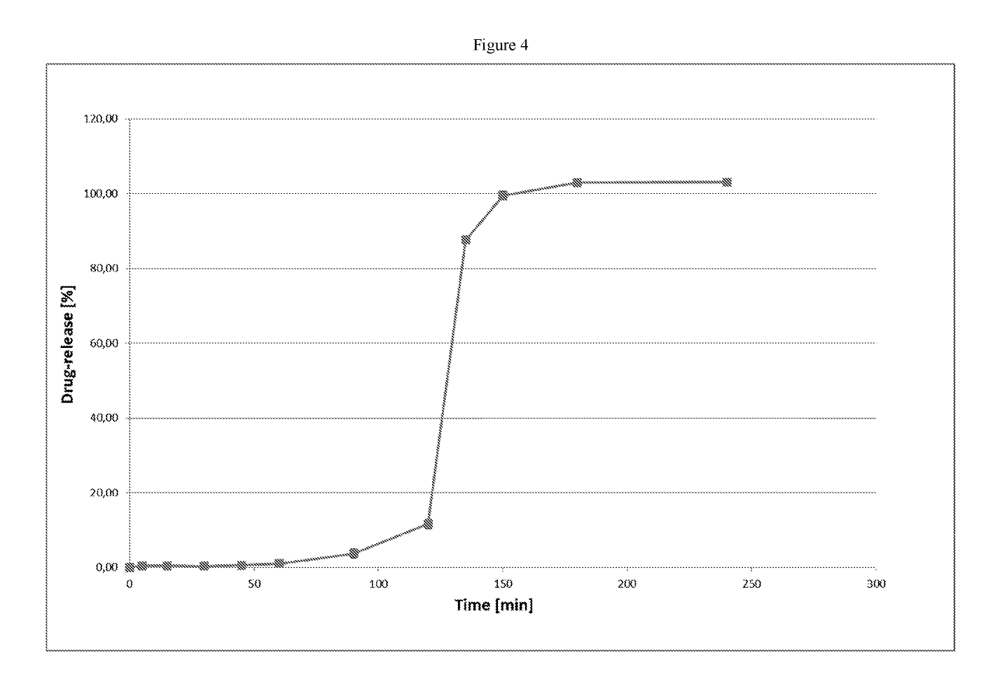

The in vitro release profile of the 20 mg DR particles with non-functional coat is shown in FIG. 4 with a pH switch of the release medium from acidic to neutral after 2 hours. In acidic media, the mean after 120 minutes was 11.64% (SD=1.24%) such that the in vitro release profile reflected a desired delayed release.

### Example 3—Controlled Release Particles Comprising Specific Enteric Coating Providing Delayed Release

In accordance with Example 2, 20 mg DR particles were manufactured comprising another functional, i.e. enteric coating. The DR particles had the following composition:

| per pellets [mg] | substance | amount [wt.-%] |
|---|---|---|
| 20.00 | amphetamine sulfate | 12.71 |
| 61.19 | polyethylene oxide 7 mio. | 38.88 |
| 14.57 | polyethylene glycol 6000 | 9.26 |
| 0.24 | alpha tocopherol | 0.15 |
| 24.00 | starch 1500 | 15.25 |
| 37.40 | DR Coating (Evonik ADD) | 23.76 |
| 157.40 | | 100.00 |

Evonik ADD is a commercially available enteric coating material. Such coating comprises an inner layer of sodium alginate followed by an outer layer of an Acrylate (e.g. Eudragit®) polymer, e.g. a methacrylic acid-ethyl acrylate copolymer (1:1) (e.g. Eudragit® L 30 D-55). Sodium alginate spray suspension (solid content: 4% w/w) may be prepared e.g. by dissolving sodium alginate in 85% water, adding 50% talc (based on sodium alginate) homogenizing separately, stirring and filtering (420 μm). Eudragit® spray suspension (solid content: 20% w/w) may prepared by first dissolving 3% polysorbate 80 (based on dry polymer) in warm water, then adding to the homogenized 50% talc and 10% triethyl citrate (both based on dry polymer), followed by mixing with the Eudragit® L 30 D-55 dispersion. The suspension may also be sieved (420 μm) before spraying.

The average individual total weight of a single particle was below 2.0 mg.

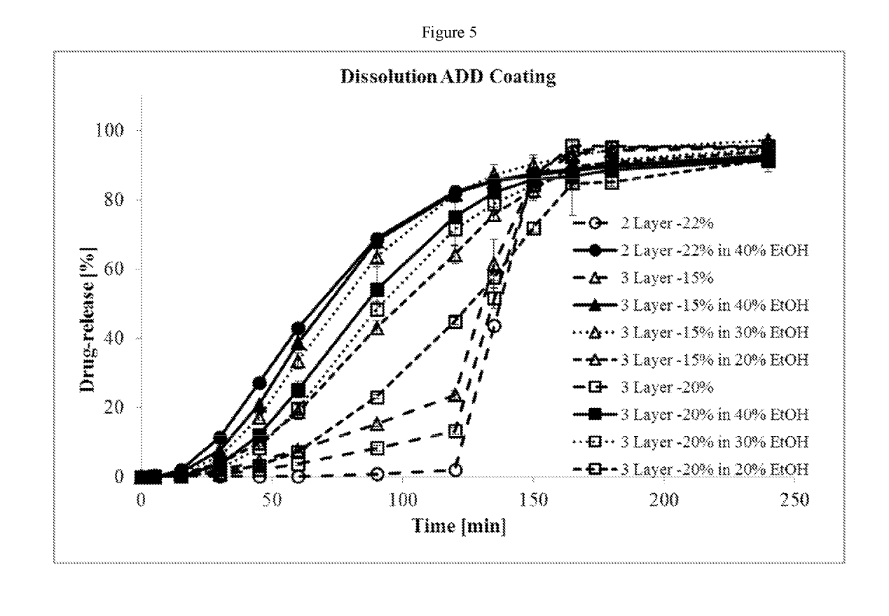

The in vitro release profile of the 20 mg DR particles is shown in FIG. 5 with a pH switch of the release medium from acidic to neutral after 2 hours. As demonstrated, the DR particles are gastric resistant and show no alcohol dose dumping.

### Example 4—Controlled Release Particle Providing Extended Release

In accordance with Examples 1 to 3, two 20 mg PR particles (cut rods) of different total weight were manufactured. The PR particles had the following composition:

| | 4-1 | | 4-2 | |
|---|---|---|---|---|
| composition | in mg | in wt.-% | in mg | in wt.-% |
| amphetamine sulfate | 20.00 | 9.30 | 20.00 | 5.71 |
| PEO 7 Mio. (Sumitomo) | 139.77 | 65.01 | 237.70 | 67.91 |
| PEG 6000 | 33.30 | 15.49 | 56.60 | 16.17 |
| HPMC | 21.50 | 10.00 | 35.00 | 10.00 |
| alpha tocopherol | 0.43 | 0.20 | 0.70 | 0.20 |

| | | |
|---|---|---|
| weight per dose (cut rod) | 215 mg | 350 mg |

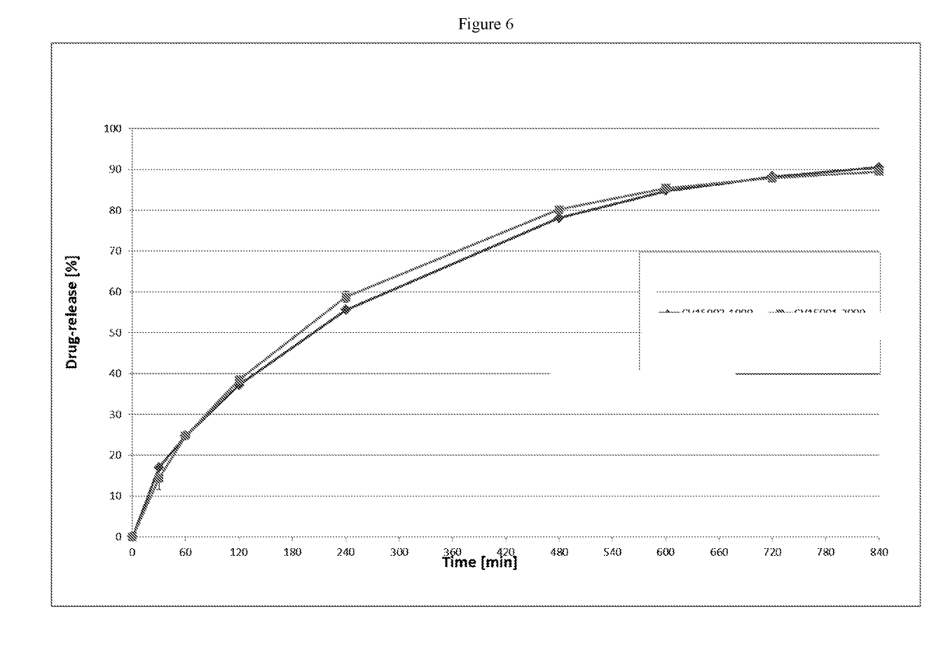

The dissolution with 50 rpm in SIF pH 6.8 of the cut rod according to Example 4-1 (215 mg; square marks) in comparison to the cut rod according to Example 4-2 (350 mg; rhomboid marks) is shown in FIG. 6. Surprisingly, but cut rods showed a similar dissolution profile.

Both cut rods were tested with respect to their abuse resistance. Both cut rods were pretreated for 2 minutes in coffee grinder and the resultant material was subjected to solvent extraction:

| | 4-1 | 4-2 |
|---|---|---|
| 1 | 14.19 | 11.26 |
| 2 | 4.29 | 8.19 |
| 3 | 14.66 | —* |
| mean [%] | 11.05 | — |
| SD [%] | 5.86 | — |

*= could not be analyzed as to less material could be drawn into syringe

### Example 5—Immediate Release Particles of Example 1 and Delayed Release Particles of Example 2

The IR particles of Example 1 were combined with the DR particles of Example 2 and filled into capsules of size 0. Thus, the capsules had the following overall composition:

| per | | | | |
|---|---|---|---|---|
| capsule | | | | |
| (Size 0) | | per capsule | | Amount |
| [mg] | form | [mg] | Substance | [%] |
| 134.30 | IR | 20.00 | amphetamine sulfate | 46.04 |
| | particles | 61.19 | polyethylene oxide 7 mio. | |
| | | 14.57 | polyethylene glycol 6000 | |
| | | 0.24 | alpha tocopherol | |
| | | 24.00 | Starch 1500 | |
| | | 14.30 | Opadry ® II clear | |
| 157.40 | DR | 20.00 | amphetamine sulfate | 53.96 |
| | particles | 61.19 | polyethylene oxide 7 mio. | |
| | | 14.57 | polyethylene glycol 6000 | |
| | | 0.24 | alpha tocopherol | |
| | | 24.00 | starch 1500 | |
| | | 37.40 | DR Coating | |
| | | | (Eudragit ® L30-D55 + | |
| | | | 3.3% TEC)¹ | |
| 291.70 | | | | 100.00 |

¹The DR coating employed in Example 5 may be considered as a standard enteric coating and in contrast to Example 3 did not contain any inner layer of sodium alginate. With respect to the avoidance of dose dumping in aqueous ethanol, the two layered coating of Example 3 is superior over the conventional coating according to Example 5.

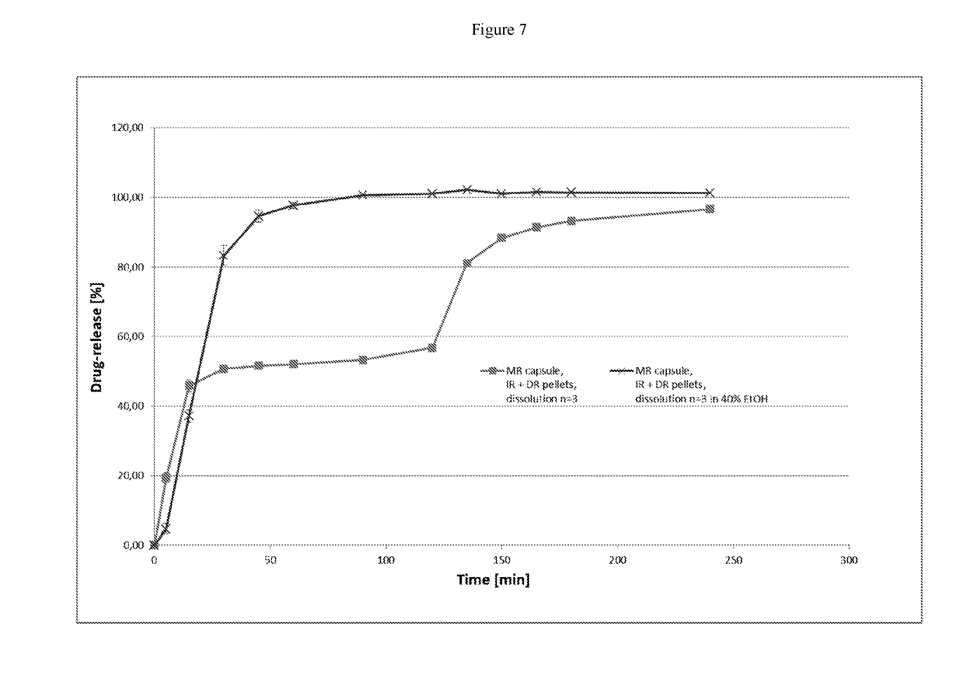

The result of measuring in vitro dissolution in 40% ethanol is shown in FIG. 7 with a pH switch of the release medium from acidic to neutral after 2 hours.

### Example 6—Immediate Release Particles of Example 1 and Controlled Release Particles of Example 4-1

The IR particles of Example 1 were combined with the PR particle of Example 4-1 (215 mg) and filled into capsules of size 0. Thus, the capsules had the following overall composition:

| per | | | | |
|---|---|---|---|---|
| capsule | | | | |
| (Size 0) | | per capsule | | Amount |
| [mg] | form | [mg] | Substance | [wt.-%] |
| 134.30 | IR | 20.00 | amphetamine sulfate | 38.45 |
| | particles | 61.19 | polyethylene oxide 7 | |
| | | | mio. | |
| | | 14.57 | polyethylene glycol 6000 | |
| | | 0.24 | alpha tocopherol | |
| | | 24.00 | starch 1500 | |
| | | 14.30 | Opadry ® II clear | |
| 215.00 | PR cut rod | 20.00 | amphetamine sulfate | 61.55 |
| | | 139.77 | polyethylene oxide 7 | |
| | | | mio. | |
| | | 21.50 | hypromellose | |
| | | 33.30 | polyethylene glycol 6000 | |
| | | 0.43 | alpha tocopherol | |
| 349.30 | | | | 100.00 |

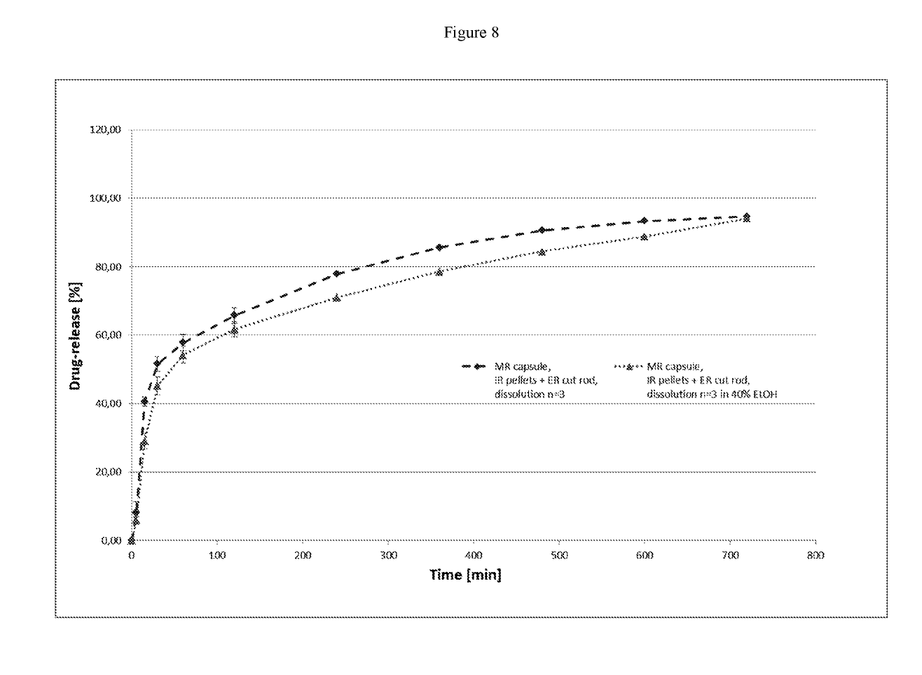

The result of measuring in vitro dissolution in 40% ethanol is shown in FIG. 8. As demonstrated, the DR approach of Example 5 shows alcohol dose dumping, which can be avoided by changing the enteric coating material, as shown in FIG. 5.

### Example 7—Immediate Release Particles Comprising Oxycodone and Different Disintegrants

Powder mixtures of the following ingredients were manufactures and subsequently hot-melt extruded under the following extrusion conditions:

| | 1-1 | 1-2 | 1-3 | 1-4 | 1-5 |
|---|---|---|---|---|---|
| per dosis | mg/wt.-% | mg/wt.-% | mg/wt.-% | mg/wt.-% | mg/wt.-% |
| Oxycodone HCl | 10.00/5.56 | 10.00/5.56 | 10.00/5.56 | 10.00/5.56 | 10.00/5.56 |
| Citric acid | 1.44/0.80 | 1.44/0.80 | 1.44/0.80 | 1.44/0.80 | 1.44/0.80 |
| Macrogol 6000 | 25.20/14.00 | 25.20/14.00 | 25.20/14.00 | 25.20/14.00 | 25.20/14.00 |
| α-Tocopherol | 0.36/0.20 | 0.36/0.20 | 0.36/0.20 | 0.36/0.20 | 0.36/0.20 |
| Xanthan Gum Type 602 | 9.00/5.00 | 9.00/5.00 | 9.00/5.00 | 9.00/5.00 | 9.00/5.00 |
| Polyethylene oxide 7 Mio. | 98.00/54.44 | 98.00/54.44 | 98.00/54.44 | 98.00/54.44 | 95.22/52.20 |
| Sodium bicarbonate | — | — | — | — | 2.78/1.54 |
| Sodium starch glycolate | 36.00/20.00 | — | — | — | — |
| Croscarmellose sodium | — | 36.00/20.00 | — | — | — |
| Starch 1500 | — | — | 36.00/20.00 | — | — |
| Maize starch | — | — | — | 36.00/20.00 | — |
| Carbomer Carbopol 71G | — | — | — | — | 36.00/20.00 |
| Σ | 180.00/100.00 | 180.00/100.00 | 180.00/100.00 | 180.00/100.00 | 180.00/100.00 |
| Speed screw [rpm] | 100 | 100 | 100 | 100 | 120 |
| Feed rate [g/min] | 16.66 | 16.66 | 16.66 | 16.66 | 16.66 |
| Melt pressure [bar] | 119 | 141 | 136 | 135 | 116 |
| melt temperature discharge | 140 | 143 | 142 | 143 | 145 |
| [° C.] | | | | | |

The in vitro dissolution test revealed the following release profiles:

| Dissolution | | | | | |
|---|---|---|---|---|---|
| Oxycodone % | 1-1 | 1-2 | 1-3 | 1-4 | 1-5 |
| after 5 min | 70 | 74 | 66 | 78 | 58 |
| after 15 min | 88 | 91 | 88 | 94 | 83 |
| after 30 min | 94 | 94 | 95 | 100 | 92 |
| after 60 min | 96 | 96 | 97 | 102 | 96 |

The test for tamper-resistance provided the following results (where all tested pellets remained intact after the breaking strength tester had reached its upper force limit):

| test battery | 1-1 | 1-2 | 1-3 | 1-4 | 1-5 |
|---|---|---|---|---|---|
| 1 | 0.00* | 1.34 | 0.00* | 22.40 | 0.00* |
| 2 | 0.00* | 3.07 | 20.20 | 30.32 | 0.00* |
| 3 | 0.00* | 1.26 | 6.03 | 18.67 | 0.00* |
| mean [%] | 0.00* | 1.89 | 8.74 | 28.80 | 0.00* |
| SD [%] | 0.00* | 1.02 | 10.37 | 5.95 | 0.00* |

*not tested, sample too jelly and could not be drawn into syringe

It becomes clear from the above experimental data that in the immediate release particles the tested disintegrants provide different performance. Under the given experimental conditions, cellulose derivatives (e.g. croscarmellose sodium) provided the best performance, followed by starch derivatives (e.g. sodium starch glycolate) and gas releasing substances (here sodium bicarbonate), followed by pregelatinized starch (e.g. starch 1500) and standard starch (e.g. native maize starch).

### Example 8—Immediate Release Particles Comprising Amphetamine and Different Disintegrants

Powder mixtures of the following ingredients were manufactured and subsequently hot-melt extruded under the following extrusion conditions:

| | 8-1 | 8-2 | 8-3 | 8-4 |
|---|---|---|---|---|
| per dosis | mg/wt.-% | mg/wt.-% | mg/wt.-% | mg/wt.-% |
| Amphetamine sulfate | 30.00/12.00 | 30.00/12.00 | 30.00/12.00 | 30.00/12.00 |
| Citric acid | 2.00/0.80 | 2.00/0.80 | — | — |
| PEG 6000 | 35.00/14.00 | 35.00/14.00 | 32.60/13.00 | 32.60/13.00 |
| α-Tocopherol | 0.50/0.20 | 0.50/0.20 | 0.50/0.20 | 0.50/0.20 |
| Xanthan Gum Type 602 | — | 12.50/5.00 | — | — |
| Polyethylene oxide 7 Mio. | 182.50/73.00 | 120.00/48.00 | 136.90/54.70 | 136.90/54.70 |
| Sodium hydrogen | — | — | — | — |
| carbonate | | | | |
| Croscarmellose sodium | — | 50/20.00 | 50.00/20.00 | — |
| Starch 1500 | — | — | — | — |
| Carboxymethyl starch | — | — | — | 50.00/20.00 |
| PVP-CL | — | — | — | — |
| Σ | 250.00/100.00 | 250.00/100.00 | 250.0/100.00 | 250.0/100.00 |
| Speed screw [rpm] | 100 | 100 | 100 | 100 |
| Extruder Load [%] | 75.00 | 75.00 | 75.00 | 75.00 |
| Melt pressure [bar] | 1 | 1 | 1 | 1 |
| melt temperature discharge | 145 | 145 | 145 | 145 |
| [° C.] | | | | |

| | 8-5 | 8-6 | 8-7 |
|---|---|---|---|
| per dosis | mg/wt.-% | mg/wt.-% | mg/wt.-% |
| Amphetamine sulfate | 30.00/12.00 | 30.00/12.00 | 30.00/12.00 |
| Citric acid | — | — | — |
| PEG 6000 | 32.60/13.00 | 32.60/13.00 | 32.60/13.04 |
| α-Tocopherol | 0.50/0.20 | 0.50/0.20 | 0.50/0.20 |
| Xanthan Gum Type | — | — | — |
| 602 | | | |
| Polyethylene oxide | 136.90/54.70 | 136.90/54.70 | 136.90/54.76 |
| 7 Mio. | | | |
| Sodium hydrogen | — | 50.00/20.00 | — |
| carbonate | | | |
| Croscarmellose | — | — | — |
| sodium | | | |
| Starch 1500 | 50.00/20.00 | — | — |
| Carboxymethyl starch | — | — | — |
| PVP-CL | — | — | 50.00/20.00 |
| Σ | 250.0/100.00 | 250.0/100.00 | 250.00/100.00 |
| Speed screw [rpm] | 100 | 100 | 100 |
| Extruder Load [%] | 75.00 | 75.00 | 75.00 |
| Melt pressure [bar] | 1 | 1 | 1 |
| melt temperature | 145 | 145 | 145 |
| discharge [° C.] | | | |

The in vitro dissolution test revealed the following release profiles:

| Dissolution Amphetamine sulfate % | 8-1 | 8-2 | 8-3 | 8-4 | 8-5 | 8-6 | 8-7 |
|---|---|---|---|---|---|---|---|
| after 5 min | 67 | 61 | 51 | 48 | 62 | 45 | 63 |
| after 15 min | 90 | 90 | 85 | 81 | 83 | 70 | 87 |
| after 30 min | 96 | 97 | 94 | 93 | 94 | 80 | 93 |
| after 60 min | 98 | 99 | 97 | 97 | 98 | 84 | 96 |

The test for tamper-resistance provided the following results (where all tested pellets remained intact after the breaking strength tester had reached its upper force limit):

| test battery | 8-1 | 8-2 | 8-3 | 8-4 | 8-5 | 8-6 | 8-7 |
|---|---|---|---|---|---|---|---|
| 1 | 38.41 | 32.54 | 6.11 | 11.31 | 4.57 | 8.23 | 44.80 |
| 2 | 28.83 | 33.63 | 11.43 | 8.18 | 0.00* | 8.61 | 51.17 |
| 3 | 23.67 | 12.16 | 14.56 | 5.20 | 0.00* | 12.77 | 50.96 |
| mean [%] | 30.30 | 26.11 | 10.70 | 8.23 | 0.00* | 9.87 | 48.98 |
| SD [%] | 7.48 | 12.09 | 4.27 | 3.06 | 0.00* | 2.52 | 3.62 |

*not tested, sample too jelly and could not be drawn into syringe

It becomes clear from the above experimental data that in the immediate release particles, the tested disintegrants provide an improved resistance against solvent extraction. Croscarmellose sodium (8-2, 8-3), carboxymethyl starch (8-4), starch 1500 (8-5) and sodium hydrogen carbonate provided the best results, whereas PVP-CL (8-7) did not show an advantage over the comparative composition (8-1).

### Example 9—Immediate Release Particles Comprising Gelling Agent and Disintegrant

The influence of the presence and absence of gelling agent as well as the influence of the presence and absence of disintegrant was investigated in analogy to Examples 7 and 8. The following compositions A to F were each prepared for Oxycodone, Hydrocodone, Morphine sulfate and Hydromorphone, respectively:

| | 9-A | | 9-B | | 9-C | | 9-D | | 9-E | | 9-F | |
|---|---|---|---|---|---|---|---|---|---|---|---|---|
| Substance | mg | wt.-% | mg | wt.-% | mg | wt.-% | mg | wt.-% | mg | wt.-% | mg | wt.-% |
| API¹ | 10.00 | 5.56 | 10.00 | 5.56 | 10.00 | 5.56 | 10.00 | 5.56 | 10.00 | 5.56 | 10.00 | 5.56 |
| Citric | 1.44 | 0.80 | 1.44 | 0.80 | 1.44 | 0.80 | 1.44 | 0.80 | 1.44 | 0.80 | 1.44 | 0.80 |
| acid | | | | | | | | | | | | |
| PEG | 25.20 | 14.00 | 25.20 | 14.00 | 25.20 | 14.00 | 25.20 | 14.00 | 25.20 | 14.00 | 25.20 | 14.00 |
| α-Toc. | 0.36 | 0.20 | 0.36 | 0.20 | 0.36 | 0.20 | 0.36 | 0.20 | 0.36 | 0.20 | 0.36 | 0.20 |
| PEO | 143.0 | 79.44 | 107.0 | 59.44 | 107.0 | 59.44 | 134.0 | 74.44 | 98.00 | 54.44 | 98.00 | 54.44 |
| Carhopol | — | — | 36.00 | 20.00 | 27.00 | 15.00 | — | — | | | — | — |
| Xanthan | — | — | — | — | 9.00 | 5.00 | 9.00 | 5.00 | 9.00 | 5.00 | 9.00 | 5.00 |
| Carb. MS | — | — | — | — | — | — | — | — | 36.00 | 20.00 | — | — |
| CrosCS | — | — | — | — | — | — | — | — | — | — | 36.00 | 20.00 |
| Σ | 180 | 100 | 180 | 100 | 180 | 100 | 180 | 100 | 180 | 100 | 180 | 100 |

¹The compositions A to F containing Hydromorphone as API were modified in that they contained 8.00 mg Hydromorphone only. The difference of 2.00 mg was replaced by the corresponding amount of PEO
API = pharmacologically active compound;
PEG = Polyethylene glycol 6000;
α-Toc. = α-Tocopherole;
PEO = polyethylene oxide 7 Mio;
Carbopol = Carbopol 71G;
Xanthan = Xanthan gum;
Carb. MS = Carboxy methyl starch;
CrosCS = Croscarmellose sodium

In vitro release as well as resistance against solvent extraction were determined in accordance with the invention. The results for the different pharmacologically active compounds are shown in the table here below:

| | Oxycodone | | Hydrocodone | | Morphine sulfate | | Hydromorphone | |
|---|---|---|---|---|---|---|---|---|
| Formulation | extract. | diss. | extract. | diss. | extract. | diss. | extract. | diss. |
| 9-A | 50% | 73% | 40% | 87% | 34% | 87% | 49% | 84% |
| 9-B | 40% | 90% | 0% | 91% | 9% | 83% | 29% | 87% |
| 9-C | 28% | 90% | 0% | 95% | 3% | 82% | 26% | 89% |
| 9-D | 12% | 91% | 32% | 75% | 14% | 88% | 33% | 91% |
| 9-E | 0% | 94% | 5% | 92% | 0% | 90% | 14% | 91% |
| 9-F | 2% | 94% | 1% | 103% | — | — | 7% | 91% |

extract. = extracted in solvent;
diss = dissolution after 30 minutes

It becomes clear from the above comparative data that the disintegrants in formulations E and F provide best performance with respect to immediate drug release and resistance against solvent extraction for all tested pharmacologically active compounds, whereas the formulations A, B, C and D only provided partial effects for some of the tested pharmacologically active compounds.

### Example 10—Quantity of Disintegrant Part I

The influence of the content of disintegrant was investigated in analogy to Examples 7 to 9. Compositions 10-1 to 10-3 were prepared and in vitro dissolution as well as resistance against solvent extraction were determined.

| Substance | 10-1 | | 10-2 | | 10-3 | |
|---|---|---|---|---|---|---|
| per dose | mg | wt.-% | mg | wt.-% | mg | wt.-% |
| Oxycodone | 10.00 | 5.56 | 10.00 | 5.56 | 10.00 | 5.56 |
| HCl | | | | | | |
| Citric acid | 1.44 | 0.80 | 1.44 | 0.80 | 1.44 | 0.80 |
| PEG 6000 | 27.51 | 15.28 | 25.20 | 14.00 | 27.51 | 15.28 |
| α-Tocopherol | 0.36 | 0.20 | 0.36 | 0.20 | 0.36 | 0.20 |
| Xanthan Gum | 9.00 | 5.00 | 9.00 | 5.00 | 9.00 | 5.00 |
| Type 602 | | | | | | |
| PEO 7 Mio. | 104.69 | 58.16 | 98.00 | 54.44 | 91.31 | 50.73 |
| Sodium starch | 27.00 | 15.00 | 36.00 | 20.00 | 45.00 | 25.00 |
| glycolate | | | | | | |
| | 180.00 | 100.00 | 180.00 | 100.00 | 180.00 | 100.00 |

| Dissolution | | | |
|---|---|---|---|
| (n = 3): | | | |
| 0 | 0.00 | 0.00 | 0.00 |
| 5 | 64.46 | 69.73 | 62.04 |
| 15 | 78.42 | 87.57 | 81.83 |
| 30 | 91.24 | 94.44 | 91.76 |
| 60 | 94.82 | 96.49 | 95.12 |
| extraction | | | |
| without | | | |
| milling: | | | |
| mean [%] | 10.10 | 0.00* | 16.37 |
| SD [%] | 4.67 | 0.00* | 12.67 |

*not tested, sample too jelly and could not be drawn into syringe

It becomes clear from the above comparative data that under the given conditions the best results could be achieved at a content of 20 wt.-% disintegrant (here sodium starch glycolate).

### Example 11—Quantity of Disintegrant Part II

The influence of the content of disintegrant was investigated in analogy to Examples 1 to 7. Compositions 11-1 to 11-4 were prepared and in vitro dissolution as well as resistance against solvent extraction were determined.

| | 11-1 | | 11-2 | | 11-3 | | 11-4 | |
|---|---|---|---|---|---|---|---|---|
| per dose | mg | wt.-% | mg | wt.-% | mg | wt.-% | mg | wt.-% |
| Amphetamine sulfate | 30.00 | 13.95 | 30.00 | 16.67 | 30.00 | 13.95 | 30.00 | 16.67 |
| PEG 6000 | 27.20 | 12.65 | 21.85 | 12.14 | 27.20 | 12.65 | 21.85 | 12.14 |
| α-Tocopherol | 0.43 | 0.20 | 0.36 | 0.20 | 0.43 | 0.20 | 0.36 | 0.20 |
| Polyethylene oxide 7 Mio. | 114.37 | 53.20 | 91.79 | 50.99 | 114.37 | 53.20 | 91.79 | 50.99 |
| Croscarmellose sodium | 43.00 | 20.00 | 36.00 | 20.00 | | | | |
| Starch 1500 | | | | | 43.00 | 20.00 | 36.00 | 20.00 |
| Σ | 215.00 | 100.00 | 180.00 | 100.00 | 215.00 | 100.00 | 180.00 | 100.00 |

| | | | | |
|---|---|---|---|---|
| Speed screw [rpm] | 100 | 100 | 100 | 100 |
| Extruder Load [%] | 75.00 | 75.00 | 75.00 | 75.00 |
| Melt pressure [bar] | 1 | 1 | 1 | 1 |
| melt temperature discharge | 145 | 145 | 145 | 145 |
| [° C.] | | | | |

The in vitro dissolution test revealed the following release profiles:

| Dissolution | | | | |
|---|---|---|---|---|
| Amphetamine sulfate % | 11-1 | 11-2 | 11-3 | 11-4 |
| after 5 min | 60 | 74 | 75 | 78 |
| after 15 min | 91 | 94 | 82 | 81 |
| after 30 min | 97 | 99 | 84 | 87 |
| after 60 min | 97 | 99 | 85 | 88 |

The test for tamper-resistance provided the following results (where all tested pellets remained intact after the breaking strength tester had reached its upper force limit):

| test battery | 11-1 | 11-2 | 11-3 | 11-4 |
|---|---|---|---|---|
| 1 | 7.92 | 17.51 | 0.00* | 6.42 |
| 2 | 7.74 | 12.79 | 0.00* | 3.66 |
| 3 | 8.49 | 16.85 | 0.00* | 1.83 |
| mean [%] | 8.05 | 15.72 | 0.00* | 3.97 |
| SD [%] | 0.39 | 2.56 | 0.00* | 2.31 |

*not tested, sample too jelly and could not be drawn into syringe

It becomes clear from the above comparative data that under the given conditions lower contents of disintegrant provide an improved resistance against solvent extraction.

### Example 12—Immediate Release Particles Coated with Non-Enteric Coating which does not Delay In Vitro Dissolution

In accordance with Example 1, pellets providing immediate release of amphetamine sulfate were manufactured by hot-melt extrusion. The thus obtained extruded pellets were coated with a non-functional (non-enteric) protection coating which does not delay in vitro dissolution to avoid sticking of pellets.

The pellets (multitude of immediate release particles) contained 20 mg amphetamine sulfate. The IR particles had the following composition (see Example 1):

| per dosis [mg] | substance | amount [wt.-%] |
|---|---|---|
| 20.00 | amphetamine sulfate | 14.89 |
| 61.19 | polyethylene oxide 7 mio. | 45.56 |
| 14.57 | polyethylene glycol 6000 | 10.85 |
| 0.24 | alpha tocopherol | 0.18 |
| 24.00 | starch 1500 | 17.87 |
| 14.30 | Opadry ® II clear | 10.65 |
| 134.30 | | 100.00 |

Opadry ® II clear: a non-enteric coating which does not delay in vitro dissolution.

Powder mixtures of the ingredients were manufactured and subsequently hot-melt extruded in accordance with Example 1. The thus extruded pellets were coated with a non-enteric coating which does not delay in vitro dissolution having the following composition:

| Substance | Amount [wt.-%] |
|---|---|
| Opadry ® II clear | 15.00 |
| water | 85.00 |
| | 100.00 |

The average individual total weight of a single particle was below 2.0 mg.

### Example 13—Controlled Release Particles Comprising Specific Enteric Coating Providing Delayed Release

In accordance with Example 3, 20 mg DR particles were manufactured comprising a functional, i.e. enteric coating. A hot melt extruded pellet core was subsequently provided with three coating layers, namely 5.5 wt.-% of an inner layer based on Opadry® pink (DR Coating Layer 1), 30.1 wt.-% of an intermediate layer based on alginate (polymer amount 20%) (DR Coating Layer 2), and 36.7 wt.-% of Eudragit® L30-D55 (polymer amount=22%) (DR Coating Layer 3).

The DR coated pellets had the following composition:

| per dosis | | Amount |
|---|---|---|
| [mg] | Substance | [wt.-%] |
| 20.00 | amphetamine sulfate | 8.88 |
| 61.19 | polyethylene oxide 7 mio. | 27.18 |
| 14.57 | polyethylene glycol 6000 | 6.47 |
| 0.24 | α-tocopherol | 0.11 |
| 24.00 | Starch 1500 | 10.66 |
| 6.60 | DR Coating Layer 1 = Opadry ® II pink | 2.93 |
| 38.11 | DR Coating Layer 2 = alginate | 16.93 |
| 60.45 | DR Coating Layer 3 = Eudragit ® L30-D55 + TEC | 26.85 |
| 225.16 | | 100.00 |

The DR Coating Layer 1 had the following composition:

| Substance | Amount [wt.-%] |
|---|---|
| Opadry ® II pink | 20.00 |
| water | 80.00 |
| | 100.00 |

The DR Coating Layer 2 had the following composition:

| Substance | Amount [wt.-%] |
|---|---|
| Protanal ® CR8133 (alginate) | 3.50 |
| talcum | 1.75 |
| water | 94.75 |
| | 100.00 |

The DR Coating Layer 3 had the following composition:

| Substance | Amount [wt.-%] |
|---|---|
| Eudragit ® L30-D55 | 40.79 |
| triethylcitrat | 2.00 |
| talcum | 6.12 |
| water | 51.09 |
| | 100.00 |

The average individual total weight of a single coated particle was below 2.0 mg.

### Example 14—Controlled Release Particle Providing Extended Release

In accordance with Example 4, 20 mg PR particles (cut rods) of a total weight amounting to 350 mg were manufactured. The PR particles had the following composition:

| | | Amount |
|---|---|---|
| per dosis [mg] | Substance | [wt.-%] |
| 20.00 | amphetamine sulfate | 5.71 |
| 237.70 | polyethylene oxide 7 mio. | 67.91 |
| 35.00 | hypromellose | 10.00 |
| 56.60 | polyethylene glycol 6000 | 16.17 |
| 0.70 | α-tocopherol | 0.20 |
| 350.00 | | 100.00 |

The breaking strength (resistance to crushing) of the particles was measured. In none of altogether ten measurements, the particles broke at a force of 1000 N.

### Example 15—Immediate Release Particles of Example 12 and Delayed Release Particles of Example 13

In accordance with Example 5, the IR particles of Example 12 were combined with the DR particles of Example 13 and filled into capsules of size 0. Thus, the capsules had the following overall composition:

| per capsule (Size 0) | | per capsule | | Amount |
|---|---|---|---|---|
| [mg] | form | [mg] | Substance | [wt.-%] |
| 129.90 | IR coated | 20.00 | amphetamine sulfate | 36.59 |
| | pellets | 61.19 | polyethylene oxide 7 | |
| | of Example 12 | | mio | |
| | | 14.57 | polyethylene glycol | |
| | | | 6000 | |
| | | 0.24 | α-tocopherol | |
| | | 24.00 | Starch 1500 | |
| | | 9.96 | Opadry ® II clear | |
| 225.16 | DR coated | 20.00 | amphetamine sulfate | 63.41 |
| | pellets | 61.19 | polyethylene oxide 7 | |
| | of Example 13 | | mio. | |
| | | 14.57 | polyethylene glycol | |
| | | | 6000 | |
| | | 0.24 | α-tocopherol | |
| | | 24.00 | Starch 1500 | |
| | | 6.60 | DR Coating Layer 1 = | |
| | | | Opadry ® pink | |
| | | 38.11 | DR Coating Layer 2 = | |
| | | | Alginate | |
| | | 60.45 | DR Coating Layer 3 = | |
| | | | Eudragit ® L30-D55 + | |
| | | | TEC | |
| 355.06 | | | | 100.00 |

In order to assess the tamper resistance of the thus obtained capsules, the capsules were manually opened and the content of the capsules was isolated. Subsequently, the following tampering attempts were conducted and the following results were achieved:

Extraction for the Purpose of Intravenous Administration:

| | content of capsule of Example 15 |
|---|---|
| | (after milling in coffee grinder) |
| 1 | 7.62 |
| 2 | 7.04 |
| 3 | 5.98 |
| mean [%] | 6.88 |
| SD [%] | 12.09 |

Extraction in Different Media (30 ml)

| | content of capsule of Example 15 |
|---|---|
| 30 mL water | |
| 1 | 49.17 |
| 2 | 48.60 |
| 3 | 50.69 |
| mean [%] | 49.49 |
| 30 mL boiling water | |
| 1 | 57.98 |
| 2 | 58.71 |
| 3 | 54.82 |
| mean [%] | 57.17 |
| 30 mL 40% EtOH | |
| 1 | 41.08 |
| 2 | 42.72 |
| 3 | 41.00 |
| mean [%] | 41.60 |

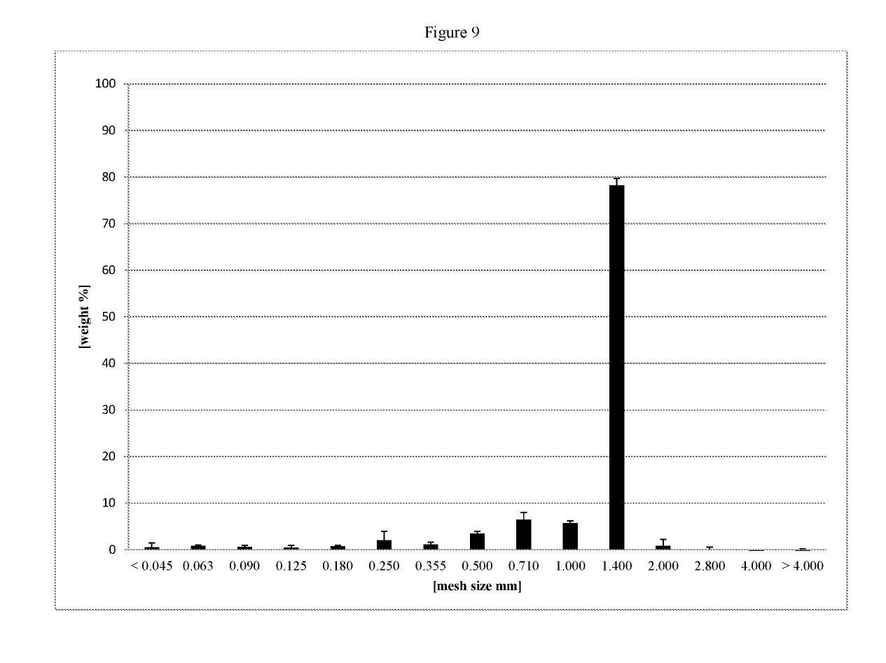

Sieve analysis: the content of the capsules was milled 2 minutes with a coffee grinder and the particle size distribution was determined by sieve analysis. The results are shown in FIG. 9.

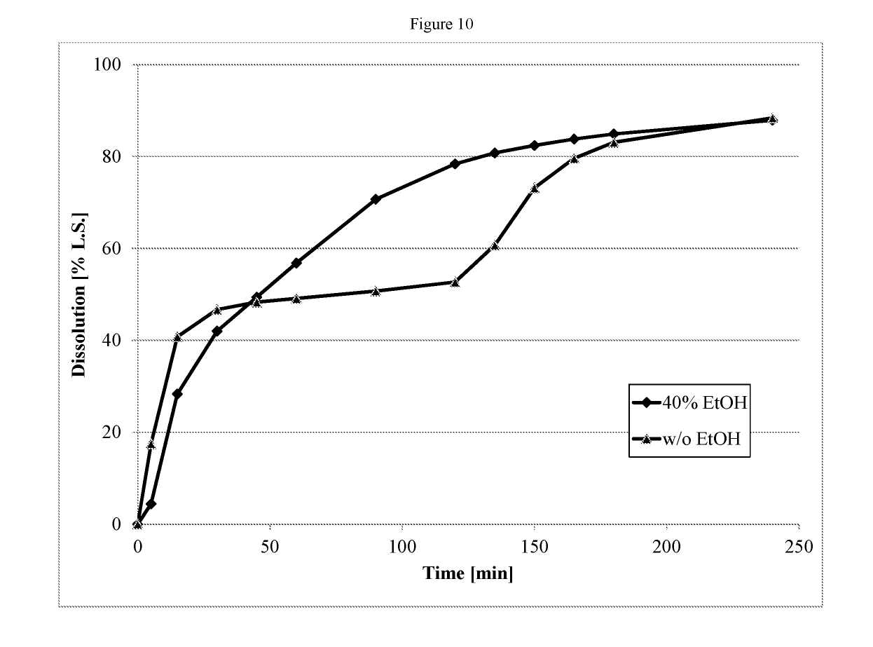

FIG. 10 shows the in vitro release profile without ethanol and with ethanol.

### Example 16—Immediate Release Particles of Example 12 and Controlled Release Particles of Example 14

The IR particles of Example 12 were combined with the PR particle of Example 14 and filled into capsules of size 0. Thus, the capsules had the following overall composition:

| per capsule | | per | | |
|---|---|---|---|---|
| (Size 0) | | capsule | | Amount |
| [mg] | form | [mg] | Substance | [wt.-%] |
| 129.90 | IR coated | 20.00 | amphetamine sulfate | 27.07 |
| | pellets of | 61.19 | polyethylene oxide 7 | |
| | Example 12 | | mio. | |
| | | 14.57 | polyethylene glycol | |
| | | | 6000 | |
| | | 0.24 | α-tocopherol | |
| | | 24.00 | Starch 1500 | |
| | | 9.96 | Opadry ® II clear | |
| 350.00 | PR particle of | 20.00 | amphetamine sulfate | 72.93 |
| | Example 14 | 237.70 | polyethylene oxide 7 | |
| | | | mio. | |
| | | 35.00 | hypromellose | |
| | | 56.60 | polyethylene glycol | |
| | | | 6000 | |
| | | 0.70 | α-tocopherol | |
| 479.90 | | | | 100.00 |

The breaking strength (resistance to crushing) of the cut rods was measured. In none of altogether ten measurements, the cut rods broke at a force of 1000 N.

In order to assess the tamper resistance of the thus obtained capsules, the capsules were manually opened and the content of the capsules was isolated. Subsequently, the following tampering attempts were conducted and the following results were achieved:

Extraction for the Purpose of Intravenous Administration:

| | content of capsule of Example 16 |
|---|---|
| | (after milling in coffee grinder) |
| 1 | —* |
| 2 | —* |
| 3 | —* |
| mean [%] | —* |
| SD [%] | —* |

*= could not be analyzed as to less material could be drawn into syringe

Extraction in Different Media (30 ml)

| | content of capsule of Example 16 |
|---|---|
| 30 mL water | |
| 1 | 56.94 |
| 2 | 55.51 |
| 3 | 56.83 |
| mean [%] | 56.43 |
| 30 mL boiling water | |
| 1 | 64.65 |
| 2 | 60.89 |
| 3 | 60.49 |
| mean [%] | 62.01 |
| 30 mL 40% EtOH | |
| 1 | 46.35 |
| 2 | 48.35 |
| 3 | 47.38 |
| mean [%] | 47.36 |

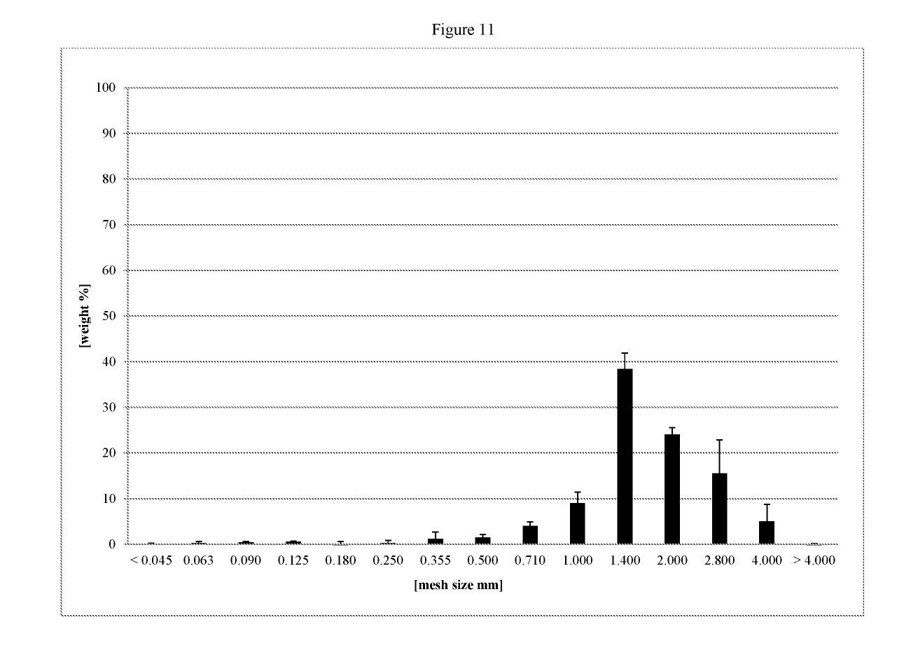

Sieve analysis: the content of the capsules was milled 2 minutes with a coffee grinder and the particle size distribution was determined by sieve analysis. The results are shown in FIG. 11.

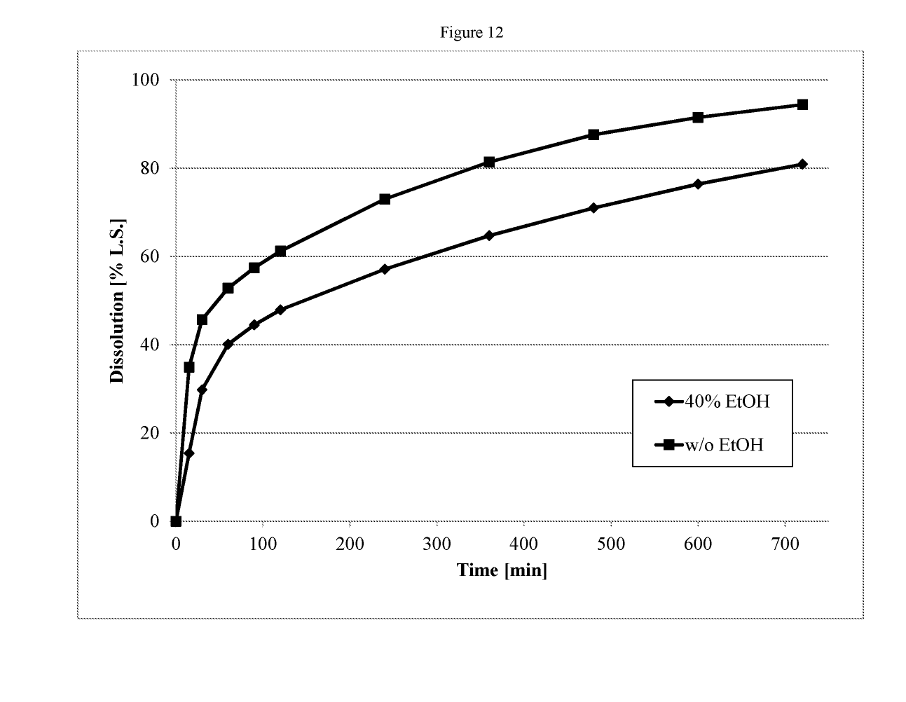

FIG. 12 shows the in vitro release profile without ethanol and with ethanol.

### Example 17 Sintering Process as an Alternative to Hot-Melt Extrusion

Based on composition 4-2, six 6*15 mm oblong tablets were prepared via a sintering process.

An increase in the volume of the tablets was observed after sintering.

The breaking strength (resistance to crushing) of the tablets was measured. None of the tablets broke at a force of 1000 N.

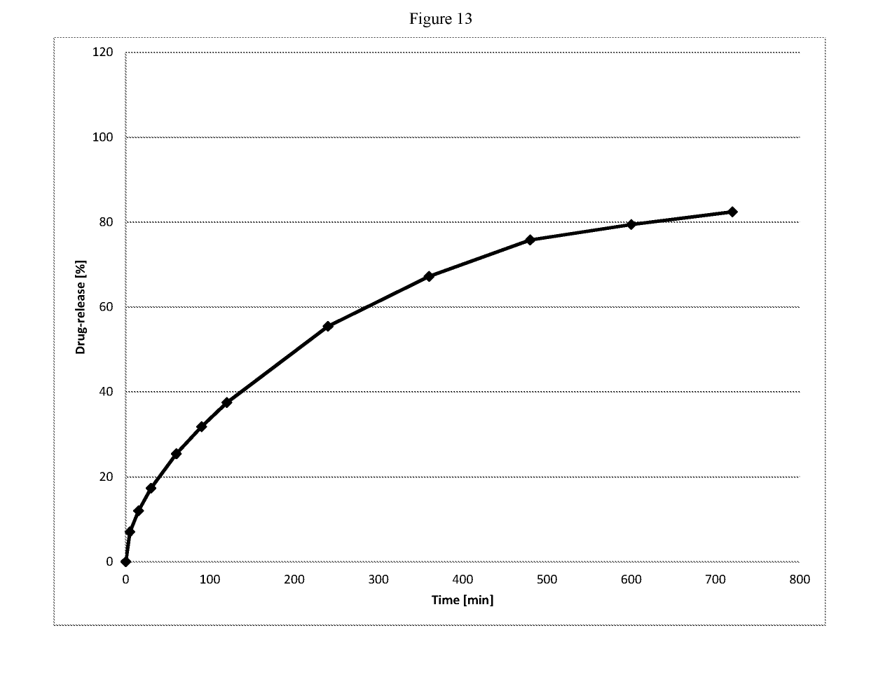

FIG. 13 shows the mean in vitro release profile of the tablets.

### Example 18—Immediate Release Particles Coated with Non-Enteric Coating which does not Delay In Vitro Dissolution

In accordance with Example 1, pellets providing immediate release of amphetamine sulfate were manufactured by hot-melt extrusion. The thus obtained extruded pellets were coated with a non-functional (non-enteric) protection coating which does not delay in vitro dissolution to avoid sticking of pellets.

The pellets (multitude of immediate release particles) contained 20 mg amphetamine sulfate. The IR particles had the following composition (see Example 1):

| per pellets | | amount |
|---|---|---|
| [mg] | substance | [wt.-%] |
| 10.00 | amphetamine sulfate | 15.38 |
| 33.52 | polyethylene oxide 7 mio. | 51.57 |
| 7.98 | polyethylene glycol 6000 | 12.28 |
| 0.12 | alpha tocopherol | 0.18 |
| 8.38 | starch 1500 | 12.89 |
| 5.00 | Opadry ® II clear | 7.69 |
| 65.00 | | 100.00 |

Opadry ® II clear: a non-enteric coating which does not delay in vitro dissolution.

Powder mixtures of the ingredients were manufactured and subsequently hot-melt extruded under the following extrusion conditions:

Leistritz extruder type

| | TSE18 | TSE27 |
|---|---|---|
| Speed screw [rpm] | 100 | 200 |
| Feed rate [g/min] | 16.66 | 250 |
| Melt pressure [bar] | 90-185 | 110-150 |
| melt temperature discharge [° C.] | 140-145 | 100-120 |

The thus extruded pellets were coated with a non-enteric coating which does not delay in vitro dissolution having the following composition:

| Substance | Amount [%] |
|---|---|
| Opadry ® II clear | 10.00 |
| water | 90.00 |
| | 100.00 |

The average individual total weight of a single particle was below 2.0 mg.

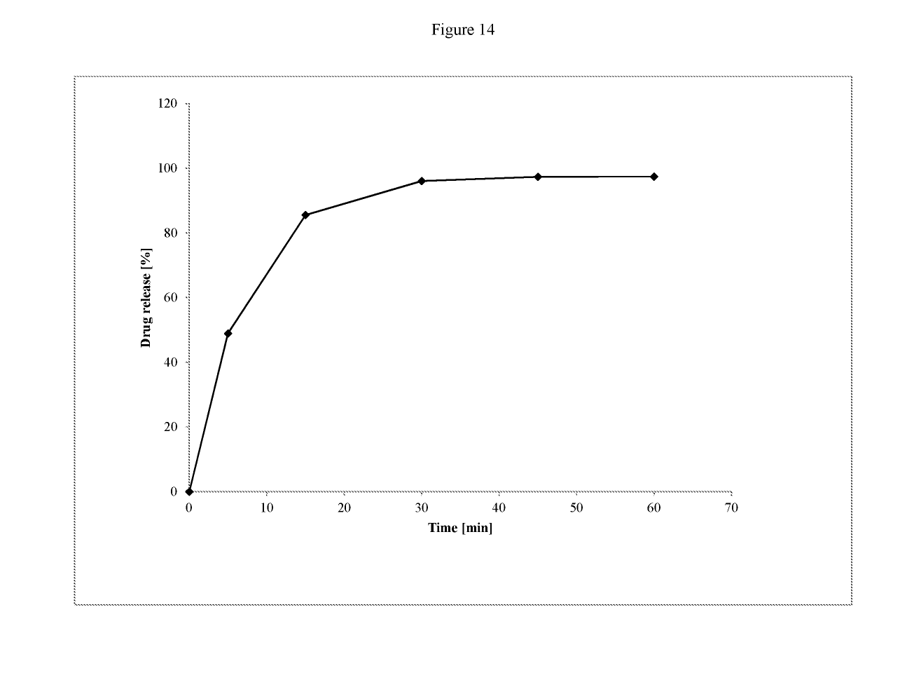

FIG. 14 shows the in vitro release profile of the 20 mg IR particles with non-functional coat.

### Example 19—Controlled Release Particles Comprising Specific Enteric Coating Providing Delayed Release

In accordance with Example 3, 20 mg DR particles were manufactured comprising a functional, i.e. enteric coating. A hot melt extruded pellet core was subsequently provided with two or three coating layers, namely optionally an inner layer based on Opadry® pink (DR Coating Layer 1), an intermediate layer based on alginate (DR Coating Layer 2, composition DR-1 or DR-2), and an outer layer based on Eudragit® L30-D55 (DR Coating Layer 3).

The DR coated pellets had the following composition:

| 19-1 | 19-2 | 19-3 | | | | |
|---|---|---|---|---|---|---|
| per | per | per | | 19-1 | 19-2 | 19-3 |
| dosis | dosis | dosis | | Amount | Amount | Amount |
| [mg] | [mg] | [mg] | Substance | [wt.-%] | [wt.-%] | [wt.-%] |
| 10.00 | 10.00 | 10.00 | amphetamine sulfate | 6.23 | 6.12 | 7.36 |
| 33.52 | 33.52 | 33.52 | polyethylene oxide 7 mio. | 20.87 | 20.51 | 24.67 |
| 7.98 | 7.98 | 7.98 | polyethylene glycol 6000 | 4.97 | 4.88 | 5.87 |
| 0.12 | 0.12 | 0.12 | α-tocopherol | 0.07 | 0.07 | 0.09 |
| 8.38 | 8.38 | 8.38 | Starch 1500 | 5.22 | 5.13 | 6.17 |
| 9.2 | 9.2 | — | DR Coating Layer 1 = Opadry ® | 5.73 | 5.63 | — |
| | | | II pink | | | |
| 30.30 | — | 23.30 | DR Coating Layer 2 = Alginate. | 18.87 | — | 17.14 |
| | | | DR-1 | | | |
| — | 31.30 | — | DR Coating Layer 2 = Alginate. | — | 19.16 | — |
| | | | DR-2 | | | |
| 61.10 | 62.90 | 52.60 | DR Coating Layer 3 = | 38.04 | 23.08 | 38.49 |
| | | | Eudragit ® L30-D55 + TEC | | | |
| 160.6 | 163.4 | 135.90 | | 100.00 | 100.00 | 100.00 |

The DR Coating Layer 1 had the following composition:

| Substance | Amount [wt.-%] |
|---|---|
| Opadry ® II pink | 20.00 |
| water | 80.00 |
| | 100.00 |

The DR Coating Layer 2 had the following composition:

| | DR-1 | DR-2 |
|---|---|---|
| Substance | Amount [wt.-%] | Amount [wt.-%] |
| Protanal ® CR8133 (Alginate) | 3.50 | 5.25 |
| talcum | 1.75 | 2.63 |
| water | 94.75 | 92.12 |
| | 100.00 | 100.00 |

The DR Coating Layer 3 had the following composition:

| Substance | Amount [wt.-%] |
|---|---|
| Eudragit ® L30-D55 | 40.79 |
| triethylcitrat | 2.00 |
| talcum | 6.12 |
| water | 51.09 |
| | 100.00 |

The average individual total weight of a single coated particle was below 2.0 mg.

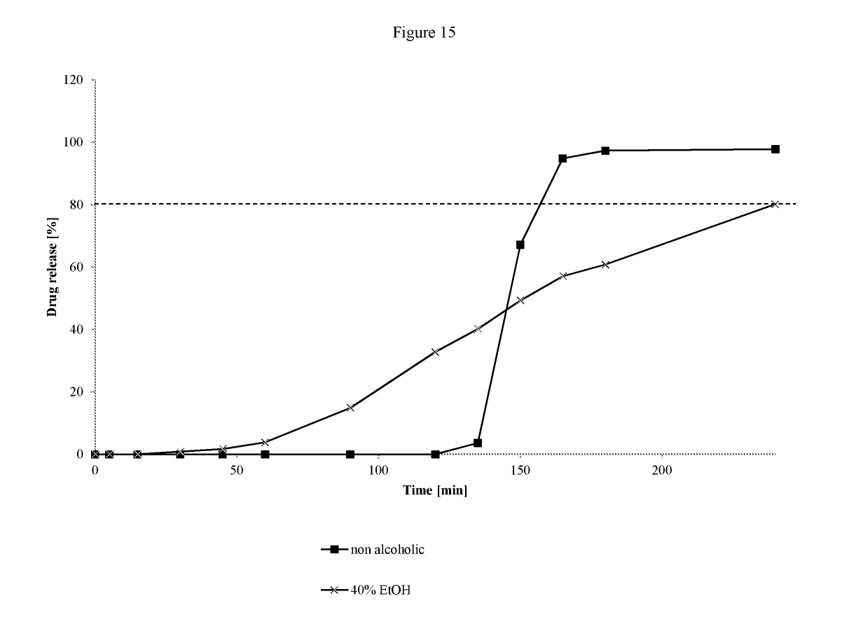

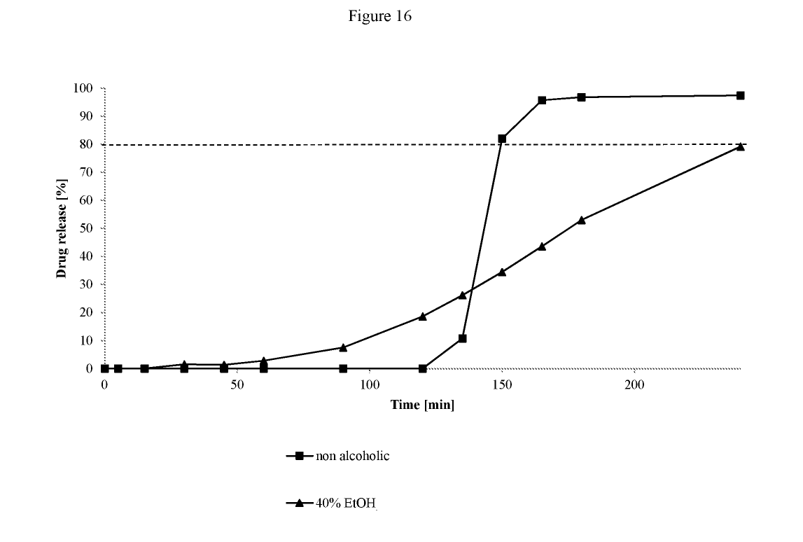

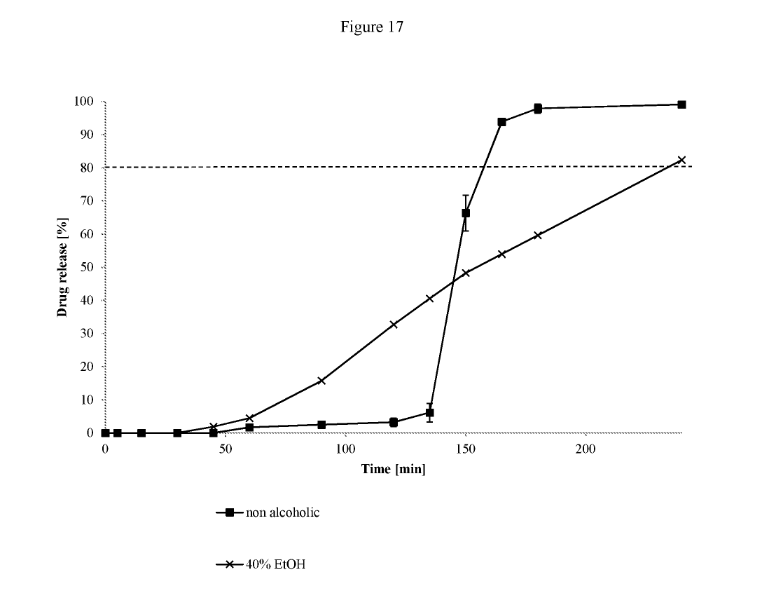

FIG. 15 shows the dissolution curves for the pellets of example 19-1, FIG. 16 shows the dissolution curves for the pellets of example 19-2, and FIG. 17 shows the dissolution curves for the pellets of example 19-3.

The compositions of the controlled release particles comprising specific enteric coating providing delayed release according to examples 2, 3, 13 and 19 are compared with one another in the following table:

| substance content [wt.-%] | 2 | 3 | 13 | 19-1 | 19-2 | 19-3 |
|---|---|---|---|---|---|---|
| amphetamine sulfate | 12.71 | 12.71 | 8.88 | 6.23 | 6.12 | 7.36 |
| polyethylene oxide 7 mio. | 38.88 | 38.88 | 27.18 | 20.87 | 20.51 | 24.67 |
| polyethylene glycol 6000 | 9.26 | 9.26 | 6.47 | 4.97 | 4.88 | 5.87 |
| alpha tocopherol | 0.15 | 0.15 | 0.11 | 0.07 | 0.07 | 0.09 |
| starch 1500 | 15.25 | 15.25 | 10.66 | 5.22 | 5.13 | 6.17 |
| Opadry ® II pink | — | — | 2.93 | 5.73 | 5.63 | — |
| Alginate, DR-1 (3.50 wt.-% alginate, 1.75 | — | — | 16.93 | 18.87 | — | 17.14 |
| wt.-% talcum) | | | | | | |
| Alginate, DR-2 (5.25 wt.-% alginate, 2.63 | — | — | — | — | 19.16 | — |
| wt.-% talcum) | | | | | | |
| Eudragit ® L30-D55 + 3.3% TEC | 23.76 | — | — | — | — | — |
| Evonik ADD | — | 23.76 | — | — | — | — |
| Eudragit ® L30-D55 + TEC (40.79 wt.-% | — | — | 26.85 | 38.04 | 38.49 | 38.70 |
| Eudragit ® L30-D55, 2.00 wt.-% | | | | | | |
| triethylcitrate, 6.12 wt.-% talcum) | | | | | | |

It becomes clear from a comparison of examples 2, 3, 13, and 19 that especially an increased weight of the layer that is based on acrylate copolymer (Eudragit®) further improves resistance against ethanolic dose dumping. Best results are achieved when the weight of the layer that is based on acrylate polymer is at least twice as high as the weight of the layer that is based on sodium alginate (or another salt of alginic acid).

The weight of the layer that is based on sodium alginate (or another salt of alginic acid) should preferably increase the weight of the core, which is optionally coated with a non-enteric coating (Opadry® II pink), by at least 20 wt.-%, preferably at least 30 wt.-%, relative to the weight of the core, which is optionally coated with a non-enteric coating. The weight of the layer that is based on acrylate polymer should preferably increase the weight of the core, which is coated with the layer that is based on sodium alginate (or another salt of alginic acid) and which is optionally coated with a non-enteric coating (Opadry® II pink), by at least 20 wt.-%, preferably at least 30 wt.-%, relative to the weight of the core, which is coated with the layer that is based on sodium alginate (or another salt of alginic acid) and which is optionally coated with a non-enteric coating.

Relative to the total weight of the fully coated particles, the weight content of the layer that is based on sodium alginate (or another salt of alginic acid) should be preferably be at least 13 wt.-%, more preferably at least 15 wt.-%, still more preferably at least 17 wt.-%; and the weight content of the layer that is based on acrylate polymer should preferably be at least 19 wt.-%, more preferably at least 21 wt.-%, and still more preferably at least 23 wt.-%.

### Example 20—Immediate Release Particles and Delayed Release Particles

In accordance with the above examples, capsules were filed with the following amounts (in mg) of IR particles coated with a non-enteric coating (Opadry® II clear) which does not delay in vitro dissolution and of DR particles coating with an enteric coating:

| | 2.5 mg/ | 5.0 mg/ | 7.5 mg/ | 10 mg/ | 15 mg/ | 20 mg/ |
|---|---|---|---|---|---|---|
| [mg] | 2.5 mg | 5.0 mg | 7.5 mg | 10 mg | 15 mg | 20 mg |
| coated IR | | | | | | |
| particles: | | | | | | |
| amphetamine | 2.50 | 5.00 | 7.50 | 10.00 | 15.00 | 20.00 |
| sulfate | | | | | | |
| PEO | 69.22 | 67.75 | 66.27 | 33.38 | 50.07 | 66.76 |
| starch 1500 | 19.51 | 19.09 | 18.68 | 9.41 | 14.11 | 18.81 |
| PEG* | 28.52 | 27.91 | 27.30 | 13.75 | 20.63 | 27.51 |
| vitamin-E* | 0.26 | 0.25 | 0.25 | 0.12 | 0.19 | 0.25 |
| Opadry ® II | 8.40 | 9.20 | 11.30 | 5.00 | 7.50 | 10.00 |
| clear | | | | | | |
| Sum [mg] | 128.40 | 129.20 | 131.30 | 71.66 | 107.50 | 143.33 |
| coated DR | | | | | | |
| particles: | | | | | | |
| amphetamine | 2.50 | 5.00 | 7.50 | 10.00 | 15.00 | 20.00 |
| sulfate | | | | | | |
| PEO | 69.22 | 67.75 | 66.27 | 33.38 | 50.07 | 66.76 |
| starch 1500 | 19.51 | 19.09 | 18.68 | 9.41 | 14.11 | 18.81 |
| PEG* | 28.52 | 27.91 | 27.30 | 13.75 | 20.63 | 27.51 |
| vitamin-E* | 0.26 | 0.25 | 0.25 | 0.12 | 0.19 | 0.25 |
| Opadry pink | 17.22 | 8.00 | 8.50 | 8.82 | 4.88 | 6.51 |
| Protanal ® | 40.14 | 41.16 | 27.12 | 21.73 | 19.54 | 26.05 |
| talcum | 20.11 | 20.62 | 13.58 | 10.89 | 9.79 | 13.05 |
| Eudragit ® | 89.16 | 127.52 | 52.04 | 47.96 | 37.72 | 50.29 |
| L30 D55 | | | | | | |
| triethylcitrate | 4.37 | 6.25 | 2.55 | 2.35 | 1.85 | 2.47 |
| talcum | 13.38 | 19.13 | 7.81 | 7.20 | 5.66 | 7.55 |
| Sum [mg] | 304.38 | 342.69 | 231.60 | 165.61 | 179.44 | 239.25 |

The relative weight content (in wt.-%) of all constituents is compiled in the table here below:

| | 2.5 mg/ | 5.0 mg/ | 7.5 mg/ | 10 mg/ | 15 mg/ | 20 mg/ |
|---|---|---|---|---|---|---|
| [wt.-%] | 2.5 mg | 5.0 mg | 7.5 mg | 10 mg | 15 mg | 20 mg |
| coated IR | | | | | | |
| particles: | | | | | | |
| amphetamine | 1.95 | 3.87 | 5.71 | 13.95 | 13.95 | 13.95 |
| sulfate | | | | | | |
| PEO | 53.91 | 52.44 | 50.48 | 46.58 | 46.58 | 46.58 |
| Starch 1500 | 15.19 | 14.78 | 14.22 | 13.13 | 13.13 | 13.13 |
| PEG* | 22.21 | 21.60 | 20.79 | 19.19 | 19.19 | 19.19 |
| vitamin-E* | 0.20 | 0.20 | 0.19 | 0.17 | 0.17 | 0.17 |
| Opadry ® II | 6.54 | 7.12 | 8.61 | 6.98 | 6.98 | 6.98 |
| clear | | | | | | |
| Sum [%] | 100.00 | 100.00 | 100.00 | 100.00 | 100.00 | 100.00 |
| coated DR | | | | | | |
| particles: | | | | | | |
| amphetamine | 0.82 | 1.46 | 3.24 | 6.04 | 8.36 | 8.36 |
| sulfate | | | | | | |
| PEO | 22.74 | 19.77 | 28.62 | 20.15 | 27.91 | 27.90 |
| Starch 1500 | 6.41 | 5.57 | 8.06 | 5.68 | 7.86 | 7.86 |
| PEG* | 9.37 | 8.14 | 11.79 | 8.30 | 11.50 | 11.50 |
| vitamin-E* | 0.08 | 0.07 | 0.11 | 0.08 | 0.10 | 0.10 |
| Opadry ® pink | 5.66 | 2.33 | 3.67 | 5.33 | 2.72 | 2.72 |
| Protanal | 13.19 | 12.01 | 11.71 | 13.12 | 10.89 | 10.89 |
| talcum | 6.61 | 6.02 | 5.87 | 6.57 | 5.46 | 5.46 |
| Eudragit ® | 29.29 | 37.21 | 22.47 | 28.96 | 21.02 | 21.02 |
| L30 D55 | | | | | | |
| triethylcitrate | 1.44 | 1.82 | 1.10 | 1.42 | 1.03 | 1.03 |
| talcum | 4.39 | 5.58 | 3.37 | 4.35 | 3.15 | 3.15 |
| Sum [%] | 100.00 | 100.00 | 100.00 | 100.00 | 100.00 | 100.00 |

In the above table 2.5 mg/2.5 mg means that the IR particles were employed in an amount such that the capsule contained a dose of 2.5 mg amphetamine sulfate in the total quantity of all IR particles and that the DR particles were employed in an amount such that the capsule contained a dose of 2.5 mg amphetamine sulfate in the total quantity of all DR particles as well.

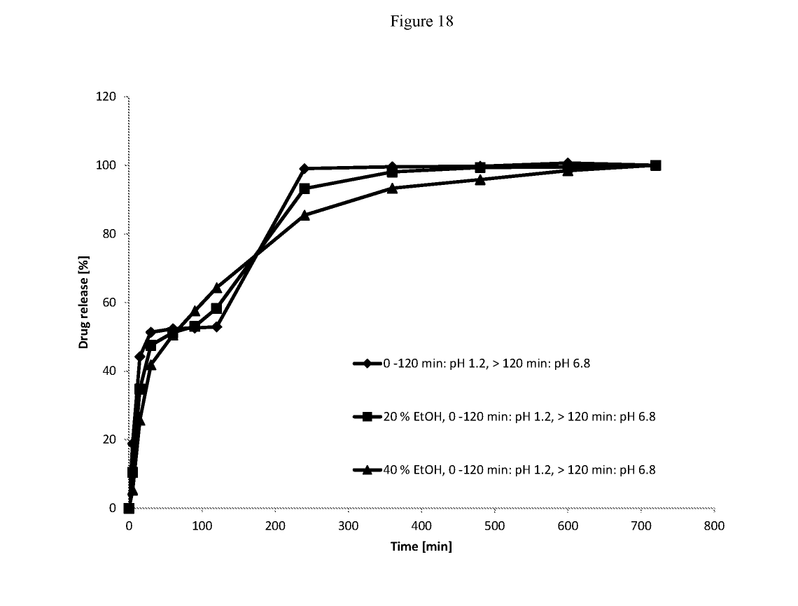

The in vitro dissolution of example 20 mg/20 mg was tested in different dissolution media (non-alcoholic, 20 vol.-% ethanol and 40 vol.-%, in either case pH switch after 120 min from pH 1.2 to pH 6.8). The results are displayed in FIG. 18.

It becomes clear from FIG. 18 that it takes a longer period of time in ethanolic medium than in non-alcoholic medium until 50 wt.-% of the pharmacologically active compound of the capsule filling (IR particles) have been released. Likewise, in ethanolic medium 100 wt.-% release of the pharmacologically active compound are achieved later than in non-alcoholic medium.

### Example 21—Immediate Release Particles Coated with Non-Enteric Coating which does not Delay In Vitro Dissolution

In accordance with Example 1, pellets providing immediate release of amphetamine sulfate were manufactured by hot-melt extrusion. The thus obtained extruded pellets were coated with a non-functional (non-enteric) protection coating which does not delay in vitro dissolution to avoid sticking of pellets.

The pellets (multitude of immediate release particles) contained 10 mg amphetamine sulfate. The IR particles had the following composition (see Example 1):

| per pellets | | amount |
|---|---|---|
| [mg] | substance | [wt.-%] |
| 10.00 | amphetamine sulfate | 13.98 |
| 33.39 | polyethylene oxide 7 mio. | 46.70 |
| 13.75 | polyethylene glycol 6000 | 19.23 |
| 0.12 | alpha tocopherol | 0.17 |
| 9.41 | starch 1500 | 13.16 |
| 4.83 | Opadry ® II clear 85F | 6.76 |
| 71.5 | sum | 100.00 |

Opadry ® II clear: a non-enteric coating which does not delay in vitro dissolution.

### Example 22—Immediate Release Particles Comprising Non-Enteric Coating Providing Fast Release (Fast Release Particles)

In accordance with Example 1, pellets providing immediate release of amphetamine sulfate were manufactured by hot-melt extrusion. The thus obtained extruded pellets were coated with a non-enteric coating which slightly retards in vitro dissolution thereby providing fast release particles.

The pellets (multitude of fast release particles) contained 10 mg amphetamine sulfate. The FR particles had the following composition:

| per pellets | | amount |
|---|---|---|
| [mg] | substance | [wt.-%] |
| 10.00 | amphetamine sulfate | 13.21 |
| 33.39 | polyethylene oxide 7 mio. | 44.11 |
| 13.75 | polyethylene glycol 6000 | 18.17 |
| 0.12 | alpha tocopherol | 0.16 |
| 9.41 | starch 1500 | 12.43 |
| 9.02 | FR Coating | 11.92 |
| (7.52) | Eudragit ® L30-D55 | (9.94) |
| (0.37) | triethyl citrate | (0.49) |
| (1.13) | talcum | (1.49) |
| 75.69 | sum | 100.00 |

Values in parentheses refer to the constituents of the coating

The average individual total weight of a single particle was below 2.0 mg.

### Example 23—Immediate Release Particles Comprising Non-Enteric Coating Providing Fast Release (Fast Release Particles)

In accordance with Example 1, pellets providing immediate release of amphetamine sulfate were manufactured by hot-melt extrusion. The thus obtained extruded pellets were subsequently provided with two coating layers, namely an inner layer based on Opadry® (FR Coating Layer 1) and an outer layer based on Eudragit® L30-D55 (FR Coating Layer 2), which slightly retards in vitro dissolution thereby providing fast release particles.

The pellets (multitude of fast release particles) contained 10 mg amphetamine sulfate. The FR particles had the following composition:

| per pellets | | amount |
|---|---|---|
| [mg] | substance | [wt.-%] |
| 10.00 | amphetamine sulfate | 12.13 |
| 33.39 | polyethylene oxide 7 mio. | 40.50 |
| 13.75 | polyethylene glycol 6000 | 16.68 |
| 0.12 | alpha tocopherol | 0.15 |
| 9.41 | starch 1500 | 11.41 |
| 5.41 | FR Coating Layer 1 | 6.56 |
| (5.41) | Opadry | (6.56) |
| 10.36 | FR Coating Layer 2 | 12.57 |
| (8.64) | Eudragit L30-D55 | (10.48) |
| (0.42) | triethyl citrate | (0.51) |
| (1.30) | talcum | (1.58) |
| 82.44 | sum | 100.00 |

Values in parentheses refer to the constituents of the coatings

Values in parentheses refer to the constituents of the coatings

The average individual total weight of a single particle was below 2.0 mg.

### Example 24—Controlled Release Particles Comprising Specific Enteric Coating Providing Delayed Release

In accordance with Example 3, 10 mg DR particles were manufactured comprising a functional, i.e. enteric coating. A hot melt extruded pellet core was subsequently provided with two or three coating layers, namely optionally an inner layer based on Opadry® pink (DR Coating Layer 1), an intermediate layer based on alginate (DR Coating Layer 2), and an outer layer based on Eudragit® L30-D55 (DR Coating Layer 3).

The DR coated pellets had the following composition:

| per pellets | | Amount |
|---|---|---|
| [mg] | Substance | [wt.-%] |
| 10.00 | amphetamine sulfate | 6.23 |
| 33.52 | polyethylene oxide 7 mio. | 20.87 |
| 7.98 | polyethylene glycol 6000 | 4.97 |
| 0.12 | α-tocopherol | 0.07 |
| 8.38 | Starch 1500 | 5.22 |
| 9.20 | DR Coating Layer 1 | 5.73 |
| (9.20) | Opadry ® II pink | (5.73) |
| 30.30 | DR Coating Layer 2 | 18.87 |
| (20.19) | alginate Protanal ® CR 8133 (FMC) | (12.57) |
| (10.11) | talcum | (6.30) |
| 61.10 | DR Coating Layer 3 | 38.04 |
| (50.96) | Eudragit L30-D55 | (31.73) |
| (2.50) | triethyl citrate | (1.56) |
| (7.64) | talcum | (4.76) |
| 160.60 | sum | 100.00 |

Values in parentheses refer to the constituents of the coatings

Values in parentheses refer to the constituents of the coatings

### Example 25—Controlled Release Particles Comprising Specific Enteric Coating Providing Postponed Release

In accordance with Example 3, 10 mg postponed release pellets (OR particles) were manufactured comprising a specific functional, i.e. enteric coating. A hot melt extruded pellet core was subsequently provided with three coating layers, namely an inner layer based on Opadry® pink (OR Coating Layer 1), an intermediate layer based on alginate (OR Coating Layer 2), and an outer layer based on a combination of 90% Eudragit® FS and 10% Eudragit® L30-D55 (OR Coating Layer 3).

The OR coated pellets had the following composition:

| per pellets | | Amount |
|---|---|---|
| [mg] | Substance | [wt.-%] |
| 10.00 | amphetamine sulfate | 5.76 |
| 33.39 | polyethylene oxide 7 mio. | 19.25 |
| 13.75 | polyethylene glycol 6000 | 7.93 |
| 0.12 | α-tocopherol | 0.07 |
| 9.41 | Starch 1500 | 5.42 |
| 8.97 | OR Coating Layer 1 | 5.17 |
| (8.97) | Opadry ® II pink | (5.17) |
| 33.50 | OR Coating Layer 2 | 19.31 |
| (21.54) | alginate Protanal ® CR 8133 (FMC) | (12.42) |
| (11.96) | talcum | (6.89) |
| 64.35 | OR Coating Layer 3 | 37.09 |
| (38.53) | 90% Eudragit FS + 10% Eudragit L30-D55 | (22.20) |
| (6.56) | triethyl citrate | (3.78) |
| (19.27) | talcum | (11.11) |
| 173.49 | sum | 100.00 |

Values in parentheses refer to the constituents of the coatings

Values in parentheses refer to the constituents of the coatings

The average individual total weight of a single coated particle was below 2.0 mg.

### Example 26—Immediate Release Particles of Example 21 and Postponed Release Particles of Example 25

The IR particles of Example 21 were combined with the OR particle of Example 24 and filled into capsules of size 0. Thus, the capsules had the following overall composition:

| per | | per | | | | |
|---|---|---|---|---|---|---|
| capsule | | form | | | content | content |
| [mg] | form | [mg] | Ingredient (according to Ph. Eur.) | | [wt.-%] | [wt.-%] |
| 71.50 | IR particles | 10.00 | amphetamine sulfate | | 29.18 | 4.08 |
| | according to | 33.39 | polyethylene oxide 7 Mio. | | | 13.63 |
| | Example 21 | 13.75 | Macrogol 6000 | | | 5.61 |
| | | 0.12 | alpha-tocopherol | | | 0.05 |
| | | 9.41 | Starch 1500 | | | 3.84 |
| | | 4.83 | Opadry II clear 85F | | | 1.97 |
| 173.49 | OR particles | 10.00 | amphetamine sulfate | | 70.82 | 4.08 |
| | according to | 33.39 | polyethylene oxide 7 Mio. | | | 13.63 |
| | Example 25 | 13.75 | Macrogol 6000 | | | 5.61 |
| | | 0.12 | alpha-tocopherol | | | 0.05 |
| | | 9.41 | Starch 1500 | | | 3.84 |
| | | 8.97 | OR coating layer 1 = Opadry pink | | | 3.66 |
| | | 33.50 | 21.54 | OR coating layer 2 = alginate | | 8.79 |
| | | | | Protanal CR 8133 (FMC) | | |
| | | | 11.96 | OR coating layer 2 = talcum | | 4.88 |
| | | 64.35 | 38.53 | OR coating layer 3 = 90% | | 15.73 |
| | | | | Eudragit FS + 10% Eudragit L30- | | |
| | | | | D55 | | |
| | | | 6.56 | OR coating layer 3 = triethyl | | 2.68 |
| | | | | citrate | | |
| | | | 19.27 | OR coating layer 3 = talcum | | 7.87 |
| 244.99 | | | | | 100.00 | 100.00 |

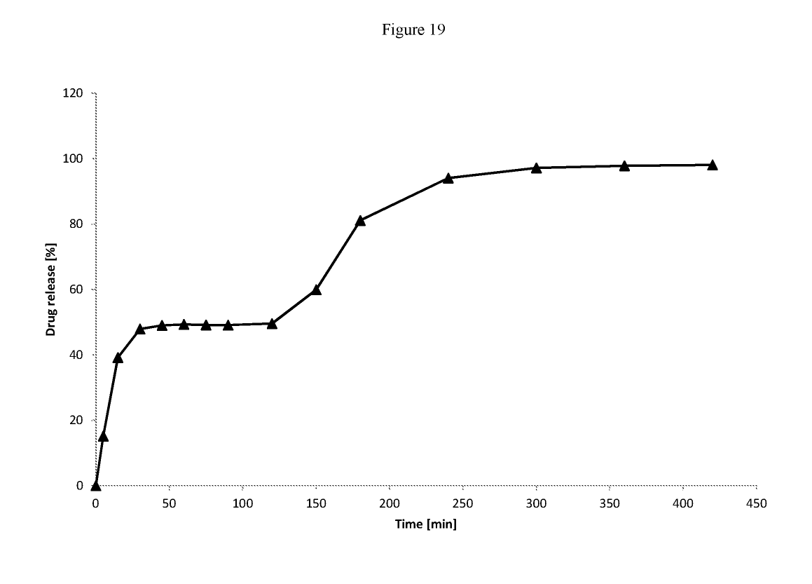

FIG. 19 shows the in vitro dissolution in non-ethanolic medium with a pH switch from pH 1 to pH 6.8 after 60 minutes.

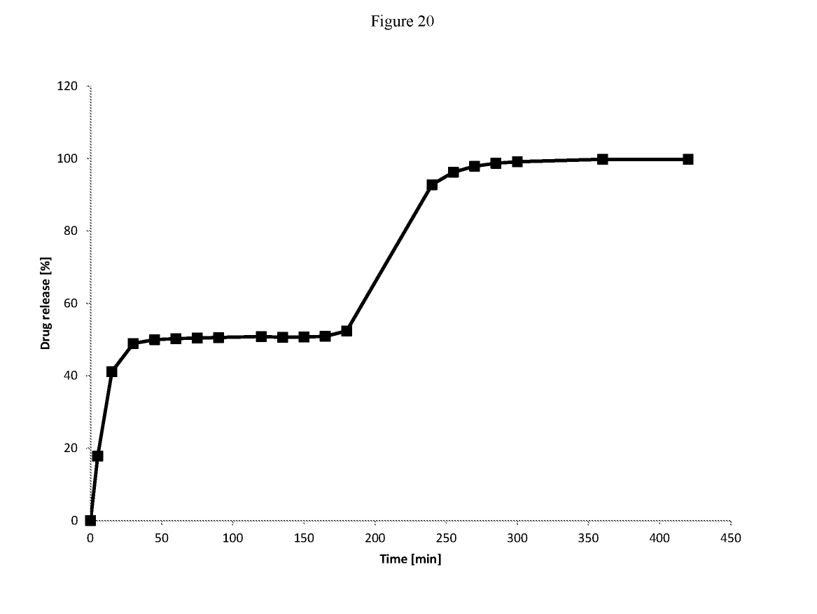

FIG. 20 shows the in vitro dissolution in non-ethanolic medium with a pH switch from pH 1 to pH 6.8 after 120 minutes.

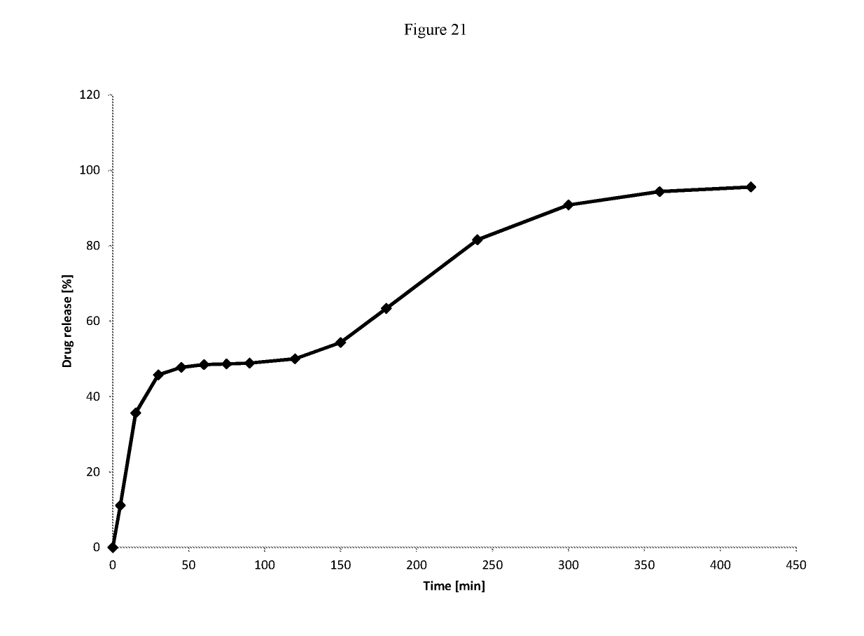

FIG. 21 shows the in vitro dissolution in non-ethanolic medium at pH 6.8.

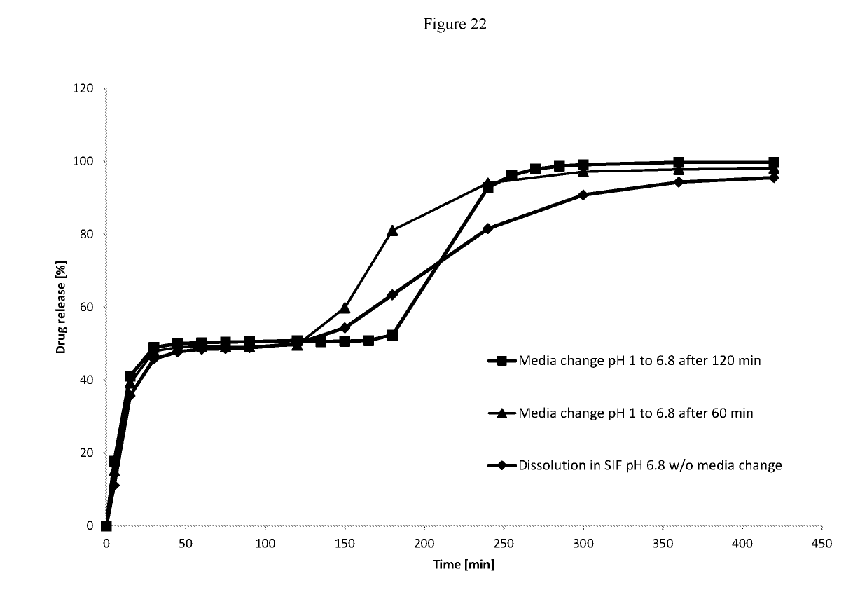

FIG. 22 shows an overlay plot of FIGS. 19, 20 and 21.

### Example 27—Fast Release Particles of Example 23 and Delayed Release Particles of Example 24

The FR particles of Example 23 were combined with the DR particles of Example 24 and filled into capsules of size 0. Thus, the capsules had the following overall composition:

| per | | per | | | | |
|---|---|---|---|---|---|---|
| capsule | | form | | | content | content |
| [mg] | form | [mg] | Ingredient (according to Ph. Eur.) | | [wt.-%] | [wt.-%] |
| 82.44 | FR particles | 10.00 | amphetamine sulfate | | 33.92 | 4.11 |
| | according to | 33.39 | polyethylene oxide 7 Mio. | | | 13.74 |
| | Example 23 | 13.75 | Macrogol 6000 | | | 5.66 |
| | | 0.12 | alpha-tocopherol | | | 0.05 |
| | | 9.41 | Starch 1500 | | | 3.87 |
| | | 5.41 | FR Coating Layer 1 - Opadry | | | 2.23 |
| | | 10.36 | 8.64 | FR Coating Layer 2 - Eudragit | | 3.55 |
| | | | | L30-D55 | | |
| | | | 0.42 | FR Coating Layer 2 -triethyl | | 0.17 |
| | | | | citrate | | |
| | | | 1.30 | FR Coating Layer 2 - talcum | | 0.53 |
| 160.60 | DR particles | 10.00 | amphetamine sulfate | | 66.08 | 4.11 |
| | according to | 33.52 | polyethylene oxide 7 Mio. | | | 13.79 |
| | Example 24 | 7.98 | Macrogol 6000 | | | 3.28 |
| | | 0.12 | alpha-tocopherol | | | 0.05 |
| | | 8.38 | Starch 1500 | | | 3.45 |
| | | 9.20 | DR Coating Layer 1 = Opadry pink | | | 3.79 |
| | | 30.30 | 20.19 | DR Coating Layer 2 = alginate | | 8.31 |
| | | | | Protanal CR 8133 (FMC) | | |
| | | | 10.11 | DR Coating Layer 2 = talcum | | 4.16 |
| | | 61.10 | 50.96 | DR Coating Layer 3 = Eudragit | | 20.97 |
| | | | | L30-D55 | | |
| | | | 2.50 | DR Coating Layer 3 = triethyl | | 1.03 |
| | | | | citrate | | |
| | | | 7.64 | DR Coating Layer 3 = talcum | | 3.14 |
| 243.04 | | | | | 100.00 | 100.00 |

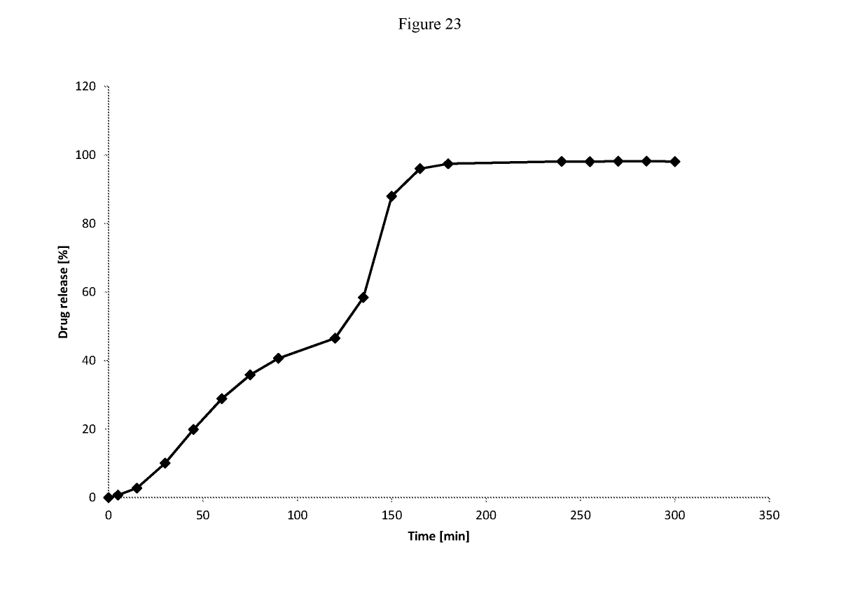

FIG. 23 shows the in vitro dissolution in non-ethanolic medium with a pH switch from pH 1 to pH 6.8 after 120 minutes.

### Example 28—Fast Release Particles According to Example 22 and Delayed Release Particles According to Example 24

The FR particles of Example 22 were combined with the DR particles of Example 24 and filled into capsules of size 0. Thus, the capsules had the following overall composition:

| per | | per | | | | |
|---|---|---|---|---|---|---|
| capsule | | form | | | content | content |
| [mg] | form | [mg] | Ingredient (according to Ph. Eur.) | | [wt.-%] | [wt.-%] |
| 75.69 | FR particles | 10.00 | amphetamine sulfate | | 32.03 | 4.23 |
| | according to | 33.39 | polyethylene oxide 7 Mio. | | | 14.13 |
| | Example 22 | 13.75 | Macrogol 6000 | | | 5.82 |
| | | 0.12 | alpha-tocopherol | | | 0.05 |
| | | 9.41 | Starch 1500 | | | 3.98 |
| | | 9.02 | 7.52 | FR Coating - Eudragit L30-D55 | | 3.18 |
| | | | 0.37 | FR Coating -triethyl citrate | | 0.16 |
| | | | 1.13 | FR Coating - talcum | | 0.48 |
| 160.60 | DR particles | 10.00 | amphetamine sulfate | | 67.97 | 4.23 |
| | according to | 33.52 | polyethylene oxide 7 Mio. | | | 14.19 |
| | Example 24 | 7.98 | Macrogol 6000 | | | 3.38 |
| | | 0.12 | alpha-tocopherol | | | 0.05 |
| | | 8.38 | Starch 1500 | | | 3.55 |
| | | 9.20 | DR Coating Layer 1 = Opadry pink | | | 3.89 |
| | | 30.30 | 20.19 | DR Coating Layer 2 = alginate | | 8.54 |
| | | | | Protanal CR 8133 (FMC) | | |
| | | | 10.11 | DR Coating Layer 2 = talcum | | 4.28 |
| | | 61.10 | 50.96 | DR Coating Layer 3 = Eudragit L30- | | 21.57 |
| | | | | D55 | | |
| | | | 2.50 | DR Coating Layer 3 = triethyl citrate | | 1.06 |
| | | | 7.64 | DR Coating Layer 3 = talcum | | 3.23 |
| 236.29 | | | | | 100.00 | 100.00 |

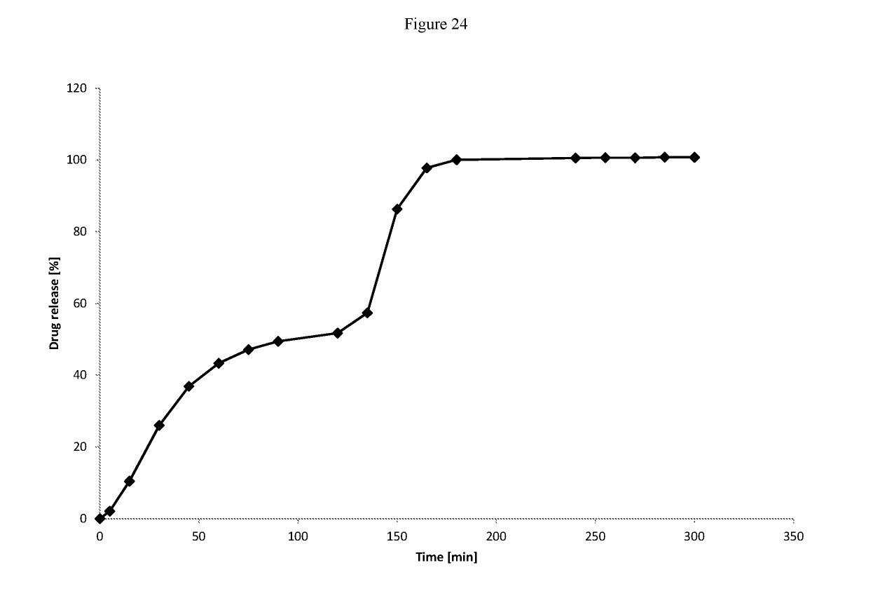

FIG. 24 shows the in vitro dissolution in non-ethanolic medium with a pH switch from pH 1 to pH 6.8 after 120 minutes.

### Example 29—Fast Release Particles According to Example 23 and Postponed Release Particles According to Example 25

The FR particles of Example 23 were combined with the OR particles of Example 25 and filled into capsules of size 0. Thus, the capsules had the following overall composition:

| per | | per | | | | |
|---|---|---|---|---|---|---|
| capsule | | form | | | content | content |
| [mg] | form | [mg] | Ingredient (according to Ph. Eur.) | | [wt.-%] | [wt.-%] |
| 82.44 | FR particles | 10.00 | amphetamine sulfate | | 32.21 | 3.91 |
| | according to | 33.39 | polyethylene oxide 7 Mio. | | | 13.05 |
| | Example 23 | 13.75 | Macrogol 6000 | | | 5.37 |
| | | 0.12 | alpha-tocopherol | | | 0.05 |
| | | 9.41 | Starch 1500 | | | 3.68 |
| | | 5.41 | FR Coating Layer 1 - Opadry | | | 2.11 |
| | | 10.36 | 8.64 | FR Coating Layer 2 - Eudragit L30- | | 3.38 |
| | | | | D55 | | |
| | | | 0.42 | FR Coating Layer 2 -triethyl citrate | | 0.16 |
| | | | 1.30 | FR Coating Layer 2 - talcum | | 0.51 |
| 173.49 | OR particles | 10.00 | amphetamine sulfate | | 67.79 | 3.91 |
| | according to | 33.39 | polyethylene oxide 7 Mio. | | | 13.05 |
| | Example 25 | 13.75 | Macrogol 6000 | | | 5.37 |
| | | 0.12 | alpha-tocopherol | | | 0.05 |
| | | 9.41 | Starch 1500 | | | 3.68 |
| | | 8.97 | OR Coating Layer 1 = Opadry pink | | | 3.50 |
| | | 33.50 | 21.54 | OR Coating Layer 2 = alginate | | 8.42 |
| | | | | Protanal CR 8133 (PMC) | | |
| | | | 11.96 | OR Coating Layer 2 = talcum | | 4.67 |
| | | 64.35 | 38.53 | OR Coating Layer 3 = 90 Eudragit | | 15.05 |
| | | | | FS + 10 Eudragit L30-D55 | | |
| | | | 6.56 | OR Coating Layer 3 = triethyl | | 2.56 |
| | | | | citrate | | |
| | | | 19.27 | OR Coating Layer 3 = talcum | | 7.53 |
| 255.93 | | | | | 100.00 | 100.00 |

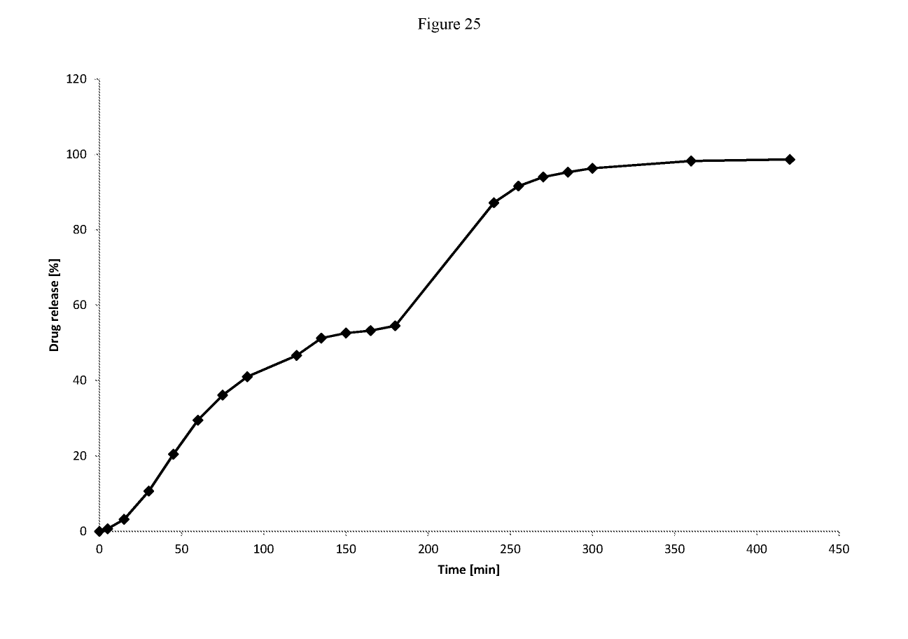

FIG. 25 shows the in vitro dissolution in non-ethanolic medium with a pH switch from pH 1 to pH 6.8 after 120 minutes.

### Example 30—Fast Release Particles According to Example 22 and Postponed Release Particles According to Example 25

The FR particles of Example 22 were combined with the OR particles of Example 25 and filled into capsules of size 0. Thus, the capsules had the following overall composition:

| per | | per | | | | |
|---|---|---|---|---|---|---|
| capsule | | form | | | content | content |
| [mg] | form | [mg] | Ingredient (according to Ph. Eur.) | | [wt.-%] | [wt.-%] |
| 75.69 | PR particles | 10.00 | amphetamine sulfate | | 30.38 | 4.01 |
| | according to | 33.39 | polyethylene oxide 7 Mio. | | | 13.40 |
| | Example 22 | 13.75 | Macrogol 6000 | | | 5.52 |
| | | 0.12 | alpha-tocopherol | | | 0.05 |
| | | 9.41 | Starch 1500 | | | 3.78 |
| | | 9.02 | 7.52 | FR Coating - Eudragit L30- | | 3.02 |
| | | | | D55 | | |
| | | | 0.37 | FR Coating -triethyl citrate | | 0.15 |
| | | | 1.13 | FR Coating - talcum | | 0.45 |
| 173.49 | OR particles | 10.00 | amphetamine sulfate | | 69.62 | 4.01 |
| | according to | 33.39 | polyethylene oxide 7 Mio. | | | 13.40 |
| | Example 25 | 13.75 | Macrogol 6000 | | | 5.52 |
| | | 0.12 | alpha-tocopherol | | | 0.05 |
| | | 9.41 | Starch 1500 | | | 3.78 |
| | | 8.97 | OR Coating Layer 1 = Opadry pink | | | 3.60 |
| | | 33.50 | 21.54 | OR Coating Layer 2 = | | 8.64 |
| | | | | alginate Protanal CR 8133 | | |
| | | | | (FMC) | | |
| | | | 11.96 | OR Coating Layer 2 = | | 4.80 |
| | | | | talcum | | |
| | | 64.35 | 38.53 | OR Coating Layer 3 = 90 | | 15.46 |
| | | | | Eudragit FS + 10 Eudragit | | |
| | | | | L30-D55 | | |
| | | | 6.56 | OR Coating Layer 3 = | | 2.63 |
| | | | | triethyl citrate | | |
| | | | 19.27 | OR Coating Layer 3 = | | 7.73 |
| | | | | talcum | | |
| 249.18 | | | | | 100.00 | 100.00 |

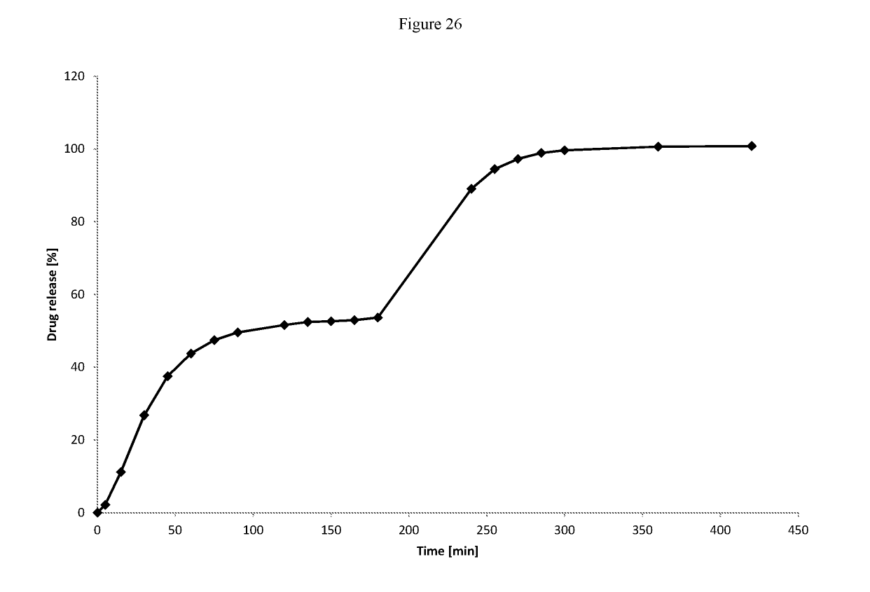

FIG. 26 shows the in vitro dissolution in non-ethanolic medium with a pH switch from pH 1 to pH 6.8 after 120 minutes.

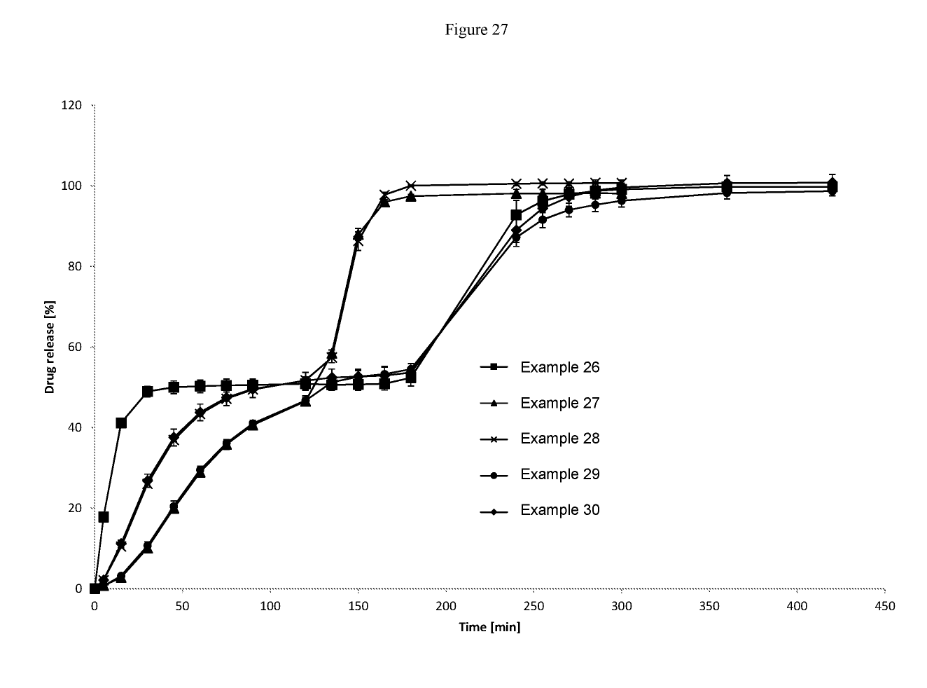

FIG. 27 is an overlay plot of Figures to 20 and 23 to 26.

### Example 31—Immediate Release Particles Comprising Non-Enteric Coating Providing Fast Release (Fast Release Particles)

In accordance with Example 23, pellets providing immediate release of amphetamine sulfate were manufactured by hot-melt extrusion. The thus obtained extruded pellets were subsequently provided with two coating layers, namely an inner layer based on Opadry® (FR Coating Layer 1) and an outer layer based on Eudragit® L30-D55 (FR Coating Layer 2), which slightly retards in vitro dissolution thereby providing fast release particles. Three different types of FR particles were coated with the same amount of Coating Layer 1 (7.5 wt.-%) and with different amounts of Coating Layer 2:
FR particles type 1: 3.2 wt.-%,
FR particles type 2: 6.54 wt.-%, and
FR particles type 3: 8.28 wt.-%.

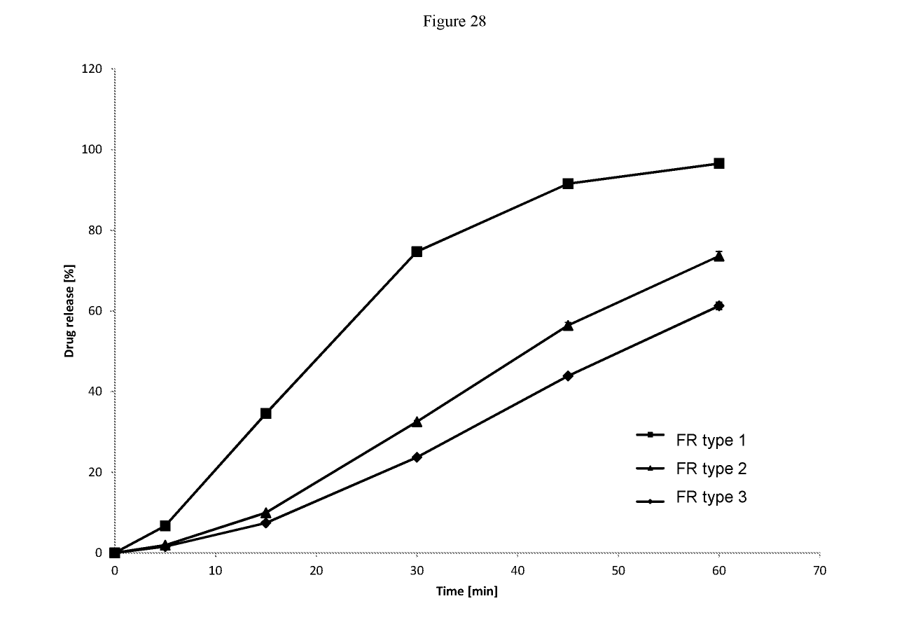

FIG. 28 shows an overlay of the in vitro dissolution profiles in non-ethanolic medium at pH 1.

### Example 32—In Vitro Release from DR Particles Vs. OR Particles in Different Release Media

Particles were coated with a non-enteric coating based on Opadry (applied at a content of 15 wt.-%), followed by an enteric coating comprising an inner layer based on sodium alginate (applied at a content of 30 wt.-%) and an outer layer of different compositions:
OR particles type 1: combination of 90 wt.-% Eudragit® FS(=first acrylate polymer) with 10 wt.-% Eudragit® L (=second acrylate polymer), both applied at a total content of 30 wt.-%;
OR particles type 2: combination of 95 wt.-% Eudragit® FS(=first acrylate polymer) with 5 wt.-% Eudragit® L (=second acrylate polymer), both applied at a total content of 30 wt.-%;
DR particles type 1: Eudragit® L, applied at a total content of 15 wt.-%;
DR particles type 2: Eudragit® L, applied at a total content of 22.5 wt.-%; and
DR particles type 3: Eudragit® L, applied at a total content of 30 wt.-%.

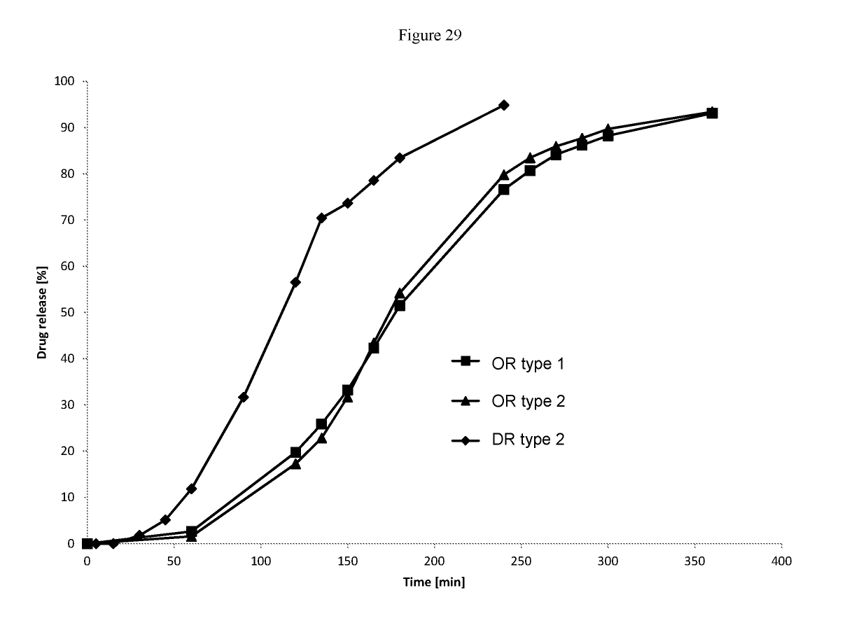

FIG. 29 shows the in vitro dissolution in ethanolic medium with a pH switch from pH 1 to pH 6.8 after 120 minutes.

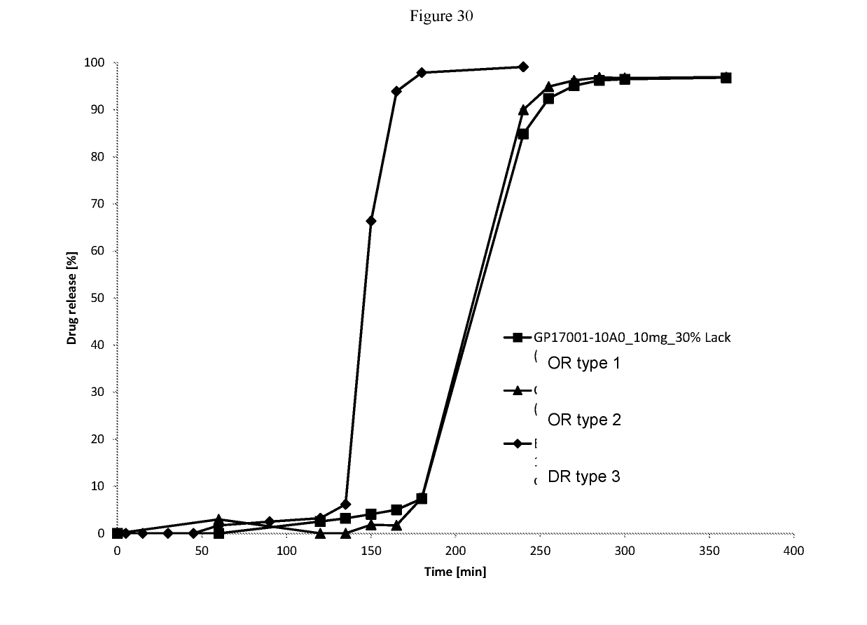

FIG. 30 shows the in vitro dissolution in ethanolic medium with a pH switch from pH 1 to pH 6.8 after 120 minutes.

### Example 33—Immediate Release Particles Coated with Non-Enteric Coating which does not Delay In Vitro Dissolution

In accordance with Example 1, pellets providing immediate release of amphetamine sulfate were manufactured by hot-melt extrusion. The thus obtained extruded pellets were coated with a non-functional (non-enteric) protection coating which does not delay in vitro dissolution to avoid sticking of pellets.

The pellets (multitude of immediate release particles) contained 10 mg amphetamine sulfate. The IR particles had the following composition:

| | per dosis | | amount |
|---|---|---|---|
| | [mg] | substance | [wt.-%] |
| core pellet | 10.00 | amphetamine sulfate | 13.88 |
| | 33.05 | polyethylene oxide 7 mio. | 45.88 |
| | 12.97 | polyethylene glycol 6000 | 18.01 |
| | 0.907 | alpha tocopherol/PEG 6000 powder | 1.26 |
| | 0.333 | citric acid | 0.46 |
| | 9.41 | starch 1500 | 13.06 |
| coating | 5 | Opadry ® II clear | 6.94 |
| lubrication | 0.36 | Talcum | 0.50 |
| | 72.03 | | 100.00 |

Opadry ® II clear: a non-enteric coating which does not delay in vitro dissolution.

The average individual total weight of a single particle was below 2.0 mg.

### Example 34—Controlled Release Particle Providing Extended Release

In accordance with Example 4, 10 mg PR particles (cut rods) of a total weight amounting to 175 mg were manufactured. The PR particles had the following composition:

| per dosis | | Amount |
|---|---|---|
| [mg] | Substance | [wt.-%] |
| 10.00 | amphetamine sulfate | 5.714 |
| 117.98 | polyethylene oxide 7 mio. | 67.416 |
| 17.50 | hypromellose type 2208 | 10.00 |
| 26.15 | polyethylene glycol 6000 | 14.94 |
| 2.50 | α-tocopherol/PEG 6000 powder | 1.43 |
| 0.875 | citric acid | 0.50 |
| 175 | | 100 |

The average individual total weight of a single coated particle was below 2.0 mg.

The breaking strength (resistance to crushing) of the particles was measured. In none of altogether ten measurements, the particles broke at a force of 1000 N.

### Example 35—Immediate Release Particles Comprising Non-Enteric Coating Providing Fast Release (Fast Release Particles)

In accordance with Example 23, pellets providing immediate release of amphetamine sulfate were manufactured by hot-melt extrusion.

The pellets (multitude of immediate release particles) contained 10 mg amphetamine sulfate. The IR particles had the following composition:

| | per dosis | | amount |
|---|---|---|---|
| | [mg] | substance | [wt.-%] |
| core | 10.00 | amphetamine sulfate | 15.00 |
| pellet | 33.05 | polyethylene oxide 7 mio. | 49.57 |
| | 12.97 | polyethylene glycol 6000 | 19.46 |
| | 0.907 | alpha tocopherol/PEG 6000 powder | 1.36 |
| | 0.333 | citric acid | 0.5 |
| | 9.41 | starch 1500 | 14.11 |
| | 66.67 | | 100.00 |

The thus obtained extruded pellets were subsequently provided with layers having the following compositions 35-1 to 35-4 (the various weight gains are also listed for different coated pellets):

| | 35-1 | | 35-2 | | 35-3 | | 35-4 | |
|---|---|---|---|---|---|---|---|---|
| | wt.-% | mg | wt.-% | mg | wt.-% | mg | wt.-% | mg |
| Eudragit L 30 D 55 | 40.79 | 3.12 | 40.79 | 3.12 | 40.79 | 4.81 | 40.79 | 6.01 |
| Triethylcitrate | 2.00 | 0.507 | 2.00 | 0.550 | 2.00 | 0.79 | 2.00 | 0.98 |
| Talcum micronized | 6.12 | 1.55 | 6.12 | 1.68 | 6.12 | 2.41 | 6.12 | 3.01 |
| Water purified | 51.09 | — | 51.09 | — | 51.09 | — | 51.09 | — |
| Sum | 100 | 71.86 | 100 | 72.27 | 100 | 74.67 | 100 | 76.67 |
| Talcum | — | — | 0.5 | 0.361 | — | — | — | — |
| Sum | 100 | 71.86 | 100 | 72.63 | 100 | 74.67¹ | 100 | 76.67² |

| | | | | |
|---|---|---|---|---|
| weight gain 1 | 7.79% | 8.4% | 8.4% | 10% |
| weight gain 2 | — | — | 10% | 15% |
| weight gain 3 | — | — | 12% | — |

¹based on 12% coating weight gain
²based on 15% coating weight gain

The average individual total weight of a single coated particle was below 2.0 mg.

The content of amphetamine (% assay), the content of impurities Imp A, Imp B and Imp C were determined for pellets 35-3 and pellets 35-4:

| | weight | | | | | |
|---|---|---|---|---|---|---|
| | gain | replicates | % assay | Imp A | Imp B | Imp C |
| 35-3 | 8.4% | 1 | 99.6 | 0.06 | 0.05 | 0.28 |
| | | 2 | 99.0 | 0.06 | 0.06 | 0.28 |
| | 10% | 1 | 98.3 | 0.06 | 0.05 | 0.36 |
| | | 2 | 98.3 | 0.06 | 0.06 | 0.36 |
| | 12% | 1 | 98.4 | 0.06 | 0.05 | 0.41 |
| | | 2 | 98.7 | 0.05 | 0.05 | 0.40 |
| 35-4 | 10% | 1 | 97.1 | <0.1 | <0.1 | 0.41 |
| | | 2 | 96.9 | | | |
| | 15% | 1 | 96.2 | | | 0.61 |
| | | 2 | 95.9 | | | |

The average individual total weight of a single coated particle was below 2.0 mg.

### Example 36—Controlled Release Particles Comprising Specific Enteric Coating Providing Delayed Release

In accordance with Example 3, 10 mg DR particles were manufactured comprising a functional, i.e. enteric coating. A hot melt extruded pellet core was subsequently provided with three coating layers, namely an inner layer based on Opadry® II pink (DR Coating Layer 1), an intermediate layer based on alginate (DR Coating Layer 2), and an outer layer based on Eudragit® L30-D55 (DR Coating Layer 3).

The DR coated pellets had the following composition:

| per dosis | | Amount |
|---|---|---|
| [mg] | Substance | [wt.-%] |
| 19.00 | amphetamine sulfate | 15.00 |
| 33.05 | polyethylene oxide 7 mio. | 49.57 |
| 12.97 | polyethylene glycol 6000 | 19.46 |
| 0.907 | α-tocopherol/PEG 6000 powder | 1.36 |
| 0.333 | Citric acid | 0.50 |
| 9.41 | Starch 1500 | 14.11 |
| 66.67 | | 100.00 |

DR Coating Layer 1, DR Coating Layer 2, and DR Coating Layer 3 had the following compositions:

| | | 36-1 | | 36-2 | | 36-3 | |
|---|---|---|---|---|---|---|---|
| | | wt.-% | mg | wt.-% | mg | wt.-% | mg |
| Coating Layer | Opadry II pink 85F24141 | 20.00 | 8.98 | 15.00 | 10.00 | 15.00 | 10.00 |
| 1 | Water purified | 80.00 | — | 85.00 | — | 85.00 | — |
| | Sum | 100 | 75.63 | 100 | 76.67 | 100 | 76.67 |
| | Weight gain | 13.4%⁴ | | 15%⁵ | | 15%⁵ | |
| Coating Layer | Sodium alginate | 4.50 | 22.33 | — | — | — | — |
| 2 | (Protanal CR 8133)² | | | | | | |
| | Sodium alginate | | | 5.25 | 22.48 | 5.25 | 22.48 |
| | (Protanal B LFR 5/60 | | | | | | |
| | NF)³ | | | | | | |
| | Talcum micronized | 2.25 | 11.17 | 2.63 | 11.26 | 2.63 | 11.26 |
| | Water purified | 93.25 | — | 92.12 | — | 92.12 | — |
| | Sum | 100 | 109.13 | 100 | 110.41 | 100 | 110.41 |
| | Weight gain | 44.2%⁴ | | 44%⁵ | | 44%⁵ | |
| Coating Layer | Eudragit FS30D | 10.78 | 4.59 | 36.71 | 29.88 | 36.71 | 29.88 |
| 3 | Eudragit L30 D55 | 1.20 | 41.33 | 4.08 | 3.26 | 4.08 | 3.26 |
| | Triethylcitrate | 2.04 | 2.34 | 2.00 | 5.43 | 2.00 | 5.43 |
| | Talcum | 5.99 | 6.89 | 6.12 | 16.63 | 6.12 | 16.63 |
| | Water purified | 80.00 | — | 51.09 | — | 51.09 | — |
| | Sum | 100 | 164.28 | 100 | 165.62¹ | 100 | 165.62¹ |

| | | | |
|---|---|---|---|
| Weight gain | 50.5%⁴ | 50%⁵ | 50%⁵ and 55%⁵ |

¹based on 50% coating weight gain
²high viscosity grade (10,000 mPa · s), difficult to process
³low viscosity grade (140 mPa · s), easier to process
⁴effective weight gain
⁵theoretical weight gain

The average individual total weight of a single coated particle was below 2.0 mg.

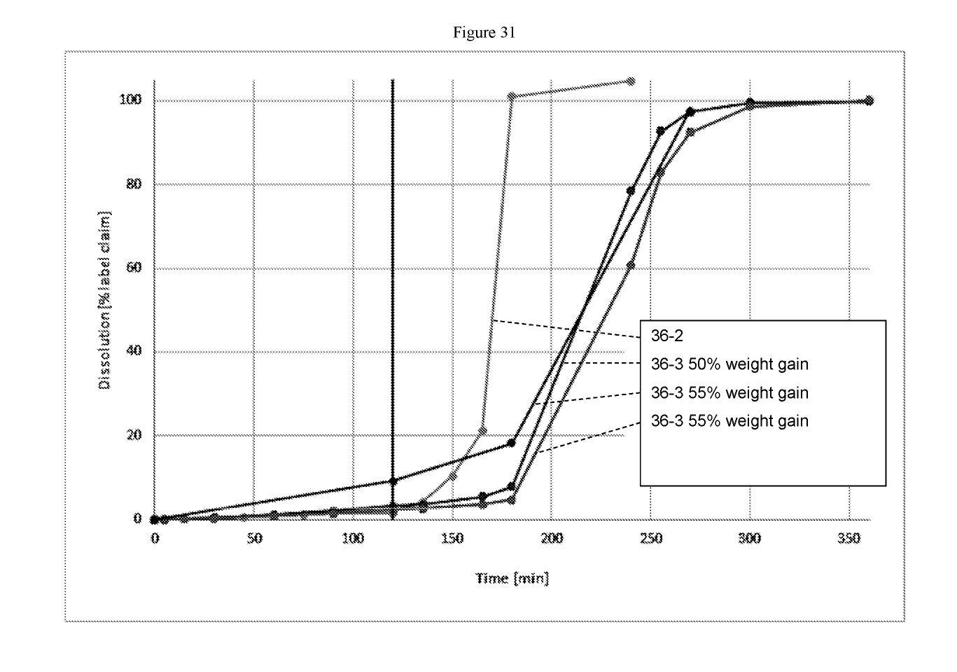

FIG. 31 shows the in vitro dissolution of the particles with a pH switch from pH 1 to pH 6.8 after 120 minutes in non-ethanolic medium.

1. A pharmaceutical dosage form for oral administration comprising a pharmacologically active compound;
wherein a portion of said pharmacologically active compound is contained in a multitude of immediate release particles providing immediate release of the pharmacologically active compound;
wherein another portion of said pharmacologically active compound is contained in at least one controlled release particle providing controlled release of the pharmacologically active compound; and
wherein the breaking strength of each of the immediate release particles and/or of the at least one controlled release particle is at least 300 N.

2. The pharmaceutical dosage form according to claim 1, wherein said another portion of said pharmacologically active compound is contained in a single controlled release particle.

3. The pharmaceutical dosage form according to claim 2, wherein said single controlled release particle has a total weight of at least 20 mg.

4.-7. (canceled)

8. The pharmaceutical dosage form according to claim 2, wherein said single controlled release particle comprises an acid, optionally citric acid.

9.-10. (canceled)

11. The pharmaceutical dosage form according to claim 1, wherein said another portion of said pharmacologically active compound is contained in a multitude of controlled release particles.

12. The pharmaceutical dosage form according to claim 11, wherein each of said controlled release particles is coated with an enteric coating.

13.-54. (canceled)

55. The pharmaceutical dosage form according to claim 11, wherein said controlled release particles comprise an acid, optionally citric acid.

56.-87. (canceled)

88. The pharmaceutical dosage form according to claim 1, wherein the pharmacologically active compound belongs to the group of psychoanaleptics [N06], or to the group of psychostimulants, agents used for ADHD, and nootropics [N06B], or to the group of centrally acting sympathomimetics [N06BA].

89.-90. (canceled)

91. The pharmaceutical dosage form according to claim 88, wherein the pharmacologically active compound is selected from the group consisting of amphetamine, dexamphetamine, metamphetamine, methylphenidate, pemoline, fencamfamin, modafinil, fenozolone, atomoxetine, fenetylline, dexmethylphenidate, lisdexamphetamine, armodafinil, and the physiologically acceptable salts of any of the foregoing.

92. The pharmaceutical dosage form according to claim 91, wherein the pharmacologically active compound is amphetamine sulfate.

93. The pharmaceutical dosage form according to claim 91, wherein the pharmacologically active compound is methylphenidate.

94.-95. (canceled)

96. The pharmaceutical dosage form according to claim 1, wherein said multitude of immediate release particles and/or said at least one controlled release particle comprises a polyalkylene oxide.

97.-99. (canceled)

100. The pharmaceutical dosage form according to claim 96, wherein the content of the polyalkylene oxide is at least 25 wt.-%, based on the total weight of said multitude of immediate release particles and/or based on the total weight of said at least one controlled release particle, respectively.

101. (canceled)

102. The pharmaceutical dosage form according to claim 1, wherein each of said immediate release particles and/or the at least one controlled release particle comprises a disintegrant.

103.-106. (canceled)

107. The pharmaceutical dosage form according to claim 1, which additionally comprises a gelling agent.

108.-109. (canceled)

110. The pharmaceutical dosage form according to claim 1 which is a capsule or a tablet.

111.-126. (canceled)

127. A method for treating attention deficit hyperactivity disorder (ADHD) in a patient in need thereof, said method comprising administering to said patient an effective amount therefor of at least one pharmaceutical dosage form according to claim 88.
